(19) United States
(12) Patent Application Publication
(10) Pub. No.: US 2007/0184115 A1
(43) Pub. Date: Aug. 9, 2007

(54) Modified release formulations of tramadol and uses thereof

(75) Inventors: Salim Mamajiwalla, Mississauga (CA); Steve Frisbee, Chantilly, VA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Biovail Laboratories International S.R.L., Mississauga (CA)

(21) Appl. No.: 11/640,494

(22) Filed: Dec. 18, 2006

Related U.S. Application Data
(60) Provisional application No. 60/754,631, filed on Dec. 30, 2005.
(60) Provisional application No. 60/754,634, filed on Dec. 30, 2005.
(60) Provisional application No. 60/754,637, filed on Dec. 30, 2005.

(51) Int. Cl. A61K 9/22; A61K 9/32; A61K 9/42

(52) U.S. Cl. 424/486; 424/468; 424/484; 424/490

(57) ABSTRACT

The present invention relates to specific types of controlled and modified release dosage forms containing tramadol or at least one pharmaceutically acceptable salt, enantiomer, or metabolite thereof that possess specific pharmacokinetic properties and which desirably are not subject to dose dumping, e.g., induced by food or alcohol. The invention also relates to methods of making and using these controlled and modified release dosage forms in therapeutic regimens wherein tramadol is therapeutically effective.

The present application claims priority from U.S. provisional application 60/754,631, 60/754,634, and 60/754,637, filed Dec. 30, 2005. The March 2003 Guidance for Industry Bioavailability and Bioequivalence Studies for Orally Administered Drug Products General Considerations, U.S. Department of Health and Human Services, Food and Drug Administration, Center for Drug Evaluation and Research (CDER) and the label published Sep. 8, 2005 enclosed therein are incorporated herein by reference in their entirety. The present invention provides novel solid modified release formulations containing tramadol or a pharmaceutically acceptable salt thereof and methods of use for prophylaxis and therapeutics wherein tramadol is effective.

### DEFINITIONS

In order to describe the present invention the following definitions are provided. Otherwise all terms are to be accorded their ordinary meaning as they would be construed by one skilled in the relevant art, i.e., drug formulation and therapy.

The term “dosage form” as used herein is defined to mean a solid oral pharmaceutical preparation or system in which doses of medicine or active drug are included. A dosage form will desirably comprise, for example, at least one modified release dosage form, at least one osmosis controlled-release dosage form, at least one erosion controlled-release dosage form, at least one dissolution controlled-release dosage form, at least one diffusion controlled-release dosage form, at least one controlled-release matrix core, at least one controlled-release matrix core coated with at least one release-slowing coat, at least one enteric coated dosage form, at least one dosage form surrounded by at least one release-slowing coat, at least one dosage form surrounded by at least one delayed-release coat, capsules, minitablets, caplets, uncoated microparticles, microparticles coated with at least one release-slowing coat, microparticles coated with at least one delayed-release coat or any combination thereof. Within the context of this application, the dosage forms described herein mean a dosage form as defined above comprising an effective amount of tramadol for treatment of moderately to moderately severe pain.

“Active moiety” as used herein is defined to mean the molecule or ion, excluding those appended portions of the molecule that cause the drug to be an ester, salt (including a salt with hydrogen or coordination bonds), of the molecule, responsible for the physiological or pharmacological action of the drug substance.

“Active drug” as used herein is defined to mean the molecule or ion, including those appended portions of the molecule that cause the drug to be an ester, salt (including a salt with hydrogen or coordination bonds), of the molecule.

“Tramadol” as used herein is defined to mean at least one form of tramadol chosen from tramadol base, the individually optically active enantiomers of tramadol, such as for example, (+)-tramadol or (−)-tramadol, racemic mixtures thereof, active metabolites, pharmaceutically acceptable salts thereof, such as for example, acid addition or base addition salts of tramadol. Acids commonly employed to form acid addition salts are inorganic acids, such as for example, hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutylate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutylate, citrate, lactate, g-hydroxybutylate, glycolate, tartrate, methanesulfonate, propanesulfonate, naphthalene-1-sulfonate, napththalene-2-sulfonate, mandelate and the like. Base addition salts include those derived from inorganic bases, such as for example, ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like.

“Pharmaceutically acceptable” is defined herein refers to compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio. Examples of pharmaceutically acceptable compounds, materials, compositions, and/or dosage forms can be found in pharmaceutical compendiums such as the United States Pharmacopia and future editions thereof or the Handbook of Pharmaceutical Excipients. 4^{th} Edition (2003). Ed. Rowe et al. Pharmaceutical Press and American Pharmaceutical Association and future editions thereof.

An amount of tramadol which provides a “therapeutic benefit”, is “pharmaceutically effective”, or is present in an “effective amount” is defined here in to mean the amount or quantity of tramadol, which is enough for the required or desired therapeutic response or the amount which is sufficient to elicit an appreciable biological response, when administered to a patient in need of administration of tramadol. With respect to the dosage forms described herein, the amount of tramadol, which provides a therapeutic benefit present in the dosage forms described herein is the amount sufficient for the treatment of moderately to moderately severe pain.

The term “controlled-release” as used herein is defined to mean a substantially gradual rate of release of the tramadol in the first once daily controlled-release dosage form or the at least one means for controllably releasing the tramadol in a substantially controlled manner per unit time in-vivo. The rate of release of the tramadol is controlled by features of the dosage form and/or in combination with physiologic or environmental conditions rather than by physiologic or environmental conditions alone. The first once daily controlled-release dosage form or the at least one means for controllably releasing the tramadol of the invention will desirably be contrasted to immediate-release dosage forms, which typically produce large maximum/minimum plasma drug concentrations (Cₘₐₓ/Cₘᵢₙ) due to rapid absorption of the drug into the body i.e., in-vivo, relative to the drug's therapeutic index i.e., the ratio of the maximum drug concentration needed to produce and maintain a desirable pharmacological response. In immediate-release dosage forms, the drug content is released into the gastrointestinal tract within a short period of time, and plasma drug levels peak shortly after dosing. The design of immediate-release dosage forms is generally based on getting the fastest possible rate of drug release, and therefore absorbed, often at the risk of creating undesirable dose related side effects. The controlled-release dosage forms of the invention, on the other hand, improve the therapeutic value of the active drug by reducing the ratio of the maximum/minimum plasma drug concentration (Cₘₐₓ/Cₘᵢₙ) while maintaining drug plasma levels within the therapeutic window. The first once daily controlled-release dosage form or the at least one means for controllably releasing the tramadol of the invention attempt to deliver therapeutically effective amount of tramadol at constant effective levels to provide therapeutic benefit over a about a 24-hour period. The first once daily controlled-release dosage form or the at least one means for controllably releasing the tramadol of the invention, therefore, avoid large peak-to-trough fluctuations normally seen with immediate-release dosage forms and provide a substantially flat serum concentration curve throughout the therapeutic period.

The term “core” as used herein is defined to mean a solid vehicle in which tramadol is uniformly or non-uniformly dispersed. The core will desirably be formed by methods and materials well known in the art, such as for example by compressing, fusing, or extruding the tramadol together with at least one pharmaceutically acceptable excipient. The core will desirably be manufactured into a homogenous or non-homogenous unitary core or a plurality of multiparticulates compressed into a core. The core(s) will desirably be coated with at least one release-slowing coat, semi-permeable coat or membrane, non-functional coat, or any combination of coats thereof.

The term “controlled-release matrix core” as used herein is defined to mean a core in which tramadol is dispersed within a matrix which controls or delays the release of the tramadol over about a 24-hour period so as to allow a composition comprising the controlled-release matrix core to be administered as a once-a-day composition. The release rate of the tramadol from the controlled-release matrix core will desirably be modified by the porosity of the matrix, i.e. its pore structure. The addition of pore-forming hydrophilic salts, solutes, wicking agents, or wetting aids will desirably influence the release rate, as will desirably the manipulation of processing parameters. For example, the compression force used in the manufacture of the controlled-release matrix core will desirably alter the porosity of the matrix core and hence the rate of release of the tramadol. It will be understood by one of ordinary skill in the art of drug delivery that a more rigid matrix will be less porous and hence release tramadol more slowly compared to a less rigid controlled-release matrix core. The controlled-release matrix core will desirably comprise insoluble or inert matrix dosage forms, swellable matrix dosage forms, swellable and erodable matrix dosage form, hydrophobic matrix dosage forms, hydrophilic matrix dosage forms, erodable matrix dosage forms, reservoir dosage forms, or any combination thereof. The controlled-release matrix core of the invention refer to the at least one substantially insoluble matrix, at least substantially one swellable or swellable and erodable matrix, at least one substantially hydrophobic matrix, at least one substantially hydrophilic matrix, at least one substantially erodable matrix, or a combination thereof in which the rate of release is substantially slower than that of uncoated immediate-release dosage forms. Controlled-release matrix cores will desirably be coated with at least one “release-slowing coat” to further slow the release of the tramadol from the controlled-release matrix core. Such coated controlled-release matrix cores will desirably exhibit “modified-release”, controlled-release”, sustained-release”, “extended-release”, “prolonged-release”, “bi-phasic release”, “delayed-release” or combinations thereof of the tramadol. Controlled-release matrix cores will desirably also be coated with a non-functional soluble coat. The controlled-release matrix cores as defined herein do not encompass controlled-release matrix cores wherein the matrix material that predominantly regulates drug release comprises a cross-linked high amylose starch such as the matrix described in U.S. Pat. No. 6,607,748.

The term “normal release matrix core” and “immediate-release matrix core” as used herein are defined to mean a core in which tramadol is dispersed within a matrix, which matrix will desirably be either substantially insoluble, substantially soluble, substantially swellable or substantially swellable and erodable, or combinations thereof. The normal release matrix does not comprise starch derivatives and water-soluble materials such as, for example, gelatin, polyvinylpyrrolidone, polyvinyl alcohol, hydroxypropylmethylcellulose, hydroxypropylcellulose, xanthan gum, carbomers, and caragheen. Normal release matrix cores will desirably be manufactured such that the release of the tramadol substantially mimics the release rate of an uncoated non-matrix or immediate-release dosage form comprising the tramadol. The release rate from normal release matrix core will desirably be substantially slowed down, controlled, delayed or modified in conjunction with a “release-slowing coat” or a “delayed-release coat”. In the absence of such coats the release of tramadol from a normal release matrix core is substantially immediate.

“Controlled-release dosage forms” or dosage forms which exhibit a “controlled-release” of tramadol as used herein is defined to mean dosage forms administered once daily that release drug at a relatively constant rate and provide plasma concentrations of the active drug that remain substantially invariant with time within the therapeutic range of the active drug over about a 24-hour period. The first once daily controlled-release dosage forms of the invention include, for example, at least one osmotic dosage form, at least one swellable dosage form, at least one swellable and erodable dosage form, at least one erodable dosage form, at least one insoluble dosage form, at least one hydrophobic dosage form, at least one hydrophilic dosage form, at least one lipid or wax dosage form; at least one release-slowing coat, at least one insoluble coat, at least one swellable coat, at least one erodable coat, at least one swellable and erodable coat, at least one extended-release dosage form, at least one delayed-release dosage form, at least one modified-release dosage form, at least one sustained-release dosage form, at least one prolonged-release dosage form, at least one bi-phasic release dosage form, at least one normal release matrix core coated with at least one release-slowing coat, at least one normal release matrix core coated with at least one aqueous insoluble coat, at least one normal release matrix core coated with at least one swellable coat, at least one normal release matrix core coated with at least one swellable and erodable coat, at least one normal release matrix core coated with at least one erodable coat, or any combination thereof.

“Sustained-release dosage forms” or dosage forms which exhibit a “sustained-release” of the tramadol as used herein is defined to mean dosage forms administered once daily that provide a release of the tramadol sufficient to provide a therapeutic dose after administration, and then a gradual release over an extended period of time such that the sustained-release dosage form provides therapeutic benefit over a 24-hour period. Sustained-release dosage forms will desirably be coated with a delayed-release coat to delay release followed by a sustained-release of the tramadol.

“Extended-release dosage forms” or dosage forms which exhibit an “extended release” of tramadol as used herein is defined to mean dosage forms administered once daily that release drug slowly, so that plasma concentrations of the tramadol are maintained at a therapeutic level for an extended period of time such that the sustained-release dosage form provides therapeutic benefit over a 24-hour period. Extended-release dosage forms will desirably be coated with a delayed-release coat to delay release followed by a extended-release of the tramadol.

“Prolonged-release dosage forms” or dosage forms which exhibit a “prolonged release” of tramadol as used herein is defined to mean dosage forms administered once daily which provide for absorption of the tramadol over a longer period of time than from an immediate-release dosage form and which provide therapeutic benefit over a 24-hour period. Prolonged-release dosage forms will desirably be coated with a delayed-release coat to delay release followed by a prolonged-release of the tramadol.

“Delayed-release dosage forms” or dosage forms which exhibit a “delayed-release” of tramadol as used herein is defined to mean dosage forms administered once daily that do not substantially release drug immediately following administration but at a later time. Delayed-release dosage forms provide a time delay prior to the commencement of drug-absorption. Such dosage forms will desirably be coated with a delayed-release coat. This time delay is referred to as “lag time” and should not be confused with “onset time” which represents latency, that is, the time required for the drug to reach minimum effective concentration.

“Enhanced absorption dosage forms” or dosage forms which exhibit an “enhanced absorption” of the tramadol as used herein is defined to mean dosage forms that when exposed to like conditions, will show higher release and/or higher absorption of the tramadol as compared to other dosage forms with the same or higher amount of tramadol. The same therapeutic effect will desirably be achieved with less tramadol in the enhanced absorption dosage form as compared to other dosage forms.

“Modified-release dosage forms” or dosage forms which exhibit a “modified-release” of tramadol as used herein is defined to mean dosage forms whose drug release characteristics of time course and/or location are designed to accomplish therapeutic or convenience objectives not offered by an immediate-release dosage forms. Modified-release dosage forms or dosage forms are typically designed to provide a quick increase in the plasma concentration of the tramadol which remains substantially constant within the therapeutic range of tramadol for at least a 24-hour period. Alternatively, modified-release dosage forms will desirably be designed to provide a quick increase in the plasma concentration of tramadol, which although may not remain constant, declines at rate such that the plasma concentration remains within the therapeutic range for at least a 24-hour period. It will be apparent to the one of ordinary skill in the drug delivery arts that the above description of modified-release dosage forms encompasses “sustained-release”, controlled-release”, “extended-release” and “prolonged-release”, and “enhanced absorption” dosage forms.

The term “osmotic dosage form”, “osmotic delivery device”, “controlled-release osmotic dosage form” or “osmosis-controlled extended-release systems” as used herein is defined to mean dosage forms which forcibly dispense tramadol all or in part by pressure created by osmosis or diffusion of fluid into a core which forces tramadol to be dispensed from the osmotic dosage form. The term “osmotic dosage form”, “osmotic delivery device” or “controlled-release osmotic dosage form” also encompasses such forms that will desirably be coated with at least one “release-slowing coat.

The terms “osmagent”, “osmotically effective solute”, “osmotic enhancer” “osmotically effective compounds”, “osmotic solutes”, or “osmotic fluid imbibing agents” are all used interchangeably herein and are defined to mean any material that functions to increase the osmotic pressure of the core, thus, increasing the hydrostatic pressure inside the osmotic dosage form. The osmagent will desirably be either soluble or swellable and be totally or partially solubilized. Osmagents will desirably comprise tramadol.

The term “osmopolymer” as used herein is defined to mean any polymer that will desirably interact with, and consequently swell and retain water and/or an aqueous biological fluid and thereby increase the osmotic pressure of the core. The osmopolymer will desirably be slightly cross-linked or uncross-linked.

The term “osmotic subcoat” as used herein is defined to mean a coat that comprises at least one osmagent and at least one “osmotic deposition vehicle” in amounts sufficient to achieve an osmotic pressure gradient across one or more release-slowing or delayed-release coats for the transport of aqueous fluid (e.g., water, dissolution media, gastric, or intestinal fluid) from the external environment of use into the rate controlled-release dosage form, and the transport of tramadol solution from the core into the external environment of use. When applied rate controlled-release dosage forms alone or in combination with other coats, the osmotic subcoat will desirably modify the rate and/or extent of release of the tramadol from the core of the rate controlled-release dosage forms. For example, the osmotic subcoat will desirably provide increased release and/or substantially full release of the tramadol from the core. The osmotic subcoat surrounds the core of the rate controlled-release dosage form of the present invention, and will desirably in turn be surrounded by at least release-slowing or delayed-release coat. The osmotic subcoat will desirably optionally comprise additional materials that will desirably alter the functionality of the osmotic subcoat. The term “increased release” as used herein when referring to a rate controlled-release dosage form of the present invention, means that the rate and/or extent of drug release into the dissolution medium by a composition of the present invention comprising an osmotic subcoat, is greater than the rate and/or extent of drug release of an otherwise similar composition that does not comprise an osmotic subcoat, under similar conditions and similar dissolution media. “Substantially full release” in reference to rate controlled-release dosage forms of the invention comprising an osmotic subcoat refers to the extent of drug release into the dissolution medium whereby not less than about 90% of the total amount of tramadol is released during the dissolution period.

“Osmotic deposition vehicle” as used herein is defined to mean a carrier for the osmagent and will desirably be any substantially hydrophilic material.

A “release-slowing coat” as used herein is defined to mean a coat which can, for example, comprise at least one pH independent polymer; pH dependent polymer (such as for example enteric or reverse enteric types); soluble material, such as for example, a soluble polymer; insoluble material (aqueous insoluble coat), such as for example, an insoluble polymer; swellable material, such as for example, a swellable polymer; swellable and erodable material, such as for example, a swellable and erodable polymer; hydrophobic material, or combinations thereof which when applied onto an uncoated normal release matrix core or controlled-release matrix core will desirably slow, modify, further slow, or further modify the rate of release of tramadol. The at least one release-slowing coat will desirably be designed such that when the at least one coat is applied to a normal release matrix core or controlled-release matrix core, the dosage form in conjunction with the at least one release-slowing coat will desirably exhibit the release of tramadol, such as for example, as a “modified-release”, “controlled-release”, “sustained-release”, “extended-release”, “prolonged-release” or combinations thereof. The “release-slowing coat” will desirably optionally comprise additional materials that may alter the functionality of the release-slowing coat.

A “delayed-release coat” as used herein is defined to mean a functional coat which will desirably for example comprise at least one pH dependent polymer, such as for example, enteric or reverse enteric types, but can, in addition comprise at least one pH independent polymer; soluble material, such as for example a soluble polymer; insoluble material, such as for example an insoluble polymer; swellable material, such as for example, a swellable polymer; lipids; waxy materials; hydrophobic materials; hydrophilic materials; or combinations thereof. The delayed-release coat when applied onto a pharmaceutical composition does not allow appreciable drug release immediately following administration but at a later time. Delayed-release coats provide a time delay prior to the commencement of drug release, which delay is different form “lag time” as defined else where herein. For example, a delayed-release coat will desirably be applied onto a controlled-release matrix core such that after administration, the coat, either by dissolving slowly or disruption under certain pH conditions, allows release from the controlled-release matrix core to begin not in the stomach but in some predetermined region of the small intestine or even further down the intestinal tract, such as for example, in the colon. Coats comprising enteric materials, such as for example, enteric polymers, will desirably fall under the definition of a delayed-release coat. A delayed-release coat will desirably be applied to modified-release dosage forms so as to delay the release of the tramadol followed by a modified release of the tramadol.

An “immediate release” coat, as used herein, is defined to mean a coat, which has substantially or appreciably no influence on the rate of release of tramadol from the dosage form in-vitro or in-vivo. The excipients comprising the immediate release coat have no substantial controlled-release, swelling, erosion, dissolution, or erosion and swelling properties, which means that the composition of the coat has no substantial influence on the rate of release of the tramadol.

“Enteric polymers” as used herein is defined to mean polymeric substances which are substantially insoluble or stable under acidic conditions exhibiting a pH of less than about 5 and which are substantially soluble or decompose under conditions exhibiting a pH of about 5 or more. Examples of such enteric polymers include carboxymethylethylcellulose, cellulose acetate phthalate, cellulose acetate succinate, methylcellulose phthalate, hydroxymethylethylcellulose phthalate, hydroxypropylmethylcellulose phthalate, hydroxypropylmethylcellulose acetate succinate, polyvinyl alcohol phthalate, polyvinyl butylate phthalate, polyvinyl acetal phthalate, a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate and a copolymer of methacrylic acid/methyl methacrylate. Enteric polymers will desirably be used individually or in combination with other hydrophobic or hydrophilic polymers in a controlled-release or normal release matrix core and/or in a release-slowing coat and/and/or delayed-release coat. Enteric polymers will desirably be combined with other pharmaceutically acceptable excipients to either facilitate processing of a coat comprising the enteric polymer or to alter the functionality of the coat.

A “non-functional soluble coat” as used herein is defined to mean a coating that does not substantially affect the rate of release in-vitro or in-vivo, but will desirably enhance the chemical, biological, physical stability characteristics, or the physical appearance of the controlled-release dosage form.

The “second orally administrable dosage form” refers to New Drug Application No. 21-692 and its corresponding publicly available FDA label. A description and method of making the second orally administrable dosage form is described in International Patent Application Number PCT/US03/04866 published on Sep. 4, 2003 as WO 03/072025, of which examples 1 through 11 therein are incorporated herein by reference in their entirety.

The term “multiparticulate” or “microparticle” as used herein is defined to mean a plurality of drug-containing units, such as for example microspheres, spherical particles, microcapsules, particles, microparticles, granules, spheroids, beads, pellets, or spherules.

“Bioequivalence” is defined as there being about a 90% or greater probability that the bioavailability (AUC) of tramadol as determined by standard methods is about 80 to about 125% of the second orally administrable dosage form comprising the same dose of tramadol and that there is a about 90% or greater probability that the maximum blood plasma concentration (Cₘₐₓ) of tramadol as measured by standard methods is about 80 to about 125% of the second orally administrable dosage form. For example, the reader is referred to the final version of the guidance approved by the US Food and Drug Administration at the time of filing of this patent application i.e., the March 2003 Guidance for Industry Bioavailability and Bioequivalence Studies for Orally Administered Drug Products General Considerations, U.S. Department of Health and Human Services, Food and Drug Administration, Center for Drug Evaluation and Research (CDER), for a detailed discussion on bioequivalence.

Dissolution tests in-vitro measure the rate and extent of dissolution of tramadol in an aqueous medium. In embodiments where the “in-vitro release rate(s)” or “dissolution rate(s)” of tramadol from the controlled-release dosage forms of the invention are determined, the in-vitro release rate or dissolution rate is, for example, measured using a USP Type I, II, or III apparatus in 900 ml 0.1N HCl, water, 0.1N HCl+0.1% Cetrimide, USP Buffer pH 1.5, Acetate Buffer pH 4.5, Phosphate Buffer pH 6.5, or Phosphate Buffer pH7.4 at 75 rpm at 37°±0.5° C. The tramadol released into the dissolution medium is, for example, assayed in a 10 ml UV cell at 271 nm.

The term “dose dumping” as used herein includes “alcohol induced dose dumping” and “food induced dose dumping” and is defined to mean the unintended fluctuation of drug release, e.g., the rapid drug release of drug in a short period of time of the entire amount or a significant fraction of the drug contained in a controlled-release or modified-release dosage form in a fixed time relative to the release of drug that occurs when the same controlled or modified release dosage form is not subject to the conditions which induce “dose dumping”, e.g., alcohol or food. This may be evaluated in vitro or in vivo. For example controlled or modified release dosage forms subject to: “dose dumping” when evaluated under in vitro dissolution conditions which induce the “dose dumping” such as ethanol, e.g., from about 5 to about 40% ethanol, may release the drug differently over the initial about 2 hours after administration, or the initial about 4 hours after administration, or the initial about 6 hours after administration or over the initial about 24 hours after administration in comparison to when in vitro dissolution of the same controlled or modified release dosage form is effected in the absence of or at lower alcohol concentration, e.g., lower ethanol concentration.

The term “alcohol induced dose dumping” in particular as used herein is defined to mean the unintended increase in drug release over a period of at least about 2 hours, e.g., from about 0-2 hours, or about 0-4 hours, or about 0-6 hours, or over the initial about 24 hours after administration which may be determined in vitro or in vivo. This can be determined in vitro e.g., by effecting dissolution of the controlled or modified release dosage form in about 900 ml of Alcohol USP comprising dissolution media e.g., using USP Apparatus I at 75 rpm at 37° C. over a time period of about 2 hours, or about 4 hours, or about 6 hours or longer, e.g., up to about 24 hours as compared to the release of drug that occurs under in vitro dissolution conditions and in an in vitro dissolution medium which does not include an alcohol, e.g., ethanol, or which contains a reduced amount of alcohol, e.g., ethanol. The release of drug from dosage forms that are subject to “alcohol induced dose dumping” according to the invention will fluctuate, e.g., typically be increased, over a set time period, generally over about a 2 hour period, or over about a 4 hour time period, or over about a 6 hour time period by at least about 10%, or by at least about 10-30%, or by at least about 30-50%, or greater than about 50-70% higher when dissolution is effected in an “alcohol USP Comprising Dissolution Medium” relative to the amount of drug that is released from the same controlled or modified release dosage form when this dosage form is in a dissolution medium lacking alcohol, e.g., ethanol, or containing a reduced amount of alcohol, e.g., ethanol, for the same amount of time, e.g., about 2 hours, about 4 hours, or over about a 6 hour time period. In some embodiments controlled or modified release dosage forms which are subject to “alcohol induced dose dumping” may release the drug in vivo during the initial hours after administration, e.g., the initial about 2 hours, or the initial about 4 hours, or the initial about 6 hours after administration comparably to an immediate release dosage form, e.g., a substantial portion is released from the controlled or modified release dosage form in the presence of alcohol over the first about 2 hours, e.g., an amount greater than about 50 mg is released over the first about 2 hours after administration and the release of drug is substantially less in the absence of or at reduced in vivo alcohol concentrations in the blood.

“Alcohol USP comprising dissolution media” is defined to mean any dissolution media comprising about 5% to about 40% (v/v) of Alcohol USP. An exemplary dissolution medium contains about 40% alcohol, e.g., ethanol.

As used herein, the term “plasticizer” is defined to mean any material capable of plasticizing or softening a polymer or binder used in invention. Once a coat or core has been manufactured, certain plasticizers will desirably function to increase the hydrophilicity or hydrophobicity of the coat(s) and/or the core of the first once daily controlled-release dosage forms in the environment of use. During manufacture of the coat, the plasticizer should be able to lower the melting temperature or glass transition temperature (softening point temperature) of the polymer or binder. Plasticizers, such as low molecular weight PEG, generally broaden the average molecular weight of a polymer system in which they are included, and lower its glass transition temperature or softening point. Plasticizers also generally reduce the viscosity of a polymer.

The terms “flux enhancing agent” or “channeling agent” as used herein is used to define any material(s), which is soluble in an aqueous medium and will desirably leach from a controlled-release dosage form or a means for controllably releasing the tramadol. Tramadol itself will desirably be a flux enhancing or channeling agent. The flux enhancing or channeling agent will desirably also function as a means for the exit of tramadol.

The term “gel modifier” as used herein is defined to mean any material, which when incorporated into the first once daily controlled-release dosage form or the at least one means for controllably releasing the tramadol will desirably modify the diffusional characteristics of a gel layer formed upon hydration of a swellable or swellable and erodable dosage form or at least one means for controllably releasing the tramadol wherein the at least one means comprises a swellable or swellable and erodable matrix core. The gel modifier often enhances drug diffusion and hence release of the tramadol.

Unless otherwise indicated, all numbers expressing quantities of ingredients, properties such as molecular weight, reaction conditions, dissolution rates, pharmacokinetic parameters, and so forth, either as percentages or in absolute amounts, used in the specification and claims are modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Other terms are defined as they appear in the following description and should be construed in context with which they appear.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered to a patient in need of such administration under fed or fasted conditions will desirably be bioequivalent according to FDA guidelines to a second orally administrable dosage form comprising the same dose of tramadol also suitable for once daily administration in the fed or fasted state.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, has reduced potential for alcohol induced dose dumping and will desirably be bioequivalent in the fed or fasted state according to FDA guidelines to a second orally administrable dosage form comprising the same dose of tramadol also suitable for once daily administration in the fed or fasted state.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered to a patient in need of such administration will desirably exhibit following single-dose administration: (i) a Cₘₐₓ of from about 75 to about 338 ng/ml of the tramadol, (ii) an AUC_{0-∞} of from about 2725 to about 7581 ng·hr/ml of tramadol under fed conditions, and will desirably be bioequivalent according to FDA guidelines to a second orally administrable dosage form comprising the same dose of tramadol also suitable for once daily administration in the fed state.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered to a patient in need of such administration will desirably exhibit following single-dose administration: (i) a Cₘₐₓ of from about 75 to about 338 ng/ml of the tramadol, (ii) an AUC_{0-∞} of from about 2725 to about 7581 ng·hr/ml of tramadol under fed conditions, has reduced potential for alcohol induced dose dumping and will desirably be bioequivalent according to FDA guidelines to a second orally administrable dosage form comprising the same dose of tramadol also suitable for once daily administration in the fed state.

In at least one embodiment of the invention, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after 2 about hours from about 0 to about 22% by weight of tramadol is released, after about 4 hours from about 5 to about 30% by weight of tramadol is released, after 6 hours, from about 15 to about 38% by weight of tramadol is released, and after 8 about hours, more than about 40% by weight of tramadol is released, and when said first once daily controlled-release dosage form is administered to a patient in need of such administration will desirably exhibit following single-dose administration a Cₘₐₓ of, for example about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270 about, 275, about 280, about 285, about 290, about 295, about 300, about 305, about 310, about 315, about 320, about 325, about 330, about 335 or about 338 ng/ml of the tramadol in the fed state and will desirably be bioequivalent according to FDA guidelines to a second orally administrable dosage form comprising the same dose of tramadol also suitable for once daily administration in the fed state.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released 1, after about 6 hours, from about 15 to about 38% by weight of tramadol is released, and after about 8 hours, more than about 40% by weight of tramadol is released, and when said first once daily controlled-release dosage form is administered to a patient in need of such administration will desirably exhibit following single-dose administration an AUC_{0-∞} of, for example, about 2725, about 2750, about 2900, about 3050, about 3200, about 3350 about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions, and will desirably be bioequivalent according to FDA guidelines to a second orally administrable dosage form comprising the same dose of tramadol also suitable for once daily administration in the fed state.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to about 22% by weight of tramadol is released, after 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration: (i) a Cₘₐₓ of from about 180 to about 333 ng/ml of tramadol, (ii) an AUC_{0-∞} of from about 3740 to about 7600 ng·hr/ml of tramadol under fasting conditions, and will desirably be bioequivalent according to FDA guidelines to a second orally administrable dosage form comprising the same dose of tramadol also suitable for once daily administration in the fasted state.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to 2 about 2% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration: (i) a Cₘₐₓ of from about 180 to about 333 ng/ml of tramadol, (ii) an AUC_{0-∞} of from about 3740 to about 7600 ng·hr/ml of tramadol under fasting conditions, has reduced potential for alcohol induced dose dumping and will desirably be bioequivalent according to FDA guidelines to a second orally administrable dosage form comprising the same dose of tramadol also suitable for once daily administration in the fasted state.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to about 22% by weight of tramadol is released, after about 4 hours from 5 about to about 30% by weight of tramadol is released, after 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration a Cₘₐₓ of, for example, about 180, about 190, about 200, about 210, about 220, about 230, about 240, about 250, about 260, about 270, about 280, about 290, about 300, about 310, about 320, about 330, or about 333 ng/ml of tramadol in the fasted state and will desirably be bioequivalent according to FDA guidelines to a second orally administrable dosage form comprising the same dose of tramadol also suitable for once daily administration in the fasted state.

In at least one embodiment, alternative once daily controlled-release dosage forms of the invention comprising tramadol will desirably comprise a first once daily controlled-release dosage form comprising at least one means for controllably releasing tramadol such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550 about, 4600, about 4650, about 4700, about 4750, about 4800, about 4850, about 4900, about 4950, about 5000, about 5050, about 5100, about 5150, about 5200, about 5250, about 5300, about 5350, about 5400, about 5450, 5500, 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600, ng·hr/ml of tramadol under fasting conditions and will desirably be bioequivalent according to FDA guidelines to a second orally administrable dosage form comprising the same dose of tramadol also suitable for once daily administration in the fasted state.

In at least one embodiment of the invention, the in-vitro dissolution of the first once daily controlled-release dosage form comprising the at least one means for controllably releasing the tramadol is measured using a USP Type I, II, or III apparatus in dissolution medium chosen from 900 ml 0.1N HCl, water, 0.1N HCl+0.1% Cetrimide, USP Buffer pH 1.5, Acetate Buffer pH 4.5, Phosphate Buffer pH 6.5, or Phosphate Buffer pH7.4 at 75 rpm at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising the at least one means for controllably releasing the tramadol is measured using a USP Type I, II, or III apparatus in 900 ml 0.1N HCl 75 rpm at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising the at least one means for controllably releasing the tramadol is measured using a USP Type I, II, or III apparatus in water at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising the at least one means for controllably releasing the tramadol is measured using a USP Type I, II, or III apparatus in 0.1N HCl+0.1% Cetrimide at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising the at least one means for controllably releasing the tramadol is measured using a USP Type I, II, or III apparatus in USP Buffer pH 1.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising the at least one means for controllably releasing the tramadol is measured using a USP Type I, II, or III apparatus in Acetate buffer pH 4.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising the at least one means for controllably releasing the tramadol is measured using a USP Type I, II, or III apparatus in Phosphate Buffer pH 6.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising the at least one means for controllably releasing the tramadol is measured using a USP Type I, II, or III apparatus in Phosphate Buffer pH 7.4 at 37°±0.5° C. and the tramadol released into the dissolution medium is assayed in a 10 ml UV cell at 271 nm.

In at least one embodiment of the invention, the first once daily controlled-release dosage form comprising at least one means for controllably releasing tramadol will desirably exhibit in the fed state a Tₘₐₓ of tramadol from about 4 to about 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr following single-dose administration and will desirably be bioequivalent according to FDA guidelines to the second orally administrable dosage form comprising the same dose of tramadol also suitable for once daily administration.

In at least one embodiment of the invention, the first once daily controlled-release dosage form comprising at least one means for controllably releasing tramadol will desirably further exhibit at steady state the following pharmacokinetic parameters in-vivo under fasting conditions: (i) an AUC₀₋₂₄ from about 1635 to about 21000, for example, from about 1635 to about 3920, about 3610 to about 9120, or about 9455 to about 20965 ng·h/ml, and (ii) a Cₘₐₓ from about 117 to about 1230, for example, about 117 to about 245, about 230 to about 590, or about 590 to about 1230 ng/ml and will desirably be bioequivalent according to FDA guidelines to the second orally administrable dosage form comprising the same dose of tramadol also suitable for once daily administration.

In at least one embodiment of the invention, the first once daily controlled-release dosage form comprising at least one means for controllably releasing tramadol will desirably exhibit under fasting conditions a Tₘₐₓ of, for example, about 9, about 10, about 11, about 12, about 13, or about 14 hours at steady state and will desirably be bioequivalent according to FDA guidelines to the second orally administrable dosage form comprising the same dose of tramadol also suitable for once daily administration.

In at least one embodiment of the invention, the first once daily controlled-release dosage form comprising at least one means for controllably releasing tramadol will desirably exhibit under fasting conditions a degree of fluctuation (%) of, for example, about 43 to about 141, about 43 to about 120, about 58 to about 132, or about 57 to about 141 at steady state and will desirably be bioequivalent according to FDA guidelines to the second orally administrable dosage form comprising the same dose of tramadol also suitable for once daily administration.

In at least one embodiment of the invention, the first once daily controlled-release dosage form comprising at least one means for controllably releasing tramadol will desirably exhibit under fasting conditions a Cₘᵢₙ of, for example, from about 31 to about 652, about 31 to about 117, about 96 to about 241, or about 226 to about 652 ng/ml at steady state and will desirably be bioequivalent according to FDA guidelines to the second orally administrable dosage form comprising the same dose of tramadol also suitable for once daily administration.

In at least one embodiment of the invention, the first once daily controlled-release dosage form comprising the at least one means for controllably releasing tramadol will desirably exhibit under fasting conditions a Tₘₐₓ of, for example, about 9, about 10, about 11, about 12, about 13, or about 14 hours, a degree of fluctuation (%) of, for example, about 43 to about 141, about 43 to about 120, about 58 to about 132, or about 57 to about 141, and a Cₘₐₓ of, for example, from about 31 to about 652, about 31 to about 117, about 96 to about 241, or about 226 to about 652 ng/ml at steady state and will desirably be bioequivalent according to FDA guidelines to the second orally administrable dosage form comprising the same dose of tramadol also suitable for once daily administration.

In at least one embodiment of the invention, the first once daily controlled-release dosage form comprising at least one means for controllably releasing tramadol will desirably exhibit following single-dose administration a Tₘₐₓ of tramadol from about 10 to about 20 hr, for example, about 10, about 11, about 12, about 13, about 14, about 15, about 16, about 17, about 18, about 19, or about 20 hr in the fasting state and be bioequivalent according to FDA guidelines to the second orally administrable dosage form comprising the same dose of tramadol also suitable for once daily administration.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form and will desirably be bioequivalent in the fed or fasted state according to FDA guidelines to a second orally administrable dosage form comprising the same dose of tramadol also suitable for once daily administration in the fed or fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, has reduced potential for alcohol induced dose dumping and will desirably be bioequivalent in the fed or fasted state according to FDA guidelines to a second orally administrable dosage form comprising the same dose of tramadol also suitable for once daily administration in the fed or fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, and will desirably be bioequivalent according to FDA guidelines to a second orally administrable dosage form comprising the same dose of tramadol also suitable for once daily administration in the fed state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, has reduced potential for alcohol induced dose dumping and will desirably be bioequivalent according to FDA guidelines to a second orally administrable dosage form comprising the same dose of tramadol also suitable for once daily administration in the fed state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit: (i) a Cₘₐₓ of from about 180 to about 333 ng/ml of tramadol, (ii) an AUC_{0-∞} of from about 3740 to about 7600 ng·hr/ml of tramadol under fasting conditions, and will desirably be bioequivalent according to FDA guidelines to a second orally administrable dosage form comprising the same dose of tramadol also suitable for once daily administration in the fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit: (i) a Cₘₐₓ of from about 180 to about 333 ng/ml of tramadol, (ii) an AUC_{0-∞} of from about 3740 to about 7600 ng·hr/ml of tramadol under fasting conditions, has reduced potential for alcohol induced dose dumping and will desirably be bioequivalent according to FDA guidelines to a second orally administrable dosage form comprising the same dose of tramadol also suitable for once daily administration in the fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration a Cₘₐₓ of, for example about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335 or about 338 ng/ml of tramadol in the fed state and will desirably be bioequivalent according to FDA guidelines to a second orally administrable dosage form comprising the same dose of tramadol also suitable for once daily administration in the fed state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration an AUC_{0-∞} of, for example, about 2725, about 2750, about 2900, about 3050, about 3200, about 3350 about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions and will desirably be bioequivalent according to FDA guidelines to a second orally administrable dosage form comprising the same dose of tramadol also suitable for once daily administration in the fed state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit a Cₘₐₓ, of, for example, about 180, about 190, about 200, about 210, about 220, about 230, about 240, about 250, about 260, about 270, about 280, about 290, about 300, about 310, about 320, about 330, or about 333 ng/ml of tramadol in the fasted state and will desirably be bioequivalent according to FDA guidelines to a second orally administrable dosage form comprising the same dose of tramadol also suitable for once daily administration in the fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550 about, 4600, about 4650, about 4700, about 4750, about 4800, about 4850, about 4900, about 4950, about 5000, about 5050, about 5100, about 5150, about 5200, about 5250, about 5300, about 5350, about 5400, about 5450, 5500, 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600 ng·hr/ml of tramadol under fasting conditions and will desirably be bioequivalent according to FDA guidelines to a second orally administrable dosage form comprising the same dose of tramadol also suitable for once daily administration in the fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit in the fed state a Tₘₐₓ of tramadol from about 4 to about 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr after single-dose administration and will desirably be bioequivalent according to FDA guidelines to a second orally administrable dosage form comprising tramadol also suitable for once daily administration

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit at steady state the following pharmacokinetic parameters in-vivo under fasting conditions: (i) an AUC₀₋₂₄ from about 1635 to about 21000, for example, from about 1635 to about 3920, about 3610 to about 9120, or about 9455 to about 20965 ng·h/ml, and (ii) a Cₘₐₓ from about 117 to about 1230, for example, about 117 to about 245, about 230 to about 590, or about 590 to about 1230 ng/ml and will desirably be bioequivalent according to FDA guidelines to a second orally administrable dosage form comprising the same dose of tramadol also suitable for once daily administration in the fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit under fasting conditions a Tₘₐₓ of, for example, about 9, about 10, about 11, about 12, about 13, or about 14 hours and will desirably be bioequivalent according to FDA guidelines to a second orally administrable dosage form comprising the same dose of tramadol also suitable for once daily administration in the fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit under fasting conditions a degree of fluctuation (%) of, for example, about 43 to about 141, about 43 to about 120, about 58 to about 132, or about 57 to 1 about 41 and will desirably be bioequivalent according to FDA guidelines to a second orally administrable dosage form comprising the same dose of tramadol also suitable for once daily administration in the fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit under fasting conditions a Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

The invention, in at least one embodiment, is directed to a method for the treatment of moderate to moderately severe pain, said method comprising administering a first once daily controlled-release dosage form comprising at least one means for controllably releasing a therapeutically effective amount of tramadol to a human such that said first once daily controlled-release dosage form will desirably exhibit under fasting conditions a Tₘₐₓ of, for example, about 9, about 10, about 11, about 12, about 13, or about 14 hours, a degree of fluctuation (%) of, for example, about 43 to about 141, about 43 to about 120, about 58 to about 132, or about 57 to about 141, and Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

The at least one means for controllably releasing tramadol will desirably be chosen from at least one controlled-release matrix core; at least one insoluble matrix core; at least one swellable matrix core; at least one swellable and erodable matrix core; at least one hydrophobic matrix core; at least one hydrophilic matrix core; at least one lipid or wax matrix core; at least one erodable matrix core; at least one release-slowing coat; at least one delayed release coat; at least one release-slowing coat comprising at least one pH independent polymer; at least one release-slowing coat comprising at least one pH dependent polymer (such as for example enteric or reverse enteric types); at least one release-slowing coat comprising at least one soluble material, such as for example, a soluble polymer; at least one release-slowing coat comprising at least one insoluble material (aqueous insoluble coat), such as for example, an insoluble polymer; at least one release-slowing coat comprising at least one swellable material, such as for example, a swellable polymer; at least one release-slowing coat comprising at least one hydrophobic material, such as for example, a hydrophobic polymer; at least one release-slowing coat comprising at least one hydrophilic material, such as for example, a hydrophilic polymer; at least one means for the exit of tramadol; at least one release-slowing coat comprising an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol; at least one release-slowing coat comprising at least one water-insoluble water-permeable film-forming polymer and at least one water-soluble polymer; a release-slowing coat comprising at least one water-insoluble water-permeable film-forming polymer, at least one water-soluble polymer and at least one plasticizer; at least one means for increasing the hydrostatic pressure of the first once daily controlled-release dosage form comprising tramadol; at least one means for forcibly dispensing tramadol from the first once daily controlled-release dosage form; or any combination of thereof.

In at least one embodiment of the invention, the first once daily controlled-release dosage form comprising at least one means for controllably releasing the tramadol comprises an effective amount of tramadol for the management of moderate to moderately severe pain.

In at least one embodiment of the invention, the first once daily controlled-release dosage form comprising at least one means for controllably releasing the tramadol comprises from about 25 mg to about 800 mg of tramadol.

In at least one embodiment of the invention, the first once daily controlled-release dosage form comprising at least one means for controllably releasing the tramadol comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the first once daily controlled-release dosage form comprising at least one means for controllably releasing the tramadol comprises tramadol hydrochloride.

In embodiments where the first once daily controlled-release dosage forms comprise at least one means for controllably releasing tramadol, the first once daily controlled-release dosage forms will desirably comprise a mixture of an effective amount of at least two different tramadol salts, wherein one of the salts comprises tramadol hydrochloride.

In embodiments where the first once daily controlled-release dosage forms comprise at least one means for controllably releasing tramadol, the first once daily controlled-release dosage forms comprises 70 to 90% by weight of the core dry weight tramadol.

In embodiments where the first once daily controlled-release dosage forms comprise at least one means for controllably releasing tramadol, the first once daily controlled-release dosage forms comprises an immediate release coat comprising tramadol.

In embodiments where the first once daily controlled-release dosage forms comprise at least one means for controllably releasing tramadol, the first once daily controlled-release dosage forms comprises an immediate release coat comprising a salt of tramadol which is different from the salt of tramadol present in the core of the first once daily controlled-release dosage form.

In at least one embodiment of the invention, the at least one means for controllably releasing tramadol comprises at least one controlled-release matrix core.

In at least one embodiment of the invention, the first once daily controlled-release dosage form comprising at least one controlled-release matrix core comprises an effective amount of tramadol for the management of moderate to moderately severe pain.

In at least one embodiment of the invention, the first once daily controlled-release dosage form comprising at least one controlled-release matrix core comprises from about 25 mg to about 800 mg of tramadol.

In at least one embodiment of the invention, the first once daily controlled-release dosage form comprising at least one controlled-release matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the first once daily controlled-release dosage form comprising at least one controlled-release matrix core comprises tramadol hydrochloride.

In at least one embodiment of the invention, the at least one means for controllably releasing tramadol comprising the at least one controlled-release matrix core will desirably comprise a mixture of an effective amount of at least two different tramadol salts.

In at least one embodiment of the invention, the at least one means for controllably releasing tramadol comprising the at least one controlled-release matrix core will desirably comprise a mixture of an effective amount of at least two different tramadol salts, wherein on salt comprises tramadol hydrochloride.

In at least one embodiment of the invention, the at least one means for controllably releasing tramadol comprising the at least one controlled-release matrix core comprises about 70 to about 90% by weight of the core dry weight tramadol.

In at least one embodiment of the invention, the at least one means for controllably releasing tramadol comprising the at least one controlled-release matrix core comprises an immediate release coat comprising tramadol.

In at least one embodiment of the invention, the at least one means for controllably releasing tramadol comprising the at least one controlled-release matrix core comprises an immediate release coat comprising a salt of tramadol which is different from the salt of tramadol present in said controlled-release matrix core.

In at least one embodiment of the invention, the at least one means for controllably releasing tramadol comprising the at least one controlled-release matrix core comprises at least one release-slowing coat.

In at least one embodiment of the invention, the at least one means for controllably releasing tramadol comprising the at least one controlled-release matrix core comprises at least one delayed-release coat.

In at least one embodiment of the invention, the at least one means for controllably releasing tramadol comprising the at least one controlled-release matrix core comprises an osmotic subcoat.

In at least one embodiment of the invention, the at least one means for controllably releasing tramadol comprising the at least one controlled-release matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising the at least one controlled-release matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising the at least one controlled-release matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising the at least one controlled-release matrix core comprises at least one release-slowing coat, which coat comprises at least one enteric polymer.

In at least one embodiment of the invention, the at least one means for controllably releasing tramadol comprising the at least one controlled-release matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one controlled-release matrix core comprises at least one delayed-release coat, which coat comprises at least one pH dependent polymer.

In at least one embodiment of the invention, the at least one means for controllably releasing tramadol comprising the at least one controlled-release matrix core comprises at least one non-functional soluble coat.

In at least one embodiment of the invention, the first once daily rate controlled-release dosage form comprising the at least one means for controllably releasing tramadol comprising, which comprises at least one controlled-release matrix core is in the form of a tablet.

In at least one embodiment of the invention, the first once daily rate controlled-release dosage form comprising the at least one means for controllably releasing tramadol comprising, which comprises at least one controlled-release matrix core is in the form of a capsule.

In at least one embodiment of the invention, the first once daily rate controlled-release dosage form comprising the at least one means for controllably releasing tramadol comprising, which comprises at least one controlled-release matrix core is in the form of a microparticle.

In at least one embodiment of the invention, the at least one means for controllably releasing tramadol comprising the at least one controlled-release matrix core is comprised of a plurality of microparticles, wherein each microparticle comprises at least one controlled-release matrix core.

In at least one embodiment of the invention, the at least one means for controllably releasing tramadol comprising the at least one controlled-release matrix core is comprised of at least one unitary core.

In at least one embodiment of the invention, the at least one means for controllably releasing tramadol comprising the at least one controlled-release matrix core further comprises two or more coats, wherein one coat comprises a release-slowing coat.

In at least one embodiment of the invention, the at least one means for controllably releasing tramadol comprising the at least one controlled-release matrix core further comprises two or more coats, wherein one coat comprises a delayed-release coat.

In at least one embodiment of the invention, the at least one means for controllably releasing tramadol comprising the at least one controlled-release matrix core is comprised of a plurality of coated cores.

In at least one embodiment of the invention, the at least one means for controllably releasing tramadol comprises at least one insoluble matrix core.

In at least one embodiment of the invention, the first once daily controlled-release dosage form comprising at least one insoluble matrix core comprises an effective amount of tramadol for the management of moderate to moderately severe pain.

In at least one embodiment of the invention, the first once daily controlled-release dosage form comprising at least one insoluble matrix core comprises from about 25 mg to about 800 mg of tramadol.

In at least one embodiment of the invention, the first once daily controlled-release dosage form comprising at least one insoluble matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the first once daily controlled-release dosage form comprising at least one insoluble matrix core comprises tramadol hydrochloride.

In at least one embodiment of the invention, the at least one means for controllably releasing tramadol comprising the at least one insoluble matrix core will desirably comprise a mixture of an effective amount of at least two different tramadol salts, wherein one of the salts comprises tramadol hydrochloride.

In at least one embodiment of the invention, the at least one means for controllably releasing tramadol comprising the at least one insoluble matrix core comprises about 70 to about 90% by weight of the core dry weight tramadol.

In at least one embodiment of the invention, the at least one means for controllably releasing tramadol comprising the at least one insoluble matrix core comprises an immediate release coat comprising tramadol.

In at least one embodiment of the invention, the at least one means for controllably releasing tramadol comprising the at least one insoluble matrix core comprises an immediate release coat comprising a salt of tramadol which is different from the salt of tramadol present in said insoluble matrix core.

In at least one embodiment of the invention, the at least one means for controllably releasing tramadol comprising the at least one insoluble matrix core comprises at least one release-slowing coat.

In at least one embodiment of the invention, the at least one means for controllably releasing tramadol comprising the at least one insoluble matrix core comprises at least one delayed-release coat.

In at least one embodiment of the invention, the at least one means for controllably releasing tramadol comprising the at least one insoluble matrix core comprises an osmotic subcoat.

In at least one embodiment of the invention, the at least one means for controllably releasing tramadol comprising the at least one insoluble matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising the at least one insoluble matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising the at least one insoluble matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising the at least one insoluble matrix core comprises at least one release-slowing coat, which coat comprises at least one enteric polymer.

In at least one embodiment of the invention, the at least one means for controllably releasing tramadol comprising the at least one insoluble matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one insoluble matrix core comprises at least delayed-release coat, which coat comprises at least one pH dependent polymer.

In at least one embodiment of the invention, the at least one means for controllably releasing tramadol comprising the at least one insoluble matrix core comprises at least one non-functional soluble coat.

In at least one embodiment of the invention, the first once daily rate controlled-release dosage form comprising the at least one means for controllably releasing tramadol is comprised of at least one insoluble matrix core is in the form of a tablet.

In at least one embodiment of the invention, the first once daily rate controlled-release dosage form comprising the at least one means for controllably releasing tramadol is comprised of at least one insoluble matrix core is in the form of a capsule.

In at least one embodiment of the invention, the first once daily rate controlled-release dosage form comprising the at least one means for controllably releasing tramadol is comprised of at least one insoluble matrix core is in the form of a microparticle.

In at least one embodiment of the invention, the at least one means for controllably releasing tramadol comprising the at least one insoluble matrix core is comprised of a plurality of microparticles, wherein each microparticle comprises at least one insoluble matrix core.

In at least one embodiment of the invention, the at least one means for controllably releasing tramadol comprising the at least one insoluble matrix core is comprised of at least one unitary core.

In at least one embodiment of the invention, the at least one means for controllably releasing tramadol comprising the at least one insoluble matrix core further comprises two or more coats, wherein one coat comprises a release-slowing coat.

In at least one embodiment of the invention, the at least one means for controllably releasing tramadol comprising the at least one insoluble matrix core further comprises two or more coats, wherein one coat comprises a delayed-release coat.

In at least one embodiment of the invention, the at least one means for controllably releasing tramadol comprising the at least one insoluble matrix core is comprised of a plurality of coated cores.

In at least one embodiment of the invention, the at least one means for controllably releasing tramadol comprises at least one swellable matrix core.

In at least one embodiment of the invention, the first once daily controlled-release dosage form comprising at least one swellable matrix core comprises an effective amount of tramadol for the management of moderate to moderately severe pain.

In at least one embodiment of the invention, the first once daily controlled-release dosage form comprising at least one swellable matrix core comprises from about 25 mg to about 800 mg of tramadol.

In at least one embodiment of the invention, the first once daily controlled-release dosage form comprising at least one swellable matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the first once daily controlled-release dosage form comprising at least one swellable matrix core comprises tramadol hydrochloride.

In at least one embodiment of the invention, the at least one means for controllably releasing tramadol comprising the at least one swellable matrix core will desirably comprise a mixture of an effective amount of at least two different tramadol salts.

In at least one embodiment of the invention, the at least one means for controllably releasing tramadol comprising the at least one swellable matrix core comprises about 70 to about 90% by weight of the core dry weight tramadol.

In at least one embodiment of the invention, the at least one means for controllably releasing tramadol comprising the at least one swellable matrix core comprises an immediate release coat comprising tramadol.

In at least one embodiment of the invention, the at least one means for controllably releasing tramadol comprising the at least one swellable matrix core comprises an immediate release coat comprising a salt of tramadol which is different from the salt of tramadol present in said swellable matrix core.

In at least one embodiment of the invention, the at least one means for controllably releasing tramadol comprising the at least one swellable matrix core comprises at least one release-slowing coat.

In at least one embodiment of the invention, the at least one means for controllably releasing tramadol comprising the at least one swellable matrix core comprises at least one delayed-release coat.

In at least one embodiment of the invention, the at least one means for controllably releasing tramadol comprising the at least one swellable matrix core comprises an osmotic subcoat.

In at least one embodiment of the invention, the at least one means for controllably releasing tramadol comprising the at least one swellable matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising the at least one swellable matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising the at least one swellable matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising the at least one swellable matrix core comprises at least one release-slowing coat, which coat comprises at least one enteric polymer.

In at least one embodiment of the invention, the at least one means for controllably releasing tramadol comprising the at least one swellable matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one swellable matrix core comprises at least delayed-release coat, which coat comprises at least one pH dependent polymer.

In at least one embodiment of the invention, the at least one means for controllably releasing tramadol comprising the at least one swellable matrix core comprises at least one non-functional soluble coat.

In at least one embodiment of the invention, the first once daily rate controlled-release dosage form comprising the at least one means for controllably releasing tramadol is comprised of at least one swellable matrix core is in the form of a tablet.

In at least one embodiment of the invention, the first once daily rate controlled-release dosage form comprising the at least one means for controllably releasing tramadol is comprised of at least one swellable matrix core is in the form of a capsule.

In at least one embodiment of the invention, once daily rate controlled-release dosage form comprising the at least one means for controllably releasing tramadol is comprised of at least one swellable matrix core is in the form of a microparticle.

In at least one embodiment of the invention, the first once daily rate controlled-release dosage form comprising the at least one means for controllably releasing tramadol is comprised of at least one swellable matrix core comprises a plurality of microparticles, wherein each microparticle comprises at least one swellable matrix core.

In at least one embodiment of the invention, the at least one means for controllably releasing tramadol comprising the at least one swellable matrix core is comprised of at least one unitary core.

In at least one embodiment of the invention, the at least one means for controllably releasing tramadol comprising the at least one swellable matrix core further comprises two or more coats, wherein one coat comprises a release-slowing coat.

In at least one embodiment of the invention, the at least one means for controllably releasing tramadol comprising the at least one swellable matrix core further comprises two or more coats, wherein one coat comprises a delayed-release coat.

In at least one embodiment of the invention, the at least one means for controllably releasing tramadol comprising the at least one swellable matrix core is comprised of a plurality of coated cores.

In at least one embodiment of the invention, the at least one means for controllably releasing tramadol comprises at least one swellable and erodable matrix core.

In at least one embodiment of the invention, the first once daily controlled-release dosage form comprising at least one swellable and erodable matrix core comprises an effective amount of tramadol for the management of moderate to moderately severe pain.

In at least one embodiment of the invention, the first once daily controlled-release dosage form comprising at least one swellable and erodable matrix core comprises from about 25 mg to about 800 mg of tramadol.

In at least one embodiment of the invention, the first once daily controlled-release dosage form comprising at least one swellable and erodable matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the first once daily controlled-release dosage form comprising at least one swellable and erodable matrix core comprises tramadol hydrochloride.

In at least one embodiment of the invention, the at least one means for controllably releasing tramadol comprising the at least one swellable and erodable matrix core will desirably comprise a mixture of an effective amount of at least two different tramadol salts, wherein one of the salts comprises tramadol hydrochloride.

In at least one embodiment of the invention, the at least one means for controllably releasing tramadol comprising the at least one swellable and erodable matrix core comprises about 70 to about 90% by weight of the core dry weight tramadol.

In at least one embodiment of the invention, the at least one means for controllably releasing tramadol comprising the at least one swellable and erodable matrix core comprises an immediate release coat comprising tramadol.

In at least one embodiment of the invention, the at least one means for controllably releasing tramadol comprising the at least one swellable and erodable matrix core comprises an immediate release coat comprising a salt of tramadol which is different from the salt of tramadol present in said swellable and erodable matrix core.

In at least one embodiment of the invention, the at least one means for controllably releasing tramadol comprising the at least one swellable and erodable matrix core comprises at least one release-slowing coat.

In at least one embodiment of the invention, the at least one means for controllably releasing tramadol comprising the at least one swellable and erodable matrix core comprises at least one delayed-release coat.

In at least one embodiment of the invention, the at least one means for controllably releasing tramadol comprising the at least one swellable and erodable matrix core comprises an osmotic subcoat.

In at least one embodiment of the invention, the at least one means for controllably releasing tramadol comprising the at least one swellable and erodable matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising the at least one swellable and erodable matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising the at least one swellable and erodable matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising the at least one swellable and erodable matrix core comprises at least one release-slowing coat, which coat comprises at least one enteric polymer.

In at least one embodiment of the invention, the at least one means for controllably releasing tramadol comprising the at least one swellable and erodable matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one swellable and erodable matrix core comprises at least delayed-release coat, which coat comprises at least one pH dependent polymer.

In at least one embodiment of the invention, the at least one means for controllably releasing tramadol comprising the at least one swellable and erodable matrix core comprises at least one non-functional soluble coat.

In at least one embodiment of the invention, the first once daily rate controlled-release dosage form comprising the at least one means for controllably releasing tramadol comprises at least one swellable and erodable matrix core is in the form of a tablet.

In at least one embodiment of the invention, the first once daily rate controlled-release dosage form comprising the at least one means for controllably releasing tramadol comprises the at least one swellable and erodable matrix core is in the form of a capsule.

In at least one embodiment of the invention, the first once daily rate controlled-release dosage form comprising the at least one means for controllably releasing tramadol comprises at least one swellable and erodable matrix core is in the form of a microparticle.

In at least one embodiment of the invention, the first once daily rate controlled-release dosage form comprising the at least one means for controllably releasing tramadol comprises at least one swellable and erodable matrix core is in the form of a plurality of microparticles, wherein each microparticle comprises at least one swellable and erodable matrix core.

In at least one embodiment of the invention, the at least one means for controllably releasing tramadol comprising the at least one swellable and erodable matrix core is comprised of at least one unitary core.

In at least one embodiment of the invention, the at least one means for controllably releasing tramadol comprising the at least one swellable and erodable matrix core further comprises two or more coats, wherein one coat comprises a release-slowing coat.

In at least one embodiment of the invention, the at least one means for controllably releasing tramadol comprising the at least one swellable and erodable matrix core further comprises two or more coats, wherein one coat comprises a delayed-release coat.

In at least one embodiment of the invention, the first once daily rate controlled-release dosage form comprising the at least one means for controllably releasing tramadol comprises at least one swellable and erodable matrix core is in the form of a plurality of coated cores.

In at least one embodiment of the invention, the at least one means for controllably releasing tramadol comprises at least one hydrophobic matrix core.

In at least one embodiment of the invention, the first once daily controlled-release dosage form comprising at least one hydrophobic matrix core comprises an effective amount of tramadol for the management of moderate to moderately severe pain.

In at least one embodiment of the invention, the first once daily controlled-release dosage form comprising at least one hydrophobic matrix core comprises from about 25 mg to about 800 mg of tramadol.

In at least one embodiment of the invention, the first once daily controlled-release dosage form comprising at least one hydrophobic matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the first once daily controlled-release dosage form comprising at least one hydrophobic matrix core comprises tramadol hydrochloride.

In at least one embodiment of the invention, the at least one means for controllably releasing tramadol comprising the at least one hydrophobic matrix core will desirably comprise a mixture of an effective amount of at least two different tramadol salts, wherein one salt comprises tramadol hydrochloride.

In at least one embodiment of the invention, the at least one means for controllably releasing tramadol comprising the at least one hydrophobic matrix core comprises 70 to 90% by weight of the core dry weight tramadol.

In at least one embodiment of the invention, the at least one means for controllably releasing tramadol comprising the at least one hydrophobic matrix core comprises an immediate release coat comprising tramadol.

In at least one embodiment of the invention, the at least one means for controllably releasing tramadol comprising the at least one hydrophobic matrix core comprises an immediate release coat comprising a salt of tramadol which is different from the salt of tramadol present in said hydrophobic matrix core.

In at least one embodiment of the invention, the at least one means for controllably releasing tramadol comprising the at least one hydrophobic matrix core comprises at least one release-slowing coat.

In at least one embodiment of the invention, the at least one means for controllably releasing tramadol comprising the at least one hydrophobic matrix core comprises at least one delayed-release coat.

In at least one embodiment of the invention, the at least one means for controllably releasing tramadol comprising the at least one hydrophobic matrix core comprises an osmotic subcoat.

In at least one embodiment of the invention, the at least one means for controllably releasing tramadol comprising the at least one hydrophobic matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising the at least one hydrophobic matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising the at least one hydrophobic matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising the at least one hydrophobic matrix core comprises at least one release-slowing coat, which coat comprises at least one enteric polymer.

In at least one embodiment of the invention, the at least one means for controllably releasing tramadol comprising the at least one hydrophobic matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one hydrophobic matrix core comprises at least delayed-release coat, which coat comprises at least one pH dependent polymer.

In at least one embodiment of the invention, the at least one means for controllably releasing tramadol comprising the at least one hydrophobic matrix core comprises at least one non-functional soluble coat.

In at least one embodiment of the invention, the first once daily rate controlled-release dosage form comprising the at least one means for controllably releasing tramadol comprises at least one hydrophobic matrix core is in the form of a tablet.

In at least one embodiment of the invention, the first once daily rate controlled-release dosage form comprising the at least one means for controllably releasing tramadol comprises at least one hydrophobic matrix core is in the form of a capsule.

In at least one embodiment of the invention, the first once daily rate controlled-release dosage form comprising the at least one means for controllably releasing tramadol comprises at least one hydrophobic matrix core is in the form of a microparticle.

In at least one embodiment of the invention, the first once daily rate controlled-release dosage form comprising the at least one means for controllably releasing tramadol comprises at least one hydrophobic matrix core is in the form a plurality of microparticles, wherein each microparticle comprises at least one hydrophobic matrix core.

In at least one embodiment of the invention, the at least one means for controllably releasing tramadol comprising the at least one hydrophobic matrix core is comprised of at least one unitary core.

In at least one embodiment of the invention, the at least one means for controllably releasing tramadol comprising the at least one hydrophobic matrix core further comprises two or more coats, wherein one coat comprises a release-slowing coat.

In at least one embodiment of the invention, the at least one means for controllably releasing tramadol comprising the at least one hydrophobic matrix core further comprises two or more coats, wherein one coat comprises a delayed-release coat.

In at least one embodiment of the invention, the first once daily rate controlled-release dosage form comprising at least one means for controllably releasing tramadol comprises the at least one hydrophobic matrix core is in the form of a plurality of coated cores.

In at least one embodiment of the invention, the at least one means for controllably releasing tramadol comprises at least one hydrophilic matrix core.

In at least one embodiment of the invention, the first once daily controlled-release dosage form comprising at least one hydrophilic matrix core comprises an effective amount of tramadol for the management of moderate to moderately severe pain.

In at least one embodiment of the invention, the first once daily controlled-release dosage form comprising at least one hydrophilic matrix core comprises from about 25 mg to about 800 mg of tramadol.

In at least one embodiment of the invention, the first once daily controlled-release dosage form comprising at least one hydrophilic matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the at least one means for controllably releasing tramadol comprising the at least one hydrophilic matrix core will desirably comprise a mixture of an effective amount of at least two different tramadol salts.

In at least one embodiment of the invention, the at least one means for controllably releasing tramadol comprising the at least one hydrophilic matrix core comprises about 70 to about 90% by weight of the core dry weight tramadol.

In at least one embodiment of the invention, the at least one means for controllably releasing tramadol comprising the at least one hydrophilic matrix core comprises an immediate release coat comprising tramadol.

In at least one embodiment of the invention, the at least one means for controllably releasing tramadol comprising the at least one hydrophilic matrix core comprises an immediate release coat comprising a salt of tramadol which is different from the salt of tramadol present in said hydrophilic matrix core.

In at least one embodiment of the invention, the at least one means for controllably releasing tramadol comprising the at least one hydrophilic matrix core comprises at least one release-slowing coat.

In at least one embodiment of the invention, the at least one means for controllably releasing tramadol comprising the at least one hydrophilic matrix core comprises at least one delayed-release coat.

In at least one embodiment of the invention, the at least one means for controllably releasing tramadol comprising the at least one hydrophilic matrix core comprises an osmotic subcoat.

In at least one embodiment of the invention, the at least one means for controllably releasing tramadol comprising the at least one hydrophilic matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising the at least one hydrophilic matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising the at least one hydrophilic matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising the at least one hydrophilic matrix core comprises at least one release-slowing coat, which coat comprises at least one enteric polymer.

In at least one embodiment of the invention, the at least one means for controllably releasing tramadol comprising the at least one hydrophilic matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one hydrophilic matrix core comprises at least delayed-release coat, which coat comprises at least one pH dependent polymer.

In at least one embodiment of the invention, the at least one means for controllably releasing tramadol comprising the at least one hydrophilic matrix core comprises at least one non-functional soluble coat.

In at least one embodiment of the invention, the first once daily rate controlled-release dosage form comprising the at least one means for controllably releasing tramadol comprises at least one hydrophilic matrix core is in the form of a tablet.

In at least one embodiment of the invention, the first once daily rate controlled-release dosage form comprising the at least one means for controllably releasing tramadol comprises at least one hydrophilic matrix core is in the form of a capsule.

In at least one embodiment of the invention, the first once daily rate controlled-release dosage form comprising the at least one means for controllably releasing tramadol comprises at least one hydrophilic matrix core is in the form of a microparticle.

In at least one embodiment of the invention, the first once daily rate controlled-release dosage form comprising the at least one means for controllably releasing tramadol comprises at least one hydrophilic matrix core is in the form a plurality of microparticles, wherein each microparticle comprises at least one hydrophilic matrix core.

In at least one embodiment of the invention, the at least one means for controllably releasing tramadol comprising the at least one hydrophilic matrix core is comprised of at least one unitary core.

In at least one embodiment of the invention, the at least one means for controllably releasing tramadol comprising the at least one hydrophilic matrix core further comprises two or more coats, wherein one coat comprises a release-slowing coat.

In at least one embodiment of the invention, the at least one means for controllably releasing tramadol comprising the at least one hydrophilic matrix core further comprises two or more coats, wherein one coat comprises a delayed-release coat.

In at least one embodiment of the invention, the first once daily rate controlled-release dosage form comprising at least one means for controllably releasing tramadol comprises the at least one hydrophilic matrix core is in the form of a plurality of coated cores.

In at least one embodiment of the invention, the at least one means for controllably releasing tramadol comprises a combination of a hydrophobic and hydrophilic matrix core.

In at least one embodiment of the invention, the first once daily controlled-release dosage form comprising a combination of a hydrophobic and hydrophilic matrix core comprises an effective amount of tramadol for the management of moderate to moderately severe pain.

In at least one embodiment of the invention, the first once daily controlled-release dosage form comprising a combination of a hydrophobic and hydrophilic matrix core comprises from about 25 mg to about 800 mg of tramadol.

In at least one embodiment of the invention, the first once daily controlled-release dosage form comprising a combination of a hydrophobic and hydrophilic matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising a combination of a hydrophobic and hydrophilic matrix core comprises tramadol hydrochloride.

In at least one embodiment of the invention, the at least one means for controllably releasing tramadol comprising a combination of a hydrophobic and hydrophilic matrix core will desirably comprise a mixture of an effective amount of at least two different tramadol salts, wherein one salt comprises tramadol hydrochloride.

In at least one embodiment of the invention, the at least one means for controllably releasing tramadol comprising a combination of a hydrophobic and hydrophilic matrix core comprises about 70 to about 90% by weight of the core dry weight tramadol.

In at least one embodiment of the invention, the at least one means for controllably releasing tramadol comprising a combination of a hydrophobic and hydrophilic matrix core comprises an immediate release coat comprising tramadol.

In at least one embodiment of the invention, the at least one means for controllably releasing tramadol comprising a combination of a hydrophobic and hydrophilic matrix core comprises an immediate release coat comprising a salt of tramadol which is different from the salt of tramadol present in said combination of the hydrophobic and hydrophilic matrix core.

In at least one embodiment of the invention, the at least one means for controllably releasing tramadol comprising a combination of a hydrophobic and hydrophilic matrix core comprises at least one release-slowing coat.

In at least one embodiment of the invention, the at least one means for controllably releasing tramadol comprising a combination of a hydrophobic and hydrophilic matrix core comprises at least one delayed-release coat.

In at least one embodiment of the invention, the at least one means for controllably releasing tramadol comprising a combination of a hydrophobic and hydrophilic matrix core comprises an osmotic subcoat.

In at least one embodiment of the invention, the at least one means for controllably releasing tramadol comprising a combination of a hydrophobic and hydrophilic matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising a combination of a hydrophobic and hydrophilic matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising a combination of a hydrophobic and hydrophilic matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising a combination of a hydrophobic and hydrophilic matrix core comprises at least one release-slowing coat, which coat comprises at least one enteric polymer.

In at least one embodiment of the invention, the at least one means for controllably releasing tramadol comprising a combination of a hydrophobic and hydrophilic matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising a combination of a hydrophobic and hydrophilic matrix core comprises at least delayed-release coat, which coat comprises at least one pH dependent polymer.

In at least one embodiment of the invention, the at least one means for controllably releasing tramadol comprising a combination of a hydrophobic and hydrophilic matrix core comprises at least one non-functional soluble coat.

In at least one embodiment of the invention, the first once daily rate controlled-release dosage form comprising the at least one means for controllably releasing tramadol comprises a combination of a hydrophobic and hydrophilic matrix core is in the form of a tablet.

In at least one embodiment of the invention, the first once daily rate controlled-release dosage form comprising the at least one means for controllably releasing tramadol comprises a combination of a hydrophobic and hydrophilic matrix core is in the form of a capsule.

In at least one embodiment of the invention, the first once daily rate controlled-release dosage form comprising the at least one means for controllably releasing tramadol comprises a combination of a hydrophobic and hydrophilic matrix core is in the form of a microparticle.

In at least one embodiment of the invention, the first once daily rate controlled-release dosage form comprising the at least one means for controllably releasing tramadol comprises a combination of a hydrophobic and hydrophilic matrix core is in the form a plurality of microparticles, wherein each microparticle comprises a combination of the hydrophobic and hydrophilic matrix core.

In at least one embodiment of the invention, the at least one means for controllably releasing tramadol comprising a combination of a hydrophobic and hydrophilic matrix core is comprised of at least one unitary core.

In at least one embodiment of the invention, the at least one means for controllably releasing tramadol comprising a combination of a hydrophobic and hydrophilic matrix core further comprises two or more coats, wherein one coat comprises a release-slowing coat.

In at least one embodiment of the invention, the at least one means for controllably releasing tramadol comprising a combination of a hydrophobic and hydrophilic matrix core further comprises two or more coats, wherein one coat comprises a delayed-release coat.

In at least one embodiment of the invention, the first once daily rate controlled-release dosage form comprising at least one means for controllably releasing tramadol comprises a combination of a hydrophobic and hydrophilic matrix core is in the form of a plurality of coated cores.

In at least one embodiment of the invention, the at least one means for controllably releasing tramadol comprises a lipid or wax matrix core.

In at least one embodiment of the invention, the first once daily controlled-release dosage form comprising a lipid or wax matrix core comprises an effective amount of tramadol for the management of moderate to moderately severe pain.

In at least one embodiment of the invention, the first once daily controlled-release dosage form comprising a lipid or wax matrix core comprises from about 25 mg to about 800 mg of tramadol.

In at least one embodiment of the invention, the first once daily controlled-release dosage form comprising a lipid or wax matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising a lipid or wax matrix core comprises tramadol hydrochloride.

In at least one embodiment of the invention, the at least one means for controllably releasing tramadol comprising a lipid or wax matrix core will desirably comprise a mixture of an effective amount of at least two different tramadol salts, wherein one salt comprises tramadol hydrochloride.

In at least one embodiment of the invention, the at least one means for controllably releasing tramadol comprising a lipid or wax matrix core comprises about 70 to about 90% by weight of the core dry weight tramadol.

In at least one embodiment of the invention, the at least one means for controllably releasing tramadol comprising a lipid or wax matrix core comprises an immediate release coat comprising tramadol.

In at least one embodiment of the invention, the at least one means for controllably releasing tramadol comprising a lipid or wax matrix core comprises an immediate release coat comprising a salt of tramadol which is different from the salt of tramadol present in said lipid or wax matrix core.

In at least one embodiment of the invention, the at least one means for controllably releasing tramadol comprising a lipid or wax matrix core comprises at least one release-slowing coat.

In at least one embodiment of the invention, the at least one means for controllably releasing tramadol comprising a lipid or wax matrix core comprises at least one delayed-release coat.

In at least one embodiment of the invention, the at least one means for controllably releasing tramadol comprising a lipid or wax matrix core comprises an osmotic subcoat.

In at least one embodiment of the invention, the at least one means for controllably releasing tramadol comprising a lipid or wax matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising a lipid or wax matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising a lipid or wax matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising a lipid or wax matrix core comprises at least one release-slowing coat, which coat comprises at least one enteric polymer.

In at least one embodiment of the invention, the at least one means for controllably releasing tramadol comprising a lipid or wax matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising a lipid or wax matrix core comprises at least delayed-release coat, which coat comprises at least one pH dependent polymer.

In at least one embodiment of the invention, the at least one means for controllably releasing tramadol comprising a lipid or wax matrix core comprises at least one non-functional soluble coat.

In at least one embodiment of the invention, the first once daily rate controlled-release dosage form comprising the at least one means for controllably releasing tramadol comprises a lipid or wax matrix core is in the form of a tablet.

In at least one embodiment of the invention, the first once daily rate controlled-release dosage form comprising the at least one means for controllably releasing tramadol comprises a lipid or wax matrix core is in the form of a capsule.

In at least one embodiment of the invention, the first once daily rate controlled-release dosage form comprising the at least one means for controllably releasing tramadol comprises a lipid or wax matrix core is in the form of a microparticle.

In at least one embodiment of the invention, the first once daily rate controlled-release dosage form comprising the at least one means for controllably releasing tramadol comprises a lipid or wax matrix core is in the form a plurality of microparticles, wherein each microparticle comprises a lipid or wax matrix core.

In at least one embodiment of the invention, the at least one means for controllably releasing tramadol comprising a lipid or wax matrix core is comprised of at least one unitary core.

In at least one embodiment of the invention, the at least one means for controllably releasing tramadol comprising a lipid or wax matrix core further comprises two or more coats, wherein one coat comprises a release-slowing coat.

In at least one embodiment of the invention, the at least one means for controllably releasing tramadol comprising a lipid or wax matrix core further comprises two or more coats, wherein one coat comprises a delayed-release coat.

In at least one embodiment of the invention, the first once daily rate controlled-release dosage form comprising at least one means for controllably releasing tramadol comprises a lipid or wax matrix core is in the form of a plurality of coated cores.

In at least one embodiment of the invention, the at least one means for controllably releasing tramadol comprises at least one erodable matrix core.

In at least one embodiment of the invention, the first once daily controlled-release dosage form comprising at least one erodable matrix core comprises an effective amount of tramadol for the management of moderate to moderately severe pain.

In at least one embodiment of the invention, the first once daily controlled-release dosage form comprising at least one erodable matrix core comprises from about 25 mg to about 800 mg of tramadol.

In at least one embodiment of the invention, the first once daily controlled-release dosage form comprising at least one erodable matrix core comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising at least one erodable matrix core comprises tramadol hydrochloride.

In at least one embodiment of the invention, the at least one means for controllably releasing tramadol comprising the at least one erodable matrix core will desirably comprise a mixture of an effective amount of at least two different tramadol salts, wherein one salt comprises tramadol hydrochloride.

In at least one embodiment of the invention, the at least one means for controllably releasing tramadol comprising the at least one erodable matrix core comprises about 70 to about 90% by weight of the core dry weight tramadol.

In at least one embodiment of the invention, the at least one means for controllably releasing tramadol comprising the at least one erodable matrix core comprises an immediate release coat comprising tramadol.

In at least one embodiment of the invention, the at least one means for controllably releasing tramadol comprising the at least one erodable matrix core comprises an immediate release coat comprising a salt of tramadol which is different from the salt of tramadol present in said erodable matrix core.

In at least one embodiment of the invention, the at least one means for controllably releasing tramadol comprising the at least one erodable matrix core comprises at least one release-slowing coat.

In at least one embodiment of the invention, the at least one means for controllably releasing tramadol comprising the at least one erodable matrix core comprises at least one delayed-release coat.

In at least one embodiment of the invention, the at least one means for controllably releasing tramadol comprising the at least one erodable matrix core comprises an osmotic subcoat.

In at least one embodiment of the invention, the at least one means for controllably releasing tramadol comprising the at least one erodable matrix core comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising the at least one erodable matrix core comprises at least one release-slowing coat, which coat comprises at least one water-insoluble water-permeable film-forming polymer and at least one water-soluble polymer.

In at least one embodiment of the invention, the at least one means for controllably releasing tramadol comprising the at least one erodable matrix core comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprising the at least one erodable matrix core comprises at least one release-slowing coat, which coat comprises at least one enteric polymer.

In at least one embodiment of the invention, the at least one means for controllably releasing tramadol comprising the at least one erodable matrix core comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one erodable matrix core comprises at least delayed-release coat, which coat comprises at least one pH dependent polymer.

In at least one embodiment of the invention, the at least one means for controllably releasing tramadol comprising the at least one erodable matrix core comprises at least one non-functional soluble coat.

In at least one embodiment of the invention, the first once daily rate controlled-release dosage form comprising the at least one means for controllably releasing tramadol comprises at least one erodable matrix core is in the form of a tablet.

In at least one embodiment of the invention, the first once daily rate controlled-release dosage form comprising the at least one means for controllably releasing tramadol comprises at least one erodable matrix core is in the form of a capsule.

In at least one embodiment of the invention, the first once daily rate controlled-release dosage form comprising the at least one means for controllably releasing tramadol comprises at least one erodable matrix core is in the form of a microparticle.

In at least one embodiment of the invention, the first once daily rate controlled-release dosage form comprising the at least one means for controllably releasing tramadol comprises at least one erodable matrix core is in the form of a plurality of microparticles, wherein each microparticle comprises at least one erodable matrix core.

In at least one embodiment of the invention, the at least one means for controllably releasing tramadol comprising the at least one erodable matrix core is comprised of a unitary core

In at least one embodiment of the invention, the at least one means for controllably releasing tramadol comprising the at least one erodable matrix core further comprises two or more coats, wherein one coat comprises a release-slowing coat.

In at least one embodiment of the invention, the at least one means for controllably releasing tramadol comprising the at least one erodable matrix core further comprises two or more coats, wherein one coat comprises a delayed-release coat.

In at least one embodiment of the invention, the first once daily rate controlled-release dosage form comprising the at least one means for controllably releasing tramadol comprises at least one erodable matrix core is in the form of a plurality of coated cores.

In at least one embodiment of the invention, the at least one means for controllably releasing tramadol comprises at least one release-slowing coat.

In at least one embodiment of the invention, the first once daily controlled-release dosage form comprising at least one release-slowing coat comprises an effective amount of tramadol for the management of moderate to moderately severe pain.

In at least one embodiment of the invention, the first once daily controlled-release dosage form comprising at least one release-slowing coat comprises from 25 mg to about 800 mg of tramadol.

In at least one embodiment of the invention, the first once daily controlled-release dosage form comprising at least one release-slowing coat comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the first once daily controlled-release dosage form comprising at least one release-slowing coat comprises tramadol hydrochloride.

In at least one embodiment of the invention, the first once daily controlled-release dosage form comprising the at least one release-slowing coat will desirably comprise a mixture of an effective amount of at least two different tramadol salts, wherein one salt is tramadol hydrochloride.

In at least one embodiment of the invention, the first once daily controlled-release dosage form comprising the at least one release-slowing coat and controlled-release matrix core will desirably comprise about 70 to about 90% by weight of the core dry weight tramadol.

In at least one embodiment of the invention, the first once daily controlled-release dosage form comprising the at least one release-slowing coat will desirably comprise comprises an immediate release coat, wherein said immediate release coat comprises tramadol.

In at least one embodiment of the invention, the first once daily controlled-release dosage form comprising the at least one release-slowing coat and controlled-release matrix core comprises an immediate release coat, wherein said immediate release coat comprises a salt or form of tramadol, which is different from the salt or form of tramadol present in said controlled-release matrix core.

In at least one embodiment of the invention, the at least one means for controllably releasing tramadol comprising the at least one release-slowing coat comprises at least one controlled-release matrix core.

In at least one embodiment of the invention, the at least one means for controllably releasing tramadol comprising the at least one release-slowing coat comprises at least one erodible matrix core.

In at least one embodiment of the invention, the at least one means for controllably releasing tramadol comprising the at least one release-slowing coat comprises at least one swellable matrix core.

In at least one embodiment of the invention, the at least one means for controllably releasing tramadol comprising the at least one release-slowing coat comprises at least one erodible and swellable matrix core.

In at least one embodiment of the invention, the at least one means for controllably releasing tramadol comprising the at least one release-slowing coat comprises at least one hydrophobic matrix core.

In at least one embodiment of the invention, the at least one means for controllably releasing tramadol comprising the at least one release-slowing coat comprises at least one insoluble polymer matrix core.

In at least one embodiment of the invention, the at least one means for controllably releasing tramadol comprising the at least one release-slowing coat comprises at least one insoluble matrix core.

In at least one embodiment of the invention, the at least one means for controllably releasing tramadol comprising the at least one release-slowing coat comprises at least one delayed-release coat.

In at least one embodiment of the invention, the at least one means for controllably releasing tramadol comprising the at least one release-slowing coat comprises an osmotic subcoat.

In at least one embodiment of the invention, the at least one means for controllably releasing tramadol comprises the at least one release-slowing coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprises the at least one release-slowing coat, which coat comprises at least one water-insoluble water-permeable film-forming polymer and at least one water-soluble polymer.

In at least one embodiment of the invention, the at least one means for controllably releasing tramadol comprises the at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprises the at least one release-slowing coat, which coat comprises at least one enteric polymer.

In at least one embodiment of the invention, the at least one means for controllably releasing tramadol comprises the at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the least one release-slowing coat, comprises at least one delayed-release coat, which delayed-release coat comprises at least one pH dependent polymer.

In at least one embodiment of the invention, the at least one means for controllably releasing tramadol comprising the at least one release-slowing coat comprises at least one non-functional soluble coat.

In at least one embodiment of the invention, the first once daily rate controlled-release dosage form comprising the at least one means for controllably releasing tramadol comprises at least one release-slowing coat, wherein said once daily rate controlled-release dosage form is in the form of a tablet.

In at least one embodiment of the invention, the first once daily rate controlled-release dosage form comprising the at least one means for controllably releasing tramadol comprises at least one release-slowing coat, wherein said once daily rate controlled-release dosage form is in the form of a capsule.

In at least one embodiment of the invention, the first once daily rate controlled-release dosage form comprising the at least one means for controllably releasing tramadol comprises at least one release-slowing coat, wherein said once daily rate controlled-release dosage form is in the form of a microparticle.

In at least one embodiment of the invention, the first once daily rate controlled-release dosage form comprising the at least one means for controllably releasing tramadol comprises least one release-slowing coat in the form of a plurality of microparticles, wherein each microparticle comprises at least one release-slowing coat.

In at least one embodiment of the invention, the at least one means for controllably releasing tramadol comprising at least one release-slowing coat is comprised of a unitary core.

In at least one embodiment of the invention, the at least one means for controllably releasing tramadol comprising the at least one release-slowing coat comprises two or more coats, wherein one coat comprises a delayed-release coat.

In at least one embodiment of the invention, the first once daily rate controlled-release dosage form comprising the at least one means for controllably releasing tramadol comprises at least one release-slowing coat in the form a plurality of coated cores.

In at least one embodiment of the invention, the at least one means for controllably releasing tramadol comprises at least one delayed-release coat.

In at least one embodiment of the invention, the first once daily controlled-release dosage form comprising at least one delayed release coat comprises an effective amount of tramadol for the management of moderate to moderately severe pain.

In at least one embodiment of the invention, the first once daily controlled-release dosage form comprising at least one delayed-release coat comprises from about 25 mg to about 800 mg of tramadol.

In at least one embodiment of the invention, the first once daily controlled-release dosage form comprising at least one delayed-release coat comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the first once daily controlled-release dosage form comprising at least one delayed-release coat comprises tramadol hydrochloride.

In at least one embodiment of the invention, the first once daily controlled-release dosage form comprising the at least one delayed-release coat will desirably comprise a mixture of an effective amount of at least two different tramadol salts, wherein one of the salts is tramadol hydrochloride.

In at least one embodiment of the invention, the first once daily controlled-release dosage form comprising the at least one delayed-release coat and controlled-release matrix core will desirably comprise about 70 to about 90% by weight of the core dry weight tramadol.

In at least one embodiment of the invention, the first once daily controlled-release dosage form comprising the at least one delayed-release coat will desirably comprise comprises an immediate release coat, wherein said immediate release coat comprises tramadol.

In at least one embodiment of the invention, the first once daily controlled-release dosage form comprising the at least one delayed-release coat and controlled-release matrix core comprises an immediate release coat, wherein said immediate release coat comprises a salt or form of tramadol, which is different from the salt or form of tramadol present in said controlled-release matrix core.

In at least one embodiment of the invention, the at least one means for controllably releasing tramadol comprising the at least one delayed-release coat comprises at least one controlled-release matrix core.

In at least one embodiment of the invention, the at least one means for controllably releasing tramadol comprising the at least one delayed-release coat comprises at least one erodible matrix core.

In at least one embodiment of the invention, the at least one means for controllably releasing tramadol comprising the at least one delayed-release coat comprises at least one swellable and erodible matrix core.

In at least one embodiment of the invention, the at least one means for controllably releasing tramadol comprising the at least one delayed-release coat comprises at least one swellable matrix core.

In at least one embodiment of the invention, the at least one means for controllably releasing tramadol comprising the at least one delayed-release coat comprises at least one hydrophobic matrix core.

In at least one embodiment of the invention, the at least one means for controllably releasing tramadol comprising the at least one delayed-release coat comprises at least one insoluble matrix core.

In at least one embodiment of the invention, the at least one means for controllably releasing tramadol comprising the at least one delayed-release coat comprises at least one insoluble polymer matrix core.

In at least one embodiment of the invention, the at least one means for controllably releasing tramadol comprising the at least one delayed-release coat comprises at least one release-slowing coat.

In at least one embodiment of the invention, the at least one means for controllably releasing tramadol comprising the at least one delayed-release coat comprises an osmotic subcoat.

In at least one embodiment of the invention, the at least one means for controllably releasing tramadol comprising the at least one delayed-release coat comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprising the at least one delayed-release coat comprises at least one release-slowing coat, which release slowing coat comprises at least one water-insoluble water-permeable film-forming polymer and at least one water-soluble polymer.

In at least one embodiment of the invention, the at least one means for controllably releasing tramadol comprising the at least one delayed-release coat comprises at least one release-slowing coat, which release slowing coat comprises at least one water-insoluble water-permeable film-forming polymer, at least one water-soluble polymer, and at least one plasticizer.

In at least one embodiment of the invention, the at least one means for controllably releasing tramadol comprising the at least one delayed-release coat comprises at least one enteric polymer.

In at least one embodiment of the invention, the at least one means for controllably releasing tramadol comprising the at least one delayed-release coat comprises at least one release-slowing coat, which release slowing coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one delayed-release coat comprises at least one pH dependent polymer.

In at least one embodiment of the invention, the at least one means for controllably releasing tramadol comprising the at least one delayed-release coat comprises at least one non-functional soluble coat.

In at least one embodiment of the invention, the first once daily rate controlled-release dosage form comprises at least one delayed-release coat in the form of a tablet.

In at least one embodiment of the invention, the first once daily rate controlled-release dosage form comprises at least one delayed-release coat in the form of a capsule.

In at least one embodiment of the invention, the first once daily rate controlled-release dosage form comprises at least one delayed-release coat in the form of a microparticle.

In at least one embodiment of the invention, the first once daily rate controlled-release dosage form comprises at least one delayed-release coat in the form of a plurality of microparticles, wherein each microparticle comprises at least one delayed-release coat.

In at least one embodiment of the invention, the at least one means for controllably releasing tramadol comprising the at least one delayed-release coat is comprised of a unitary core.

In at least one embodiment of the invention, the at least one means for controllably releasing tramadol comprising the at least one delayed-release coat comprises two or more coats, wherein one coat comprises a release-slowing coat.

In at least one embodiment of the invention, the first once daily rate controlled-release dosage form comprises at least one delayed-release coat in the form of a plurality of coated cores.

In at least one embodiment of the invention, the at least one means for controllably releasing tramadol comprises at least one release-slowing coat, which coat comprises at least one pH independent polymer.

In at least one embodiment of the invention, the at least one means for controllably releasing tramadol comprising the at least one release-slowing coat, which coat comprises at least one pH independent polymer, further comprises at least one controlled-release matrix core.

In at least one embodiment of the invention, the at least one means for controllably releasing tramadol comprising the at least one release-slowing coat, which coat comprises at least one pH independent polymer, further comprises at least one erodible matrix core.

In at least one embodiment of the invention, the at least one means for controllably releasing tramadol comprising the at least one release-slowing coat, which coat comprises at least one pH independent polymer, further comprises at least one swellable matrix core.

In at least one embodiment of the invention, the at least one means for controllably releasing tramadol comprising the at least one release-slowing coat, which coat comprises at least one pH independent polymer, further comprises at least one erodible and swellable matrix core.

In at least one embodiment of the invention, the at least one means for controllably releasing tramadol comprising the at least one release-slowing coat, which coat comprises at least one pH independent polymer, further comprises at least one hydrophobic matrix core.

In at least one embodiment of the invention, the at least one means for controllably releasing tramadol comprising the at least one release-slowing coat, which coat comprises at least one pH independent polymer, further comprises at least one insoluble matrix core.

In at least one embodiment of the invention, the at least one means for controllably releasing tramadol comprising the at least one release-slowing coat, which coat comprises at least one pH independent polymer, further comprises at least one insoluble polymer matrix core.

In at least one embodiment of the invention, the at least one means for controllably releasing tramadol comprising the at least one release-slowing coat, which coat comprises at least one pH independent polymer, further comprises at least one water-insoluble water-permeable film-forming polymer and at least one water-soluble polymer.

In at least one embodiment of the invention, the at least one means for controllably releasing tramadol comprising the at least one release-slowing coat, which coat comprises at least one pH independent polymer, further comprises at least one water-insoluble water-permeable film-forming polymer, at least one water-soluble polymer, and at least one plasticizer.

In at least one embodiment of the invention, the at least one means for controllably releasing tramadol comprising the least one release-slowing coat, which coat comprises at least one pH independent polymer, comprises at least one pH dependent polymer.

In at least one embodiment of the invention, the at least one means for controllably releasing tramadol comprising the least one release-slowing coat, which coat comprises at least one pH independent polymer, comprises an osmotic subcoat.

In at least one embodiment of the invention, the at least one means for controllably releasing tramadol comprising the at least one release-slowing coat, which coat comprises at least one pH independent polymer, further comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one release-slowing coat, which coat comprises at least one pH independent polymer, and comprises at least one non-functional soluble coat.

In at least one embodiment of the invention, the at least one means for controllably releasing tramadol comprising the at least one release-slowing coat, which coat comprises at least one pH independent polymer, and comprises at least one enhance absorption coat.

In at least one embodiment of the invention, the at least one means for controllably releasing tramadol comprising the at least one release-slowing coat, which coat comprises at least one pH independent polymer, and comprises at least one delayed-release coat.

In at least one embodiment of the invention, the once daily rate-controlled release dosage form comprising the at least one means for controllably releasing tramadol comprises at least one release-slowing coat, which coat comprises at least one pH independent polymer, wherein said once daily rate controlled-release dosage form is in the form of a tablet.

In at least one embodiment of the invention, the once daily rate-controlled release dosage form comprising the at least one means for controllably releasing tramadol comprises at least one release-slowing coat, which coat comprises at least one pH independent polymer, wherein said once daily rate controlled-release dosage form is in the form of a capsule.

In at least one embodiment of the invention, the once daily rate-controlled release dosage form comprising the at least one means for controllably releasing tramadol comprises at least one release-slowing coat, which coat comprises at least one pH independent polymer, wherein said once daily rate controlled-release dosage form is in the form of a microparticle.

In at least one embodiment of the invention, the once daily rate-controlled release dosage form comprising the at least one means for controllably releasing tramadol comprises at least one release-slowing coat, which coat comprises at least one pH independent polymer, wherein said once daily rate controlled-release dosage form is in the form of a plurality of microparticles, wherein each microparticle comprises at least one release-slowing coat.

In at least one embodiment of the invention, the once daily rate-controlled release dosage form comprising the at least one means for controllably releasing tramadol comprises at least one release-slowing coat, which coat comprises at least one pH independent polymer, is comprised of at least one unitary core.

In at least one embodiment of the invention, the at least one means for controllably releasing tramadol comprises at least one release-slowing coat, which coat comprises at least one pH independent polymer, comprises two or more coats, wherein one coat comprises a delayed-release coat.

In at least one embodiment of the invention, the once daily rate-controlled release dosage form comprising the at least one means for controllably releasing tramadol comprises at least one release-slowing coat, which coat comprises at least one pH independent polymer, wherein said once daily rate controlled-release dosage form is in the form of a plurality of coated cores.

In at least one embodiment of the invention, the at least one means for controllably releasing tramadol comprises at least one release-slowing coat comprising at least one pH dependent polymer (such as for example enteric or reverse enteric types).

In at least one embodiment of the invention, the at least one means for controllably releasing tramadol comprising the at least one release-slowing coat, which coat comprises at least one pH dependent polymer, further comprises at least one controlled-release matrix core.

In at least one embodiment of the invention, the at least one means for controllably releasing tramadol comprising the at least one release-slowing coat, which coat comprises at least one pH dependent polymer, further comprises at least one erodable matrix core.

In at least one embodiment of the invention, the at least one means for controllably releasing tramadol comprising the at least one release-slowing coat, which coat comprises at least one pH dependent polymer, further comprises at least one swellable matrix core.

In at least one embodiment of the invention, the at least one means for controllably releasing tramadol comprising the at least one release-slowing coat, which coat comprises at least one pH dependent polymer, further comprises at least one erodible and swellable matrix core.

In at least one embodiment of the invention, the at least one means for controllably releasing tramadol comprising the at least one release-slowing coat, which coat comprises at least one pH dependent polymer, further comprises at least one insoluble matrix core.

In at least one embodiment of the invention, the at least one means for controllably releasing tramadol comprising the at least one release-slowing coat, which coat comprises at least one pH dependent polymer, further comprises at least one hydrophobic matrix core.

In at least one embodiment of the invention, the at least one means for controllably releasing tramadol comprising the at least one release-slowing coat, which coat comprises at least one pH dependent polymer, further comprises at least one insoluble polymer matrix core.

In at least one embodiment of the invention, the at least one means for controllably releasing tramadol comprising the at least one release-slowing coat, which coat comprises at least one pH dependent polymer, further comprises at least one water-insoluble water-permeable film-forming polymer and at least one water-soluble polymer.

In at least one embodiment of the invention, the at least one means for controllably releasing tramadol comprising the at least one release-slowing coat, which coat comprises at least one pH dependent polymer, further comprises at least one water-insoluble water-permeable film-forming polymer, at least one water-soluble polymer, and at least one plasticizer.

In at least one embodiment of the invention, the at least one means for controllably releasing tramadol comprising the at least one release-slowing coat, which coat comprises at least one pH dependent polymer, further comprises an osmotic subcoat.

In at least one embodiment of the invention, the at least one means for controllably releasing tramadol comprising the least one release-slowing coat, which coat comprises at least one pH dependent polymer, comprises at least one wherein said pH independent polymer.

In at least one embodiment of the invention, the at least one means for controllably releasing tramadol comprising the at least one release-slowing coat, which coat comprises at least one pH dependent polymer, further comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one release-slowing coat, which coat comprises at least one pH dependent polymer, and comprises at least one non-functional soluble coat.

In at least one embodiment of the invention, the at least one means for controllably releasing tramadol comprising the at least one release-slowing coat, which coat comprises at least one pH dependent polymer, and comprises at least one enhanced-absorption coat.

In at least one embodiment of the invention, the at least one means for controllably releasing tramadol comprising the at least one release-slowing coat, which coat comprises at least one pH dependent polymer, and comprises at least one delayed-release coat.

In at least one embodiment of the invention, the once daily rate-controlled release dosage form comprising the at least one means for controllably releasing tramadol comprises at least one release-slowing coat, which coat comprises at least one pH dependent polymer, wherein said once daily rate controlled-release dosage form is in the form of a tablet.

In at least one embodiment of the invention, the once daily rate-controlled release dosage form comprising the at least one means for controllably releasing tramadol comprises at least one release-slowing coat, which coat comprises at least one pH dependent polymer, wherein said once daily rate controlled-release dosage form is in the form of a capsule.

In at least one embodiment of the invention, the once daily rate-controlled release dosage form comprising the at least one means for controllably releasing tramadol comprises at least one release-slowing coat, which coat comprises at least one pH dependent polymer, wherein said once daily rate controlled-release dosage form is in the form of a microparticle.

In at least one embodiment of the invention, the once daily rate-controlled release dosage form comprising the at least one means for controllably releasing tramadol comprises at least one release-slowing coat, which coat comprises at least one pH dependent polymer, wherein said once daily rate controlled-release dosage form is in the form of a plurality of microparticles, wherein each microparticle comprises at least one release-slowing coat.

In at least one embodiment of the invention, the once daily rate-controlled release dosage form comprising the at least one means for controllably releasing tramadol comprises at least one release-slowing coat, which coat comprises at least one pH dependent polymer, is comprised of at least one unitary core.

In at least one embodiment of the invention, the at least one means for controllably releasing tramadol comprises at least one release-slowing coat, which coat comprises at least one pH dependent polymer, comprises two or more coats, wherein one coat comprises a delayed-release coat.

In at least one embodiment of the invention, the once daily rate-controlled release dosage form comprising the at least one means for controllably releasing tramadol comprises at least one release-slowing coat, which coat comprises at least one pH dependent polymer, wherein said once daily rate controlled-release dosage form is in the form of a plurality of coated cores.

In at least one embodiment of the invention, the at least one means for controllably releasing tramadol comprises at least one release-slowing, which coat comprises at least one soluble polymer.

In at least one embodiment of the invention, the at least one means for controllably releasing tramadol comprising the at least one release-slowing coat, which coat comprises at least one soluble polymer, further comprises at least one controlled-release matrix core.

In at least one embodiment of the invention, the at least one means for controllably releasing tramadol comprising the at least one release-slowing coat, which coat comprises at least one soluble polymer, further comprises at least one erodible matrix core.

In at least one embodiment of the invention, the at least one means for controllably releasing tramadol comprising the at least one release-slowing coat, which coat comprises at least one soluble polymer, further comprises at least one swellable matrix core.

In at least one embodiment of the invention, the at least one means for controllably releasing tramadol comprising the at least one release-slowing coat, which coat comprises at least one soluble polymer, further comprises at least one erodible and swellable matrix core.

In at least one embodiment of the invention, the at least one means for controllably releasing tramadol comprising the at least one release-slowing coat, which coat comprises at least one soluble polymer, further comprises at least one insoluble matrix core.

In at least one embodiment of the invention, the at least one means for controllably releasing tramadol comprising the at least one release-slowing coat, which coat comprises at least one soluble polymer, further comprises at least one hydrophobic matrix core.

In at least one embodiment of the invention, the at least one means for controllably releasing tramadol comprising the at least one release-slowing coat, which coat comprises at least one soluble polymer, further comprises at least one insoluble polymer matrix core.

In at least one embodiment of the invention, the at least one means for controllably releasing tramadol comprising the at least one release-slowing coat, which coat comprises at least one soluble polymer, further comprises at least one water-insoluble water-permeable film-forming polymer.

In at least one embodiment of the invention, the at least one means for controllably releasing tramadol comprising the at least one release-slowing coat, which coat comprises at least one soluble polymer, further comprises at least one water-insoluble water-permeable film-forming polymer and at least one plasticizer.

In at least one embodiment of the invention, the at least one means for controllably releasing tramadol comprising the least one release-slowing coat, which coat comprises at least one soluble polymer, comprises at least one pH dependent polymer.

In at least one embodiment of the invention, the at least one means for controllably releasing tramadol comprising the at least one release-slowing coat, which coat comprises at least one soluble polymer, further comprises an osmotic subcoat.

In at least one embodiment of the invention, the at least one means for controllably releasing tramadol comprising the at least one release-slowing coat, which coat comprises at least one soluble polymer, further comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one release-slowing coat, which coat comprises at least one soluble polymer, and comprises at least one non-functional soluble coat.

In at least one embodiment of the invention, the at least one means for controllably releasing tramadol comprising the at least one release-slowing coat, which coat comprises at least one soluble polymer, and comprises at least one delayed release coat.

In at least one embodiment of the invention, the once daily rate-controlled release dosage form comprising the at least one means for controllably releasing tramadol comprises at least one release-slowing coat, which coat comprises at least one soluble polymer, wherein said once daily rate controlled-release dosage form is in the form of a tablet.

In at least one embodiment of the invention, the once daily rate-controlled release dosage form comprising the at least one means for controllably releasing tramadol comprises at least one release-slowing coat, which coat comprises at least one soluble polymer, wherein said once daily rate controlled-release dosage form is in the form of a capsule.

In at least one embodiment of the invention, the once daily rate-controlled release dosage form comprising the at least one means for controllably releasing tramadol comprises at least one release-slowing coat, which coat comprises at least one soluble polymer, wherein said once daily rate controlled-release dosage form is in the form of a microparticle.

In at least one embodiment of the invention, the once daily rate-controlled release dosage form comprising the at least one means for controllably releasing tramadol comprises at least one release-slowing coat, which coat comprises at least one soluble polymer, wherein said once daily rate controlled-release dosage form is in the form of a plurality of microparticles, wherein each microparticle comprises at least one release-slowing coat.

In at least one embodiment of the invention, the once daily rate-controlled release dosage form comprising the at least one means for controllably releasing tramadol comprises at least one release-slowing coat, which coat comprises at least one soluble polymer, is comprised of at least one unitary core.

In at least one embodiment of the invention, the at least one means for controllably releasing tramadol comprises at least one release-slowing coat, which coat comprises at least one soluble polymer, comprises two or more coats, wherein one coat comprises a delayed-release coat.

In at least one embodiment of the invention, the once daily rate-controlled release dosage form comprising the at least one means for controllably releasing tramadol comprises at least one release-slowing coat, which coat comprises at least one soluble polymer, wherein said once daily rate controlled-release dosage form is in the form of a plurality of coated cores.

In at least one embodiment of the invention, the at least one means for controllably releasing tramadol comprises at least one release-slowing coat comprising at least one insoluble polymer (aqueous insoluble coat).

In at least one embodiment of the invention, the at least one means for controllably releasing tramadol comprising the at least one release-slowing coat, which coat comprises at least one insoluble polymer, further comprises at least one controlled-release matrix core.

In at least one embodiment of the invention, the at least one means for controllably releasing tramadol comprising the at least one release-slowing coat, which coat comprises at least one insoluble polymer, further comprises at least one erodible matrix core.

In at least one embodiment of the invention, the at least one means for controllably releasing tramadol comprising the at least one release-slowing coat, which coat comprises at least one insoluble polymer, further comprises at least one swellable matrix core.

In at least one embodiment of the invention, the at least one means for controllably releasing tramadol comprising the at least one release-slowing coat, which coat comprises at least one insoluble polymer, further comprises at least one erodible and swellable matrix core.

In at least one embodiment of the invention, the at least one means for controllably releasing tramadol comprising the at least one release-slowing coat, which coat comprises at least one insoluble polymer, further comprises at least one insoluble matrix core.

In at least one embodiment of the invention, the at least one means for controllably releasing tramadol comprising the at least one release-slowing coat, which coat comprises at least one insoluble polymer, further comprises at least one hydrophobic matrix core.

In at least one embodiment of the invention, the at least one means for controllably releasing tramadol comprising the at least one release-slowing coat, which coat comprises at least one insoluble polymer, further comprises at least one insoluble polymer matrix core.

In at least one embodiment of the invention, the at least one means for controllably releasing tramadol comprising the at least one release-slowing coat, which coat comprises at least one insoluble polymer, further comprises at least one water-insoluble water-permeable film-forming polymer.

In at least one embodiment of the invention, the at least one means for controllably releasing tramadol comprising the at least one release-slowing coat, which coat comprises at least one insoluble polymer, further comprises at least one water-insoluble water-permeable film-forming polymer and at least one plasticizer.

In at least one embodiment of the invention, the at least one means for controllably releasing tramadol comprising the least one release-slowing coat, which coat comprises at least one insoluble polymer, comprises at least one pH dependent polymer.

In at least one embodiment of the invention, the at least one means for controllably releasing tramadol comprising the at least one release-slowing coat, which coat comprises at least one insoluble polymer, further comprises an osmotic subcoat.

In at least one embodiment of the invention, the at least one means for controllably releasing tramadol comprising the at least one release-slowing coat, which coat comprises at least one insoluble polymer, further comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one release-slowing coat, which coat comprises at least one insoluble polymer, and comprises at least one non-functional soluble coat.

In at least one embodiment of the invention, the at least one means for controllably releasing tramadol comprising the at least one release-slowing coat, which coat comprises at least one insoluble polymer, and comprises at least one delayed-release coat.

In at least one embodiment of the invention, the once daily rate-controlled release dosage form comprising the at least one means for controllably releasing tramadol comprises at least one release-slowing coat, which coat comprises at least one insoluble polymer, wherein said once daily rate controlled-release dosage form is in the form of a tablet.

In at least one embodiment of the invention, the once daily rate-controlled release dosage form comprising the at least one means for controllably releasing tramadol comprises at least one release-slowing coat, which coat comprises at least one insoluble polymer, wherein said once daily rate controlled-release dosage form is in the form of a capsule.

In at least one embodiment of the invention, the once daily rate-controlled release dosage form comprising the at least one means for controllably releasing tramadol comprises at least one release-slowing coat, which coat comprises at least one insoluble polymer, wherein said once daily rate controlled-release dosage form is in the form of a microparticle.

In at least one embodiment of the invention, the once daily rate-controlled release dosage form comprising the at least one means for controllably releasing tramadol comprises at least one release-slowing coat, which coat comprises at least one insoluble polymer, wherein said once daily rate controlled-release dosage form is in the form of a plurality of microparticles, wherein each microparticle comprises at least one release-slowing coat.

In at least one embodiment of the invention, the once daily rate-controlled release dosage form comprising the at least one means for controllably releasing tramadol comprises at least one release-slowing coat, which coat comprises at least one insoluble polymer, is comprised of at least one unitary core.

In at least one embodiment of the invention, the at least one means for controllably releasing tramadol comprises at least one release-slowing coat, which coat comprises at least one insoluble polymer, comprises two or more coats, wherein one coat comprises a delayed-release coat.

In at least one embodiment of the invention, the once daily rate-controlled release dosage form comprising the at least one means for controllably releasing tramadol comprises at least one release-slowing coat, which coat comprises at least one insoluble polymer, wherein said once daily rate controlled-release dosage form is in the form of a plurality of coated cores.

In at least one embodiment of the invention, the at least one means for controllably releasing tramadol comprises at least one release-slowing coat, which coat comprises at least one swellable polymer.

In at least one embodiment of the invention, the at least one means for controllably releasing tramadol comprising the at least one release-slowing coat, which coat comprises at least one swellable polymer, further comprises at least one controlled-release matrix core.

In at least one embodiment of the invention, the at least one means for controllably releasing tramadol comprising the at least one release-slowing coat, which coat comprises at least one swellable polymer, further comprises at least one erodable matrix core.

In at least one embodiment of the invention, the at least one means for controllably releasing tramadol comprising the at least one release-slowing coat, which coat comprises at least one swellable polymer, further comprises at least one erodible and swellable matrix core.

In at least one embodiment of the invention, the at least one means for controllably releasing tramadol comprising the at least one release-slowing coat, which coat comprises at least one swellable polymer, further comprises at least one swellable matrix core.

In at least one embodiment of the invention, the at least one means for controllably releasing tramadol comprising the at least one release-slowing coat, which coat comprises at least one swellable polymer, further comprises at least one insoluble matrix core.

In at least one embodiment of the invention, the at least one means for controllably releasing tramadol comprising the at least one release-slowing coat, which coat comprises at least one swellable polymer, further comprises at least one hydrophobic matrix core.

In at least one embodiment of the invention, the at least one means for controllably releasing tramadol comprising the at least one release-slowing coat, which coat comprises at least one swellable polymer, further comprises at least one insoluble polymer matrix core.

In at least one embodiment of the invention, the at least one means for controllably releasing tramadol comprising the at least one release-slowing coat, which coat comprises at least one swellable polymer, further comprises at least one water-insoluble water-permeable film-forming polymer and at least one water-soluble polymer.

In at least one embodiment of the invention, the at least one means for controllably releasing tramadol comprising the at least one release-slowing coat, which coat comprises at least one swellable polymer, further comprises at least one water-insoluble water-permeable film-forming polymer, at least one water-soluble polymer, and at least one plasticizer.

In at least one embodiment of the invention, the at least one means for controllably releasing tramadol comprising the at least one release-slowing coat, which coat comprises at least one swellable polymer, further comprises an osmotic subcoat.

In at least one embodiment of the invention, the at least one means for controllably releasing tramadol comprising the least one release-slowing coat, which coat comprises at least one swellable polymer, comprises at least one pH independent polymer.

In at least one embodiment of the invention, the at least one means for controllably releasing tramadol comprising the at least one release-slowing coat, which coat comprises at least one swellable polymer, further comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one release-slowing coat, which coat comprises at least one swellable polymer, and comprises at least one non-functional soluble coat.

In at least one embodiment of the invention, the at least one means for controllably releasing tramadol comprising the at least one release-slowing coat, which coat comprises at least one swellable polymer, and comprises at least one delayed-release coat.

In at least one embodiment of the invention, the once daily rate-controlled release dosage form comprising the at least one means for controllably releasing tramadol comprises at least one release-slowing coat, which coat comprises at least one swellable polymer, wherein said once daily rate controlled-release dosage form is in the form of a tablet.

In at least one embodiment of the invention, the once daily rate-controlled release dosage form comprising the at least one means for controllably releasing tramadol comprises at least one release-slowing coat, which coat comprises at least one swellable polymer, wherein said once daily rate controlled-release dosage form is in the form of a capsule.

In at least one embodiment of the invention, the once daily rate-controlled release dosage form comprising the at least one means for controllably releasing tramadol comprises at least one release-slowing coat, which coat comprises at least one swellable polymer, wherein said once daily rate controlled-release dosage form is in the form of a microparticle.

In at least one embodiment of the invention, the once daily rate-controlled release dosage form comprising the at least one means for controllably releasing tramadol comprises at least one release-slowing coat, which coat comprises at least one swellable polymer, wherein said once daily rate controlled-release dosage form is in the form of a plurality of microparticles, wherein each microparticle comprises at least one release-slowing coat.

In at least one embodiment of the invention, the once daily rate-controlled release dosage form comprising the at least one means for controllably releasing tramadol comprises at least one release-slowing coat, which coat comprises at least one swellable polymer, is comprised of at least one unitary core.

In at least one embodiment of the invention, the at least one means for controllably releasing tramadol comprises at least one release-slowing coat, which coat comprises at least one swellable polymer, comprises two or more coats, wherein one coat comprises a delayed-release coat.

In at least one embodiment of the invention, the once daily rate-controlled release dosage form comprising the at least one means for controllably releasing tramadol comprises at least one release-slowing coat, which coat comprises at least one swellable polymer, wherein said once daily rate controlled-release dosage form is in the form of a plurality of coated cores.

In at least one embodiment of the invention, the at least one means for controllably releasing tramadol comprises at least one release-slowing coat, which coat comprises at least one hydrophobic material.

In at least one embodiment of the invention, the at least one means for controllably releasing tramadol comprising the at least one release-slowing coat, which coat comprises at least one hydrophobic material, further comprises at least one controlled-release matrix core.

In at least one embodiment of the invention, the at least one means for controllably releasing tramadol comprising the at least one release-slowing coat, which coat comprises at least one hydrophobic material, further comprises at least one erodable matrix core.

In at least one embodiment of the invention, the at least one means for controllably releasing tramadol comprising the at least one release-slowing coat, which coat comprises at least one hydrophobic material, further comprises at least one swellable matrix core.

In at least one embodiment of the invention, the at least one means for controllably releasing tramadol comprising the at least one release-slowing coat, which coat comprises at least one hydrophobic material, further comprises at least one erodible and swellable matrix core.

In at least one embodiment of the invention, the at least one means for controllably releasing tramadol comprising the at least one release-slowing coat, which coat comprises at least one hydrophobic material, further comprises at least one insoluble matrix core.

In at least one embodiment of the invention, the at least one means for controllably releasing tramadol comprising the at least one release-slowing coat, which coat comprises at least one hydrophobic material, further comprises at least one hydrophobic matrix core.

In at least one embodiment of the invention, the at least one means for controllably releasing tramadol comprising the at least one release-slowing coat, which coat comprises at least one hydrophobic material, further comprises at least one insoluble polymer matrix core.

In at least one embodiment of the invention, the at least one means for controllably releasing tramadol comprising the at least one release-slowing coat, which coat comprises at least one hydrophobic material, further comprises at least one water-insoluble water-permeable film-forming polymer and at least one water-soluble polymer.

In at least one embodiment of the invention, the at least one means for controllably releasing tramadol comprising the at least one release-slowing coat, which coat comprises at least one hydrophobic material, further comprises at least one water-insoluble water-permeable film-forming polymer, at least one water-soluble polymer, and at least one plasticizer.

In at least one embodiment of the invention, the at least one means for controllably releasing tramadol comprising the at least one release-slowing coat, which coat comprises at least one hydrophobic material, further comprises an osmotic subcoat.

In at least one embodiment of the invention, the at least one means for controllably releasing tramadol comprising the least one release-slowing coat, which coat comprises at least one hydrophobic material, comprises at least one pH independent polymer.

In at least one embodiment of the invention, the at least one means for controllably releasing tramadol comprising the at least one release-slowing coat, which coat comprises at least one hydrophobic material, further comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprising the at least one release-slowing coat, which coat comprises at least one hydrophobic material, and comprises at least one non-functional soluble coat.

In at least one embodiment of the invention, the at least one means for controllably releasing tramadol comprising the at least one release-slowing coat, which coat comprises at least one hydrophobic material, and comprises at least one delayed-release coat.

In at least one embodiment of the invention, the once daily rate-controlled release dosage form comprising the at least one means for controllably releasing tramadol comprises at least one release-slowing coat, which coat comprises at least one hydrophobic material, wherein said once daily rate controlled-release dosage form is in the form of a tablet.

In at least one embodiment of the invention, the once daily rate-controlled release dosage form comprising the at least one means for controllably releasing tramadol comprises at least one release-slowing coat, which coat comprises at least one hydrophobic material, wherein said once daily rate controlled-release dosage form is in the form of a capsule.

In at least one embodiment of the invention, the once daily rate-controlled release dosage form comprising the at least one means for controllably releasing tramadol comprises at least one release-slowing coat, which coat comprises at least one hydrophobic material, wherein said once daily rate controlled-release dosage form is in the form of a microparticle.

In at least one embodiment of the invention, the once daily rate-controlled release dosage form comprising the at least one means for controllably releasing tramadol comprises at least one release-slowing coat, which coat comprises at least one hydrophobic material, wherein said once daily rate controlled-release dosage form is in the form of a plurality of microparticles, wherein each microparticle comprises at least one release-slowing coat.

In at least one embodiment of the invention, the once daily rate-controlled release dosage form comprising the at least one means for controllably releasing tramadol comprises at least one release-slowing coat, which coat comprises at least one hydrophobic material, is comprised of at least one unitary core.

In at least one embodiment of the invention, the at least one means for controllably releasing tramadol comprises at least one release-slowing coat, which coat comprises at least one hydrophobic material, comprises two or more coats, wherein one coat comprises a delayed-release coat.

In at least one embodiment of the invention, the once daily rate-controlled release dosage form comprising the at least one means for controllably releasing tramadol comprises at least one release-slowing coat, which coat comprises at least one hydrophobic material, wherein said once daily rate controlled-release dosage form is in the form of a plurality of coated cores.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form.

In at least one embodiment of the invention, the at least one means for controllably releasing tramadol comprising the at least one means for the exit of tramadol from the first once daily rate controlled-release dosage form, the at least one means for increasing the hydrostatic pressure of the first once daily controlled-release dosage form, and the at least one means for forcibly dispensing tramadol from the first once daily controlled-release dosage comprises an effective amount of tramadol for the management of moderate to moderately severe pain.

In at least one embodiment of the invention, the at least one means for controllably releasing tramadol comprising the at least one means for the exit of tramadol from the first once daily rate controlled-release dosage form, the at least one means for increasing the hydrostatic pressure of the first once daily controlled-release dosage form, and the at least one means for forcibly dispensing tramadol from the first once daily controlled-release dosage comprises from about 25 mg to about 800 mg of tramadol.

In at least one embodiment of the invention, the at least one means for controllably releasing tramadol comprising the at least one means for the exit of tramadol from the first once daily rate controlled-release dosage form, the at least one means for increasing the hydrostatic pressure of the first once daily controlled-release dosage form, and the at least one means for forcibly dispensing tramadol from the first once daily controlled-release dosage comprises about 25, about 50, about 75, about 100, about 125, about 150, about 175, about 200, about 225, about 250, about 275, about 300, about 325, about 350, about 375, about 400, about 425, about 450, about 475, about 500, about 525, 550, about 575, about 600, about 625, about 650, about 675, about 700, about 725, about 750, about 775, or about 800 mg of tramadol.

In at least one embodiment of the invention, the at least one means for controllably releasing tramadol comprising the at least one means for the exit of tramadol from the first once daily rate controlled-release dosage form, the at least one means for increasing the hydrostatic pressure of the first once daily controlled-release dosage form, and the at least one means for forcibly dispensing tramadol from the first once daily controlled-release dosage comprises tramadol hydrochloride.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least one release-slowing coat.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least one delayed-release coat.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least one release-slowing coat, which coat comprises at least one water-insoluble water-permeable film-forming polymer and at least one water-soluble polymer.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least one release-slowing coat, which coat comprises at least one water-insoluble water-permeable film-forming polymer, at least one water-soluble polymer, and at least one plasticizer.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least one release-slowing coat, which coat comprises at least one enteric polymer.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least delayed-release coat, which coat comprises at least one pH dependent polymer.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form comprises at least one non-functional soluble coat.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form is in the form of a tablet.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form is in the form of a capsule.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form is in the form of a microparticle.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form is comprised of a plurality of microparticles, wherein each microparticle comprises the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form is comprised of at least one unitary core.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form further comprises two or more coats, wherein one coat comprises a release-slowing coat.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form further comprises two or more coats, wherein one coat comprises a delayed-release coat.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form is comprised of a plurality of coated cores.

In at least one embodiment of the invention, the at least one means for controllably releasing tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form further comprises an osmotic subcoat.

“Controlled Release Dosage Form” Embodiments

In at least one embodiment, alternative once daily controlled-release dosage forms of the invention comprising tramadol will desirably comprise a first once daily controlled-release dosage form such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and will desirably be bioequivalent in the fed or fasted state according to FDA guidelines to a second orally administrable dosage form comprising the same dose of tramadol also suitable for once daily administration in the fed or fasted state.

In at least one embodiment, alternative once daily controlled-release dosage forms of the invention comprising tramadol will desirably comprise a first once daily controlled-release dosage form such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, has reduced potential for alcohol induced dose dumping and will desirably be bioequivalent in the fed or fasted state according to FDA guidelines to a second orally administrable dosage form comprising the same dose of tramadol also suitable for once daily administration in the fed or fasted state.

In at least one embodiment, alternative once daily controlled-release dosage forms of the invention comprising tramadol will desirably comprise a first once daily controlled-release dosage form such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, and will desirably be bioequivalent according to FDA guidelines to a second orally administrable dosage form comprising the same dose of tramadol also suitable for once daily administration in the fed state.

In at least one embodiment, alternative once daily controlled-release dosage forms of the invention comprising tramadol will desirably comprise a first once daily controlled-release dosage form such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol, and when said first once daily controlled-release dosage form is administered to a patient in need of such administration will desirably exhibit following single-dose administration: (i) a Cₘₐₓ of from about 75 to about 338 ng/ml of the tramadol, (ii) an AUC_{0-∞} of from about 2725 to about 7581 ng·hr/ml of tramadol under fed conditions, has reduced potential for alcohol induced dose dumping and will desirably be bioequivalent according to FDA guidelines to a second orally administrable dosage form comprising the same dose of tramadol also suitable for once daily administration in the fed state.

In at least one embodiment, alternative once daily controlled-release dosage forms of the invention comprising tramadol will desirably comprise a first once daily controlled-release dosage form such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol, and when said first once daily controlled-release dosage form is administered to a patient in need of such administration will desirably exhibit following single-dose administration a Cₘₐₓ of, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335 or about 338 ng/ml of the tramadol under fed conditions and will desirably be bioequivalent according to FDA guidelines to a second orally administrable dosage form comprising the same dose of tramadol also suitable for once daily administration in the fed state.

In at least one embodiment, alternative once daily controlled-release dosage forms of the invention comprising tramadol will desirably comprise a first once daily controlled-release dosage form such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol, and when said first once daily controlled-release dosage form is administered to a patient in need of such administration will desirably exhibit following single-dose administration an AUC_{0-∞} of, for example, about 2725, about 2750, about 2900, about 3050, about 3200, about 3350, about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions and will desirably be bioequivalent according to FDA guidelines to a second orally administrable dosage form comprising the same dose of tramadol also suitable for once daily administration in the fed state.

In at least one embodiment, alternative once daily controlled-release dosage forms of the invention comprising tramadol will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol, and when said first once daily controlled-release dosage form is administered once daily to a patient in need of such administration will desirably exhibit following single-dose administration: (i) a Cₘₐₓ of from about 180 to about 333 ng/ml of tramadol, (ii) an AUC_{0-∞} of from about 3740 to about 7600 ng·hr/ml of tramadol under fasting conditions, and will desirably be bioequivalent according to FDA guidelines to a second orally administrable dosage form comprising the same dose of tramadol also suitable for once daily administration in the fasted state.

In at least one embodiment, alternative once daily controlled-release dosage forms of the invention comprising tramadol will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol, and when said first once daily controlled-release dosage form is administered once daily to a patient in need of such administration will desirably exhibit following single-dose administration: (i) a Cₘₐₓ of from about 180 to about 333 ng/ml of tramadol, (ii) an AUC_{0-∞} of from about 3740 to about 7600 ng·hr/ml of tramadol under fasting conditions, has reduced potential for alcohol induced dose dumping and will desirably be bioequivalent according to FDA guidelines to a second orally administrable dosage form comprising the same dose of tramadol also suitable for once daily administration in the fasted state.

In at least one embodiment, alternative once daily controlled-release dosage forms of the invention comprising tramadol will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol, and when said first once daily controlled-release dosage form is administered once daily to a patient in need of such administration will desirably exhibit following single-dose administration a Cₘₐₓ of, for example, about 180, about 190, about 200, about 210, about 220, about 230, about 240, about 250, about 260, about 270, about 280, about 290, about 300, about 310, about 320, about 330, or about 333 ng/ml of tramadol under fasting conditions and will desirably be bioequivalent according to FDA guidelines to a second orally administrable dosage form comprising the same dose of tramadol also suitable for once daily administration in the fasted state.

In at least one embodiment, alternative once daily controlled-release dosage forms of the invention comprising tramadol will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol, and when said first once daily controlled-release dosage form is administered once daily to a patient in need of such administration will desirably exhibit following single-dose administration an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550 about, 4600, about 4650, about 4700, about 4750, about 4800, about 4850, about 4900, about 4950, about 5000, about 5050, about 5100, about 5150, about 5200, about 5250, about 5300, about 5350, about 5400, about 5450, 5500, 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600 ng·hr/ml of tramadol under fasting conditions and will desirably be bioequivalent according to FDA guidelines to a second orally administrable dosage form comprising the same dose of tramadol also suitable for once daily administration in the fasted state.

In at least one embodiment of the invention, the first once daily controlled-release dosage form will desirably exhibit in the fed state a Tₘₐₓ of tramadol from 4 to 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr following single-dose administration and will desirably be bioequivalent according to FDA guidelines to the second orally administrable dosage form comprising the same dose of tramadol also suitable for once daily administration.

In at least one embodiment of the invention, the first once daily controlled-release dosage form will desirably further exhibit at steady state the following pharmacokinetic parameters in-vivo under fasting conditions: (i) an AUC₀₋₂₄ from about 1635 to about 21000, for example, from about 1635 to about 3920, about 3610 to about 9120, or about 9455 to about 20965 ng·h/ml, and (ii) a Cₘₐₓ from about 117 to about 1230, for example, about 117 to about 245, about 230 to about 590, or about 590 to about 1230 ng/ml and will desirably be bioequivalent according to FDA guidelines to the second orally administrable dosage form comprising tramadol also suitable for once daily administration.

In at least one embodiment of the invention, the first once daily controlled-release dosage form will desirably exhibit under fasting conditions a Tₘₐₓ of, for example, about 9, about 10, about 11, about 12, about 13, or about 14 hours at steady state and will desirably be bioequivalent according to FDA guidelines to the second orally administrable dosage form comprising the same dose of tramadol also suitable for once daily administration.

In at least one embodiment of the invention, the first once daily controlled-release dosage form will desirably exhibit under fasting conditions a degree of fluctuation (%) of, for example, about 43 to about 141, about 43 to about 120, about 58 to about 132, or about 57 to about 141 at steady state and will desirably be bioequivalent according to FDA guidelines to the second orally administrable dosage form comprising the same dose of tramadol also suitable for once daily administration.

In at least one embodiment of the invention, the first once daily controlled-release dosage form will desirably exhibit under fasting conditions a Cₘᵢₙ of, for example, from about 31 to about 652, about 31 to about 117, about 96 to about 241, or about 226 to about 652 ng/ml at steady state and will desirably be bioequivalent according to FDA guidelines to the second orally administrable dosage form comprising the same dose of tramadol also suitable for once daily administration.

In at least one embodiment of the invention, the first once daily controlled-release dosage form will desirably exhibit under fasting conditions a Tₘₐₓ of, for example, 9, 10, 11, 12, 13, or 14 hours, a degree of fluctuation (%) of, for example, about 43 to about 141, about 43 to about 120, about 58 to about 132, or about 57 to about 141, and a Cₘₐₓ of, for example, from about 31 to about 652, about 31 to about 117, about 96 to about 241, or about 226 to about 652 ng/ml at steady state and will desirably be bioequivalent according to FDA guidelines to the second orally administrable dosage form comprising the same dose of tramadol also suitable for once daily administration.

In at least one embodiment of the invention, the first once daily controlled-release dosage form will desirably exhibit following single-dose administration a Tₘₐₓ of tramadol from about 10 to about 20 hr, for example, about 10, about 11, about 12, about 13, about 14, about 15, about 16, about 17, about 18, about 19, or about 20 hr in the fasting state and be bioequivalent according to FDA guidelines to the second orally administrable dosage form comprising the same dose tramadol also suitable for once daily administration.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and will desirably be bioequivalent according to FDA guidelines to a second orally administrable dosage form comprising the same dose of tramadol also suitable for once daily administration in the fed state or fasted.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, has reduced potential for alcohol induced dose dumping and will desirably be bioequivalent according to FDA guidelines to a second orally administrable dosage form comprising the same dose of tramadol also suitable for once daily administration in the fed or fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, has reduced potential for alcohol induced dose dumping and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration a Cₘₐₓ of, for example about 75, about 80, about 85, about 90, about 95, about 100, about 105, about 110, about 115, about 120, about 125, about 130, about 135, 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335 or about 338 ng/ml of tramadol under fed conditions and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration an AUC_{0-∞} of from about 2725 to about 7581 ng·hr/ml, for example, about 2725, about 2750, about 2900, about 3050, about 3200, about 3350 about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol, after about 4 hours from about 5 to about 30% by weight of tramadol, after about 6 hours, from about 15 to about 38% by weight of tramadol, and after about 8 hours, more than about 40% by weight of tramadol is released from the first once daily controlled-release dosage form, and when said first once daily controlled-release dosage form is administered to the human in need of such administration will desirably exhibit: (i) a Cₘₐₓ of from about 180 to about 333 ng/ml of tramadol, (ii) an AUC_{0-∞} of from about 3740 to about 7600 ng·hr/ml of tramadol under fasting conditions, and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol, after about 4 hours from about 5 to about 30% by weight of tramadol, after about 6 hours, from about 15 to about 38% by weight of tramadol, and after about 8 hours, more than about 40% by weight of tramadol is released from the first once daily controlled-release dosage form, and when said first once daily controlled-release dosage form is administered to the human in need of such administration will desirably exhibit: (i) a Cₘₐₓ of from about 180 to about 333 ng/ml of tramadol, (ii) an AUC_{0-∞} of from about 3740 to about 7600 ng·hr/ml of tramadol under fasting conditions, has reduced potential for alcohol induced dose dumping and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol, after about 4 hours from about 5 to about 30% by weight of tramadol, after about 6 hours, from about 15 to about 38% by weight of tramadol, and after about 8 hours, more than about 40% by weight of tramadol is released from the first once daily controlled-release dosage form, and when said first once daily controlled-release dosage form is administered to the human in need of such administration will desirably exhibit a Cₘₐₓ of from about 180 to about 333 ng/ml under fasting conditions and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form, wherein said first once daily controlled-release dosage form will desirably exhibit an in-vitro release rate such that after about 2 hours from about 0 to about 22% by weight of tramadol, after about 4 hours from about 5 to about 30% by weight of tramadol, after about 6 hours, from about 15 to about 38% by weight of tramadol, and after about 8 hours, more than about 40% by weight of tramadol is released from the first once daily controlled-release dosage form, and when said first once daily controlled-release dosage form is administered to the human in need of such administration will desirably exhibit an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550 about, 4600, about 4650, about 4700, about 4750, about 4800, about 4850, about 4900, about 4950, about 5000, about 5050, about 5100, about 5150, about 5200, about 5250, about 5300, about 5350, about 5400, about 5450, 5500, 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600 ng·hr/ml of tramadol under fasting conditions and will desirably be bioequivalent according to FDA guidelines to a second orally administrable dosage form comprising the same dose of tramadol also suitable for once daily administration in the fasted state.

In at least one embodiment of the invention where the first once daily controlled-release dosage form is directed to a method for administering a therapeutically effective amount of tramadol to a human for the treatment or management of moderately to moderately severe pain, the once daily controlled-release dosage form will desirably further exhibit in the fed state a Tₘₐₓ of tramadol from 4 to 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr after single-dose administration and will desirably be bioequivalent according to FDA guidelines to a second orally administrable dosage form comprising the same dose of tramadol also suitable for once daily administration.

In at least one embodiment of the invention where the first once daily controlled-release dosage form is directed to a method for administering a therapeutically effective amount of tramadol to a human for the treatment or management of moderately to moderately severe pain, the once daily controlled-release dosage form will desirably further exhibit at steady state the following pharmacokinetic parameters in-vivo under fasting conditions: (i) an AUC₀₋₂₄ from 1635 to 21000, for example, from 1635 to 3920, 3610 to 9120, or 9455 to 20965 ng·h/ml, and (ii) a Cₘₐₓ from 117 to 1230, for example, 117 to 245, 230 to 590, or 590 to 1230 ng/ml and will desirably be bioequivalent according to FDA guidelines to a second orally administrable dosage form comprising the same dose of tramadol also suitable for once daily administration in the fasted state.

In at least one embodiment of the invention where the first once daily controlled-release dosage form is directed to a method for administering a therapeutically effective amount of tramadol to a human for the treatment or management of moderately to moderately severe pain, the once daily controlled-release dosage form will desirably also exhibit under fasting conditions a Tₘₐₓ of, for example, 9, 10, 11, 12, 13, or 14 hours and will desirably be bioequivalent according to FDA guidelines to a second orally administrable dosage form comprising the same dose of tramadol also suitable for once daily administration in the fasted state.

In at least one embodiment of the invention where the first once daily controlled-release dosage form is directed to a method for administering a therapeutically effective amount of tramadol to a human for the treatment or management of moderately to moderately severe pain, the once daily controlled-release dosage form will desirably also exhibit under fasting conditions a degree of fluctuation (%) of, for example, 43 to 141, 43 to 120, 58 to 132, or 57 to 141 and will desirably be bioequivalent according to FDA guidelines to a second orally administrable dosage form comprising the same dose of tramadol also suitable for once daily administration in the fasted state.

In at least one embodiment of the invention where the first once daily controlled-release dosage form is directed to a method for administering a therapeutically effective amount of tramadol to a human for the treatment or management of moderately to moderately severe pain, the once daily controlled-release dosage form will desirably also exhibit under fasting conditions a Cₘᵢₙ of, for example, from 31 to 652, 31 to 117, 96 to 241, or 226 to 652 ng/ml at steady state and will desirably be bioequivalent according to FDA guidelines to a second orally administrable dosage form comprising the same dose of tramadol also suitable for once daily administration in the fasted state.

In at least one embodiment of the invention where the first once daily controlled-release dosage form is directed to a method for administering a therapeutically effective amount of tramadol to a human for the treatment or management of moderately to moderately severe pain, the once daily controlled-release dosage form will desirably also exhibit under fasting conditions a Tₘₐₓ of, for example, 9, 10, 11, 12, 13, or 14 hours, a degree of fluctuation (%) of, for example, 43 to 141, 43 to 120, 58 to 132, or 57 to 141, and Cₘᵢₙ of, for example, from 31 to 652, 31 to 117, 96 to 241, or 226 to 652 ng/ml at steady state and will desirably be bioequivalent according to FDA guidelines to a second orally administrable dosage form comprising the same dose of tramadol also suitable for once daily administration in the fasted state.

In at least one embodiment of the invention, the in-vitro dissolution of the first once daily controlled-release dosage form is measured using a USP Type I, II, or III apparatus in dissolution medium chosen from 900 ml 0.1N HCl, water, 0.1N HCl+0.1% Cetrimide, USP Buffer pH 1.5, Acetate Buffer pH 4.5, Phosphate Buffer pH 6.5, or Phosphate Buffer pH7.4 at 75 rpm at 37°±0.5° C. and the tramadol released into the dissolution medium is assayed in a 10 ml UV cell at 271 nm.

In at least one embodiment of the invention, the in-vitro dissolution of the first once daily controlled-release dosage form is measured using a USP Type I, II, or III apparatus in 900 ml 0.1N HCl 75 rpm at 37°±0.5° C. and the tramadol released into the dissolution medium is assayed in a 10 ml UV cell at 271 nm.

In at least one embodiment of the invention, the in-vitro dissolution of the first once daily controlled-release dosage form is measured using a USP Type I, II, or III apparatus in water at 37°±0.5° C. and the tramadol released into the dissolution medium is assayed in a 10 ml UV cell at 271 nm.

In at least one embodiment of the invention, the in-vitro dissolution of the first once daily controlled-release dosage form is measured using a USP Type I, II, or III apparatus in 0.1N HCl+0.1% Cetrimide at 37°±0.5° C. and the tramadol released into the dissolution medium is assayed in a 10 ml UV cell at 271 nm.

In at least one embodiment of the invention, the in-vitro dissolution of the first once daily controlled-release dosage form is measured using a USP Type I, II, or III apparatus in USP Buffer pH 1.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form is measured using a USP Type I, II, or III apparatus in Acetate buffer pH 4.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form is measured using a USP Type I, II, or III apparatus in Phosphate Buffer pH 6.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form is measured using a USP Type I, II, or III apparatus in Phosphate Buffer pH 7.4 at 37°±0.5° C. and the tramadol released into the dissolution medium is assayed in a 10 ml UV cell at 271 nm.

The first once daily controlled-release dosage form will desirably be chosen from at least one osmotic dosage form, at least one swellable dosage form, at least one swellable and erodable dosage form, at least one erodable dosage form, at least one insoluble dosage form, at least one hydrophobic dosage form, at least one hydrophilic dosage form, at least one release-slowing coat, at least one insoluble coat, at least one swellable coat, at least one erodable coat, at least one swellable and erodable coat, at least one extended-release dosage form, at least one delayed-release dosage form, at least one modified-release dosage form, at least one sustained-release dosage form, at least one prolonged-release dosage form, at least one bi-phasic release dosage form, at least one normal release matrix core coated with at least one release-slowing coat, at least one normal release matrix core coated with at least one aqueous insoluble coat, at least one normal release matrix core coated with at least one swellable coat, at least one normal release matrix core coated with at least one swellable and erodable coat, at least one normal release matrix core coated with at least one erodable coat, or any combination thereof.

In at least one embodiment of the invention, the first once daily controlled-release dosage form comprises an effective amount of tramadol for the management of moderate to moderately severe pain.

In at least one embodiment of the invention, the first once daily controlled-release dosage form comprises from about 25 mg to about 800 mg of tramadol.

In at least one embodiment of the invention, the first once daily controlled-release dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprises tramadol hydrochloride.

In at least one embodiment of the invention, the first once daily controlled-release dosage form comprises a mixture of an effective amount of at least two different tramadol salts, wherein one of the salts is tramadol hydrochloride.

In at least one embodiment of the invention, the first once daily controlled-release dosage form comprises from 70 to 90% by weight of the core dry weight tramadol.

In at least one embodiment of the invention, the first once daily controlled-release dosage form comprises an immediate release coat, wherein said immediate release coat comprises tramadol.

In at least one embodiment of the invention, the first once daily controlled-release dosage form comprises an immediate release coat, wherein said immediate release coat comprises a salt or form of tramadol, which is different from the salt or form of tramadol present in the remainder of the first once daily controlled release dosage form.

In at least one embodiment of the invention, the first-once daily controlled-release dosage form comprises at least one release-slowing coat.

In at least one embodiment of the invention, the first-once daily controlled-release dosage form comprises at least one at least one delayed-release coat.

In at least one embodiment of the invention, the first-once daily controlled-release dosage form comprises an osmotic subcoat.

In at least one embodiment of the invention, the first-once daily controlled-release dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprises at least one release-slowing coat, which coat comprises at least one water-insoluble water-permeable film-forming polymer and at least one water-soluble polymer.

In at least one embodiment of the invention, the first-once daily controlled-release dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprises at least one release-slowing coat, which coat comprises at least one enteric polymer.

In at least one embodiment of the invention, the first-once daily controlled-release dosage comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprises at least one delayed-release coat, which coat comprises at least one pH dependent polymer.

In at least one embodiment of the invention, the first-once daily controlled-release dosage form comprises at least one non-functional soluble coat.

In at least one embodiment of the invention, the first-once daily controlled-release dosage form is in the form of a tablet.

In at least one embodiment of the invention, the first-once daily controlled-release dosage form is in the form of a capsule.

In at least one embodiment of the invention, the first-once daily controlled-release dosage form is in the form of a microparticle.

In at least one embodiment of the invention, the first-once daily controlled-release dosage form is in the form of a plurality of microparticles, wherein each microparticle comprises a controlled-release matrix core.

In at least one embodiment of the invention, the first-once daily controlled-release dosage form comprises a unitary core.

In at least one embodiment of the invention, the first-once daily controlled-release dosage form comprises two or more coats, wherein one coat comprises a delayed-release coat.

In at least one embodiment of the invention, the first-once daily controlled-release dosage form comprises two or more coats, wherein one coat comprises a release-slowing coat.

In at least one embodiment of the invention, the first-once daily controlled-release dosage form comprises two or more coats, wherein one coat which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprises a plurality of coated cores.

In at least one embodiment of the invention, the first once daily controlled-release dosage form comprises at least one osmotic dosage form.

In at least one embodiment of the invention, the first once daily controlled-release dosage form comprising the at least one osmotic dosage form comprises an effective amount of tramadol for the management of moderate to moderately severe pain.

In at least one embodiment of the invention, the first once daily controlled-release dosage form comprising the at least one osmotic dosage form comprises from about 25 mg to about 800 mg of tramadol.

In at least one embodiment of the invention, the first once daily controlled-release dosage form comprising the at least one osmotic dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one osmotic dosage form comprises tramadol hydrochloride

In at least one embodiment of the invention, the first once daily controlled-release dosage form comprising the at least one osmotic dosage form will desirably comprise a mixture of an effective amount of at least two different tramadol salts, wherein one of the salts is tramadol hydrochloride.

In at least one embodiment of the invention, the first once daily controlled-release dosage form comprising the at least one osmotic dosage form will desirably comprise about 70 to about 90% by weight of the core dry weight tramadol.

In at least one embodiment of the invention, the first once daily controlled-release dosage form comprising the at least one osmotic dosage form comprises an immediate release coat, wherein said immediate release coat comprises tramadol.

In at least one embodiment of the invention, the first once daily controlled-release dosage form comprising the at least one osmotic dosage form comprises an immediate release coat, wherein said immediate release coat comprises a salt or form of tramadol, which is different from the salt or form of tramadol present in the remainder of the osmotic dosage form.

In at least one embodiment of the invention, the first once daily controlled-release dosage form comprising the at least one osmotic dosage form comprises at least one release-slowing coat.

In at least one embodiment of the invention, the first once daily controlled-release dosage form comprising the at least one osmotic dosage form comprises at least one delayed-release coat.

In at least one embodiment of the invention, the first once daily controlled-release dosage form comprising the at least one osmotic dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprising the at least one osmotic dosage form comprises at least one delayed-release coat, which coat comprises at least one water-insoluble water-permeable film-forming polymer and at least one water-soluble polymer.

In at least one embodiment of the invention, the first once daily controlled-release dosage form comprising the at least one osmotic dosage form comprises at least one release-slowing coat, which coat comprises at least one water-insoluble water-permeable film-forming polymer, at least one water-soluble polymer, and at least one plasticizer.

In at least one embodiment of the invention, the first once daily controlled-release dosage form comprising the at least one osmotic dosage form comprises at least one release-slowing coat, which coat comprises at least one enteric polymer.

In at least one embodiment of the invention, the first once daily controlled-release dosage form comprising the at least one osmotic dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one osmotic dosage form comprises at least one delayed-release coat, which coat comprises at least one pH dependent polymer.

In at least one embodiment of the invention, the first once daily controlled-release dosage form comprising the at least one osmotic dosage form comprises two or more coats, wherein one coat comprises a release-slowing coat.

In at least one embodiment of the invention, the first once daily controlled-release dosage form comprising the at least one osmotic dosage form comprises two or more coats, wherein one coat comprises a delayed-release coat.

In at least one embodiment of the invention, the first once daily controlled-release dosage form comprising the at least one osmotic dosage form comprises two or more coats, wherein one coat comprises an enhanced-absorption coat.

In at least one embodiment of the invention, the first once daily controlled-release dosage form comprising the at least one osmotic dosage form comprises two or more coats, wherein one coat comprises aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one osmotic dosage form comprises at least one non-functional soluble coat.

In at least one embodiment of the invention, the first once daily controlled-release dosage form comprising the at least one osmotic dosage form comprises at least one osmotic subcoat.

In at least one embodiment of the invention, the first once daily controlled-release dosage form comprising the at least one osmotic dosage form comprises at least one means for the exit of tramadol from the first once daily rate controlled-release dosage form.

In at least one embodiment of the invention, the first once daily controlled-release dosage form comprising the at least one osmotic dosage form comprises at least one means for increasing the hydrostatic pressure of the first once daily controlled-release dosage form.

In at least one embodiment of the invention, the first once daily controlled-release dosage form comprising the at least one osmotic dosage form comprises at least one means for forcibly dispensing tramadol from the first once daily controlled-release dosage form.

In at least one embodiment of the invention, the first once daily controlled-release dosage form comprising the at least one osmotic dosage form is in the form of a tablet.

In at least one embodiment of the invention, the first once daily controlled-release dosage form comprising the at least one osmotic dosage form is in the form of a capsule.

In at least one embodiment of the invention, the first once daily controlled-release dosage form comprising the at least one osmotic dosage form of a microparticle.

In at least one embodiment of the invention, the first once daily controlled-release dosage form comprising the at least one osmotic dosage is comprised of a plurality of microparticles.

In at least one embodiment of the invention, the first once daily controlled-release dosage form comprises at least one lipid or wax dosage form.

In at least one embodiment of the invention, the first once daily controlled-release dosage form comprising the at least one lipid or wax dosage form comprises at least one lipid or wax matrix core.

In at least one embodiment of the invention, the first once daily controlled-release dosage form comprising at least one lipid or wax dosage form comprises an effective amount of tramadol for the management of moderate to moderately severe pain.

In at least one embodiment of the invention, the first once daily controlled-release dosage form comprising at least one lipid or wax dosage form comprises from about 25 mg to about 800 mg of tramadol.

In at least one embodiment of the invention, the first once daily controlled-release dosage form comprising at least one lipid or wax dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising at least one lipid or wax dosage form comprises tramadol hydrochloride.

In at least one embodiment of the invention, the first once daily controlled-release dosage form comprising at least one lipid or wax dosage form will desirably comprise a mixture of an effective amount of at least two different tramadol salts, wherein one of the salts is tramadol hydrochloride.

In at least one embodiment of the invention, the first once daily controlled-release dosage form comprising at least one lipid or wax dosage form will desirably comprise about 70 to about 90% by weight of the core dry weight tramadol.

In at least one embodiment of the invention, the first once daily controlled-release dosage form comprising at least one lipid or wax dosage form comprises an immediate release coat, wherein said immediate release coat comprises tramadol.

In at least one embodiment of the invention, the first once daily controlled-release dosage form comprising at least one lipid or wax dosage form comprises an immediate release coat, wherein said immediate release coat comprises a salt or form of tramadol, which is different from the salt or form of tramadol present in the remainder of the first once daily controlled release dosage form comprising the swellable matrix core.

In at least one embodiment of the invention, the first-once daily controlled-release dosage form comprising at least one lipid or wax dosage form comprises at least one release-slowing coat.

In at least one embodiment of the invention, the first-once daily controlled-release dosage form comprising at least one lipid or wax dosage form comprises at least one at least one delayed-release coat.

In at least one embodiment of the invention, the first-once daily controlled-release dosage form comprising at least one lipid or wax dosage form comprises an osmotic subcoat.

In at least one embodiment of the invention, the first-once daily controlled-release dosage form comprising the at least one lipid or wax dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprising the at least one lipid or wax dosage form comprises at least one release-slowing coat, which coat comprises at least one water-insoluble water-permeable film-forming polymer and at least one water-soluble polymer.

In at least one embodiment of the invention, the first-once daily controlled-release dosage form comprising the at least one lipid or wax dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprising the at least one lipid or wax dosage form comprises at least one release-slowing coat, which coat comprises at least one enteric polymer.

In at least one embodiment of the invention, the first-once daily controlled-release dosage form comprising the at least one lipid or wax dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one lipid or wax dosage form comprises at least one delayed-release coat, which coat comprises at least one pH dependent polymer.

In at least one embodiment of the invention, the first-once daily controlled-release dosage form comprising the at least one lipid or wax dosage form comprises at least one non-functional soluble coat.

In at least one embodiment of the invention, the first-once daily controlled-release dosage form comprising the at least one lipid or wax dosage form is in the form of a tablet.

In at least one embodiment of the invention, the first-once daily controlled-release dosage form comprising the at least one lipid or wax dosage form is in the form of a capsule.

In at least one embodiment of the invention, the first-once daily controlled-release dosage form comprising the at least one lipid or wax dosage form is in the form of a microparticle.

In at least one embodiment of the invention, the first-once daily controlled-release dosage form comprising the at least one lipid or wax dosage form is in the form of a plurality of microparticles, wherein each microparticle comprises a controlled-release matrix core.

In at least one embodiment of the invention, the first-once daily controlled-release dosage form comprises at least one lipid or wax dosage form, wherein said at least one lipid or wax dosage form comprises a unitary core.

In at least one embodiment of the invention, the first-once daily controlled-release dosage form comprises at least one lipid or wax dosage form, wherein said at least one lipid or wax dosage form further comprises two or more coats, wherein one coat comprises a delayed-release coat.

In at least one embodiment of the invention, the first-once daily controlled-release dosage form comprises at least one lipid or wax dosage form, wherein said at least one lipid or wax dosage form further comprises two or more coats, wherein one coat comprises a release-slowing coat.

In at least one embodiment of the invention, the first-once daily controlled-release dosage form comprises at least one lipid or wax dosage form, wherein said at least one lipid or wax dosage form comprises two or more coats, wherein one coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one lipid or wax dosage form is comprised of a plurality of coated cores.

In at least one embodiment of the invention, the first once daily controlled-release dosage form comprises at least one swellable dosage form.

In at least one embodiment of the invention, the first once daily controlled-release dosage form comprising the at least one swellable dosage form comprises at least one swellable matrix core.

In at least one embodiment of the invention, the first once daily controlled-release dosage form comprising the at least one swellable dosage form comprises an effective amount of tramadol for the management of moderate to moderately severe pain.

In at least one embodiment of the invention, the first once daily controlled-release dosage form comprising the at least one swellable dosage form comprises from about 25 mg to about 800 mg of tramadol.

In at least one embodiment of the invention, the first once daily controlled-release dosage form comprising the at least one swellable dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one swellable dosage form comprises tramadol hydrochloride.

In at least one embodiment of the invention, the first once daily controlled-release dosage form comprising the at least one swellable dosage form will desirably comprise a mixture of an effective amount of at least two different tramadol salts, wherein at least one salt is tramadol hydrochloride.

In at least one embodiment of the invention, the first once daily controlled-release dosage form comprising the at least one swellable dosage form will desirably comprise about 70 to about 90% by weight of the core dry weight tramadol.

In at least one embodiment of the invention, the first once daily controlled-release dosage form comprising the at least one swellable dosage form comprises an immediate release coat, wherein said immediate release coat comprises tramadol.

In at least one embodiment of the invention, the first once daily controlled-release dosage form comprising the at least one swellable dosage form comprises an immediate release coat, wherein said immediate release coat comprises a salt or form of tramadol, which is different from the salt or form of tramadol present in the remainder of the first once daily controlled release dosage form comprising the swellable dosage form.

In at least one embodiment of the invention, the first-once daily controlled-release dosage form comprising the at least one swellable dosage form comprises at least one release-slowing coat.

In at least one embodiment of the invention, the first-once daily controlled-release dosage form comprising the at least one swellable dosage form comprises at least one at least one delayed-release coat.

In at least one embodiment of the invention, the first-once daily controlled-release dosage form comprising the at least one swellable dosage form comprises an osmotic subcoat.

In at least one embodiment of the invention, the first-once daily controlled-release dosage form comprising the at least one swellable dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprising the at least one swellable dosage form comprises at least one release-slowing coat, which coat comprises at least one water-insoluble water-permeable film-forming polymer and at least one water-soluble polymer.

In at least one embodiment of the invention, the first-once daily controlled-release dosage form comprising the at least one swellable dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprising the at least one swellable dosage form comprises at least one release-slowing coat, which coat comprises at least one enteric polymer.

In at least one embodiment of the invention, the first-once daily controlled-release dosage form comprising the at least one swellable dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one swellable dosage form comprises at least one delayed-release coat, which coat comprises at least one pH dependent polymer.

In at least one embodiment of the invention, the first-once daily controlled-release dosage form comprising the at least one swellable dosage form comprises at least one non-functional soluble coat.

In at least one embodiment of the invention, the first-once daily controlled-release dosage form comprising the at least one swellable dosage form is in the form of a tablet.

In at least one embodiment of the invention, the first-once daily controlled-release dosage form comprising the at least one swellable dosage form is in the form of a capsule.

In at least one embodiment of the invention, the first-once daily controlled-release dosage form comprising the at least one swellable dosage form is in the form of a microparticle.

In at least one embodiment of the invention, the first-once daily controlled-release dosage form comprising the at least one swellable dosage form is in the form of a plurality of microparticles, wherein each microparticle comprises a swellable dosage form.

In at least one embodiment of the invention, the first-once daily controlled-release dosage form comprises at least one swellable dosage form, wherein said swellable dosage form comprises a unitary core.

In at least one embodiment of the invention, the first-once daily controlled-release dosage form comprises at least one swellable dosage form, wherein said swellable dosage form further comprises two or more coats, wherein one coat comprises a delayed-release coat.

In at least one embodiment of the invention, the first-once daily controlled-release dosage form comprises at least one swellable dosage form, wherein said swellable dosage form further comprises two or more coats, wherein one coat comprises a release-slowing coat.

In at least one embodiment of the invention, the first-once daily controlled-release dosage form comprises at least one swellable dosage form, wherein said swellable dosage form further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one swellable dosage form is comprised of a plurality of coated cores.

In at least one embodiment of the invention, the first once daily controlled-release dosage form comprises at least one swellable and erodable dosage form.

In at least one embodiment of the invention, the first once daily controlled-release dosage form comprising the at least one swellable and erodable dosage form comprises at least one swellable and erodable matrix core.

In at least one embodiment of the invention, the first once daily controlled-release dosage form comprising the at least one swellable and erodable dosage form comprises an effective amount of tramadol for the management of moderate to moderately severe pain.

In at least one embodiment of the invention, the first once daily controlled-release dosage form comprising the at least one swellable and erodable dosage form comprises from about 25 mg to about 800 mg of tramadol.

In at least one embodiment of the invention, the first once daily controlled-release dosage form comprising the at least one swellable and erodable dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one swellable and erodable dosage form comprises tramadol hydrochloride.

In at least one embodiment of the invention, the first once daily controlled-release dosage form comprising the at least one swellable and erodable dosage form will desirably comprise a mixture of an effective amount of at least two different tramadol salts, wherein one of the salts is tramadol hydrochloride.

In at least one embodiment of the invention, the first once daily controlled-release dosage form comprising the at least one swellable and erodable dosage form will desirably comprise about 70 to about 90% by weight of the core dry weight tramadol.

In at least one embodiment of the invention, the first once daily controlled-release dosage form comprising the at least one swellable and erodable dosage form comprises an immediate release coat, wherein said immediate release coat comprises tramadol.

In at least one embodiment of the invention, the first once daily controlled-release dosage form comprising the at least one swellable and erodable dosage form comprises an immediate release coat, wherein said immediate release coat comprises a salt or form of tramadol, which is different from the salt or form of tramadol present in the remainder of the first once daily controlled release dosage form comprising the swellable and erodable dosage form.

In at least one embodiment of the invention, the first-once daily controlled-release dosage form comprising the at least one swellable and erodable dosage form comprises at least one release-slowing coat.

In at least one embodiment of the invention, the first-once daily controlled-release dosage form comprising the at least one swellable and erodable dosage form comprises at least one at least one delayed-release coat.

In at least one embodiment of the invention, the first-once daily controlled-release dosage form comprising the at least one swellable and erodable dosage form comprises an osmotic subcoat.

In at least one embodiment of the invention, the first-once daily controlled-release dosage form comprising the at least one swellable and erodable dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprising the at least one swellable and erodable dosage form comprises at least one release-slowing coat, which coat comprises at least one water-insoluble water-permeable film-forming polymer and at least one water-soluble polymer.

In at least one embodiment of the invention, the first-once daily controlled-release dosage form comprising the at least one swellable and erodable dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprising the at least one swellable and erodable dosage form comprises at least one release-slowing coat, which coat comprises at least one enteric polymer.

In at least one embodiment of the invention, the first-once daily controlled-release dosage form comprising the at least one swellable and erodable dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one swellable and erodable dosage form comprises at least one delayed-release coat, which coat comprises at least one pH dependent polymer.

In at least one embodiment of the invention, the first-once daily controlled-release dosage form comprising the at least one swellable and erodable dosage form comprises at least one non-functional soluble coat.

In at least one embodiment of the invention, the first-once daily controlled-release dosage form comprising the at least one swellable and erodable dosage form is in the form of a tablet.

In at least one embodiment of the invention, the first-once daily controlled-release dosage form comprising the at least one swellable and erodable dosage form is in the form of a capsule.

In at least one embodiment of the invention, the first-once daily controlled-release dosage form comprising the at least one swellable and erodable dosage form is in the form of a microparticle.

In at least one embodiment of the invention, the first-once daily controlled-release dosage form comprising the at least one swellable and erodable dosage form is in the form of a plurality of microparticles, wherein each microparticle comprises a swellable and erodable dosage form.

In at least one embodiment of the invention, the first-once daily controlled-release dosage form comprises at least one swellable and erodable dosage form, wherein said swellable and erodable dosage form comprises a unitary core.

In at least one embodiment of the invention, the first-once daily controlled-release dosage form comprises at least one swellable and erodable dosage form, wherein said swellable and erodable dosage form further comprises two or more coats, wherein one coat comprises a delayed-release coat.

In at least one embodiment of the invention, the first-once daily controlled-release dosage form comprises at least one swellable and erodable dosage form, wherein said swellable and erodable dosage form further comprises two or more coats, wherein one coat comprises a release-slowing coat.

In at least one embodiment of the invention, the first-once daily controlled-release dosage form comprises at least one swellable and erodable dosage form, wherein said swellable and erodable dosage form further comprises two or more coats, wherein one coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one swellable and erodable dosage form is comprised of a plurality of coated cores.

In at least one embodiment of the invention, the first once daily controlled-release dosage form comprises at least one erodable dosage form.

In at least one embodiment of the invention, the first once daily controlled-release dosage form comprising the at least one erodable dosage form comprises at least erodable matrix core.

In at least one embodiment of the invention, the first once daily controlled-release dosage form comprising the at least one erodable dosage form comprises an effective amount of tramadol for the management of moderate to moderately severe pain.

In at least one embodiment of the invention, the first once daily controlled-release dosage form comprising the at least one erodable dosage form comprises from about 25 mg to about 800 mg of tramadol.

In at least one embodiment of the invention, the first once daily controlled-release dosage form comprising the at least one erodable dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one erodable dosage form comprises tramadol hydrochloride.

In at least one embodiment of the invention, the first once daily controlled-release dosage form comprising the at least one erodable dosage form will desirably comprise a mixture of an effective amount of at least two different tramadol salts, wherein one of the salts is tramadol hydrochloride.

In at least one embodiment of the invention, the first once daily controlled-release dosage form comprising the at least one erodable dosage form will desirably comprise about 70 to about 90% by weight of the core dry weight tramadol.

In at least one embodiment of the invention, the first once daily controlled-release dosage form comprising the at least one erodable dosage form comprises an immediate release coat, wherein said immediate release coat comprises tramadol.

In at least one embodiment of the invention, the first once daily controlled-release dosage form comprising the at least one erodable dosage form comprises an immediate release coat, wherein said immediate release coat comprises a salt or form of tramadol, which is different from the salt or form of tramadol present in the remainder of the first once daily controlled release dosage form comprising the erodable dosage form.

In at least one embodiment of the invention, the first-once daily controlled-release dosage form comprising the at least one erodable dosage form comprises at least one release-slowing coat.

In at least one embodiment of the invention, the first-once daily controlled-release dosage form comprising the at least one erodable dosage form comprises at least one at least one delayed-release coat.

In at least one embodiment of the invention, the first-once daily controlled-release dosage form comprising the at least one erodable dosage form comprises an osmotic subcoat.

In at least one embodiment of the invention, the first-once daily controlled-release dosage form comprising the at least one erodable dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprising the at least one erodable dosage form comprises at least one release-slowing coat, which coat comprises at least one water-insoluble water-permeable film-forming polymer and at least one water-soluble polymer.

In at least one embodiment of the invention, the first-once daily controlled-release dosage form comprising the at least one erodable dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprising the at least one erodable dosage form comprises at least one release-slowing coat, which coat comprises at least one enteric polymer.

In at least one embodiment of the invention, the first-once daily controlled-release dosage form comprising the at least one erodable dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one erodable dosage form comprises at least one delayed-release coat, which coat comprises at least one pH dependent polymer.

In at least one embodiment of the invention, the first-once daily controlled-release dosage form comprising the at least one erodable dosage form comprises at least one non-functional soluble coat.

In at least one embodiment of the invention, the first-once daily controlled-release dosage form comprising the at least one erodable dosage form is in the form of a tablet.

In at least one embodiment of the invention, the first-once daily controlled-release dosage form comprising the at least one erodable dosage form is in the form of a capsule.

In at least one embodiment of the invention, the first-once daily controlled-release dosage form comprising the at least one erodable dosage form is in the form of a microparticle.

In at least one embodiment of the invention, the first-once daily controlled-release dosage form comprising the at least one erodable dosage form is in the form of a plurality of microparticles, wherein each microparticle comprises an erodable dosage form.

In at least one embodiment of the invention, the first-once daily controlled-release dosage form comprises at least one erodable dosage form, wherein said erodable dosage form comprises a unitary core.

In at least one embodiment of the invention, the first-once daily controlled-release dosage form comprises at least one erodable dosage form, wherein said erodable dosage form further comprises two or more coats, wherein one coat comprises a delayed-release coat.

In at least one embodiment of the invention, the first-once daily controlled-release dosage form comprises at least one erodable dosage form, wherein said erodable dosage form further comprises two or more coats, wherein one coat comprises a release-slowing coat.

In at least one embodiment of the invention, the first-once daily controlled-release dosage form comprises at least one erodable dosage form, wherein said erodable dosage form further comprises two or more coats, wherein one coat comprises an enhanced-absorption coat.

In at least one embodiment of the invention, the first-once daily controlled-release dosage form comprises at least one erodable dosage form, wherein said erodable dosage form further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one erodable dosage form is comprised of a plurality of coated cores.

In at least one embodiment of the invention, the first once daily controlled-release dosage form comprises at least one insoluble dosage form.

In at least one embodiment of the invention, the first once daily controlled-release dosage form comprising the at least one insoluble dosage form comprises at least one insoluble matrix core.

In at least one embodiment of the invention, the first once daily controlled-release dosage form comprising the at least one insoluble dosage form comprises an effective amount of tramadol for the management of moderate to moderately severe pain.

In at least one embodiment of the invention, the first once daily controlled-release dosage form comprising the at least one insoluble dosage form comprises from about 25 mg to about 800 mg of tramadol.

In at least one embodiment of the invention, the first once daily controlled-release dosage form comprising the at least one insoluble dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one insoluble dosage form comprises tramadol hydrochloride.

In at least one embodiment of the invention, the first once daily controlled-release dosage form comprising the at least one insoluble dosage form will desirably comprise a mixture of an effective amount of at least two different tramadol salts, wherein one of the salts is tramadol hydrochloride.

In at least one embodiment of the invention, the first once daily controlled-release dosage form comprising the at least one insoluble dosage form will desirably comprise about 70 to about 90% by weight of the core dry weight tramadol.

In at least one embodiment of the invention, the first once daily controlled-release dosage form comprising the at least one insoluble dosage form comprises an immediate release coat, wherein said immediate release coat comprises tramadol.

In at least one embodiment of the invention, the first once daily controlled-release dosage form comprising the at least one insoluble dosage form comprises an immediate release coat, wherein said immediate release coat comprises a salt or form of tramadol, which is different from the salt or form of tramadol present in the remainder of the first once daily controlled release dosage form comprising the insoluble dosage form.

In at least one embodiment of the invention, the first-once daily controlled-release dosage form comprising the at least one insoluble dosage form comprises at least one release-slowing coat.

In at least one embodiment of the invention, the first-once daily controlled-release dosage form comprising the at least one insoluble dosage form comprises at least one at least one delayed-release coat.

In at least one embodiment of the invention, the first-once daily controlled-release dosage form comprising the at least one insoluble dosage form comprises at least one at least one enhanced-absorption coat.

In at least one embodiment of the invention, the first-once daily controlled-release dosage form comprising the at least one insoluble dosage form comprises an osmotic subcoat.

In at least one embodiment of the invention, the first-once daily controlled-release dosage form comprising the at least one insoluble dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprising the at least one insoluble dosage form comprises at least one release-slowing coat, which coat comprises at least one water-insoluble water-permeable film-forming polymer and at least one water-soluble polymer.

In at least one embodiment of the invention, the first-once daily controlled-release dosage form comprising the at least one insoluble dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprising the at least one insoluble dosage form comprises at least one release-slowing coat, which coat comprises at least one enteric polymer.

In at least one embodiment of the invention, the first-once daily controlled-release dosage form comprising the at least one insoluble dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one insoluble dosage form comprises at least one delayed-release coat, which coat comprises at least one pH dependent polymer.

In at least one embodiment of the invention, the first-once daily controlled-release dosage form comprising the at least one insoluble dosage form comprises at least one non-functional soluble coat.

In at least one embodiment of the invention, the first-once daily controlled-release dosage form comprising the at least one insoluble dosage form is in the form of a tablet.

In at least one embodiment of the invention, the first-once daily controlled-release dosage form comprising the at least one insoluble dosage form is in the form of a capsule.

In at least one embodiment of the invention, the first-once daily controlled-release dosage form comprising the at least one insoluble dosage form is in the form of a microparticle.

In at least one embodiment of the invention, the first-once daily controlled-release dosage form comprising the at least one insoluble dosage form is in the form of a plurality of microparticles, wherein each microparticle comprises an insoluble dosage form.

In at least one embodiment of the invention, the first-once daily controlled-release dosage form comprises at least one insoluble dosage form, wherein said insoluble dosage form comprises a unitary core.

In at least one embodiment of the invention, the first-once daily controlled-release dosage form comprises at least one insoluble dosage form, wherein said insoluble dosage form further comprises two or more coats, wherein one coat comprises a delayed-release coat.

In at least one embodiment of the invention, the first-once daily controlled-release dosage form comprises at least one insoluble dosage form, wherein said insoluble dosage form further comprises two or more coats, wherein one coat comprises a release-slowing coat.

In at least one embodiment of the invention, the first-once daily controlled-release dosage form comprises at least one insoluble dosage form, wherein said insoluble dosage form further comprises two or more coats, wherein one coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one insoluble dosage form is comprised of a plurality of coated cores.

In at least one embodiment of the invention, the first once daily controlled-release dosage form comprises at least one hydrophobic dosage form.

In at least one embodiment of the invention, the first once daily controlled-release dosage form comprising the at least one hydrophobic dosage form comprises at least one hydrophobic matrix core.

In at least one embodiment of the invention, the first once daily controlled-release dosage form comprising the at least one hydrophobic dosage form comprises an effective amount of tramadol for the management of moderate to moderately severe pain.

In at least one embodiment of the invention, the first once daily controlled-release dosage form comprising the at least one hydrophobic dosage form comprises from about 25 mg to about 800 mg of tramadol.

In at least one embodiment of the invention, the first once daily controlled-release dosage form comprising the at least one hydrophobic dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one hydrophobic dosage form comprises tramadol hydrochloride.

In at least one embodiment of the invention, the first once daily controlled-release dosage form comprising the at least one hydrophobic dosage form will desirably comprise a mixture of an effective amount of at least two different tramadol salts, wherein one of the salts is tramadol hydrochloride.

In at least one embodiment of the invention, the first once daily controlled-release dosage form comprising the at least one hydrophobic dosage form will desirably comprise about 70 to about 90% by weight of the core dry weight tramadol.

In at least one embodiment of the invention, the first once daily controlled-release dosage form comprising the at least one hydrophobic dosage form comprises an immediate release coat, wherein said immediate release coat comprises tramadol.

In at least one embodiment of the invention, the first once daily controlled-release dosage form comprising the at least one hydrophobic dosage form comprises an immediate release coat, wherein said immediate release coat comprises a salt or form of tramadol, which is different from the salt or form of tramadol present in the remainder of the first once daily controlled release dosage form comprising the hydrophobic dosage form.

In at least one embodiment of the invention, the first-once daily controlled-release dosage form comprising the at least one hydrophobic dosage form comprises at least one release-slowing coat.

In at least one embodiment of the invention, the first-once daily controlled-release dosage form comprising the at least one hydrophobic dosage form comprises at least one at least one delayed-release coat.

In at least one embodiment of the invention, the first-once daily controlled-release dosage form comprising the at least one hydrophobic dosage form comprises an osmotic subcoat.

In at least one embodiment of the invention, the first-once daily controlled-release dosage form comprising the at least one hydrophobic dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprising the at least one hydrophobic dosage form comprises at least one release-slowing coat, which coat comprises at least one water-insoluble water-permeable film-forming polymer and at least one water-soluble polymer.

In at least one embodiment of the invention, the first-once daily controlled-release dosage form comprising the at least one hydrophobic dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprising the at least one hydrophobic dosage form comprises at least one release-slowing coat, which coat comprises at least one enteric polymer.

In at least one embodiment of the invention, the first-once daily controlled-release dosage form comprising the at least one hydrophobic dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one hydrophobic dosage form comprises at least one delayed-release coat, which coat comprises at least one pH dependent polymer.

In at least one embodiment of the invention, the first-once daily controlled-release dosage form comprising the at least one hydrophobic dosage form comprises at least one non-functional soluble coat.

In at least one embodiment of the invention, the first-once daily controlled-release dosage form comprising the at least one hydrophobic dosage form is in the form of a tablet.

In at least one embodiment of the invention, the first-once daily controlled-release dosage form comprising the at least one hydrophobic dosage form is in the form of a capsule.

In at least one embodiment of the invention, the first-once daily controlled-release dosage form comprising the at least one hydrophobic dosage form is in the form of a microparticle.

In at least one embodiment of the invention, the first-once daily controlled-release dosage form comprising the at least one hydrophobic dosage form is in the form of a plurality of microparticles, wherein each microparticle comprises a hydrophobic dosage form.

In at least one embodiment of the invention, the first-once daily controlled-release dosage form comprises at least one hydrophobic dosage form, wherein said hydrophobic dosage form comprises a unitary core.

In at least one embodiment of the invention, the first-once daily controlled-release dosage form comprises at least one hydrophobic dosage form, wherein said hydrophobic dosage form further comprises two or more coats, wherein one coat comprises a delayed-release coat.

In at least one embodiment of the invention, the first-once daily controlled-release dosage form comprises at least one hydrophobic dosage form, wherein said hydrophobic dosage form further comprises two or more coats, wherein one coat comprises a release-slowing coat.

In at least one embodiment of the invention, the first-once daily controlled-release dosage form comprises at least one hydrophobic dosage form, wherein said hydrophobic dosage form further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one hydrophobic dosage form is comprised of a plurality of coated cores.

In at least one embodiment of the invention, the first once daily controlled-release dosage form comprises at least one hydrophilic dosage form.

In at least one embodiment of the invention, the first once daily controlled-release dosage form comprising the at least one hydrophobic dosage form comprises at least one hydrophilic matrix core.

In at least one embodiment of the invention, the first once daily controlled-release dosage form comprising the at least one hydrophilic dosage form comprises an effective amount of tramadol for the management of moderate to moderately severe pain.

In at least one embodiment of the invention, the first once daily controlled-release dosage form comprising the at least one hydrophilic dosage form comprises from about 25 mg to about 800 mg of tramadol.

In at least one embodiment of the invention, the first once daily controlled-release dosage form comprising the at least one hydrophilic dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one hydrophilic dosage form comprises tramadol hydrochloride.

In at least one embodiment of the invention, the first once daily controlled-release dosage form comprising the at least one hydrophilic dosage form will desirably comprise a mixture of an effective amount of at least two different tramadol salts, wherein one of the salts is tramadol hydrochloride.

In at least one embodiment of the invention, the first once daily controlled-release dosage form comprising the at least one hydrophilic dosage form will desirably comprise about 70 to about 90% by weight of the core dry weight tramadol.

In at least one embodiment of the invention, the first once daily controlled-release dosage form comprising the at least one hydrophilic dosage form comprises an immediate release coat, wherein said immediate release coat comprises tramadol.

In at least one embodiment of the invention, the first once daily controlled-release dosage form comprising the at least one hydrophilic dosage form comprises an immediate release coat, wherein said immediate release coat comprises a salt or form of tramadol, which is different from the salt or form of tramadol present in the remainder of the first once daily controlled release dosage form comprising the hydrophilic dosage form.

In at least one embodiment of the invention, the first-once daily controlled-release dosage form comprising the at least one hydrophilic dosage form comprises at least one release-slowing coat.

In at least one embodiment of the invention, the first-once daily controlled-release dosage form comprising the at least one hydrophilic dosage form comprises at least one at least one delayed-release coat.

In at least one embodiment of the invention, the first-once daily controlled-release dosage form comprising the at least one hydrophilic dosage form comprises an osmotic subcoat.

In at least one embodiment of the invention, the first-once daily controlled-release dosage form comprising the at least one hydrophilic dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprising the at least one hydrophilic dosage form comprises at least one release-slowing coat, which coat comprises at least one water-insoluble water-permeable film-forming polymer and at least one water-soluble polymer.

In at least one embodiment of the invention, the first-once daily controlled-release dosage form comprising the at least one hydrophilic dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprising the at least one hydrophilic dosage form comprises at least one release-slowing coat, which coat comprises at least one enteric polymer.

In at least one embodiment of the invention, the first-once daily controlled-release dosage form comprising the at least one hydrophilic dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one hydrophilic dosage form comprises at least one delayed-release coat, which coat comprises at least one pH dependent polymer.

In at least one embodiment of the invention, the first-once daily controlled-release dosage form comprising the at least one hydrophilic dosage form comprises at least one non-functional soluble coat.

In at least one embodiment of the invention, the first-once daily controlled-release dosage form comprising the at least one hydrophilic dosage form is in the form of a tablet.

In at least one embodiment of the invention, the first-once daily controlled-release dosage form comprising the at least one hydrophilic dosage form is in the form of a capsule.

In at least one embodiment of the invention, the first-once daily controlled-release dosage form comprising the at least one hydrophilic dosage form is in the form of a microparticle.

In at least one embodiment of the invention, the first-once daily controlled-release dosage form comprising the at least one hydrophilic dosage form is in the form of a plurality of microparticles, wherein each microparticle comprises a hydrophilic dosage form.

In at least one embodiment of the invention, the first-once daily controlled-release dosage form comprises at least one hydrophilic dosage form, wherein said hydrophilic dosage form comprises a unitary core.

In at least one embodiment of the invention, the first-once daily controlled-release dosage form comprises at least one hydrophilic dosage form, wherein said hydrophilic dosage form further comprises two or more coats, wherein one coat comprises a delayed-release coat.

In at least one embodiment of the invention, the first-once daily controlled-release dosage form comprises at least one hydrophilic dosage form, wherein said hydrophilic dosage form further comprises two or more coats, wherein one coat comprises a release-slowing coat.

In at least one embodiment of the invention, the first-once daily controlled-release dosage form comprises at least one hydrophilic dosage form, wherein said hydrophilic dosage form further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one hydrophilic dosage form is comprised of a plurality of coated cores.

In at least one embodiment of the invention, the first once daily controlled-release dosage form comprises a combination of a hydrophobic and hydrophilic dosage form.

In at least one embodiment of the invention, the first once daily controlled-release dosage form comprising a combination of a hydrophobic and hydrophilic dosage form comprises a combination of a hydrophobic and hydrophilic matrix core.

In at least one embodiment of the invention, the first once daily controlled-release dosage form comprising a combination of a hydrophobic and hydrophilic dosage form comprises an effective amount of tramadol for the management of moderate to moderately severe pain.

In at least one embodiment of the invention, the first once daily controlled-release dosage form comprising a combination of a hydrophobic and hydrophilic dosage form comprises from about 25 mg to about 800 mg of tramadol.

In at least one embodiment of the invention, the first once daily controlled-release dosage form comprising a combination of a hydrophobic and hydrophilic dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising a combination of a hydrophobic and hydrophilic dosage form comprises tramadol hydrochloride.

In at least one embodiment of the invention, the first once daily controlled-release dosage form comprising a combination of a hydrophobic and hydrophilic dosage form will desirably comprise a mixture of an effective amount of at least two different tramadol salts, wherein one of the salts is tramadol hydrochloride.

In at least one embodiment of the invention, the first once daily controlled-release dosage form comprising a combination of a hydrophobic and hydrophilic dosage form comprises 70 to 90% by weight of the core dry weight tramadol.

In at least one embodiment of the invention, the first once daily controlled-release dosage form comprising a combination of a hydrophobic and hydrophilic dosage form comprises an immediate release coat comprising tramadol.

In at least one embodiment of the invention, the first once daily controlled-release dosage form comprising a combination of a hydrophobic and hydrophilic dosage form comprises an immediate release coat comprising a salt of tramadol which is different from the salt of tramadol present in said first once daily controlled-release dosage form comprising a combination of the hydrophobic and hydrophilic dosage form.

In at least one embodiment of the invention, the first once daily controlled-release dosage form comprising a combination of a hydrophobic and hydrophilic dosage form comprises at least one release-slowing coat.

In at least one embodiment of the invention, the first once daily controlled-release dosage form comprising a combination of a hydrophobic and hydrophilic dosage form comprises at least one delayed-release coat.

In at least one embodiment of the invention, the first once daily controlled-release dosage form comprising a combination of a hydrophobic and hydrophilic dosage form comprises an osmotic subcoat.

In at least one embodiment of the invention, the first once daily controlled-release dosage form comprising a combination of a hydrophobic and hydrophilic dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprising a combination of a hydrophobic and hydrophilic dosage form comprises at least one release-slowing coat, which coat comprises at least one water-insoluble water-permeable film-forming polymer and at least one water-soluble polymer.

In at least one embodiment of the invention, the first once daily controlled-release dosage form comprising a combination of a hydrophobic and hydrophilic dosage form comprises at least one release-slowing coat, which coat comprises at least one water-insoluble water-permeable film-forming polymer, at least one water-soluble polymer, and at least one plasticizer.

In at least one embodiment of the invention, the first once daily controlled-release dosage form comprising a combination of a hydrophobic and hydrophilic dosage form comprises at least one release-slowing coat, which coat comprises at least one enteric polymer.

In at least one embodiment of the invention, the first once daily controlled-release dosage form comprising a combination of a hydrophobic and hydrophilic dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising a combination of a hydrophobic and hydrophilic dosage form comprises at least delayed-release coat, which coat comprises at least one pH dependent polymer.

In at least one embodiment of the invention, the first once daily controlled-release dosage form comprising a combination of a hydrophobic and hydrophilic dosage form comprises at least one non-functional soluble coat.

In at least one embodiment of the invention, the first once daily controlled-release dosage form comprising a combination of a hydrophobic and hydrophilic dosage form is in the form of a tablet.

In at least one embodiment of the invention, the first once daily controlled-release dosage form comprising a combination of a hydrophobic and hydrophilic dosage form is in the form of a capsule.

In at least one embodiment of the invention, the first once daily controlled-release dosage form comprising a combination of a hydrophobic and hydrophilic dosage form is in the form of a microparticle.

In at least one embodiment of the invention, the first once daily controlled-release dosage form comprising a combination of a hydrophobic and hydrophilic dosage form is in the form a plurality of microparticles, wherein each microparticle comprises a combination of the hydrophobic and hydrophilic dosage form.

In at least one embodiment of the invention, the first once daily controlled-release dosage form comprising a combination of a hydrophobic and hydrophilic dosage form is comprised of at least one unitary core.

In at least one embodiment of the invention, the first once daily controlled-release dosage form comprising a combination of a hydrophobic and hydrophilic dosage form further comprises two or more coats, wherein one coat comprises a release-slowing coat.

In at least one embodiment of the invention, the first once daily controlled-release dosage form comprising a combination of a hydrophobic and hydrophilic dosage form further comprises two or more coats, wherein one coat comprises a delayed-release coat.

In at least one embodiment of the invention, the first once daily controlled-release dosage form comprising a combination of a hydrophobic and hydrophilic dosage form further comprises two or more coats, wherein one coat comprises an enhanced-absorption coat.

In at least one embodiment of the invention, the first once daily controlled-release dosage form comprising a combination of a hydrophobic and hydrophilic dosage form further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising a combination of a hydrophobic and hydrophilic dosage form is in the form of a plurality of coated cores.

In at least one embodiment of the invention, the first once daily controlled-release dosage form comprises at least one insoluble polymer dosage form.

In at least one embodiment of the invention, the first once daily controlled-release dosage form comprising the at least one insoluble polymer dosage form comprises at least one insoluble polymer matrix core.

In at least one embodiment of the invention, the first once daily controlled-release dosage form comprising the at least one insoluble polymer dosage form comprises an effective amount of tramadol for the management of moderate to moderately severe pain.

In at least one embodiment of the invention, the first once daily controlled-release dosage form comprising the at least one insoluble polymer dosage form comprises from about 25 mg to about 800 mg of tramadol.

In at least one embodiment of the invention, the first once daily controlled-release dosage form comprising the at least one insoluble polymer dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one insoluble polymer dosage form comprises tramadol hydrochloride.

In at least one embodiment of the invention, the first once daily controlled-release dosage form comprising the at least one insoluble polymer dosage form will desirably comprise a mixture of an effective amount of at least two different tramadol salts.

In at least one embodiment of the invention, the first once daily controlled-release dosage form comprising the at least one insoluble polymer dosage form will desirably comprise about 70 to about 90% by weight of the core dry weight tramadol.

In at least one embodiment of the invention, the first once daily controlled-release dosage form comprising the at least one insoluble polymer dosage form comprises an immediate release coat, wherein said immediate release coat comprises tramadol.

In at least one embodiment of the invention, the first once daily controlled-release dosage form comprising the at least one insoluble polymer dosage form comprises an immediate release coat, wherein said immediate release coat comprises a salt or form of tramadol, which is different from the salt or form of tramadol present in the remainder of the first once daily controlled release dosage form comprising the insoluble polymer dosage form.

In at least one embodiment of the invention, the first-once daily controlled-release dosage form comprising the at least one insoluble polymer dosage form comprises at least one release-slowing coat.

In at least one embodiment of the invention, the first-once daily controlled-release dosage form comprising the at least one insoluble polymer dosage form comprises at least one at least one delayed-release coat.

In at least one embodiment of the invention, the first-once daily controlled-release dosage form comprising the at least one insoluble polymer dosage form comprises at least one at least one enhanced release coat.

In at least one embodiment of the invention, the first-once daily controlled-release dosage form comprising the at least one insoluble polymer dosage form comprises an osmotic subcoat.

In at least one embodiment of the invention, the first-once daily controlled-release dosage form comprising the at least one insoluble polymer dosage form comprises at least one release-slowing coat, which release slowing coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprising the at least one insoluble polymer dosage form comprises at least one release-slowing coat, which coat comprises at least one water-insoluble water-permeable film-forming polymer and at least one water-soluble polymer.

In at least one embodiment of the invention, the first-once daily controlled-release dosage form comprising the at least one insoluble polymer dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprising the at least one insoluble polymer dosage form comprises at least one release-slowing coat, which coat comprises at least one enteric polymer.

In at least one embodiment of the invention, the first-once daily controlled-release dosage form comprising the at least one insoluble polymer dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one insoluble polymer dosage form comprises at least one delayed-release coat, which coat comprises at least one pH dependent polymer.

In at least one embodiment of the invention, the first-once daily controlled-release dosage form comprising the at least one insoluble polymer dosage form comprises at least one non-functional soluble coat.

In at least one embodiment of the invention, the first-once daily controlled-release dosage form comprising the at least one insoluble polymer dosage form is in the form of a tablet.

In at least one embodiment of the invention, the first-once daily controlled-release dosage form comprising the at least one insoluble polymer dosage form is in the form of a capsule.

In at least one embodiment of the invention, the first-once daily controlled-release dosage form comprising the at least one insoluble polymer dosage form is in the form of a microparticle.

In at least one embodiment of the invention, the first-once daily controlled-release dosage form comprising the at least one insoluble polymer dosage form is in the form of a plurality of microparticles, wherein each microparticle comprises an insoluble polymer dosage form.

In at least one embodiment of the invention, the first-once daily controlled-release dosage form comprises at least one insoluble polymer dosage form, wherein said insoluble polymer dosage form comprises a unitary core.

In at least one embodiment of the invention, the first-once daily controlled-release dosage form comprises at least one insoluble polymer dosage form, wherein said insoluble polymer dosage form further comprises two or more coats, wherein one coat comprises a delayed-release coat.

In at least one embodiment of the invention, the first-once daily controlled-release dosage form comprises at least one insoluble polymer dosage form, wherein said insoluble polymer dosage form further comprises two or more coats, wherein one coat comprises a release-slowing coat.

In at least one embodiment of the invention, the first-once daily controlled-release dosage form comprises at least one insoluble polymer dosage form, wherein said insoluble polymer dosage form further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one insoluble polymer dosage form is comprised of a plurality of coated cores.

In at least one embodiment of the invention, the first once daily controlled-release dosage form comprises at least one release-slowing coat.

In at least one embodiment of the invention, the first once daily controlled-release dosage form comprising the at least one release-slowing coat comprises an effective amount of tramadol for the management of moderate to moderately severe pain.

In at least one embodiment of the invention, the first once daily controlled-release dosage form comprising the at least one release-slowing coat comprises from about 25 mg to about 800 mg of tramadol.

In at least one embodiment of the invention, the first once daily controlled-release dosage form comprising the at least one release-slowing coat comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one release-slowing coat comprises tramadol hydrochloride.

In at least one embodiment of the invention, the first once daily controlled-release dosage form comprising the at least one release-slowing coat will desirably comprise a mixture of an effective amount of at least two different tramadol salts, wherein on of the salts is tramadol hydrochloride.

In at least one embodiment of the invention, the first once daily controlled-release dosage form comprising the at least one release-slowing coat will desirably comprise about 70 to about 90% by weight of the core dry weight tramadol.

In at least one embodiment of the invention, the first once daily controlled-release dosage form comprising the at least one release-slowing coat comprises an immediate release coat, wherein said immediate release coat comprises tramadol.

In at least one embodiment of the invention, the first once daily controlled-release dosage form comprising the at least one release-slowing coat comprises an immediate release coat, wherein said immediate release coat comprises a salt or form of tramadol, which is different from the salt or form of tramadol present in the remainder of the first once daily controlled release dosage form comprising the release-slowing coat.

In at least one embodiment of the invention, the first-once daily controlled-release dosage form comprising the at least one release-slowing coat comprises at least one controlled-release matrix core.

In at least one embodiment of the invention, the first-once daily controlled-release dosage form comprising the at least one release-slowing coat comprises at least one erodable matrix core.

In at least one embodiment of the invention, the first-once daily controlled-release dosage form comprising the at least one release-slowing coat comprises at least one swellable matrix core.

In at least one embodiment of the invention, the first-once daily controlled-release dosage form comprising the at least one release-slowing coat comprises at least one erodable matrix core.

In at least one embodiment of the invention, the first-once daily controlled-release dosage form comprising the at least one release-slowing coat comprises at least one swellable and erodable matrix core.

In at least one embodiment of the invention, the first-once daily controlled-release dosage form comprising the at least one release-slowing coat comprises at least one insoluble matrix core.

In at least one embodiment of the invention, the first-once daily controlled-release dosage form comprising the at least one release-slowing coat comprises at least one hydrophobic matrix core.

In at least one embodiment of the invention, the first-once daily controlled-release dosage form comprising the at least one release-slowing coat comprises at least one insoluble polymer matrix core.

In at least one embodiment of the invention, the first-once daily controlled-release dosage form comprising the at least one release-slowing coat comprises at least one at least one delayed-release coat.

In at least one embodiment of the invention, the first-once daily controlled-release dosage form comprising the at least one release-slowing coat comprises an osmotic subcoat.

In at least one embodiment of the invention, the first-once daily controlled-release dosage form comprises at least one release-slowing coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprises at least one release-slowing coat, which coat comprises at least one water-insoluble water-permeable film-forming polymer and at least one water-soluble polymer.

In at least one embodiment of the invention, the first-once daily controlled-release dosage form comprises at least one release-slowing coat, which coat comprises at least one water-insoluble water-permeable film-forming polymer, at least one water-soluble polymer, and at least one plasticizer.

In at least one embodiment of the invention, the first-once daily controlled-release dosage form comprises at least one release-slowing coat, which coat comprises at least one enteric polymer.

In at least one embodiment of the invention, the first-once daily controlled-release dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprises at least one release-slowing coat, comprises at least one delayed-release coat, which delayed-release coat comprises at least one pH dependent polymer.

In at least one embodiment of the invention, the first-once daily controlled-release dosage form comprising the at least one release-slowing coat comprises at least one non-functional soluble coat.

In at least one embodiment of the invention, the first-once daily controlled-release dosage form comprising the at least one release-slowing coat is in the form of a tablet.

In at least one embodiment of the invention, the first-once daily controlled-release dosage form comprising the at least one release-slowing coat is in the form of a capsule.

In at least one embodiment of the invention, the first-once daily controlled-release dosage form comprising the at least one release-slowing coat is in the form of a microparticle.

In at least one embodiment of the invention, the first-once daily controlled-release dosage form comprising the at least one release-slowing coat is in the form of a plurality of microparticles, wherein each microparticle comprises a release-slowing coat.

In at least one embodiment of the invention, the first-once daily controlled-release dosage form comprises at least one release-slowing coat, wherein said first once daily controlled-release dosage form is a unitary core.

In at least one embodiment of the invention, the first-once daily controlled-release dosage form comprises at least one release-slowing coat, wherein said first once daily controlled-release matrix core further comprises two or more coats, wherein one coat comprises a delayed-release coat.

In at least one embodiment of the invention, the first-once daily controlled-release dosage form comprises at least one release-slowing coat, wherein said first once daily controlled-release matrix core further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one release-slowing coat is comprised of a plurality of coated cores.

In at least one embodiment of the invention, the first once daily controlled-release dosage form comprises at least one insoluble coat.

In at least one embodiment of the invention, the first once daily controlled-release dosage form comprising the at least one insoluble coat comprises an effective amount of tramadol for the management of moderate to moderately severe pain.

In at least one embodiment of the invention, the first once daily controlled-release dosage form comprising the at least one insoluble coat comprises from about 25 mg to about 800 mg of tramadol.

In at least one embodiment of the invention, the first once daily controlled-release dosage form comprising the at least one insoluble coat comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one insoluble coat comprises tramadol hydrochloride.

In at least one embodiment of the invention, the first once daily controlled-release dosage form comprising the at least one insoluble coat will desirably comprise a mixture of an effective amount of at least two different tramadol salts, wherein one of the salts is tramadol hydrochloride.

In at least one embodiment of the invention, the first once daily controlled-release dosage form comprising the at least one insoluble coat will desirably comprise about 70 to about 90% by weight of the core dry weight tramadol.

In at least one embodiment of the invention, the first once daily controlled-release dosage form comprising the at least one insoluble coat comprises an immediate release coat, wherein said immediate release coat comprises tramadol.

In at least one embodiment of the invention, the first once daily controlled-release dosage form comprising the at least one insoluble coat comprises an immediate release coat, wherein said immediate release coat comprises a salt or form of tramadol, which is different from the salt or form of tramadol present in the remainder of the first once daily controlled release dosage form comprising the at least one insoluble coat.

In at least one embodiment of the invention, the first-once daily controlled-release dosage form comprising the at least one insoluble coat comprises at least one controlled-release matrix core.

In at least one embodiment of the invention, the first-once daily controlled-release dosage form comprising the at least one insoluble coat comprises at least one erodable matrix core.

In at least one embodiment of the invention, the first-once daily controlled-release dosage form comprising the at least one insoluble coat comprises at least one swellable matrix core.

In at least one embodiment of the invention, the first-once daily controlled-release dosage form comprising the at least one insoluble coat comprises at least one erodable matrix core.

In at least one embodiment of the invention, the first-once daily controlled-release dosage form comprising the at least one insoluble coat comprises at least one swellable and erodable matrix core.

In at least one embodiment of the invention, the first-once daily controlled-release dosage form comprising the at least one insoluble coat comprises at least one insoluble matrix core.

In at least one embodiment of the invention, the first-once daily controlled-release dosage form comprising the at least one insoluble coat comprises at least one hydrophobic matrix core.

In at least one embodiment of the invention, the first-once daily controlled-release dosage form comprising the at least one insoluble coat comprises at least one insoluble polymer matrix core.

In at least one embodiment of the invention, the first-once daily controlled-release dosage form comprising the at least one insoluble coat comprises at least one at least one delayed-release coat.

In at least one embodiment of the invention, the first-once daily controlled-release dosage form comprising the at least one insoluble coat comprises an osmotic subcoat.

In at least one embodiment of the invention, the first-once daily controlled-release dosage form comprises at least one insoluble coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprises at least one insoluble coat, which coat comprises at least one water-insoluble water-permeable film-forming polymer and at least one water-soluble polymer.

In at least one embodiment of the invention, the first-once daily controlled-release dosage form comprises at least one insoluble coat, which coat comprises at least one water-insoluble water-permeable film-forming polymer, at least one water-soluble polymer, and at least one plasticizer.

In at least one embodiment of the invention, the first-once daily controlled-release dosage form comprises at least one insoluble coat, which coat comprises at least one enteric polymer.

In at least one embodiment of the invention, the first-once daily controlled-release dosage form comprises at least one insoluble coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprises at least one insoluble coat, comprises at least one delayed-release coat, which delayed-release coat comprises at least one pH dependent polymer.

In at least one embodiment of the invention, the first-once daily controlled-release dosage form comprising the at least one insoluble coat comprises at least one non-functional soluble coat.

In at least one embodiment of the invention, the first-once daily controlled-release dosage form comprising the at least one insoluble coat is in the form of a tablet.

In at least one embodiment of the invention, the first-once daily controlled-release dosage form comprising the at least one insoluble coat is in the form of a capsule.

In at least one embodiment of the invention, the first-once daily controlled-release dosage form comprising the at least one insoluble coat is in the form of a microparticle.

In at least one embodiment of the invention, the first-once daily controlled-release dosage form comprising the at least one insoluble coat is in the form of a plurality of microparticles, wherein each microparticle comprises an insoluble coat.

In at least one embodiment of the invention, the first-once daily controlled-release dosage form comprises at least one insoluble coat, wherein said first once daily controlled-release dosage form is a unitary core.

In at least one embodiment of the invention, the first-once daily controlled-release dosage form comprises at least one insoluble coat, wherein said first once daily controlled-release matrix core further comprises two or more coats, wherein one coat comprises a delayed-release coat.

In at least one embodiment of the invention, the first-once daily controlled-release dosage form comprises at least one insoluble coat, wherein said first once daily controlled-release matrix core further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one insoluble coat is comprised of a plurality of coated cores.

In at least one embodiment of the invention, the first once daily controlled-release dosage form comprises at least one swellable coat.

In at least one embodiment of the invention, the first once daily controlled-release dosage form comprising the at least one swellable coat comprises an effective amount of tramadol for the management of moderate to moderately severe pain.

In at least one embodiment of the invention, the first once daily controlled-release dosage form comprising the at least one swellable coat comprises from about 25 mg to about 800 mg of tramadol.

In at least one embodiment of the invention, the first once daily controlled-release dosage form comprising the at least one swellable coat comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one swellable coat comprises tramadol hydrochloride.

In at least one embodiment of the invention, the first once daily controlled-release dosage form comprising the at least one swellable coat will desirably comprise a mixture of an effective amount of at least two different tramadol salts, wherein one of the salts is tramadol hydrochloride.

In at least one embodiment of the invention, the first once daily controlled-release dosage form comprising the at least one swellable coat will desirably comprise about 70 to about 90% by weight of the core dry weight tramadol.

In at least one embodiment of the invention, the first once daily controlled-release dosage form comprising the at least one swellable coat comprises an immediate release coat, wherein said immediate release coat comprises tramadol.

In at least one embodiment of the invention, the first once daily controlled-release dosage form comprising the at least one swellable coat comprises an immediate release coat, wherein said immediate release coat comprises a salt or form of tramadol, which is different from the salt or form of tramadol present in the remainder of the first once daily controlled release dosage form comprising the swellable coat.

In at least one embodiment of the invention, the first-once daily controlled-release dosage form comprising the at least one swellable coat comprises at least one controlled-release matrix core.

In at least one embodiment of the invention, the first-once daily controlled-release dosage form comprising the at least one swellable coat comprises at least one erodable matrix core.

In at least one embodiment of the invention, the first-once daily controlled-release dosage form comprising the at least one swellable coat comprises at least one swellable matrix core.

In at least one embodiment of the invention, the first-once daily controlled-release dosage form comprising the at least one swellable coat comprises at least one erodable matrix core.

In at least one embodiment of the invention, the first-once daily controlled-release dosage form comprising the at least one swellable coat comprises at least one swellable and erodable matrix core.

In at least one embodiment of the invention, the first-once daily controlled-release dosage form comprising the at least one swellable coat comprises at least one insoluble matrix core.

In at least one embodiment of the invention, the first-once daily controlled-release dosage form comprising the at least one swellable coat comprises at least one hydrophobic matrix core.

In at least one embodiment of the invention, the first-once daily controlled-release dosage form comprising the at least one swellable coat comprises at least one insoluble polymer matrix core.

In at least one embodiment of the invention, the first-once daily controlled-release dosage form comprising the at least one swellable coat comprises at least one at least one delayed-release coat.

In at least one embodiment of the invention, the first-once daily controlled-release dosage form comprising the at least one swellable coat comprises an osmotic subcoat.

In at least one embodiment of the invention, the first-once daily controlled-release dosage form comprises at least one swellable coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprises at least one swellable coat, which coat comprises at least one water-insoluble water-permeable film-forming polymer and at least one water-soluble polymer.

In at least one embodiment of the invention, the first-once daily controlled-release dosage form comprises at least one swellable coat, which coat comprises at least one water-insoluble water-permeable film-forming polymer, at least one water-soluble polymer, and at least one plasticizer.

In at least one embodiment of the invention, the first-once daily controlled-release dosage form comprises at least one swellable coat, which coat comprises at least one enteric polymer.

In at least one embodiment of the invention, the first-once daily controlled-release dosage form comprises at least one swellable coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprises at least one swellable coat, comprises at least one delayed-release coat, which delayed-release coat comprises at least one pH dependent polymer.

In at least one embodiment of the invention, the first-once daily controlled-release dosage form comprising the at least one swellable coat comprises at least one non-functional soluble coat.

In at least one embodiment of the invention, the first-once daily controlled-release dosage form comprising the at least one swellable coat is in the form of a tablet.

In at least one embodiment of the invention, the first-once daily controlled-release dosage form comprising the at least one swellable coat is in the form of a capsule.

In at least one embodiment of the invention, the first-once daily controlled-release dosage form comprising the at least one swellable coat is in the form of a microparticle.

In at least one embodiment of the invention, the first-once daily controlled-release dosage form comprising the at least one swellable coat is in the form of a plurality of microparticles, wherein each microparticle comprises an insoluble coat.

In at least one embodiment of the invention, the first-once daily controlled-release dosage form comprises at least one swellable coat, wherein said first once daily controlled-release dosage form is a unitary core.

In at least one embodiment of the invention, the first-once daily controlled-release dosage form comprises at least one swellable coat, wherein said first once daily controlled-release matrix core further comprises two or more coats, wherein one coat comprises a delayed-release coat.

In at least one embodiment of the invention, the first-once daily controlled-release dosage form comprises at least one insoluble coat, wherein said first once daily controlled-release matrix core further comprises two or more coats, wherein one coat comprises an enhanced-absorption coat.

In at least one embodiment of the invention, the first-once daily controlled-release dosage form comprises at least one insoluble coat, wherein said first once daily controlled-release matrix core further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one swellable coat is comprised of a plurality of coated cores.

In at least one embodiment of the invention, the first once daily controlled-release dosage form comprises at least one erodable coat.

In at least one embodiment of the invention, the first once daily controlled-release dosage form comprising the at least one erodable coat comprises an effective amount of tramadol for the management of moderate to moderately severe pain.

In at least one embodiment of the invention, the first once daily controlled-release dosage form comprising the at least one erodable coat comprises from about 25 mg to about 800 mg of tramadol.

In at least one embodiment of the invention, the first once daily controlled-release dosage form comprising the at least one erodable coat comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one erodable coat comprises tramadol hydrochloride.

In at least one embodiment of the invention, the first once daily controlled-release dosage form comprising the at least one erodable coat will desirably comprise a mixture of an effective amount of at least two different tramadol salts, wherein one of the salts is tramadol hydrochloride.

In at least one embodiment of the invention, the first once daily controlled-release dosage form comprising the at least one erodable coat will desirably comprise about 70 to about 90% by weight of the core dry weight tramadol.

In at least one embodiment of the invention, the first once daily controlled-release dosage form comprising the at least one erodable coat comprises an immediate release coat, wherein said immediate release coat comprises tramadol.

In at least one embodiment of the invention, the first once daily controlled-release dosage form comprising the at least one erodable coat comprises an immediate release coat, wherein said immediate release coat comprises a salt or form of tramadol, which is different from the salt or form of tramadol present in the remainder of the first once daily controlled release dosage form comprising the erodable coat.

In at least one embodiment of the invention, the first-once daily controlled-release dosage form comprising the at least one erodable coat comprises at least one controlled-release matrix core.

In at least one embodiment of the invention, the first-once daily controlled-release dosage form comprising the at least one erodable coat comprises at least one erodable matrix core.

In at least one embodiment of the invention, the first-once daily controlled-release dosage form comprising the at least one erodable coat comprises at least one swellable matrix core.

In at least one embodiment of the invention, the first-once daily controlled-release dosage form comprising the at least one erodable coat comprises at least one erodable matrix core.

In at least one embodiment of the invention, the first-once daily controlled-release dosage form comprising the at least one erodable coat comprises at least one swellable and erodable matrix core.

In at least one embodiment of the invention, the first-once daily controlled-release dosage form comprising the at least one erodable coat comprises at least one insoluble matrix core.

In at least one embodiment of the invention, the first-once daily controlled-release dosage form comprising the at least one erodable coat comprises at least one hydrophobic matrix core.

In at least one embodiment of the invention, the first-once daily controlled-release dosage form comprising the at least one erodable coat comprises at least one insoluble polymer matrix core.

In at least one embodiment of the invention, the first-once daily controlled-release dosage form comprising the at least one erodable coat comprises at least one at least one delayed-release coat.

In at least one embodiment of the invention, the first-once daily controlled-release dosage form comprising the at least one erodable coat comprises an osmotic subcoat.

In at least one embodiment of the invention, the first-once daily controlled-release dosage form comprises at least one erodable coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first-once daily controlled-release dosage form comprises at least one erodable coat, which coat comprises at least one water-insoluble water-permeable film-forming polymer and at least one water-soluble polymer.

In at least one embodiment of the invention, the first-once daily controlled-release dosage form comprises at least one erodable coat, which coat comprises at least one water-insoluble water-permeable film-forming polymer, at least one water-soluble polymer, and at least one plasticizer.

In at least one embodiment of the invention, the first-once daily controlled-release dosage form comprises at least one erodable coat, which coat comprises at least one enteric polymer.

In at least one embodiment of the invention, the first-once daily controlled-release dosage form comprises at least one erodable coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprises at least one erodable coat, comprises at least one delayed-release coat, which delayed-release coat comprises at least one pH dependent polymer.

In at least one embodiment of the invention, the first-once daily controlled-release dosage form comprising the at least one erodable coat comprises at least one non-functional soluble coat.

In at least one embodiment of the invention, the first-once daily controlled-release dosage form comprising the at least one erodable coat is in the form of a tablet.

In at least one embodiment of the invention, the first-once daily controlled-release dosage form comprising the at least one erodable coat is in the form of a capsule.

In at least one embodiment of the invention, the first-once daily controlled-release dosage form comprising the at least one erodable coat is in the form of a microparticle.

In at least one embodiment of the invention, the first-once daily controlled-release dosage form comprising the at least one erodable coat is in the form of a plurality of microparticles, wherein each microparticle comprises an insoluble coat.

In at least one embodiment of the invention, the first-once daily controlled-release dosage form comprises at least one erodable coat, wherein said first once daily controlled-release dosage form is a unitary core.

In at least one embodiment of the invention, the first-once daily controlled-release dosage form comprises at least one erodable coat, wherein said first once daily controlled-release dosage form further comprises two or more coats, wherein one coat comprises a delayed-release coat.

In at least one embodiment of the invention, the first-once daily controlled-release dosage form comprises at least one erodable coat, wherein said first once daily controlled-release dosage form further comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first-once daily controlled-release dosage form comprising the at least one erodable coat is comprised of a plurality of coated cores.

In at least one embodiment of the invention, the first once daily controlled-release dosage form comprises at least one extended-release dosage form.

In at least one embodiment of the invention, the first once daily controlled-release dosage form comprising the at least one extended-release dosage form comprises an effective amount of tramadol for the management of moderate to moderately severe pain.

In at least one embodiment of the invention, the first once daily controlled-release dosage form comprising the at least one extended-release dosage form comprises from about 25 mg to about 800 mg of tramadol.

In at least one embodiment of the invention, the first once daily controlled-release dosage form comprising the at least one extended-release dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one extended-release dosage form comprises tramadol hydrochloride

In at least one embodiment of the invention, the first once daily controlled-release dosage form comprising the at least one extended-release dosage form will desirably comprise a mixture of an effective amount of at least two different tramadol salts, wherein one of the salts is tramadol hydrochloride.

In at least one embodiment of the invention, the first once daily controlled-release dosage form comprising the at least one extended-release dosage form will desirably comprise about 70 to about 90% by weight of the core dry weight tramadol.

In at least one embodiment of the invention, the first once daily controlled-release dosage form comprising the at least one extended-release dosage form will desirably comprise an immediate release coat, wherein said immediate release coat comprises tramadol.

In at least one embodiment of the invention, the first once daily controlled-release dosage form comprising the at least one extended-release dosage form will desirably comprise an immediate release coat, wherein said immediate release coat comprises a salt or form of tramadol, which is different from the salt or form of tramadol present in the remainder of the extended-release dosage form.

In at least one embodiment of the invention, the first once daily controlled-release dosage form comprising the at least one extended-release dosage form comprises at least one controlled-release matrix core.

In at least one embodiment of the invention, the first once daily controlled-release dosage form comprising the at least one extended-release dosage form comprises at least one insoluble matrix core.

In at least one embodiment of the invention, the first once daily controlled-release dosage form comprising the at least one extended-release dosage form comprises at least one hydrophobic matrix core.

In at least one embodiment of the invention, the first once daily controlled-release dosage form comprising the at least one extended-release dosage form comprises at least one swellable matrix core.

In at least one embodiment of the invention, the first once daily controlled-release dosage form comprising the at least one extended-release dosage form comprises at least one erodible matrix core.

In at least one embodiment of the invention, the first once daily controlled-release dosage form comprising the at least one extended-release dosage form comprises at least one swellable and erodible matrix core.

In at least one embodiment of the invention, the first once daily controlled-release dosage form comprising the at least one extended-release dosage form comprises at least one release slowing coat.

In at least one embodiment of the invention, the first once daily controlled-release dosage form comprising the at least one extended-release dosage form comprises at least one aqueous insoluble coat.

In at least one embodiment of the invention, the first once daily controlled-release dosage form comprising the at least one extended-release dosage form comprises at least one erodable coat.

In at least one embodiment of the invention, the first once daily controlled-release dosage form comprising the at least one extended-release dosage form comprises at least one swellable coat.

In at least one embodiment of the invention, the first once daily controlled-release dosage form comprising the at least one extended-release dosage form comprises at least one swellable and erodible coat.

In at least one embodiment of the invention, the first once daily controlled-release dosage form comprising the at least one extended-release dosage form comprises at least one release-slowing coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprising the at least one extended-release dosage form comprises at least one release-slowing coat, which coat comprises at least one water-insoluble water-permeable film-forming polymer and at least one water-soluble polymer.

In at least one embodiment of the invention, the first once daily controlled-release dosage form comprising the at least one extended-release dosage form comprises at least one release-slowing coat, which coat comprises at least one water-insoluble water-permeable film-forming polymer, at least one water-soluble polymer, and at least one plasticizer.

In at least one embodiment of the invention, the first once daily controlled-release dosage form comprising the at least one extended-release dosage form comprises at least one release-slowing coat, which coat comprises at least one enteric polymer.

In at least one embodiment of the invention, the first once daily controlled-release dosage form comprising the at least one extended-release dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one extended-release dosage form comprises at least one release-slowing coat, which coat comprises at least one pH dependent polymer.

In at least one embodiment of the invention, the first once daily controlled-release dosage form comprising the at least one extended-release dosage form comprises at least one non-functional soluble coat.

In at least one embodiment of the invention, the first once daily controlled-release dosage form comprising the at least one extended-release dosage form comprises at least one delayed-release coat.

In at least one embodiment of the invention, the first once daily controlled-release dosage form comprising the at least one extended-release dosage form is in the form of a tablet.

In at least one embodiment of the invention, the first once daily controlled-release dosage form comprising the at least one extended-release dosage form is in the form of a capsule.

In at least one embodiment of the invention, the first once daily controlled-release dosage form comprising the at least one extended-release dosage form is in the form of a microparticle.

In at least one embodiment of the invention, the first once daily controlled-release dosage form comprising the at least one extended-release dosage form is in the form of a plurality of microparticles, wherein each microparticle is comprised of an extended release dosage form.

In at least one embodiment of the invention, the first once daily controlled-release dosage form comprises at least one extended-release dosage form, wherein the first once daily controlled-release dosage form comprises a unitary core.

In at least one embodiment of the invention, the first once daily controlled-release dosage form comprises at least one extended-release dosage form, wherein said first once daily controlled-release dosage form comprises two or more coats, wherein one coat comprises a delayed-release coat.

In at least one embodiment of the invention, the first once daily controlled-release dosage form comprises at least one extended-release dosage form, wherein said first once daily controlled-release dosage form comprises two or more coats, wherein one coat comprises a release slowing coat.

In at least one embodiment of the invention, the first once daily controlled-release dosage form comprises at least one extended-release dosage form, wherein said first once daily controlled-release dosage form comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one extended-release dosage form is comprised of a plurality of coated cores.

In at least one embodiment of the invention, the first once daily controlled-release dosage form comprises at least one delayed-release dosage form.

In at least one embodiment of the invention, the first once daily controlled-release dosage form comprising the at least one delayed-release dosage form comprises an effective amount of tramadol for the management of moderate to moderately severe pain.

In at least one embodiment of the invention, the first once daily controlled-release dosage form comprising the at least one delayed-release dosage form comprises from about 25 mg to about 800 mg of tramadol.

In at least one embodiment of the invention, the first once daily controlled-release dosage form comprising the at least one delayed-release dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one delayed-release dosage form comprises tramadol hydrochloride.

In at least one embodiment of the invention, the first once daily controlled-release dosage form comprising the at least one delayed-release dosage form will desirably comprise a mixture of an effective amount of at least two different tramadol salts, wherein one of the salts is tramadol hydrochloride.

In at least one embodiment of the invention, the first once daily controlled-release dosage form comprising the at least one delayed-release dosage form will desirably comprise about 70 to about 90% by weight of the core dry weight tramadol.

In at least one embodiment of the invention, the first once daily controlled-release dosage form comprising the at least one delayed-release dosage form comprises an immediate release coat, wherein said immediate release coat comprises tramadol.

In at least one embodiment of the invention, the first once daily controlled-release dosage form comprising the at least one delayed-release dosage form comprises an immediate release coat, wherein said immediate release coat comprises a salt or form of tramadol, which is different from the salt or form of tramadol present in the remainder of the delayed release dosage form.

In at least one embodiment of the invention, the first once daily controlled-release dosage form comprising the at least one delayed-release dosage form comprises at least one controlled-release matrix core.

In at least one embodiment of the invention, the first once daily controlled-release dosage form comprising the at least one delayed-release dosage form comprises at least one insoluble matrix core.

In at least one embodiment of the invention, the first once daily controlled-release dosage form comprising the at least one delayed-release dosage form comprises at least one hydrophobic matrix core.

In at least one embodiment of the invention, the first once daily controlled-release dosage form comprising the at least one delayed-release dosage form comprises at least one swellable matrix core.

In at least one embodiment of the invention, the first once daily controlled-release dosage form comprising the at least one delayed-release dosage form comprises at least one erodible matrix core.

In at least one embodiment of the invention, the first once daily controlled-release dosage form comprising the at least one delayed-release dosage form comprises at least one swellable and erodible matrix core.

In at least one embodiment of the invention, the first once daily controlled-release dosage form comprising the at least one delayed-release dosage form comprises at least one release slowing coat.

In at least one embodiment of the invention, the first once daily controlled-release dosage form comprising the at least one delayed-release dosage form comprises at least one aqueous insoluble coat.

In at least one embodiment of the invention, the first once daily controlled-release dosage form comprising the at least one delayed-release dosage form comprises at least one erodable coat.

In at least one embodiment of the invention, the first once daily controlled-release dosage form comprising the at least one delayed-release dosage form comprises at least one swellable coat.

In at least one embodiment of the invention, the first once daily controlled-release dosage form comprising the at least one delayed-release dosage form comprises at least one swellable and erodible coat.

In at least one embodiment of the invention, the first once daily controlled-release dosage form comprising the at least one delayed-release dosage form comprises at least one release-slowing coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprising the at least one delayed-release dosage form comprises at least one release-slowing coat, which coat comprises at least one water-insoluble water-permeable film-forming polymer and at least one water-soluble polymer.

In at least one embodiment of the invention, the first once daily controlled-release dosage form comprising the at least one delayed-release dosage form comprises at least one release-slowing coat, which coat comprises at least one water-insoluble water-permeable film-forming polymer, at least one water-soluble polymer, and at least one plasticizer.

In at least one embodiment of the invention, the first once daily controlled-release dosage form comprising the at least one delayed-release dosage form comprises at least one release-slowing coat, which coat comprises at least one enteric polymer.

In at least one embodiment of the invention, the first once daily controlled-release dosage form comprising the at least one delayed-release dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one delayed-release dosage form comprises at least one release-slowing coat, which coat comprises at least one pH dependent polymer.

In at least one embodiment of the invention, the first once daily controlled-release dosage form comprising the at least one delayed-release dosage form comprises at least one non-functional soluble coat.

In at least one embodiment of the invention, the first once daily controlled-release dosage form comprising the at least one delayed-release dosage form is in the form of a tablet.

In at least one embodiment of the invention, the first once daily controlled-release dosage form comprising the at least one delayed-release dosage form is in the form of a capsule.

In at least one embodiment of the invention, the first once daily controlled-release dosage form comprising the at least one delayed-release dosage form is in the form of a microparticle.

In at least one embodiment of the invention, the first once daily controlled-release dosage form comprising the at least one delayed-release dosage form is in the form of a plurality of microparticles, wherein each microparticle is comprised of a delayed-release dosage form.

In at least one embodiment of the invention, the first once daily controlled-release dosage form comprises at least one delayed-release dosage form, wherein the first once daily controlled-release dosage form comprises a unitary core.

In at least one embodiment of the invention, the first once daily controlled-release dosage form comprises at least one delayed-release dosage form, wherein said first once daily controlled-release dosage form comprises two or more coats, wherein one coat comprises a release-slowing coat.

In at least one embodiment of the invention, the first once daily controlled-release dosage form comprises at least one delayed-release dosage form, wherein said first once daily controlled-release dosage form comprises two or more coats, wherein one coat comprises a delayed-release coat.

In at least one embodiment of the invention, the first once daily controlled-release dosage form comprises at least one delayed-release dosage form, wherein said first once daily controlled-release dosage form comprises two or more coats, wherein one coat comprises an enhanced-absorption coat.

In at least one embodiment of the invention, the first once daily controlled-release dosage form comprises at least one delayed-release dosage form, wherein said first once daily controlled-release dosage form comprises two or more coats, wherein one coat comprises an enhanced-absorption coat.

In at least one embodiment of the invention, the first once daily controlled-release dosage form comprises at least one delayed-release dosage form, wherein said first once daily controlled-release dosage form comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one delayed-release dosage form is comprised of a plurality of coated cores.

In at least one embodiment of the invention, the first once daily controlled-release dosage form comprises at least one modified-release dosage form.

In at least one embodiment of the invention, the first once daily controlled-release dosage form comprising the at least one modified-release dosage form comprises an effective amount of tramadol for the management of moderate to moderately severe pain.

In at least one embodiment of the invention, the first once daily controlled-release dosage form comprising the at least one modified-release dosage form comprises from about 25 mg to about 800 mg of tramadol.

In at least one embodiment of the invention, the first once daily controlled-release dosage form comprising the at least one modified-release dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one modified-release dosage form comprises tramadol hydrochloride.

In at least one embodiment of the invention, the first once daily controlled-release dosage form comprising the at least one modified-release dosage form will desirably comprise a mixture of an effective amount of at least two different tramadol salts, wherein one of the salts is tramadol hydrochloride.

In at least one embodiment of the invention, the first once daily controlled-release dosage form comprising the at least one modified-release dosage form will desirably comprise about 70 to about 90% by weight of the core dry weight tramadol.

In at least one embodiment of the invention, the first once daily controlled-release dosage form comprising the at least one modified-release dosage form comprises an immediate release coat, wherein said immediate release coat comprises tramadol.

In at least one embodiment of the invention, the first once daily controlled-release dosage form comprising the at least one modified-release dosage form comprises an immediate release coat, wherein said immediate release coat comprises a salt or form of tramadol, which is different from the salt or form of tramadol present in the remainder of the delayed-release dosage form.

In at least one embodiment of the invention, the first once daily controlled-release dosage form comprising the at least one modified-release dosage form comprises at least one controlled-release matrix core.

In at least one embodiment of the invention, the first once daily controlled-release dosage form comprising the at least one modified-release dosage form comprises at least one insoluble matrix core.

In at least one embodiment of the invention, the first once daily controlled-release dosage form comprising the at least one modified-release dosage form comprises at least one hydrophobic matrix core.

In at least one embodiment of the invention, the first once daily controlled-release dosage form comprising the at least one modified-release dosage form comprises at least one swellable matrix core.

In at least one embodiment of the invention, the first once daily controlled-release dosage form comprising the at least one modified-release dosage form comprises at least one erodible matrix core.

In at least one embodiment of the invention, the first once daily controlled-release dosage form comprising the at least one modified-release dosage form comprises at least one swellable and erodible matrix core.

In at least one embodiment of the invention, the first once daily controlled-release dosage form comprising the at least one modified-release dosage form comprises at least one release slowing coat.

In at least one embodiment of the invention, the first once daily controlled-release dosage form comprising the at least one modified-release dosage form comprises at least one aqueous insoluble coat.

In at least one embodiment of the invention, the first once daily controlled-release dosage form comprising the at least one modified-release dosage form comprises at least one erodable coat.

In at least one embodiment of the invention, the first once daily controlled-release dosage form comprising the at least one modified-release dosage form comprises at least one swellable coat.

In at least one embodiment of the invention, the first once daily controlled-release dosage form comprising the at least one modified-release dosage form comprises at least one swellable and erodible coat.

In at least one embodiment of the invention, the first once daily controlled-release dosage form comprising the at least one modified-release dosage form comprises at least one release-slowing coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprising the at least one modified-release dosage form comprises at least one release-slowing coat, which coat comprises at least one water-insoluble water-permeable film-forming polymer and at least one water-soluble polymer.

In at least one embodiment of the invention, the first once daily controlled-release dosage form comprising the at least one modified-release dosage form comprises at least one release-slowing coat, which coat comprises at least one water-insoluble water-permeable film-forming polymer, at least one water-soluble polymer, and at least one plasticizer.

In at least one embodiment of the invention, the first once daily controlled-release dosage form comprising the at least one delayed-release dosage form comprises at least one delayed-release coat.

In at least one embodiment of the invention, the first once daily controlled-release dosage form comprising the at least one modified-release dosage form comprises at least one release-slowing coat, which coat comprises at least one enteric polymer.

In at least one embodiment of the invention, the first once daily controlled-release dosage form comprising the at least one modified-release dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one modified-release dosage form comprises at least one release-slowing coat, which coat comprises at least one pH dependent polymer.

In at least one embodiment of the invention, the first once daily controlled-release dosage form comprising the at least one modified-release dosage form comprises at least one non-functional soluble coat.

In at least one embodiment of the invention, the first once daily controlled-release dosage form comprising the at least one modified-release dosage form is in the form of a tablet.

In at least one embodiment of the invention, the first once daily controlled-release dosage form comprising the at least one modified-release dosage form is in the form of a capsule.

In at least one embodiment of the invention, the first once daily controlled-release dosage form comprising the at least one modified-release dosage form is in the form of a microparticle.

In at least one embodiment of the invention, the first once daily controlled-release dosage form comprising the at least one modified-release dosage form is in the form of a plurality of microparticles, wherein each microparticle is comprised of a modified-release dosage form.

In at least one embodiment of the invention, the first once daily controlled-release dosage form comprises at least one modified-release dosage form, wherein the first once daily controlled-release dosage form comprises a unitary core.

In at least one embodiment of the invention, the first once daily controlled-release dosage form comprises at least one modified-release dosage form, wherein said first once daily controlled-release dosage form comprises two or more coats, wherein one coat comprises a release-slowing coat.

In at least one embodiment of the invention, the first once daily controlled-release dosage form comprises at least one modified-release dosage form, wherein said first once daily controlled-release dosage form comprises two or more coats, wherein one coat comprises a delayed-release coat.

In at least one embodiment of the invention, the first once daily controlled-release dosage form comprises at least one modified-release dosage form, wherein said first once daily controlled-release dosage form comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one modified-release dosage form is comprised of a plurality of coated cores.

In at least one embodiment of the invention, the first once daily controlled-release dosage form comprises at least one sustained-release dosage form.

In at least one embodiment of the invention, the first once daily controlled-release dosage form comprising the at least one sustained-release dosage form comprises an effective amount of tramadol for the management of moderate to moderately severe pain.

In at least one embodiment of the invention, the first once daily controlled-release dosage form comprising the at least one sustained-release dosage form comprises from about 25 mg to about 800 mg of tramadol.

In at least one embodiment of the invention, the first once daily controlled-release dosage form comprising the at least one sustained-release dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one sustained-release dosage form will desirably comprise a mixture of an effective amount of at least two different tramadol salts.

In at least one embodiment of the invention, the first once daily controlled-release dosage form comprising the at least one sustained-release dosage form will desirably comprise about 70 to about 90% by weight of the core dry weight tramadol.

In at least one embodiment of the invention, the first once daily controlled-release dosage form comprising the at least one sustained-release dosage form comprises an immediate release coat, wherein said immediate release coat comprises tramadol.

In at least one embodiment of the invention, the first once daily controlled-release dosage form comprising the at least one sustained-release dosage form comprises an immediate release coat, wherein said immediate release coat comprises a salt or form of tramadol, which is different from the salt or form of tramadol present in the remainder of the sustained-release dosage form.

In at least one embodiment of the invention, the first once daily controlled-release dosage form comprising the at least one sustained-release dosage form comprises at least one controlled-release matrix core.

In at least one embodiment of the invention, the first once daily controlled-release dosage form comprising the at least one sustained-release dosage form comprises at least one insoluble matrix core.

In at least one embodiment of the invention, the first once daily controlled-release dosage form comprising the at least one sustained-release dosage form comprises at least one hydrophobic matrix core.

In at least one embodiment of the invention, the first once daily controlled-release dosage form comprising the at least one sustained-release dosage form comprises at least one swellable matrix core.

In at least one embodiment of the invention, the first once daily controlled-release dosage form comprising the at least one sustained-release dosage form comprises at least one erodible matrix core.

In at least one embodiment of the invention, the first once daily controlled-release dosage form comprising the at least one sustained-release dosage form comprises at least one swellable and erodible matrix core.

In at least one embodiment of the invention, the first once daily controlled-release dosage form comprising the at least one sustained-release dosage form comprises at least one release slowing coat.

In at least one embodiment of the invention, the first once daily controlled-release dosage form comprising the at least one sustained-release dosage form comprises at least one aqueous insoluble coat.

In at least one embodiment of the invention, the first once daily controlled-release dosage form comprising the at least one sustained-release dosage form comprises at least one erodable coat.

In at least one embodiment of the invention, the first once daily controlled-release dosage form comprising the at least one sustained-release dosage form comprises at least one swellable coat.

In at least one embodiment of the invention, the first once daily controlled-release dosage form comprising the at least one sustained-release dosage form comprises at least one swellable and erodible coat.

In at least one embodiment of the invention, the first once daily controlled-release dosage form comprising the at least one sustained-release dosage form comprises at least one release-slowing coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprising the at least one sustained-release dosage form comprises at least one release-slowing coat, which coat comprises at least one water-insoluble water-permeable film-forming polymer and at least one water-soluble polymer.

In at least one embodiment of the invention, the first once daily controlled-release dosage form comprising the at least one sustained-release dosage form comprises at least one release-slowing coat, which coat comprises at least one water-insoluble water-permeable film-forming polymer, at least one water-soluble polymer, and at least one plasticizer.

In at least one embodiment of the invention, the first once daily controlled-release dosage form comprising the at least one sustained-release dosage form comprises at least one release-slowing coat, which coat comprises at least one enteric polymer.

In at least one embodiment of the invention, the first once daily controlled-release dosage form comprising the at least one sustained-release dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one sustained-release dosage form comprises at least one release-slowing coat, which coat comprises at least one pH dependent polymer.

In at least one embodiment of the invention, the first once daily controlled-release dosage form comprising the at least one sustained-release dosage form comprises at least one non-functional soluble coat.

In at least one embodiment of the invention, the first once daily controlled-release dosage form comprising the at least one sustained-release lease dosage form is in the form of a tablet.

In at least one embodiment of the invention, the first once daily controlled-release dosage form comprising the at least one sustained-release dosage form is in the form of a capsule.

In at least one embodiment of the invention, the first once daily controlled-release dosage form comprising the at least one sustained-release dosage form is in the form of a microparticle.

In at least one embodiment of the invention, the first once daily controlled-release dosage form comprising the at least one sustained-release dosage form is in the form of a plurality of microparticles, wherein each microparticle is comprised of a sustained-release dosage form.

In at least one embodiment of the invention, the first once daily controlled-release dosage form comprises at least one sustained-release dosage form, wherein the first once daily controlled-release dosage form comprises a unitary core.

In at least one embodiment of the invention, the first once daily controlled-release dosage form comprises at least one sustained-release dosage form, wherein said first once daily controlled-release dosage form comprises two or more coats, wherein one coat comprises a release-slowing coat.

In at least one embodiment of the invention, the first once daily controlled-release dosage form comprises at least one sustained-release dosage form, wherein said first once daily controlled-release dosage form comprises two or more coats, wherein one coat comprises a delayed-release coat.

In at least one embodiment of the invention, the first once daily controlled-release dosage form comprises at least one sustained-release dosage form, wherein said first once daily controlled-release dosage form comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one sustained-release dosage form is comprised of a plurality of coated cores.

In at least one embodiment of the invention, the first once daily controlled-release dosage form comprises at least one prolonged-release dosage form.

In at least one embodiment of the invention, the first once daily controlled-release dosage form comprising the at least one prolonged-release dosage form comprises an effective amount of tramadol for the management of moderate to moderately severe pain.

In at least one embodiment of the invention, the first once daily controlled-release dosage form comprising the at least one prolonged-release dosage form comprises from about 25 mg to about 800 mg of tramadol.

In at least one embodiment of the invention, the first once daily controlled-release dosage form comprising the at least one prolonged-release dosage form comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one prolonged-release dosage form will desirably comprise a mixture of an effective amount of at least two different tramadol salts.

In at least one embodiment of the invention, the first once daily controlled-release dosage form comprising the at least one prolonged-release dosage form will desirably comprise about 70 to about 90% by weight of the core dry weight tramadol.

In at least one embodiment of the invention, the first once daily controlled-release dosage form comprising the at least one prolonged-release dosage form comprises an immediate release coat, wherein said immediate release coat comprises tramadol.

In at least one embodiment of the invention, the first once daily controlled-release dosage form comprising the at least one prolonged-release dosage form comprises an immediate release coat, wherein said immediate release coat comprises a salt or form of tramadol, which is different from the salt or form of tramadol present in the remainder of the prolonged-release dosage form.

In at least one embodiment of the invention, the first once daily controlled-release dosage form comprising the at least one prolonged-release dosage form comprises at least one controlled-release matrix core.

In at least one embodiment of the invention, the first once daily controlled-release dosage form comprising the at least one prolonged-release dosage form comprises at least one insoluble matrix core.

In at least one embodiment of the invention, the first once daily controlled-release dosage form comprising the at least one prolonged-release dosage form comprises at least one hydrophobic matrix core.

In at least one embodiment of the invention, the first once daily controlled-release dosage form comprising the at least one prolonged-release dosage form comprises at least one swellable matrix core.

In at least one embodiment of the invention, the first once daily controlled-release dosage form comprising the at least one prolonged-release dosage form comprises at least one erodible matrix core.

In at least one embodiment of the invention, the first once daily controlled-release dosage form comprising the at least one prolonged-release dosage form comprises at least one swellable and erodible matrix core.

In at least one embodiment of the invention, the first once daily controlled-release dosage form comprising the at least one prolonged-release dosage form comprises at least one release slowing coat.

In at least one embodiment of the invention, the first once daily controlled-release dosage form comprising the at least one prolonged-release dosage form comprises at least one aqueous insoluble coat.

In at least one embodiment of the invention, the first once daily controlled-release dosage form comprising the at least one prolonged-release dosage form comprises at least one erodable coat.

In at least one embodiment of the invention, the first once daily controlled-release dosage form comprising the at least one prolonged-release dosage form comprises at least one swellable coat.

In at least one embodiment of the invention, the first once daily controlled-release dosage form comprising the at least one prolonged-release dosage form comprises at least one swellable and erodible coat.

In at least one embodiment of the invention, the first once daily controlled-release dosage form comprising the at least one prolonged-release dosage form comprises at least one release-slowing coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprising the at least one prolonged-release dosage form comprises at least one release-slowing coat, which coat comprises at least one water-insoluble water-permeable film-forming polymer and at least one water-soluble polymer.

In at least one embodiment of the invention, the first once daily controlled-release dosage form comprising the at least one prolonged-release dosage form comprises at least one release-slowing coat, which coat comprises at least one water-insoluble water-permeable film-forming polymer, at least one water-soluble polymer, and at least one plasticizer.

In at least one embodiment of the invention, the first once daily controlled-release dosage form comprising the at least one prolonged-release dosage form comprises at least one release-slowing coat, which coat comprises at least one enteric polymer.

In at least one embodiment of the invention, the first once daily controlled-release dosage form comprising the at least one prolonged-release dosage form comprises at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one prolonged-release dosage form comprises at least one release-slowing coat, which coat comprises at least one pH dependent polymer.

In at least one embodiment of the invention, the first once daily controlled-release dosage form comprising the at least one prolonged-release dosage form comprises at least one non-functional soluble coat.

In at least one embodiment of the invention, the first once daily controlled-release dosage form comprising the at least one prolonged-release dosage form is in the form of a tablet.

In at least one embodiment of the invention, the first once daily controlled-release dosage form comprising the at least one prolonged-release dosage form is in the form of a capsule.

In at least one embodiment of the invention, the first once daily controlled-release dosage form comprising the at least one prolonged-release dosage form is in the form of a microparticle.

In at least one embodiment of the invention, the first once daily controlled-release dosage form comprising the at least one prolonged-release dosage form is in the form of a plurality of microparticles, wherein each microparticle is comprised of a prolonged-release dosage form.

In at least one embodiment of the invention, the first once daily controlled-release dosage form comprises at least one prolonged-release dosage form, wherein the first once daily controlled-release dosage form comprises a unitary core.

In at least one embodiment of the invention, the first once daily controlled-release dosage form comprises at least one prolonged-release dosage form, wherein said first once daily controlled-release dosage form comprises two or more coats, wherein one coat comprises a release-slowing coat.

In at least one embodiment of the invention, the first once daily controlled-release dosage form comprises at least one prolonged-release dosage form, wherein said first once daily controlled-release dosage form comprises two or more coats, wherein one coat comprises a delayed-release coat.

In at least one embodiment of the invention, the first once daily controlled-release dosage form comprises at least one prolonged-release dosage form, wherein said first once daily controlled-release dosage form comprises two or more coats, wherein one coat comprises an enhanced-absorption coat.

In at least one embodiment of the invention, the first once daily controlled-release dosage form comprises at least one prolonged-release dosage form, wherein said first once daily controlled-release dosage form comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising the at least one prolonged-release dosage form is comprised of a plurality of coated cores.

In at least one embodiment of the invention, the first once daily controlled-release dosage form comprises at least one normal release matrix core coated with at least one release-slowing coat.

In at least one embodiment of the invention, the first once daily controlled-release dosage form comprising the at least one normal release matrix core coated with at least one release-slowing coat comprises an effective amount of tramadol for the management of moderate to moderately severe pain.

In at least one embodiment of the invention, the first once daily controlled-release dosage form comprising the at least one normal release matrix core coated with at least one release-slowing coat comprises from about 25 mg to about 800 mg of tramadol.

In at least one embodiment of the invention, the first once daily controlled-release dosage form comprising the at least one normal release matrix core coated with at least one release-slowing coat comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one normal-release matrix core coated with at least one release-slowing coat will desirably comprise a mixture of an effective amount of at least two different tramadol salts.

In at least one embodiment of the invention, the first once daily controlled-release dosage form comprising the at least one normal-release matrix core coated with at least one release-slowing coat will desirably comprise about 70 to about 90% by weight of the core dry weight tramadol.

In at least one embodiment of the invention, the first once daily controlled-release dosage form comprising the at least one normal-release matrix core coated with at least one release-slowing coat will desirably comprise a mixture of an effective amount of at least two different tramadol salts comprises an immediate release coat, wherein said immediate release coat comprises tramadol.

In at least one embodiment of the invention, the first once daily controlled-release dosage form comprising the at least one normal-release matrix core coated with at least one release-slowing coat will desirably comprise a mixture of an effective amount of at least two different tramadol salts comprises an immediate release coat, wherein said immediate release coat comprises a salt or form of tramadol, which is different from the salt or form of tramadol present in the remainder of the first once daily controlled release dosage form comprising the normal release matrix core coated with at least one release-slowing coat.

In at least one embodiment of the invention, the first once daily controlled-release dosage form comprises at least one normal release matrix core coated with at least one release-slowing coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprises at least one normal release matrix core coated with at least one release-slowing coat, which coat comprises at least one water-insoluble water-permeable film-forming polymer and at least one water-soluble polymer.

In at least one embodiment of the invention, the first once daily controlled-release dosage form comprises at least one normal release matrix core coated with at least one release-slowing coat, which coat comprises at least one water-insoluble water-permeable film-forming polymer, at least one water-soluble polymer, and at least one plasticizer.

In at least one embodiment of the invention, the first once daily controlled-release dosage form comprises at least one normal release matrix core coated with at least one release-slowing coat, which coat comprises at least one enteric polymer.

In at least one embodiment of the invention, the first once daily controlled-release dosage form comprises at least one normal release matrix core coated with at least one release-slowing coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprises at least one normal release matrix core coated with at least one release-slowing coat, which coat comprises at least one pH dependent polymer.

In at least one embodiment of the invention, the first once daily controlled-release dosage form comprises at least one normal release matrix core coated with at least one release-slowing coat comprises at least one non-functional soluble coat.

In at least one embodiment of the invention, the first once daily controlled-release dosage form comprising the at least one normal release matrix core coated with at least one release-slowing coat is in the form of a tablet.

In at least one embodiment of the invention, the first once daily controlled-release dosage form comprises at least one normal release matrix core coated with at least one release-slowing coat is in the form of a capsule.

In at least one embodiment of the invention, the first once daily controlled-release dosage form comprises at least one normal release matrix core coated with at least one release-slowing coat is in the form of a microparticle.

In at least one embodiment of the invention, the first once daily controlled-release dosage form comprises at least one normal release matrix core coated with at least one release-slowing coat is in the form of a plurality of microparticles, wherein each microparticle is comprised of a normal release matrix core coated with at least one release-slowing coat.

In at least one embodiment of the invention, the first once daily controlled-release dosage form comprises at least one normal release matrix core coated with at least one release-slowing coat, wherein said first once daily controlled-release dosage form comprises a unitary core.

In at least one embodiment of the invention, the first once daily controlled-release dosage form comprises at least one normal release matrix core coated with at least one release-slowing coat, wherein said first once daily controlled-release dosage form comprises two or more coats, wherein one coat comprises a delayed-release coat.

In at least one embodiment of the invention, the first once daily controlled-release dosage form comprising at least one normal release matrix core coated with at least one release-slowing coat is comprised of a plurality of coated cores.

In at least one embodiment of the invention, the first once daily controlled-release dosage form comprises at least one normal release matrix core coated with at least one aqueous insoluble coat.

In at least one embodiment of the invention, the first once daily controlled-release dosage form comprising the at least one normal release matrix core coated with at least one insoluble coat comprises an effective amount of tramadol for the management of moderate to moderately severe pain.

In at least one embodiment of the invention, the first once daily controlled-release dosage form comprising the at least one normal release matrix core coated with at least one insoluble coat comprises from about 25 mg to about 800 mg of tramadol.

In at least one embodiment of the invention, the first once daily controlled-release dosage form comprising the at least one normal release matrix core coated with at least one insoluble coat comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one normal-release matrix core coated with at least one aqueous insoluble coat will desirably comprise a mixture of an effective amount of at least two different tramadol salts.

In at least one embodiment of the invention, the first once daily controlled-release dosage form comprising the at least one normal-release matrix core coated with at least one aqueous insoluble coat will desirably comprise about 70 to about 90% by weight of the core dry weight tramadol.

In at least one embodiment of the invention, the first once daily controlled-release dosage form comprising the at least one normal-release matrix core coated with at least one aqueous insoluble coat will desirably comprise a mixture of an effective amount of at least two different tramadol salts comprises an immediate release coat, wherein said immediate release coat comprises tramadol.

In at least one embodiment of the invention, the first once daily controlled-release dosage form comprising the at least one normal-release matrix core coated with at least one aqueous insoluble coat will desirably comprise a mixture of an effective amount of at least two different tramadol salts comprises an immediate release coat, wherein said immediate release coat comprises a salt or form of tramadol, which is different from the salt or form of tramadol present in the remainder of the first once daily controlled release dosage form comprising the normal release matrix core coated with at least one aqueous insoluble coat.

In at least one embodiment of the invention, the first once daily controlled-release dosage form comprises at least one normal release matrix core coated with at least one aqueous insoluble coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprises at least one normal release matrix core coated with at least one aqueous insoluble coat, which coat comprises at least one water-insoluble water-permeable film-forming polymer and at least one water-soluble polymer.

In at least one embodiment of the invention, the first once daily controlled-release dosage form comprises at least one normal release matrix core coated with at least one aqueous insoluble coat, which coat comprises at least one water-insoluble water-permeable film-forming polymer, at least one water-soluble polymer, and at least one plasticizer.

In at least one embodiment of the invention, the first once daily controlled-release dosage form comprises at least one normal release matrix core coated with at least one aqueous insoluble coat, which coat comprises at least one enteric polymer.

In at least one embodiment of the invention, the first once daily controlled-release dosage form comprises at least one normal release matrix core coated with at least one aqueous insoluble coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprises at least one normal release matrix core coated with at least one aqueous insoluble coat, which coat comprises at least one pH dependent polymer.

In at least one embodiment of the invention, the first once daily controlled-release dosage form comprises at least one normal release matrix core coated with at least one aqueous insoluble coat comprises at least one non-functional soluble coat.

In at least one embodiment of the invention, the first once daily controlled-release dosage form comprising the at least one normal release matrix core coated with at least one aqueous insoluble coat is in the form of a tablet.

In at least one embodiment of the invention, the first once daily controlled-release dosage form comprises at least one normal release matrix core coated with at least one aqueous insoluble coat is in the form of a capsule.

In at least one embodiment of the invention, the first once daily controlled-release dosage form comprises at least one normal release matrix core coated with at least one aqueous insoluble coat is in the form of a microparticle.

In at least one embodiment of the invention, the first once daily controlled-release dosage form comprises at least one normal release matrix core coated with at least one aqueous insoluble coat is in the form of a plurality of microparticles, wherein each microparticle is comprised of a normal release matrix core coated with at least one aqueous insoluble coat.

In at least one embodiment of the invention, the first once daily controlled-release dosage form comprises at least one normal release matrix core coated with at least one aqueous insoluble coat, wherein said first once daily controlled-release dosage form comprises a unitary core.

In at least one embodiment of the invention, the first once daily controlled-release dosage form comprises at least one normal release matrix core coated with at least one aqueous insoluble coat, wherein said first once daily controlled-release dosage form comprises two or more coats, wherein one coat comprises a delayed-release coat.

In at least one embodiment of the invention, the first once daily controlled-release dosage form comprises at least one normal release matrix core coated with at least one aqueous insoluble coat, wherein said first once daily controlled-release dosage form comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising at least one normal release matrix core coated with at least one aqueous insoluble coat is comprised of a plurality of coated cores.

In at least one embodiment of the invention, the first once daily controlled-release dosage form comprises at least one normal release matrix core coated with at least one swellable and erodable coat.

In at least one embodiment of the invention, the first once daily controlled-release dosage form comprising the at least one normal release matrix core coated with at least one swellable and erodable coat comprises an effective amount of tramadol for the management of moderate to moderately severe pain.

In at least one embodiment of the invention, the first once daily controlled-release dosage form comprising the at least one normal release matrix core coated with at least one swellable and erodable coat comprises from about 25 mg to about 800 mg of tramadol.

In at least one embodiment of the invention, the first once daily controlled-release dosage form comprising the at least one normal release matrix core coated with at least one swellable and erodable coat comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one normal-release matrix core coated with at least one swellable and erodable coat will desirably comprise a mixture of an effective amount of at least two different tramadol salts.

In at least one embodiment of the invention, the first once daily controlled-release dosage form comprising the at least one normal-release matrix core coated with at least one swellable and erodable coat will desirably comprise about 70 to about 90% by weight of the core dry weight tramadol.

In at least one embodiment of the invention, the first once daily controlled-release dosage form comprising the at least one normal-release matrix core coated with at least one swellable and erodable coat will desirably comprise a mixture of an effective amount of at least two different tramadol salts comprises an immediate release coat, wherein said immediate release coat comprises tramadol.

In at least one embodiment of the invention, the first once daily controlled-release dosage form comprising the at least one normal-release matrix core coated with at least one swellable and erodable coat will desirably comprise a mixture of an effective amount of at least two different tramadol salts comprises an immediate release coat, wherein said immediate release coat comprises a salt or form of tramadol, which is different from the salt or form of tramadol present in the remainder of the first once daily controlled release dosage form comprising the normal release matrix core coated with at least one swellable and erodable coat.

In at least one embodiment of the invention, the first once daily controlled-release dosage form comprises at least one normal release matrix core coated with at least one swellable and erodable coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprises at least one normal release matrix core coated with at least one swellable and erodable coat, which coat comprises at least one water-insoluble water-permeable film-forming polymer and at least one water-soluble polymer.

In at least one embodiment of the invention, the first once daily controlled-release dosage form comprises at least one normal release matrix core coated with at least one swellable and erodable coat, which coat comprises at least one water-insoluble water-permeable film-forming polymer, at least one water-soluble polymer, and at least one plasticizer.

In at least one embodiment of the invention, the first once daily controlled-release dosage form comprises at least one normal release matrix core coated with at least one swellable and erodable coat, which coat comprises at least one enteric polymer.

In at least one embodiment of the invention, the first once daily controlled-release dosage form comprises at least one normal release matrix core coated with at least one swellable and erodable coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprises at least one normal release matrix core coated with at least one swellable and erodable coat, which coat comprises at least one pH dependent polymer.

In at least one embodiment of the invention, the first once daily controlled-release dosage form comprises at least one normal release matrix core coated with at least one swellable and erodable coat comprises at least one non-functional soluble coat.

In at least one embodiment of the invention, the first once daily controlled-release dosage form comprising the at least one normal release matrix core coated with at least one swellable and erodable coat is in the form of a tablet.

In at least one embodiment of the invention, the first once daily controlled-release dosage form comprises at least one normal release matrix core coated with at least one swellable and erodable coat is in the form of a capsule.

In at least one embodiment of the invention, the first once daily controlled-release dosage form comprises at least one normal release matrix core coated with at least one swellable and erodable coat is in the form of a microparticle.

In at least one embodiment of the invention, the first once daily controlled-release dosage form comprises at least one normal release matrix core coated with at least one swellable and erodable coat is in the form of a plurality of microparticles, wherein each microparticle is comprised of a normal release matrix core coated with at least one swellable and erodable coat.

In at least one embodiment of the invention, the first once daily controlled-release dosage form comprises at least one normal release matrix core coated with at least one swellable and erodable coat, wherein said first once daily controlled-release dosage form comprises a unitary core.

In at least one embodiment of the invention, the first once daily controlled-release dosage form comprises at least one normal release matrix core coated with at least one swellable and erodable coat, wherein said first once daily controlled-release dosage form comprises two or more coats, wherein one coat comprises a delayed-release coat.

In at least one embodiment of the invention, the first once daily controlled-release dosage form comprises at least one normal release matrix core coated with at least one swellable and erodable coat, wherein said first once daily controlled-release dosage form comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising at least one normal release matrix core coated with at least one swellable and erodable coat is comprised of a plurality of coated cores.

In at least one embodiment of the invention, the first once daily controlled-release dosage form comprises at least one normal release matrix core coated with at least one erodable coat.

In at least one embodiment of the invention, the first once daily controlled-release dosage form comprising the at least one normal release matrix core coated with at least one erodable coat comprises an effective amount of tramadol for the management of moderate to moderately severe pain.

In at least one embodiment of the invention, the first once daily controlled-release dosage form comprising the at least one normal release matrix core coated with at least one erodable coat comprises from about 25 mg to about 800 mg of tramadol.

In at least one embodiment of the invention, the first once daily controlled-release dosage form comprising the at least one normal release matrix core coated with at least one erodable coat comprises about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg of tramadol.

In at least one embodiment of the invention, the first once daily controlled-release dosage form comprising the at least one normal-release matrix core coated with at least one erodable coat will desirably comprise a mixture of an effective amount of at least two different tramadol salts.

In at least one embodiment of the invention, the first once daily controlled-release dosage form comprising the at least one normal-release matrix core coated with at least one erodable coat will desirably comprise about 70 to about 90% by weight of the core dry weight tramadol.

In at least one embodiment of the invention, the first once daily controlled-release dosage form comprising the at least one normal-release matrix core coated with at least one erodable coat will desirably comprise a mixture of an effective amount of at least two different tramadol salts comprises an immediate release coat, wherein said immediate release coat comprises tramadol.

In at least one embodiment of the invention, the first once daily controlled-release dosage form comprising the at least one normal-release matrix core coated with at least one erodable coat will desirably comprise a mixture of an effective amount of at least two different tramadol salts comprises an immediate release coat, wherein said immediate release coat comprises a salt or form of tramadol, which is different from the salt or form of tramadol present in the remainder of the first once daily controlled release dosage form comprising the normal release matrix core coated with at least one erodable coat.

In at least one embodiment of the invention, the first once daily controlled-release dosage form comprises at least one normal release matrix core coated with at least one erodable coat, which coat comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprises at least one normal release matrix core coated with at least one erodable coat, which coat comprises at least one water-insoluble water-permeable film-forming polymer and at least one water-soluble polymer.

In at least one embodiment of the invention, the first once daily controlled-release dosage form comprises at least one normal release matrix core coated with at least one erodable coat, which coat comprises at least one water-insoluble water-permeable film-forming polymer, at least one water-soluble polymer, and at least one plasticizer.

In at least one embodiment of the invention, the first once daily controlled-release dosage form comprises at least one normal release matrix core coated with at least one erodable coat, which coat comprises at least one enteric polymer.

In at least one embodiment of the invention, the first once daily controlled-release dosage form comprises at least one normal release matrix core coated with at least one erodable coat, which coat comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprises at least one normal release matrix core coated with at least one erodable coat, which coat comprises at least one pH dependent polymer.

In at least one embodiment of the invention, the first once daily controlled-release dosage form comprises at least one normal release matrix core coated with at least one erodable coat comprises at least one non-functional soluble coat.

In at least one embodiment of the invention, the first once daily controlled-release dosage form comprising the at least one normal release matrix core coated with at least one erodable coat is in the form of a tablet.

In at least one embodiment of the invention, the first once daily controlled-release dosage form comprises at least one normal release matrix core coated with at least one erodable coat is in the form of a capsule.

In at least one embodiment of the invention, the first once daily controlled-release dosage form comprises at least one normal release matrix core coated with at least one erodable coat is in the form of a microparticle.

In at least one embodiment of the invention, the first once daily controlled-release dosage form comprises at least one normal release matrix core coated with at least one erodable coat is in the form of a plurality of microparticles, wherein each microparticle is comprised of a normal release matrix core coated with at least one erodable coat.

In at least one embodiment of the invention, the first once daily controlled-release dosage form comprises at least one normal release matrix core coated with at least one erodable coat, wherein said first once daily controlled-release dosage form comprises a unitary core.

In at least one embodiment of the invention, the first once daily controlled-release dosage form comprises at least one normal release matrix core coated with at least one erodable coat, wherein said first once daily controlled-release dosage form comprises two or more coats, wherein one coat comprises a delayed-release coat.

In at least one embodiment of the invention, the first once daily controlled-release dosage form comprises at least one normal release matrix core coated with at least one erodable coat, wherein said first once daily controlled-release dosage form comprises two or more coats, wherein one coat comprises an aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

In at least one embodiment of the invention, the first once daily controlled-release dosage form comprising at least one normal release matrix core coated with at least one erodable coat is comprised of a plurality of coated cores.

In at least one embodiment of the invention, the first once daily pharmaceutical dosage from comprises at least one osmotic dosage form. The terms “osmotic dosage forms”, “osmotic delivery devices”, “controlled-release osmotic dosage forms”, or “osmosis-controlled extended-release systems” are terms used interchangeably herein and are defined to mean dosage forms which dispense tramadol all or in part by pressure created by osmosis or osmosis and diffusion resulting from the flow of fluid into a material which expands and all or in part forces tramadol to be dispensed from the osmotic dosage form. Osmosis will desirably be defined as the flow of solvent from a compartment with a low concentration of solute to a compartment with a high concentration of solute. The two compartments are separated by a membrane, or coat, which allows flow of solvent (a liquid, aqueous media, or biological fluids) but not the solute. Examples of such membranes will desirably for example be, a semipermeable membrane, microporous, or asymmetric membrane, which asymmetric membrane will desirably be permeable, semipermeable, perforated, or unperforated and will desirably deliver tramadol by osmotic pumping, diffusion or the combined mechanisms of diffusion and osmotic pumping. Thus, in principle, osmosis controlled-release of tramadol involves osmotic transport of an aqueous media into the osmotic dosage form followed by dissolution of tramadol and the subsequent transport of the saturated solution of tramadol by osmotic pumping of the solution through at least one passageway in the semipermeable membrane or by osmosis and diffusion through the semipermeable membrane.

In at least one embodiment, the invention the first once daily controlled-release dosage form comprises at least one osmotic dosage form comprising tramadol which will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration in the fed or fasted state will desirably be bioequivalent according to FDA guidelines to a second orally administrable dosage form comprising the same dose of tramadol also suitable for once daily administration in the fed or fasted state.

In at least one embodiment, the invention the first once daily controlled-release dosage form comprises at least one osmotic dosage form comprising tramadol which will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, has reduced potential for alcohol induced dose dumping and will desirably be bioequivalent in the fed or fasted state according to FDA guidelines to a second orally administrable dosage form comprising the same dose of tramadol also suitable for once daily administration in the fed or fasted state.

In at least one embodiment, the invention the first once daily controlled-release dosage form comprises at least one osmotic dosage form comprising tramadol which will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml under fed conditions, and will desirably be bioequivalent according to FDA guidelines to a second orally administrable dosage form comprising the same dose of tramadol also suitable for once daily administration in the fed state.

In at least one embodiment, the invention the first once daily controlled-release dosage form comprises at least one osmotic dosage form comprising tramadol which will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml under fed conditions, has reduced potential for alcohol induced dose dumping and will desirably be bioequivalent according to FDA guidelines to a second orally administrable dosage form comprising the same dose of tramadol also suitable for once daily administration in the fed state.

In at least one embodiment, the invention the first once daily controlled-release dosage form comprises at least one osmotic dosage form comprising tramadol which will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration a Cₘₐₓ of, for example about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335 or about 338 ng/ml of tramadol under fed conditions and will desirably be bioequivalent according to FDA guidelines to a second orally administrable dosage form comprising the same dose of tramadol also suitable for once daily administration in the fed state.

In at least one embodiment, the invention the first once daily controlled-release dosage form comprises at least one osmotic dosage form comprising tramadol which will desirably exhibit an in-vitro release rate such that after about 2 hours from about 0 to about 22% by weight of tramadol is released, after about 4 hours from about 5 to about 30% by weight of tramadol is released, after 6 hours, from 15 to 38% by weight of tramadol is released, and after about 8 hours, more than about 40% by weight of tramadol is released, and when said first once daily controlled-release dosage form is administered once daily to a patient in need of such administration will desirably exhibit following single-dose administration an AUC_{0-∞} of, for example, about 2725, about 2750, about 2900, about 3050, about 3200, about 3350, about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions and will desirably be bioequivalent according to FDA guidelines to a second orally administrable dosage form comprising the same dose of tramadol also suitable for once daily administration in the fed state.

In at least one embodiment of the invention the first once daily controlled-release dosage form comprises at least one osmotic dosage form comprising tramadol will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration: (i) a Cₘₐₓ of from about 180 to about 333 ng/ml of tramadol, (ii) an AUC_{0-∞} of from about 3740 to about 7600 ng·hr/ml under fasting conditions, and will desirably be bioequivalent according to FDA guidelines to a second orally administrable dosage form comprising the same dose of tramadol also suitable for once daily administration in the fasted state.

In at least one embodiment of the invention the first once daily controlled-release dosage form comprises at least one osmotic dosage form comprising tramadol will desirably exhibit an in-vitro release rate such that after about 2 hours from about 0 to about 22% by weight of tramadol is released, after about 4 hours from about 5 to about 30% by weight of tramadol is released, after 6 hours, from 15 to 38% by weight of tramadol is released, and after 8 about hours, more than about 40% by weight of tramadol is released, and when said first once daily controlled-release dosage form is administered once daily to a patient in need of such administration will desirably exhibit following single-dose administration: (i) a Cₘₐₓ of from about 180 to about 333 ng/ml of tramadol, (ii) an AUC_{0-∞} of from about 3740 to about 7600 ng·hr/ml under fasting conditions, has reduced potential for alcohol induced dose dumping and will desirably be bioequivalent according to FDA guidelines to a second orally administrable dosage form comprising the same dose of tramadol also suitable for once daily administration in the fasted state.

In at least one embodiment of the invention the first once daily controlled-release dosage form comprises at least one osmotic dosage form comprising tramadol will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration a Cₘₐₓ of, for example, about 180, about 190, about 200, about 210, about 220, about 230, about 240, about 250, about 260, about 270, about 280, about 290, about 300, about 310, about 320, about 330, or about 333 ng/ml of tramadol under fasting conditions and will desirably be bioequivalent according to FDA guidelines to a second orally administrable dosage form comprising the same dose of tramadol also suitable for once daily administration in the fasted state.

In at least one embodiment of the invention the first once daily controlled-release dosage form comprises at least one osmotic dosage form comprising tramadol will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered once daily to a patient in need of such administration will desirably exhibit following single-dose administration an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600, ng·hr/ml of tramadol under fasting conditions and will desirably be bioequivalent according to FDA guidelines to a second orally administrable dosage form comprising the same dose of tramadol also suitable for once daily administration in the fasted state.

In at least one embodiment, the first once daily controlled-release dosage form comprising an osmotic dosage form will desirably exhibit following single-dose administration a Tₘₐₓ of tramadol from about 4 to about 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr in the fed state and be bioequivalent according to FDA guidelines to a second orally administrable dosage form comprising tramadol also suitable for once daily administration.

In at least one embodiment of the invention, first once daily controlled-release dosage form comprising an osmotic dosage form will desirably exhibit following single-dose administration a Tₘₐₓ of tramadol from about 10 to about 20 hr, for example, about 10, about 11, about 12, about 13, about 14, about 15, about 16, about 17, about 18, about 19, or about 20 hr in the fasting state and be bioequivalent according to FDA guidelines to a second orally administrable dosage form comprising tramadol also suitable for once daily administration.

In at least one embodiment of the invention, the first once daily controlled-release dosage form comprising an osmotic dosage form will desirably exhibit at steady state the following pharmacokinetic parameters in-vivo under fasting conditions: (i) an AUC₀₋₂₄ from about 1635 to about 21000, for example, from about 1635 to about 3920, about 3610 to about 9120, or about 9455 to about 20965 ng·h/ml, and (ii) a Cₘₐₓ from about 117 to about 1230, for example, about 117 to about 245, about 230 to about 590, or about 590 to about 1230 ng/ml, and be bioequivalent according to FDA guidelines to a second orally administrable dosage form comprising tramadol also suitable for once daily administration.

In at least one embodiment of the invention, the first once daily controlled-release dosage form comprising an osmotic dosage form will desirably exhibit a Tₘₐₓ of, for example, about 9, about 10, about 11, about 12, about 13, or 14 hours at steady state under fasting conditions and be bioequivalent according to FDA guidelines to the second orally administrable dosage form comprising tramadol also suitable for once daily administration.

In other embodiments of the invention, the first once daily controlled-release dosage form comprising an osmotic dosage form will desirably exhibit a degree of fluctuation (%) of, for example, about 43 to about 141, about 43 to about 120, about 58 to about 132, or about 57 to about 141 at steady state under fasting conditions and be bioequivalent according to FDA guidelines to the second orally administrable dosage form comprising tramadol also suitable for once daily administration.

In other embodiments of the invention, the first once daily controlled-release dosage form comprising an osmotic dosage form will desirably exhibit a Cₘᵢₙ of, for example, from about 31 to about 652, about 31 to about 117, about 96 to about 241, or about 226 to about 652 ng/ml at steady state under fasting conditions and be bioequivalent according to FDA guidelines to the second orally administrable dosage form comprising tramadol also suitable for once daily administration.

In at least one embodiment of the invention, the first once daily controlled-release dosage form comprising an osmotic dosage form will desirably further exhibit under fasting conditions a Tₘₐₓ of, for example, about 9, about 10, about 11, about 12, about 13, or about 14 hours, a degree of fluctuation (%) of, for example, about 43 to about 141, about 43 to about 120, about 58 to about 132, or about 57 to about 141, and a Cₘᵢₙ of, for example, from about 31 to about 652, about 31 to about 117, about 96 to about 241, or about 226 to about 652 ng/ml at steady state and be bioequivalent according to FDA guidelines to the second orally administrable dosage form comprising tramadol also suitable for once daily administration.

The invention, in at least one embodiment, is directed to a method for administering a therapeutically effective amount of tramadol to a human for the treatment or management of moderately to moderately severe pain, wherein the method comprises administering orally to the human a first once daily controlled-release dosage form comprising an osmotic dosage form, said osmotic dosage form comprising a therapeutically effective amount of tramadol, said first once daily controlled-release dosage form exhibiting an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably be bioequivalent in the fed or fasted state according to FDA guidelines to a second orally administrable dosage form comprising the same dose of tramadol also suitable for once daily administration in the fed state.

In at least one embodiment, is directed to a method for administering a therapeutically effective amount of tramadol to a human for the treatment or management of moderately to moderately severe pain, wherein the method comprises administering orally to the human a first once daily controlled-release dosage form comprising an osmotic dosage form, said osmotic dosage form comprising a therapeutically effective amount of tramadol, said first once daily controlled-release dosage form exhibiting an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration has reduced potential for alcohol induced dose dumping and will desirably be bioequivalent in the fed or fsated state according to FDA guidelines to a second orally administrable dosage form comprising the same dose of tramadol also suitable for once daily administration in the fed or fasted state.

The invention, in at least one embodiment, is directed to a method for administering a therapeutically effective amount of tramadol to a human for the treatment or management of moderately to moderately severe pain, wherein the method comprises administering orally to the human a first once daily controlled-release dosage form comprising an osmotic dosage form, said osmotic dosage form comprising a therapeutically effective amount of tramadol, said first once daily controlled-release dosage form exhibiting an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, and will desirably be bioequivalent according to FDA guidelines to a second orally administrable dosage form comprising the same dose of tramadol also suitable for once daily administration in the fed state.

In at least one embodiment, is directed to a method for administering a therapeutically effective amount of tramadol to a human for the treatment or management of moderately to moderately severe pain, wherein the method comprises administering orally to the human a first once daily controlled-release dosage form comprising an osmotic dosage form, said osmotic dosage form comprising a therapeutically effective amount of tramadol, said first once daily controlled-release dosage form exhibiting an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, has reduced potential for alcohol induced dose dumping and will desirably be bioequivalent according to FDA guidelines to a second orally administrable dosage form comprising the same dose of tramadol also suitable for once daily administration in the fed state.

In at least one embodiment, is directed to a method for administering a therapeutically effective amount of tramadol to a human for the treatment or management of moderately to moderately severe pain, wherein the method comprises administering orally to the human a first once daily controlled-release dosage form comprising an osmotic dosage form, said osmotic dosage form comprising a therapeutically effective amount of tramadol, said first once daily controlled-release dosage form exhibiting an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration a Cₘₐₓ of, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335 or about 338 ng/ml of tramadol under fed conditions and will desirably be bioequivalent according to FDA guidelines to a second orally administrable dosage form comprising the same dose of tramadol also suitable for once daily administration in the fed state.

In at least one embodiment, is directed to a method for administering a therapeutically effective amount of tramadol to a human for the treatment or management of moderately to moderately severe pain, wherein the method comprises administering orally to the human a first once daily controlled-release dosage form comprising an osmotic dosage form, said osmotic dosage form comprising a therapeutically effective amount of tramadol, said first once daily controlled-release dosage form exhibiting an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration an AUC_{0-∞} of, for example, about 2725, about 2750, about 2900, about 3050, about 3200, about 3350, about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderately to moderately severe pain, wherein the method comprises administering orally to the human a first once daily controlled-release dosage form comprising a therapeutically effective amount of tramadol, the first once daily controlled-release dosage form comprising an osmotic dosage form such that the first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit: (i) a Cₘₐₓ, of from about 180 to about 333 ng/ml of tramadol, (ii) an AUC_{0-∞} of from about 3740 to about 7600 ng·hr/ml of tramadol under fasting conditions, and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderately to moderately severe pain, wherein the method comprises administering orally to the human a first once daily controlled-release dosage form comprising a therapeutically effective amount of tramadol, the first once daily controlled-release dosage form comprising an osmotic dosage form such that the first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit: (i) a Cₘₐₓ of from about 180 to about 333 ng/ml of tramadol, (ii) an AUC_{0-∞} of from about 3740 to about 7600 ng·hr/ml of tramadol under fasting conditions, has reduced alcohol induced dose dumping and will desirably be bioequivalent according to FDA guidelines to a second orally administrable dosage form comprising the same dose of tramadol also suitable for once daily administration in the fasted state.

In at least one embodiment, is directed to a method for administering a therapeutically effective amount of tramadol to a human for the treatment or management of moderately to moderately severe pain, wherein the method comprises administering orally to the human a first once daily controlled-release dosage form comprising a therapeutically effective amount of tramadol, the first once daily controlled-release dosage form comprising an osmotic dosage form such that the first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit a Cₘₐₓ of, for example, about 180, about 190, about 200, about 210, about 220, about 230, about 240, about 250, about 260, about 270, about 280, about 290, about 300, about 310, about 320, about 330, or about 333 ng/ml of tramadol under fasting conditions and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderately to moderately severe pain, wherein the method comprises administering orally to the human a first once daily controlled-release dosage form comprising a therapeutically effective amount of tramadol, the first once daily controlled-release dosage form comprising an osmotic dosage form such that the first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600, ng·hr/ml of tramadol under fasting conditions and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderately to moderately severe pain, wherein the method comprises administering orally to the human a first once daily controlled-release dosage form comprising a therapeutically effective amount of tramadol, the first once daily controlled-release dosage form comprising an osmotic dosage form and will desirably exhibit in the fed state a Tₘₐₓ of tramadol from about 4 to about 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr after single-dose administration and will desirably be bioequivalent according to FDA guidelines to a second orally administrable dosage form comprising tramadol also suitable for once daily administration.

In embodiments of the invention where the osmotic dosage form is directed to a method for administering a therapeutically effective amount of tramadol to a human for the treatment or management of moderately to moderately severe pain, the osmotic dosage form will desirably further exhibit at steady state the following pharmacokinetic parameters in-vivo under fasting conditions: (i) an AUC₀₋₂₄ from about 1635 to about 21000, for example, from about 1635 to about 3920, about 3610 to about 9120, or about 9455 to about 20965 ng·h/ml, and (ii) a Cₘₐₓ from about 117 to about 1230, for example, about 117 to about 245, about 230 to about 590, or about 590 to about 1230 ng/ml and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderately to moderately severe pain, the osmotic dosage form will desirably also exhibit under fasting conditions a Tₘₐₓ of, for example, about 9, about 10, about 11, about 12, about 13, or about 14 hours and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderately to moderately severe pain, the osmotic dosage form will desirably also exhibit under fasting conditions a degree of fluctuation (%) of, for example, about 43 to about 141, about 43 to about 120, about 58 to about 132, or about 57 to about 141 and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderately to moderately severe pain, the osmotic dosage form will desirably also exhibit under fasting conditions Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderately to moderately severe pain, the osmotic dosage form will desirably also exhibit under fasting conditions a Tₘₐₓ of, for example, about 9, about 10, about 11, about 12, about 13, or about 14 hours, a degree of fluctuation (%) of, for example, about 43 to about 141, about 43 to about 120, about 58 to about 132, or about 57 to about 141, and Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderately to moderately severe pain, the osmotic dosage form will desirably further exhibit in the fasting state a Tₘₐₓ of tramadol from about 10 to about 20 hr, for example, about 10, about 11, about 12, about 13, about 14, about 15, about 16, about 17, about 18, about 19, or about 20 hr after single-dose administration and will desirably be bioequivalent according to FDA guidelines to a second orally administrable dosage form comprising the same dose of tramadol also suitable for once daily administration in the fasted state.

In at least one embodiment of the invention, the in-vitro dissolution of the first once daily controlled-release dosage form comprising an osmotic dosage form is measured using a USP Type I, II, or III apparatus in dissolution medium chosen from 900 ml 0.1N HCl, water, 0.1N HCl+0.1% Cetrimide, USP Buffer pH 1.5, Acetate Buffer pH 4.5, Phosphate Buffer pH 6.5, or Phosphate Buffer pH7.4 at 75 rpm at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising an osmotic dosage form is measured using a USP Type I, II, or III apparatus in 900 ml 0.1N HCl 75 rpm at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising an osmotic dosage form is measured using a USP Type I, II, or III apparatus in water at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising an osmotic dosage form is measured using a USP Type I, II, or III apparatus in 0.1N HCl+0.1% Cetrimide at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising an osmotic dosage form is measured a USP Type I, II, or III apparatus in USP Buffer pH 1.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising an osmotic dosage form is measured using a USP Type I, II, or III apparatus in Acetate buffer pH 4.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising an osmotic dosage form is measured using a USP Type I, II, or III apparatus in Phosphate Buffer pH 6.5 at 37°±0.5° C. and the tramadol released into the dissolution medium is assayed in a 10 ml UV cell at 271 nm.

In at least one embodiment of the invention, the in-vitro dissolution of the first once daily controlled-release dosage form comprising an osmotic dosage form is measured using a USP Type I, II, or III apparatus in Phosphate Buffer pH 7.4 at 37°±0.5° C. and the tramadol released into the dissolution medium is assayed in a 10 ml UV cell at 271 nm.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably be bioequivalent in the fed or fasted state according to FDA guidelines to a second orally administrable dosage form comprising the same dose of tramadol also suitable for once daily administration in the fed or fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration has reduced potential for alcohol induced dose dumping and will desirably be bioequivalent in the fed or fasted state according to FDA guidelines to a second orally administrable dosage form comprising the same dose of tramadol also suitable for once daily administration in the fed or fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, has reduced potential for alcohol induced dose dumping and will desirably be bioequivalent according to FDA guidelines to a second orally administrable dosage form comprising the same dose of tramadol also suitable for once daily administration in the fed state.

In at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration a Cₘₐₓ of, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335 or about 338 ng/ml of tramadol under fed conditions and will desirably be bioequivalent according to FDA guidelines to a second orally administrable dosage form comprising the same dose of tramadol also suitable for once daily administration in the fed state.

In at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration an AUC_{0-∞} of, for example, about 2725, about 2750, about 2900, about 3050, about 3200, about 3350, about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose: (i) a Cₘₐₓ of from about 180 to about 333 ng/ml of tramadol, (ii) an AUC_{0-∞} of from about 3740 to about 7600 ng·hr/ml of tramadol under fasting conditions, and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose: (i) a Cₘₐₓ of from about 180 to about 333 ng/ml of tramadol, (ii) an AUC_{0-∞} of from about 3740 to about 7600 ng·hr/ml of tramadol under fasting conditions, has reduced potential for alcohol induced dose dumping and will desirably be bioequivalent according to FDA guidelines to a second orally administrable dosage form comprising the same dose of tramadol also suitable for once daily administration in the fasted state.

In at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose a Cₘₐₓ of, for example, about 180, about 190, about 200, about 210, about 220, about 230, about 240, about 250, about 260, about 270, about 280, about 290, about 300, about 310, about 320, about 330, or about 333 ng/ml of tramadol under fasting conditions and will desirably be bioequivalent according to FDA guidelines to a second orally administrable dosage form comprising the same dose of tramadol also suitable for once daily administration in the fasted state.

In at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600, ng·hr/ml of tramadol under fasting conditions and will desirably be bioequivalent according to FDA guidelines to a second orally administrable dosage form comprising the same dose of tramadol also suitable for once daily administration in the fasted state.

In embodiments where the osmotic dosage form is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably further exhibit in the fed state a Tₘₐₓ of tramadol from about 4 to about 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr after single-dose administration and will desirably be bioequivalent according to FDA guidelines to a second orally administrable dosage form comprising tramadol also suitable for once daily administration.

In embodiments of the invention where the osmotic dosage form is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably further exhibit at steady state the following pharmacokinetic parameters in-vivo under fasting conditions: (i) an AUC₀₋₂₄ from about 1635 to about 21000, for example, from about 1635 to about 3920, about 3610 to about 9120, or about 9455 to about 20965 ng·h/ml, and (ii) a Cₘₐₓ from about 117 to about 1230, for example, about 117 to about 245, about 230 to about 590, or about 590 to about 1230 ng/ml and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that the first once daily controlled-release dosage form will desirably also exhibit under fasting conditions a Tₘₐₓ of, for example, about 9, about 10, about 11, about 12, about 13, or about 14 hours and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that the first once daily controlled-release dosage form will desirably also exhibit under fasting conditions a degree of fluctuation (%) of, for example, about 43 to about 141, about 43 to about 120, about 58 to about 132, or about 57 to about 141 and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that the first once daily controlled-release dosage form will desirably also exhibit under fasting conditions a Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that the first once daily controlled-release dosage form will desirably also exhibit under fasting conditions a Tₘₐₓ of, for example, about 9, about 10, about 11, about 12, about 13, or about 14 hours, a degree of fluctuation (%) of, for example, about 43 to about 141, about 43 to about 120, about 58 to about 132, or about 57 to about 141, and Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

In embodiments of the invention where the osmotic dosage form is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol and optionally a means for forcibly dispensing the tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that the first once daily controlled-release dosage form will desirably also exhibit under fasting conditions a Tₘₐₓ of tramadol from about 10 to about 20 hr, for example, about 10, about 11, about 12, about 13, about 14, about 15, about 16, about 17, about 18, about 19, or about 20 hr after single-dose administration and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and will desirably be bioequivalent in the fed or fasted state according to FDA guidelines to a second orally administrable dosage form comprising the same dose of tramadol also suitable for once daily administration in the fed or fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, has reduced potential for alcohol induced dose dumping and will desirably be bioequivalent in the fed or fasted state according to FDA guidelines to a second orally administrable dosage form comprising the same dose of tramadol also suitable for once daily administration in the fed or fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, has reduced potential for alcohol induced dose dumping and will desirably be bioequivalent according to FDA guidelines to a second orally administrable dosage form comprising the same dose of tramadol also suitable for once daily administration in the fed state.

In at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration a Cₘₐₓ of, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335 or about 338 ng/ml of tramadol under fed conditions and will desirably be bioequivalent according to FDA guidelines to a second orally administrable dosage form comprising the same dose of tramadol also suitable for once daily administration in the fed state.

In at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration an AUC_{0-∞} of, for example, about 2725, about 2750, about 2900, about 3050, about 3200, about 3350 about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions and will desirably be bioequivalent according to FDA guidelines to a second orally administrable dosage form comprising the same dose of tramadol also suitable for once daily administration in the fed state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose: (i) a Cₘₐₓ of from about 180 to about 333 ng/ml of tramadol, (ii) an AUC_{0-∞} of from about 3740 to about 7600 ng·hr/ml of tramadol under fasting conditions, and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose: (i) a Cₘₐₓ of from about 180 to about 333 ng/ml of tramadol, (ii) an AUC_{0-∞} of from about 3740 to about 7600 ng·hr/ml of tramadol under fasting conditions, has reduced potential for alcohol induced dose dumping and will desirably be bioequivalent according to FDA guidelines to a second orally administrable dosage form comprising the same dose of tramadol also suitable for once daily administration in the fasted state.

In at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose a Cₘₐₓ of from about 180 to about 333 ng/ml, for example about 180, about 190, about 200, about 210, about 220, about 230, about 240, about 250, about 260, about 270, about 280, about 290, about 300, about 310, about 320, about 330, or about 333 ng/ml of tramadol under fasting conditions and will desirably be bioequivalent according to FDA guidelines to a second orally administrable dosage form comprising the same dose of tramadol also suitable for once daily administration in the fasted state.

In at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably exhibit an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600 ng·hr/ml of tramadol under fasting conditions and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably further exhibit in the fed state a Tₘₐₓ of tramadol from about 4 to about 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr after single-dose administration and will desirably be bioequivalent according to FDA guidelines to a second orally administrable dosage form comprising tramadol also suitable for once daily administration.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably further exhibit at steady state the following pharmacokinetic parameters in-vivo under fasting conditions: (i) an AUC₀₋₂₄ from about 1635 to about 21000, for example, from about 1635 to about 3920, about 3610 to about 9120, or about 9455 to about 20965 ng·h/ml, and (ii) a Cₘₐₓ from about 117 to about 1230, for example, about 117 to about 245, about 230 to about 590, or about 590 to about 1230 ng/ml and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably further exhibit under fasting conditions a Tₘₐₓ of, for example, about 9, about 10, about 11, about 12, about 13, or about 14 hours and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably further exhibit under fasting conditions a degree of fluctuation (%) of, for example, about 43 to about 141, about 43 to about 120, about 58 to about 132, or about 57 to about 141 and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably further exhibit under fasting conditions a Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably further exhibit under fasting conditions a Tₘₐₓ of, for example, about 9, about 10, about 11, about 12, about 13, or about 14 hours, a degree of fluctuation (%) of, for example, about 43 to about 141, about 43 to about 120, about 58 to about 132, or about 57 to about 141, and Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

The invention, in at least one embodiment, is directed to a method for administering a therapeutically effective amount of tramadol to a human for the treatment or management of moderate to moderately severe pain, wherein the method comprises administering orally to the human a first once daily controlled-release dosage form comprising a core and a membrane surrounding said core, said core comprising a therapeutically effective amount of tramadol, a means for increasing the hydrostatic pressure within the core and optionally a means for forcibly dispensing tramadol from the first once daily controlled-release dosage form, said membrane comprising at least one means for the exit of tramadol from the first once daily controlled-release dosage form, such that said first once daily controlled-release dosage form will desirably further exhibit under fasting conditions a Tₘₐₓ of tramadol from about 10 to about 20 hr, for example, about 10, about 11, about 12, about 13, about 14, about 15, about 16, about 17, about 18, about 19, or about 20 hr after single-dose administration and will desirably be bioequivalent according to FDA guidelines to a second orally administrable dosage form comprising tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably be bioequivalent in the fed or fasted state according to FDA guidelines to a second orally administrable dosage form comprising the same dose of tramadol also suitable for once daily administration in the fed or fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, has reduced potential for alcohol induced dose dumping, and when said first once daily controlled-release dosage form is administered to the human in need of such administration will desirably be bioequivalent in the fed or fasted state according to FDA guidelines to a second orally administrable dosage form comprising the same dose of tramadol also suitable for once daily administration in the fed or fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, and will desirably be bioequivalent according to FDA guidelines to a second orally administrable dosage form comprising the same dose of tramadol also suitable for once daily administration in the fed state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration: (i) a Cₘₐₓ of from about 75 to about 338 ng/ml of tramadol, (ii) an AUC_{0-∞} of from about 2725 to about 7581 ng·hr/ml of tramadol under fed conditions, has reduced potential for alcohol induced dose dumping and will desirably be bioequivalent according to FDA guidelines to a second orally administrable dosage form comprising the same dose of tramadol also suitable for once daily administration in the fed state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration a Cₘₐₓ of, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335 or about 338 ng/ml of tramadol under fed conditions and will desirably be bioequivalent according to FDA guidelines to a second orally administrable dosage form comprising the same dose of tramadol also suitable for once daily administration in the fed state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit following single-dose administration an AUC_{0-∞} of, for example, about 2725, about 2750, about 2900, about 3050, about 3200, about 3350 about 3500, about 3650, about 3800, about 3950, about 4100, about 4250, about 4400, about 4550, about 4700, about 4850, about 5000, about 5150, about 5300, about 5450, about 5600, about 5750, about 5900, about 6050, about 6200, about 6350, about 6500, about 6750, about 6900, about 7050, about 7200, about 7350, about 7500, or about 7581 ng·hr/ml of tramadol under fed conditions and will desirably be bioequivalent according to FDA guidelines to a second orally administrable dosage form comprising the same dose of tramadol also suitable for once daily administration in the fed state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose: (i) a Cₘₐₓ of from about 180 to about 333 ng/ml of tramadol, (ii) an AUC_{0-∞} of from about 3740 to about 7600 ng·hr/ml of tramadol under fasting conditions, and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose: (i) a Cₘₐₓ of from about 180 to about 333 ng/ml of tramadol, (ii) an AUC_{0-∞} of from about 3740 to about 7600 ng·hr/ml of tramadol under fasting conditions, has reduced potential for alcohol induced dose dumping and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose a Cₘₐₓ of, for example, about 180 to about 333 ng/ml, for example about 180, about 190, about 200, about 210, about 220, about 230, about 240, about 250, about 260, about 270, about 280, about 290, about 300, about 310, about 320, about 330, or about 333 ng/ml of tramadol under fasting conditions and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released from the first once daily controlled-release dosage form, and when said first once daily controlled-release dosage form is administered to the human in need of such administration will desirably exhibit at single dose an AUC_{0-∞} of, for example, about 3740, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, about 5000, about 5050, about 5100, about 5150, about 5200, about 5250, about 5300, about 5350, about 5400, about 5450, about 5500, about 5550, about 5600, about 5650, about 5700, about 5750, about 5800, about 5850, about 5900, about 5950, about 6000, about 6050, about 6100, about 6150, about 6200, about 6250, about 6300, about 6350, about 6400, about 6450, about 6500, about 6550, about 6600, about 6650, about 6700, about 6750, about 6800, about 6850, about 6900, about 6950, about 7000, about 7050, about 7100, about 7150, about 7200, about 7250, about 7300, about 7350, about 7400, about 7450, about 7500, about 7550, or about 7600, ng·hr/ml of tramadol under fasting conditions and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that said first once daily controlled-release dosage form will desirably further exhibit in the fed state a Tₘₐₓ of tramadol from about 4 to about 24 hr, for example, about 4, about 5, about 6, about 7, about 8, about 9, about 10, about 11, about 12, about 13, about 14, about 15, about 16, about 17, about 18, about 19, about 20, about 21, about 22, about 23, or about 24 hr after single-dose administration and will desirably be bioequivalent according to FDA guidelines to a second orally administrable dosage form comprising tramadol also suitable for once daily administration.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that said first once daily controlled-release dosage form will desirably further exhibit at steady state the following pharmacokinetic parameters in-vivo under fasting conditions: (i) an AUC₀₋₂₄ from about 1635 to about 21000, for example, from about 1635 to about 3920, about 3610 to about 9120, or about 9455 to about 20965 ng·h/ml, and (ii) a Cₘₐₓ from about 117 to about 1230, for example, about 117 to about 245, about 230 to about 590, or about 590 to about 1230 ng/ml and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that said first once daily controlled-release dosage form will desirably further exhibit under fasting conditions a Tₘₐₓ of, for example, about 9, about 10, about 11, about 12, about 13, or about 14 hours and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that said first once daily controlled-release dosage form will desirably further exhibit under fasting conditions a degree of fluctuation (%) of, for example, about 43 to about 141, about 43 to about 120, about 58 to about 132, or about 57 to about 141 and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that said first once daily controlled-release dosage form will desirably further exhibit under fasting conditions Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that said first once daily controlled-release dosage form will desirably further exhibit under fasting conditions a Tₘₐₓ of, for example, about 9, about 10, about 11, about 12, about 13, or about 14 hours, a degree of fluctuation (%) of, for example, about 43 to about 141, about 43 to about 120, about 58 to about 132, or about 57 to about 141, and Cₘᵢₙ of, for example, from about 31 to about 652, about 31 to about 117, about 96 to about 241, or about 226 to about 652 ng/ml at steady state and will desirably be bioequivalent according to FDA guidelines to a second orally administrable dosage form comprising the same dose of tramadol also suitable for once daily administration in the fasted state.

In at least one embodiment of the invention, the osmotic dosage form releases a therapeutically effective amount of tramadol by forcibly dispensing the tramadol from a core via a semipermeable membrane by diffusion and osmotic pumping or by osmotic pumping through at least one passageway in the membrane all or in part by pressure created in the core by osmosis i.e., positive hydrostatic pressure of a liquid, solvent, biological fluid or aqueous media and/or all or in part by the expansion of a swellable material which forces the tramadol to be dispensed from the core of the first once daily controlled-release dosage form, such that said first once daily controlled-release dosage form will desirably further exhibit under fasting conditions a Tₘₐₓ of tramadol from about 10 to about 20 hr, for example, about 10, about 11, about 12, about 13, about 14, about 15, about 16, about 17, about 18, about 19, or about 20 hr after single-dose administration and will desirably be bioequivalent according to FDA guidelines to a second orally administrable dosage form comprising the same dose of tramadol also suitable for once daily administration in the fasted state.

In at least one embodiment of the invention, the first once daily controlled-release dosage form comprises at least one osmotic delivery device comprising at least one unitary core comprising tramadol present in a therapeutically effective amount with at least one pharmaceutically acceptable excipient, said core surrounded by a semipermeable membrane which permits entry of an aqueous liquid into the core and delivery of the tramadol from the core to the exterior of the dosage form through at least one passageway by osmotic pumping or by diffusion and osmotic pumping through the semipermeable membrane.

In at least one embodiment of the invention, the first once daily controlled-release dosage form comprises a multiparticulate dosage form, each microparticle comprising an osmotic delivery device, each microparticle comprising at least one unitary core comprising tramadol with at least one pharmaceutically acceptable excipient, said core of each microparticle surrounded by a semipermeable membrane which permits entry of an aqueous liquid into the core and delivery of the tramadol from the core to the exterior of the dosage form by osmotic pumping through a plurality of pores formed in the semipermeable membrane by inclusion of a pore forming agent in the membrane or by diffusion and osmotic pumping through the semipermeable membrane.

In at least one embodiment of the invention, the first once daily controlled-release dosage form comprises a multiparticulate dosage form, each microparticle comprising an osmotic delivery device, each microparticle comprising a homogenous solid core comprising tramadol in admixture with at least one pharmaceutically acceptable excipient, an osmagent and/or an osmopolymer, said core of each microparticle surrounded by a semipermeable membrane which permits entry of an aqueous liquid into the core and delivery of the tramadol from the core to the exterior of the dosage form by osmotic pumping through a plurality of pores formed in the semipermeable membrane by inclusion of a pore forming agent in the membrane or by diffusion and osmotic pumping through the semipermeable membrane.

In at least one embodiment of the invention, the first once daily controlled-release dosage form comprises a multiparticulate dosage form, each microparticle comprising at least one unitary core comprising a therapeutically effective amount of tramadol with at least one pharmaceutically acceptable excipient in admixture with an osmagent, and/or an osmopolymer, and/or an wetting aid, said microparticles compressed into a core together with at least one pharmaceutically acceptable excipient, said core surrounded by a semipermeable membrane which permits entry of an aqueous liquid into the core and delivery of the tramadol from the interior to the exterior of the first dosage form by osmotic pumping through at least one passageway in the semipermeable membrane or by diffusion and osmotic pumping through the semipermeable membrane.

In at least one embodiment of the invention, the first once daily controlled-release dosage form comprises a multiparticulate dosage form, each microparticle comprising a sugar sphere or nonpareil bead coated with at least one layer comprising a therapeutically effective amount of tramadol with at least one pharmaceutically acceptable excipient, said at least one layer surrounded by a semipermeable membrane which permits entry of an aqueous liquid into the layer and delivery of the tramadol from the layer to the exterior of the dosage form by osmotic pumping through a plurality of pores formed in the semipermeable membrane by inclusion of a pore forming agent in the membrane or by diffusion and osmotic pumping through the semipermeable membrane.

In at least one embodiment of the invention, the first once daily controlled-release dosage form comprises a multiparticulate dosage form, each microparticle comprising a sugar sphere or nonpareil bead coated with at least one layer comprising a therapeutically effective amount of tramadol in admixture with at least one pharmaceutically acceptable excipient, an osmagent and/or an osmopolymer, said at least one layer surrounded by a semipermeable membrane which permits entry of an aqueous liquid into the layer and delivery of the tramadol from the layer to the exterior of the dosage form by osmotic pumping through a plurality of pores formed in the semipermeable membrane by inclusion of a pore forming agent in the membrane or by diffusion and osmotic pumping through the semipermeable membrane.

In at least one embodiment of the invention, the first once daily controlled-release dosage form comprises at least one controlled-release osmotic dosage form comprising at least one unitary comprising a therapeutically effective amount of tramadol in admixture with an osmagent, and/or an osmopolymer, and/or and wetting aid, said core surrounded by a nontoxic membrane or coat, such as for example a semipermeable membrane which permits entry of an aqueous liquid into the core and delivery of the tramadol from the core to the exterior of the dosage form by osmotic pumping through at least one passageway in the semipermeable membrane or by diffusion and osmotic pumping through the semipermeable membrane.

In at least one embodiment, the invention comprises first once daily controlled-release dosage form comprising an osmotic delivery device comprising tramadol present in a therapeutically effective amount in a layered, contacting arrangement with a swellable material composition to yield a solid core with two or more layers, which core is surrounded by a nontoxic membrane or coat, such as for example a semipermeable membrane which permits entry of an aqueous liquid into the core and delivery of the tramadol from the core to the exterior of the dosage form by osmotic pumping through at least one passageway in the semipermeable membrane or by diffusion and osmotic pumping through the semipermeable membrane.

In at least one embodiment of the invention, the first once daily controlled-release dosage form comprises at least one osmotic delivery device comprising a core and a membrane surrounding said core, said core comprising a therapeutically effective amount of tramadol, at least one means for increasing the hydrostatic pressure of the core and optionally at least one means for forcibly dispensing the tramadol from the device, said membrane comprising at least one means for the exit of the tramadol from the device, said device formulated such that when the device is in an aqueous medium, the at least one means for increasing the hydrostatic pressure of the core, and optionally the at least one means for forcibly dispensing the tramadol from the device and the at least one means for the exit of the tramadol cooperatively function to release the tramadol.

In at least one embodiment of the invention, the first once daily controlled-release dosage form comprising the at least one normal-release matrix core coated with at least one release-slowing coat will desirably comprise a mixture of an effective amount of at least two different tramadol salts comprises an immediate release coat, wherein said immediate release coat comprises a salt or form of tramadol, which is different from the salt or form of tramadol present in the remainder of the first once daily controlled release dosage form comprising the normal release matrix core coated with at least one release-slowing coat.

In at least one embodiment of the invention the first once daily controlled-release dosage form comprises at least one osmotic dosage form coated with a non-functional coat.

In at least one embodiment of the invention the first once daily controlled-release dosage form comprises at least one osmotic dosage form further comprising an osmotic subcoat.

In at least one embodiment of the invention the first once daily controlled-release dosage from comprises at least one osmotic dosage from coated further coated with a release-slowing coat.

In at least one embodiment of the invention, the first once daily controlled-release dosage form comprises at least one osmotic dosage form surrounded by a release-slowing coat, said release-slowing coat comprising a material that is soluble or slowly dissolving in intestinal juices, substantially pH neutral or basic fluids or fluids having a pH higher than gastric fluid, but for the most part insoluble in gastric juices or acidic fluids.

In at least one embodiment of the invention, the first once daily controlled-release dosage form comprises at least one osmotic dosage form surrounded with at least one delayed-release coat.

In at least one embodiment of the invention, the first once daily controlled-release dosage form comprises at least one osmotic dosage form surrounded by at least one release-slowing coat, which coat comprises at least one water-insoluble water-permeable film-forming polymer and at least one water-soluble polymer.

In at least one embodiment of the invention, the first once daily controlled-release dosage form comprises at least one osmotic dosage form surrounded by at least one release-slowing coat, which coat comprises at least one water-insoluble water-permeable film-forming polymer and at least one water-soluble polymer and at least one plasticizer.

In at least one embodiment of the invention, the first once daily controlled-release dosage form comprises at least one osmotic dosage form surrounded by at least one release-slowing coat, which coat comprises at least one water-insoluble water-permeable film-forming polymer, at least one water-soluble polymer and at least one means for the exit of tramadol from the core of the osmotic dosage form.

In at least one embodiment of the invention, the first once daily controlled-release dosage form comprises at least one osmotic dosage form surrounded by a release-slowing coat, which coat comprises at least one water-insoluble water-permeable film-forming polymer, at least one water-soluble polymer and at least one passageway.

In at least one embodiment of the invention, the first once daily controlled-release dosage form comprises at least one osmotic dosage form surrounded by at least one release-slowing coat, which coat comprises at least one water-insoluble water-permeable film-forming polymer, at least one water-soluble polymer and at least one plasticizer.

In at least one embodiment of the invention, the first once daily controlled-release dosage form comprises at least one osmotic dosage form surrounded by at least one release-slowing coat, which coat comprises at least one water-insoluble water-permeable film-forming polymer, at least one water-soluble polymer, at least one plasticizer, and at least one means for the exit of tramadol from the core of the osmotic dosage form.

In at least one embodiment of the invention, the first once daily controlled-release dosage form comprises at least one osmotic dosage form surrounded by at least one release-slowing coat, which coat comprises at least one water-insoluble water-permeable film-forming polymer, at least one water-soluble polymer, at least one plasticizer, and at least one passageway.

In at least one embodiment of the invention, the first once daily controlled-release dosage form comprises at least one osmotic dosage form surrounded by at least one release-slowing coat, which coat comprises at least one aqueous dispersion of a neutral ester copolymer without any functional groups, a poly glycol having a melting point greater than 55o C, one or more pharmaceutically acceptable excipients, and optionally at least one means for the exit of tramadol form the core of the osmotic dosage form and is cured at a temperature at least equal to or greater than the melting point of the poly glycol.

In at least one embodiment of the invention, the first once daily controlled-release dosage form comprises at least one osmotic dosage form surrounded by at least one release-slowing coat, which coat comprises at least one enteric polymer.

In at least one embodiment of the invention, the first controlled-release dosage form comprises at least one osmotic dosage form coated with at least one non-functional soluble coating.

In at least one embodiment of the invention, the first once daily controlled-release dosage form comprises at least one osmotic dosage form in the form of a tablet.

In at least one embodiment of the invention, the first once daily controlled-release dosage form comprises at least one osmotic dosage form in the form of a microparticle.

In at least one embodiment of the invention, the first once daily controlled-release dosage form comprises at least one osmotic dosage form, which osmotic dosage form comprises a plurality of microparticles, wherein each microparticle is an osmotic dosage form.

In at least one embodiment of the invention, the first once daily controlled-release dosage form comprises at least one osmotic dosage form wherein the core of the osmotic dosage form comprises a unitary core.

In at least one embodiment of the invention, the first once daily controlled-release dosage form comprises at least one osmotic dosage form wherein the core of the osmotic dosage form comprises at least two layers.

In at least one embodiment of the invention, the first once daily controlled-release dosage form comprises at least one osmotic dosage form wherein the core of the osmotic dosage form comprises a plurality of coated cores.

In one embodiment of the invention, the first once daily controlled-release dosage form comprises at least one osmotic dosage form whose core comprises, for example, a unitary core or a plurality of cores. In certain embodiments, the core comprises at least one microparticle. In certain embodiments, the core comprises a plurality of microparticles compressed to form a unitary core. Methods and means of manufacture of microparticles are well know to one of skill in the pharmaceutical delivery arts and are also described else where in this disclosure.

In certain embodiments of the invention, the first once daily controlled-release dosage form comprising at least one controlled-release dosage form or at least one means for controllably releasing the tramadol will desirably comprise at least one pharmaceutically acceptable excipient. Such excipients will desirably aid in the processing of the dosage form and/or in certain embodiments modulate the rate of release of the tramadol. Depending on the intended main function, excipients to be used in tablets are subcategorized into different groups. However, one excipient will desirably affect the properties of the first controlled-release dosage form in a series of ways, and many excipients used in pharmaceutical compositions will desirably thus be described as being multifunctional.

In at least one embodiment of the invention the first once daily controlled-release dosage form or means for controllably releasing the tramadol comprises at least one binder. In certain embodiments the binder is water-insoluble. Examples of binders include hydrogenated vegetable oil, castor oil, paraffin, higher aliphatic alcohols, higher aliphatic acids, long chain fatty acids, fatty acid esters, wax-like materials such as fatty alcohols, fatty acid esters, fatty acid glycerides, hydrogenated fats, hydrocarbons, normal waxes, stearic acid, stearyl alcohol, hydrophobic and hydrophilic polymers having hydrocarbon backbones, and mixtures thereof. Examples of water-soluble polymer binders include modified starch, gelatin, polyvinylpyrrolidone, cellulose derivatives (such as for example hydroxypropyl methylcellulose (HPMC) and hydroxypropyl cellulose (HPC)), polyvinyl alcohol and mixtures thereof. In at least one embodiment, the binder will desirably be present in an amount of from about 0.1% to about 20% by weight of the first once daily controlled-release dosage form or means for controllably releasing the tramadol. For example, in certain embodiments the binder is present in an amount of from about 0.2, about 0.5, about 0.8, about 1, about 2, about 3, about 4, about 5, about 6, about 7, about 8, about 9, about 10, about 11, about 12, about 13, about 14, about 15, about 16, about 17, about 18, about 19, or about 20% by weight of the first once daily controlled-release dosage form or first once daily controlled-release dosage form comprising at least one means for controllably releasing the tramadol.

In at least one embodiment of the invention the first once daily controlled-release dosage form or means for controllably releasing the tramadol comprises at least one lubricant. Examples of lubricants include stearic acid, hydrogenated vegetable oils (such as hydrogenated cottonseed oil (Sterotex®), hydrogenated soybean oil (Sterotex® HM) and hydrogenated soybean oil & castor wax (Sterotex® K)) stearyl alcohol, leucine, polyethylene glycol (MW 1450, suitably 4000, and higher), magnesium stearate, glyceryl monostearate, stearic acid, glycerylbehenate, polyethylene glycol, ethylene oxide polymers (for example, available under the registered trademark Carbowax® from Union Carbide, Inc., Danbury, Conn.), sodium lauryl sulfate, magnesium lauryl sulfate, sodium oleate, sodium stearyl fumarate, DL-leucine, colloidal silica, and mixtures thereof. The lubricant will desirably be present in an amount of from about 0 to about 5% by weight of the first once daily controlled-release dosage form or the at least one means for controllably releasing the tramadol. For example, in certain embodiments the lubricant is present in an amount of from about 0, about 1, about 1.5, about 2, about 2.5, about 3, about 3.5, about 4, about 4.5, or about 5% by weight of the first once daily controlled-release dosage form or the at least one means for controllably releasing the tramadol.

Emulsifying agent(s) (also called emulsifiers or emulgents) will desirably be included in coatin formulations to facilitate actual emulsification during manufacture of the coat, and also to ensure emulsion stability during the shelf-life of the product. Examples of emulsifying agents will desirably include, for example, naturally occurring materials and their semi synthetic derivatives, such as the polysaccharides, as well as glycerol esters, cellulose ethers, sorbitan esters (e.g. sorbitan monooleate or Span™ 80), and polysorbates (e.g. Tween™ 80). Combinations of emulsifying agents are operable.

Anti-foaming agent(s) will desirably be included to reduce frothing or foaming during manufacture of coats. Anti-foaming agents useful for coat compositions include, but are not limited to simethicone, polyglycol, silicon oil, and mixtures thereof.

In at least one embodiment of the invention, the first once daily controlled-release dosage form or means for controllably releasing the tramadol comprises at least one plasticizer. The use of plasticizers is optional but will desirably be included in the first once daily controlled-release dosage form or means for controllably releasing the tramadol to modify the properties and characteristics of the polymers used in the coats or cores of the dosage forms for convenient processing during manufacture of the coats and/or the cores if necessary. Plasticizers useful in embodiments of the invention will desirably include, for example, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol, glycerin, ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. It is also contemplated and within the scope of the invention, that a combination of plasticizers may be used in the present formulation. The PEG based plasticizers are available commercially or will desirably be made by a variety of methods, such as disclosed in Poly(ethylene glycol) Chemistry: Biotechnical and Biomedical Applications (J. M. Harris, Ed.; Plenum Press, NY). Once the first once daily controlled-release dosage form or means for controllably releasing the tramadol is manufactured, certain plasticizers will desirably function to increase the hydrophilicity of the coat(s) and/or the core of the osmotic dosage form in the environment of use may it be in-vitro or in-vivo. Accordingly, certain plasticizers will desirably function as flux enhancers.

In certain embodiments of the invention, the first once daily controlled-release dosage form or the at least one means for controllably releasing the tramadol require the incorporation of one or more fillers or diluents, which include, for example, dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol, sucralfate, calcium hydroxyl-apatite, calcium phosphates and fatty acid salts such as magnesium stearate. In certain embodiments the diluent will desirably be added in an amount so that the combination of the diluent and the active substance comprises up to about 90% by weight of the composition.

In at least one embodiment of the invention the first once daily controlled-release dosage form or the at least one means for controllably releasing the tramadol comprises a solubilizer. The solubilizer will desirably be selected from hydrophilic surfactants or lipophilic surfactants or mixtures thereof. The surfactants will desirably be anionic, nonionic, cationic, and zwitterionic surfactants. The hydrophilic non-ionic surfactants will desirably be selected from the group comprised of, but not limited to: polyethylene glycol sorbitan fatty acid esters and hydrophilic transesterification products of a polyol with at least one member of the group from triglycerides, vegetable oils, and hydrogenated vegetable oils such as glycerol, ethylene glycol, polyethylene glycol, sorbitol, propylene glycol, pentaerythritol, or a saccharide, d-α-tocopheryl polyethylene glycol 1000 succinate. The ionic surfactants will desirably be selected from the group comprised of, but not limited to: alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono-and di-acetylated tartaric acid esters of mono-and di-glycerides; succinylated mono-and di-glycerides; citric acid esters of mono-and di-glycerides; and mixtures thereof. The lipophilic surfactants will desirably be selected from the group comprised of, but not limited to: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono-and di-glycerides; hydrophobic transesterification products of a polyol with at least one member of the group from glycerides, vegetable oils, hydrogenated vegetable oils, fatty acids and sterols; oil-soluble vitamins/vitamin derivatives; PEG sorbitan fatty acid esters, PEG glycerol fatty acid esters, polyglycerized fatty acid, polyoxyethylene-polyoxypropylene block copolymers, sorbitan fatty acid esters; and mixtures thereof. In at least one embodiment the solubilizer will desirably be selected from: PEG-20-glyceryl stearate (Capmul® by Abitec), PEG-40 hydrogenated castor oil (Cremophor RH 40® by BASF), PEG 6 corn oil (Labrafil® by Gattefosse), lauryl macrogol-32 glyceride (Gelucire44/14® by Gattefosse) stearoyl macrogol glyceride (Gelucire50/13® by Gattefosse), polyglyceryl-10 mono dioleate (Caprol® PEG860 by Abitec), propylene glycol oleate (Lutrol® by BASF), Propylene glycol dioctanoate (Captex® by Abitec), Propylene glycol caprylate/caprate (Labrafac® by Gattefosse), Glyceryl monooleate (Peceol® by Gattefrosse), Glycerol monolinoleate (Maisine® by Gattefrosse), Glycerol monostearate (Capmul® by Abitec), PEG-20 sorbitan monolaurate (Tween20® by ICI), PEG-4 lauryl ether (Brij30® by ICI), Sucrose distearate (Sucroester7® by Gattefosse), Sucrose monopalmitate (Sucroester15® by Gattefosse), polyoxyethylene-polyoxypropylene block copolymer (Lutrol® series BASF), polyethylene glycol 660 hydroxystearate, (Solutol® by BASF), Sodium lauryl sulfate, Sodium dodecyl sulphate, Dioctyl suphosuccinate, L-hydroxypropyl cellulose, hydroxylethylcellulose, hydroxylpropylcellulose, Propylene glycol alginate, sodium taurocholate, sodium glycocholate, sodium deoxycholate, betains, polyethylene glycol (Carbowax® by DOW), d-α-tocopheryl polyethylene glycol 1000 succinate, (Vitamin E TPGS® by Eastman), and mixtures thereof. In at least one other embodiment the solubilizer will desirably be selected from PEG-40 hydrogenated castor oil (Cremophor RH 40® by BASF), lauryl macrogol-32 glyceride (Gelucire44/14® by Gattefosse) stearoyl macrogol glyceride (Gelucire 50/13® by Gattefosse), PEG-20 sorbitan monolaurate (Tween 20® by ICI), PEG-4 lauryl ether (Brij30® by ICI), polyoxyethylene-polyoxypropylene block copolymer (Lutrol® series BASF), Sodium lauryl sulphate, Sodium dodecyl sulphate, polyethylene glycol (Carbowax® by DOW), and mixtures thereof.

In at least one embodiment of the invention the first once daily controlled-release dosage form or the at least one means for controllably releasing the tramadol comprises a swelling enhancer. At lower concentrations, these excipients will desirably be used as superdisintegrants; however at concentrations above about 5% w/w these agents will desirably function as swelling enhancers and help increase the size of the first once daily controlled-release dosage form or the at least one means for controllably releasing the tramadol. Examples of swelling enhancers include but are not limited to: low-substituted hydroxypropyl cellulose, microcrystalline cellulose, cross-linked sodium or calcium carboxymethylcellulose, cellulose fiber, cross-linked polyvinyl pyrrolidone, cross-linked polyacrylic acid, cross-linked Amberlite resin, alginates, colloidal magnesium-aluminum silicate, corn starch granules, rice starch granules, potato starch granules, pregelatinised starch, sodium carboxymethyl starch and mixtures thereof. In at least one embodiment of the matrix dosage forms, the swelling enhancer is cross-linked polyvinyl pyrrolidone. The content of the swelling enhancer will desirably be from about 5% to about 90% by weight of the matrix dosage form. For example, in certain embodiments the swelling enhancer is present at about 5, about 10, about 15, about 20, about 25, about 30, about 35, about 40, about 45, about 50, about 55, about 60, about 65, about 70, about 75, about 80, about 85, or about 90% by weight of the first once daily controlled-release dosage form or the at least one means for controllably releasing the tramadol.

In another embodiment of the invention the first once daily controlled-release dosage form or the at least one means for controllably releasing the tramadol comprises at least one disintegrant or superdisintegrant. Examples of disintegrants for use in the first once daily controlled-release dosage form or the at least one means for controllably releasing the tramadol include crosscarmellose sodium, crosspovidone, alginic acid, sodium alginate, methacrylic acid DVB, cross-linked PVP, microcrystalline cellulose, polacrilin potassium, sodium starch glycolate, starch, pregelatinized starch and the like. In at least one embodiment the disintegrant is chosen from cross-linked polyvinylpyrrolidone (e.g. Kollidon® CL), cross-linked sodium carboxymethylcellulose (e.g. Ac-Di-Sol), starch or starch derivatives such as sodium starch glycolate (e.g. Explotab®), or combinations with starch (e.g. Primojel™), swellable ion-exchange resins, such as Amberlite IRP 88, formaldehyde-casein (e.g. Esma Spreng™), and mixtures thereof. The disintegrant will desirably be present in an amount of from about 0 to about 20% of the total weight of the first once daily controlled-release dosage form or the at least one means for controllably releasing the tramadol matrix. For example, the disintegrant will desirably be present at about 0, about 2, about 4, about 6, about 8, about 10, about 12, about 14, about 16, about 18, or about 20% of the total weight of the first once daily controlled-release dosage form or the at least one means for controllably releasing the tramadol matrix.

In another embodiment of the invention the first once daily controlled-release dosage form or the at least one means for controllably releasing the tramadol comprises at least one glidant. Examples of glidants include, without limitation, colloidal silicon dioxide, magnesium trisilicate, powdered cellulose, starch, talc, and tribasic calcium phosphate. The glidant will desirably be present in amount form about 0% to about 20%, such as for example, about 0, about 2, about 4, about 6, about 8, about 10, about 12, about 14, about 16, about 18 or about 20% of the total weight of the first once daily controlled-release dosage form or the at least one means for controllably releasing the tramadol matrix.

In another embodiment of the invention the first once daily controlled-release dosage form or the at least one means for controllably releasing the tramadol comprises at least one flux enhancing, channeling agent or pore former. Examples of flux enhancing, channeling agents or pore formers include, for example, sodium chloride, potassium chloride, sucrose, sorbitol, mannitol, polyethylene glycol, propylene glycol, hydroxypropyl cellulose, hydroxypropyl methylcellulose, hydroxypropyl methylcellulose phthalate, cellulose acetate phthalate, polyvinyl alcohols, methacrylic copolymers, and combinations thereof. Some plasticizers will desirably also function as flux enhancers by increasing the hydrophilicity of the first once daily controlled-release dosage form or the at least one means for controllably releasing the tramadol. Flux enhancers or channeling agents will desirably also function as a means for the exit of tramadol from the core if the flux enhancing or channeling agent is used in a sufficient amount. The amount of flux enhancer or channeling agent will desirably be from about 0 to about 20% of the total dosage form, such as for example, about 0, about 2, about 4, about 6, about 8, about 10, about 12, about 14, about 16, about 18 or about 20% of the total dosage form.

In another embodiment of the invention the first once daily controlled-release dosage form or the at least one means for controllably releasing the tramadol comprises at least one hydroattractant. Examples of hydroattractants include, for example, water-insoluble polymers such as low substituted hydroxypropyl cellulose, ion exchange resins, microcrystalline cellulose, cross-linked sodium or calcium carboxymethylcellulose, cellulose fiber, cross-linked polyvinyl pyrrolidone, cross-linked polyacrylic acid, cross-linked Amberlite resin, alginates, such as for example, algin, sodium alginate, potassium alginate, chitin, colloidal magnesium-aluminum silicate, corn starch granules, wheat starch granules, rice starch granules, potato starch granules, zein, soya polysaccharide, and sodium carboxymethyl starch, inorganic and organic salts and sugars, such as for example, magnesium sulfate, magnesium chloride, sodium chloride, lithium chloride, potassium sulfate, sodium carbonate, sodium sulfite, lithium sulfate, potassium chloride, calcium carbonate, sodium sulfate, calcium sulfate, potassium acid phosphate, calcium lactate, d-mannitol, urea, inositol, magnesium succinate, tartaric acid, water-soluble acids, alcohols, surfactants, and carbohydrates such as raffinose, sucrose, glucose, lactose, fructose, carrageenan, fucoridan, furcellaran, laminaran, hypnea, gum arabic, gum ghatti, gum karaya, locust bean gum, pectin, starch or combinations thereof.

In another embodiment of the invention the first once daily controlled-release dosage form or the at least one means for controllably releasing the tramadol comprises at least one gel modifier. Example of gel modifiers include, for example, sugars, polyols and soluble salts.

In other embodiments of the invention, first once daily controlled-release dosage form or the at least one means for controllably releasing the tramadol comprises one or more pharmaceutically acceptable excipients such as, for example, granulating aids or agents, pH adjusters, and like excipients conventionally used in pharmaceutical compositions.

In certain embodiments of the invention, the first once daily controlled-release dosage form comprises at least one osmotic dosage form, the core of said osmotic dosage form comprising wetting aid, such as for example, fatty acid based monoglycerides; medium chain triglycerides; glyceryl monostearates; glyceryl monooleate; glyceryl palmitostearate; surfactants, such as for example, sodium lauryl sulfate, sodium taurocholate, poloxamers; sorbitan esters, such as for example, polyoxyethylene sorbitan fatty acid esters; chelating agents such as citric acid; glycerides, such as for example, Gelucire®; TPGS; or any combination thereof. The core comprises about 0-about 20% of the wetting aid based on the total weight of the core, such as for example, about 0, about 2, about 4, about 6, about 8, about 10, about 12, about 14, about 16, about 18, or about 20% of the total weight of the core.

In certain embodiments of the invention, the first once daily controlled-release dosage form comprises at least one osmotic dosage form, the core of said osmotic dosage form comprising at least one means for increasing the hydrostatic pressure of the core. The membrane or coat will desirably be a semipermeable membrane, a release-slowing coat, a water-soluble coat, an osmotic subcoat, and any combination thereof. The core must have an effective osmotic pressure greater than that of the surrounding fluid in the environment of use so that there is a net driving force for water to enter the core. The at least one means for increasing the hydrostatic pressure of the core will desirably be any material that increases the osmotic pressure of the core of the osmotic dosage form. The at least one means for increasing the hydrostatic pressure of the core will desirably be, for example, tramadol, an osmagent, any material which will desirably interact with or facilitate water uptake r and/or an aqueous biological fluid, swell and retain water within their structure, such as for example an osmopolymer, and any combination thereof. The osmagent will desirably be soluble or swellable. Examples of osmotically effective solutes are water-insoluble polymers such as low substituted hydroxypropyl cellulose, ion exchange resins, microcrystalline cellulose, cross-linked sodium or calcium carboxymethylcellulose, cellulose fiber, cross-linked polyvinyl pyrrolidone, cross-linked polyacrylic acid, cross-linked Amberlite resin, alginates, such as for example, algin, sodium alginate, potassium alginate, chitin, colloidal magnesium-aluminum silicate, corn starch granules, wheat starch granules, rice starch granules, potato starch granules, zein, soya polysaccharide, and sodium carboxymethyl starch, inorganic and organic salts and sugars, such as for example, magnesium sulfate, magnesium chloride, sodium chloride, lithium chloride, potassium sulfate, sodium carbonate, sodium sulfite, lithium sulfate, potassium chloride, calcium carbonate, sodium sulfate, calcium sulfate, potassium acid phosphate, calcium lactate, d-mannitol, urea, inositol, magnesium succinate, tartaric acid, water-soluble acids, alcohols, surfactants, and carbohydrates such as raffinose, sucrose, glucose, lactose, fructose, carrageenan, fucoridan, furcellaran, laminaran, hypnea, gum arabic, gum ghatti, gum karaya, locust bean gum, pectin, starch or combinations thereof. In certain embodiments the amount of osmagent will desirably range from, for example, about 15, about 20, about 25, about 30, about 35, about 40, about 45, about 50, about 55, about 60, about 65, about 70, about 75, about 80, about 85, about 90, or about 95% of the core dry weight. Tramadol will desirably itself be an osmagent or will desirably be combined, with one or more other osmagents.

In certain embodiments of the invention, the at least one means for increasing the hydrostatic pressure will desirably comprise, in addition to an osmagent, any material which will desirably interact with water and/or an aqueous biological fluid, swell and retain water within their structure. In certain embodiments where the at least one means for increasing the hydrostatic pressure is an osmopolymer, which will desirably be slightly cross-linked or uncross-linked. The uncross-linked polymers to be used as osmopolymers, when in contact with water and/or aqueous biological fluid, should not dissolve in water, hence maintaining their physical integrity. Osmopolymers will desirably be, for example, chosen from the group of polyacrylic acid derivatives (e.g., polyacrylates, poly-methyl methacrylate, poly(acrylic acid) higher alkyl esters, poly(ethylmethacrylate), poly(hexadecyl methacrylate-co-methylmethacrylate), poly(methylacrylate-co-styrene), poly(n-butyl methacrylate), poly(n-butyl-acrylate), poly(cyclododecyl acrylate), poly(benzyl acrylate), poly(butylacrylate), poly(secbutylacrylate), poly(hexyl acrylate), poly(octyl acrylate), poly(decyl acrylate), poly(dodecyl acrylate), poly(2-methyl butyl acrylate), poly(adamantyl methacrylate), poly(benzyl methacrylate), poly(butyl methacrylate), poly(2-ethylhexyl methacrylate), poly(octyl methacrylate), acrylic resins), polyacrylamides, poly(hydroxy ethyl methacrylate), poly(vinyl alcohol), poly(ethylene oxide), poly N-vinyl-2-pyrrolidone, naturally occurring resins such as polysaccharides (e.g., dextrans, water-soluble gums, starches, chemically modified starches), cellulose derivatives (e.g., cellulose esters, cellulose ethers, chemically modified cellulose, microcrystalline cellulose, sodium carboxymethylcellulose and methylcellulose), starches, Carbopol™, acidic carboxy polymer, Cyanamer™, polyacrylamides, cross-linked water-swellable indene-maleic anhydride polymers, Good-rite™, polyacrylic acid, polyethyleneoxide, starch graft copolymers, Aqua-Keeps™, acrylate polymer, diester cross-linked polyglucan, and any combination thereof. The amount of osmopolymer will desirably range from about 0 to about 40%, such as for example, about 0, about 5, about 10, about 15, about 20, about 25, about 30, about 35, or about 40% of the total dosage form.

In certain embodiments of the invention, the first once daily controlled-release dosage form comprises at least one osmotic dosage form, said osmotic dosage form comprising a core, which further comprises a means for forcibly dispensing tramadol from the core to the exterior of the dosage form. The at least one means for forcibly dispensing tramadol will desirably be any material which will desirably swell in water and/or aqueous biological fluid and retain a significant fraction of water within its structure, and will not dissolve in water and/or aqueous biological fluid, a means for generating a gas, an osmotically effective solute or any combination thereof which will desirably optionally be surrounded by a membrane or coat depending on the particular means used. The membrane or coat will desirably be, for example, a membrane or coat that is essentially impermeable to the passage of tramadol, gas and compounds, and is permeable to the passage of water and/or aqueous biological fluids. Such a coat or membrane comprises, for example, a semipermeable membrane, microporous membrane, asymmetric membrane, which asymmetric membrane will desirably be permeable, semipermeable, perforated, or unperforated. In at least one embodiment, the at least one means for forcibly dispensing tramadol from the core of the osmotic dosage form comprises a means for generating gas, which means for generating gas is surrounded by, for example, a semipermeable membrane. In operation, when the gas generating means imbibes water and/or aqueous biological fluids, the means for generating gas reacts and generates gas, thereby enlarging and expanding the at least one means for forcibly dispensing tramadol unidirectionally or multidirectionally. The means for generating a gas comprises at least one compound, which will desirably produce effervescence, such as for example, at least one solid acid compound and at least one solid basic compound, which in the presence of a fluid will desirably react to form a gas, such as for example, carbon dioxide. Examples of acid compounds include, organic acids such as malic, fumaric, tartaric, itaconic, maleic, citric, adipic, succinic and mesaconic, and inorganic acids such as sulfamic or phosphoric, also acid salts such as monosodium citrate, potassium acid tartrate and potassium bitartrate. The basic compounds include, for example, metal carbonates and bicarbonates salts, such as alkali metal carbonates and bicarbonates. The acid and base materials will desirably be used in any convenient proportion between about 1 to about 200 parts of the at least one acid compound to the at least one basic compound or about 1 to about 200 parts of the at least one basic compound to the at least one acid compound. Guidance for the manufacture and use for the means for generating gas is provided, for example, in U.S. Pat. No. 4,235,236.

In at least one embodiment of the invention, the first once daily controlled-release dosage form comprises at least one osmotic dosage form, which comprises at least one means for forcibly dispensing tramadol from the core of the osmotic dosage form. The at least one means for forcibly dispensing tramadol comprises at least one material which will desirably swell in water and/or aqueous biological fluid and retain a significant fraction of water within its structure, and will not dissolve in water and/or aqueous biological fluid, such as for example, a hydrogel or water swellable polymer. Hydrogels or water swellable polymers include, for example, lightly cross-linked hydrophilic polymers, which swell in the presence of fluid to a high degree without dissolution, usually exhibiting a about 5 to about 50-fold volume increase. Examples of hydrogels include any hydrophilic polymer or combination of hydrophilic polymers, which when hydrated will desirably form a network of polymer fibrils that will desirably form a chemically (covalent) crosslinked, physically crosslinked (non-covalent) or combination of chemically or physically crosslinked polymeric gel structure. Such hydrophilic polymers are well known to one of ordinary skill in the art and include, for example, cellulose polymers and their derivatives (such as for example, hydroxyethylcellulose, hydroxypropylcellulose, carboxymethylcellulose, and microcrystalline cellulose), polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly (2-ethyl-2-oxazoline), poly(ethyleneimine), polyurethane hydrogels, and crosslinked polyacrylic acids and their derivatives, and mixtures thereof. Further examples include copolymers of the polymers listed in the preceding sentence, including block copolymers and grafted polymers. Specific examples of copolymers include PLURONIC® and TECTONIC®, which are polyethylene oxide-polypropylene oxide block copolymers. The terms “cellulose” and “cellulosic”, will desirably denote a linear polymer of anhydroglucose. Examples of cellulosic polymers include alkyl-substituted cellulosic polymers that ultimately dissolve in the gastrointestinal (GI) tract in a predictably delayed manner, such as for example, methylcellulose, hydroxymethylcellulose, hydroxyethylcellulose (NATRASOL® 250HX NF), hydroxypropylcellulose, hydroxypropylmethylcellulose, carboxymethylcellulose, and mixtures thereof. In terms of their viscosities, one class of alkyl-substituted celluloses includes those whose viscosity is within the range of about 000 to about 110,000 centipoise as a about 2% aqueous solution at about 20° C. Another class includes those whose viscosity is within the range of about 1,000 to about 4,000 centipoise as a about 1% aqueous solution at about 20° C. Polyalkylene oxides that will desirably be used include those having the properties described above for alkyl-substituted cellulose polymers. In at least one embodiment the polyalkylene oxide is poly(ethylene oxide), which term is used herein to denote a linear polymer of unsubstituted ethylene oxide. In at least one embodiment the poly(ethylene oxide) polymers have molecular weights of about 4,000,000 and higher. For example, in certain embodiment the poly(ethylene oxide) polymers have molecular weights within the range of about 4,500,000 to about 10,000,000, and in other embodiments have molecular weights within the range of about 5,000,000 to about 8,000,000. In certain embodiments the poly(ethylene oxide)s are those with a weight-average molecular weight within the range of about 1×10⁵ to about 1×10⁷, and in other embodiments within the range of about 9×10⁵ to about 8×10⁶. Poly(ethylene oxide)s are often characterized by their viscosity in solution. For example, in certain embodiments the poly(ethylene oxide)s have a viscosity range of about 50 to about 2,000,000 centipoise for a about 2% aqueous solution at about 20° C. In at least one embodiment the poly(ethylene oxide) is one or more of POLYOX® NF, grade WSR Coagulant, molecular weight 5 million, and grade WSR 303, molecular weight 7 million, and will desirably be used individually or in combination. Polysaccharide gums, both natural and modified (semi-synthetic) will desirably include for example, dextran, xanthan gum, gellan gum, welan gum, rhamsan gum, and mixtures thereof. Crosslinked polyacrylic acids that will desirably be include those whose properties are the same as those described above for alkyl-substituted cellulose and polyalkylene oxide polymers. In certain embodiments the crosslinked polyacrylic acids are those with a viscosity ranging from about 4,000 to about 40,000 centipoise for a about 1% aqueous solution at about 25° C. Examples of suitable crosslinked polyacrylic acids include CARBOPOL® NF grades 971P, 974P and 934P. Further examples of suitable crosslinked polyacrylic acids include polymers known as WATER LOCK®, which are starch/acrylates/acrylamide copolymers. isobutylene cross-linked with from about 0.001 to about 0.5 moles of a polyunsaturated cross-linking agent per mole of maleic anhydride in the copolymer as disclosed in U.S. Pat. No. 3,989,586, the water-swellable polymers or N-vinyl lactams as disclosed in U.S. Pat. No. 3,992,652, semi-solid cross-linked poly(vinyl pyrrolidone), diester cross-linked polyglucan hydrogels as described in U.S. Pat. No. 4,002,173, the anionic hydrogels of heterocyclic N-vinyl monomers as disclosed in U.S. Pat. No. 4,036,788, the ionogenic hydrophilic gels as described in J. Biomedical Mater, Res., Vol. 7, pages 123 to 126, 1973, and the like. The skill person will appreciate that some osmopolymers and hydrogels are inter-changeable.

It is within the purview of the skilled artisan, without undue experimentation, to determine the amount of the hydrogel or hydrophilic swellable polymer relative to the tramadol and other pharmaceutically acceptable excipients to be used to manufacture the first once daily controlled-release dosage form comprising the at least one means for forcibly dispensing tramadol from the core of the osmotic dosage form such that the desired in-vitro dissolution rate and the in-vivo pharmacokinetic parameters are met.

The hydrophilic water-swellable polymers will desirably be used individually or in combination. Certain combinations will often provide a more controlled release of the drug than their components when used individually. Examples include cellulose-based polymers combined with gums, such as hydroxyethylcellulose or hydroxypropyl cellulose combined with xanthan gum. Another example is poly(ethylene oxide) combined with xanthan gum.

The at least one means for forcibly dispensing tramadol from the core of the osmotic dosage form comprising the first once daily controlled-release dosage form will desirably optionally be covered by a membrane or coat impermeable to the passage of tramadol, and compounds, and is permeable to the passage of water and/or aqueous biological fluids. Such a coat or membrane comprises, for example, a semipermeable membrane, microporous membrane, asymmetric membrane, which asymmetric membrane will desirably be permeable, semipermeable, perforated, or unperforated.

In at least one embodiment of the invention, the first once daily controlled-release dosage form comprises at least one osmotic dosage form comprising at least one means for forcibly dispensing tramadol from the core of the osmotic dosage form. The at least one means for forcibly dispensing tramadol from the core of the once daily osmotic dosage form comprises at least one osmotically effective solute surrounded by a membrane or coat impermeable to the passage of tramadol, and compounds, and is permeable to the passage of water and/or aqueous biological fluids such that the osmotically effective solute will desirably exhibit an osmotic pressure gradient across a membrane or coat. Such coat or membrane comprises, for example, a semipermeable membrane, microporous membrane, asymmetric membrane, which asymmetric membrane will desirably be permeable, semipermeable, perforated, or unperforated. The osmotically effective solutes include, for example, the osmagents described above.

In embodiments of the invention where the means for forcibly dispensing tramadol is surrounded by a membrane or coat, at least one plasticizer will desirably be added to the membrane composition to impart flexibility and stretchability to the membrane or coat. In embodiments of the invention where the means for forcibly dispensing tramadol comprises a means for generating a gas, the membrane or coat should be stretchable so as to prevent rupturing of the membrane or coat during the period of delivery of tramadol. Guidance for the manufacture and use of such a membrane or coat is provided, for example, in U.S. Pat. No. 4,235,236.

The at least one means for forcibly dispensing tramadol from the core of the osmotic dosage form will desirably be located such that it is approximately centrally located within the core of the osmotic dosage form and is surrounded by a layer comprising tramadol. Guidance for the use and manufacture of such dosage forms is provided in U.S. Pat. No. 6,352,721. Alternatively, the core of the osmotic dosage form comprises at least two layers in which the first layer comprises tramadol, osmagent and/or osmopolymer and optionally at least one pharmaceutically acceptable excipient adjacent to a second layer comprising the means for forcibly dispensing tramadol. Alternatively, the core of the osmotic dosage form comprises a multilayered structure in which the layer comprising tramadol is sandwiched between two layers of the means for forcibly dispensing tramadol from the osmotic dosage from. Alternatively a layer comprising the means for forcibly dispensing tramadol from the osmotic dosage form will desirably surround a core comprising tramadol. The at least one means for the exit of the tramadol will desirably extend from the surface of the osmotic dosage form to the interior of the core comprising the tramadol.

The membrane or coat is permeable to the passage of aqueous media but not to the passage of tramadol present in the core. The membrane will desirably be, for example, a semipermeable membrane or an asymmetric membrane, which will desirably be permeable, semipermeable, perforated, or unperforated and will desirably deliver tramadol by osmotic pumping, diffusion or the combined mechanisms of diffusion and osmotic pumping. The structural integrity of such membranes should remain intact during the period of delivery of tramadol. By “intact”, in context of a semipermeable membrane for osmotic dosage forms, it is meant that the semipermeable property of the membrane is not compromised during the period of delivery of tramadol. The “period of delivery” is the duration during which the desired pharmacokinetic properties are achieved in-vivo. The in-vivo period will desirably be determined from the in-vitro dissolution rates if an In-Vivo In-Vitro Correlation (IVIVC) is established.

In certain embodiments of the invention, the first once daily controlled-release dosage forms comprising at least one controlled-release dosage form comprising at least one controlled-release matrix core or the at least one means for controllably releasing the tramadol, wherein the at least one means comprises at least one controlled-release matrix core or at least one normal release matrix core surrounded by at least one release-slowing coat, at least one delayed release coat, or a combination thereof, provide kinetics of drug release from the first once daily controlled-release dosage form is dependent at least in part upon the diffusion, dissolution, swelling, erosion or swelling and erosion properties of excipients within the composition. In such embodiments the first once daily controlled-release dosage forms comprise an effective amount of tramadol and at least one pharmaceutically acceptable excipient. The effective amount of tramadol present in the first once daily controlled-release dosage form for the management of moderate to moderately severe pain will desirably vary from about 25 mg to about 800 mg. For example, in certain embodiments, the amount of tramadol present will desirably be about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, or about 800 mg. The amount of tramadol present in the controlled-release or normal release matrix core comprising the first once daily controlled-release dosage form will desirably vary in an amount of from about 70% to about 98% by weight of the core dry weight. For example, in certain embodiments the tramadol is present at about 72, about 74, about 76, about 78, about 80, about 82, about 84, about 86, about 88, about 90, about 92, about 94, about 96, or about 98% by weight of the core dry weight. The first once daily controlled-release dosage form will desirably comprise multiparticulates or a unitary core, and will desirably optionally be coated with at least one release-slowing coat, delayed release coat, non-functional soluble coat, or an immediate release coating comprising tramadol. Release-slowing coatings include, by way of example, coatings which comprise at least one pH independent polymer, pH dependent polymer (such as for example enteric or reverse enteric types), soluble polymer, insoluble polymer (aqueous insoluble coat), swellable polymer, swellable and erodable polymer, hydrophobic material, hydrophilic material, or combinations thereof which when applied onto an uncoated normal release matrix core or controlled-release matrix core will desirably slow, modify, further slow, or further modify the rate of release of tramadol. The first once daily controlled-release dosage forms will desirably exhibit a modified-release, a controlled-release, a sustained release, a delayed release, a prolonged release, an extended release, or a bi-phasic release of the tramadol. The first once daily controlled-release dosage forms will desirably also exhibit an immediate release of the tramadol by virtue of the dosage form comprising an immediate-release coating, which immediate release coating comprises the tramadol. Accordingly, the first once daily controlled-release dosage forms will desirably exhibit an immediate release of the tramadol followed by a modified-release, controlled-release, sustained-release, prolonged-release, delayed release, or bi-phasic release of the tramadol.

The tramadol present in the cores of the first once daily controlled-release dosage forms will desirably be a mixture of different salts, active metabolites, enantiomers of tramadol. For example, in at least one embodiment of the invention, where the core of the first once daily controlled-release dosage form, comprises at least one controlled-release matrix core, at least one normal-release matrix core, or a combination thereof, and where the total amount of tramadol present is about 90% of the core, about 10, about 20, about 30, about 40, about 50, about 60, about 70, or about 80% of the tramadol will desirably be tramadol hydrochloride with the remainder of the tramadol being a different salt, active metabolite, or enantiomers of tramadol. Similarly, in embodiments where the first once daily controlled-release dosage forms comprise an immediate-release coating, the form of the tramadol present in the immediate release coating and the core, or even within the core of the first once daily controlled-release dosage form need not be of the same form. For example, in dosage forms comprising a controlled-release matrix core surrounded by at least one release slowing coat and an immediate release coat surrounding the release-slowing coat, the tramadol present in the controlled-release matrix core and the immediate-release coat will desirably be a different salt i.e., the controlled-release matrix core will desirably comprise tramadol hydrochloride and the immediate release coating will desirably comprise a salt of tramadol other than the hydrochloride salt of tramadol.

In at least one embodiment of the invention, the first once daily controlled-release dosage forms comprise at least one controlled-release dosage form or at least one means for controllably releasing the tramadol will desirably comprise a slowly dissolving salt or a low solubility salt of tramadol, such as for example tramadol saccharinate. The manufacture and use of tramadol saccharinate is disclosed, for example, in U.S. Pat. No. 6,576,260.

Non-functional soluble coatings are coatings that do not affect the rate of release of tramadol in-vitro or in-vivo, but will desirably enhance the chemical, biological, physical stability characteristics, or the physical appearance of the first once daily controlled-release dosage form.

In certain embodiments of the invention, the first once daily controlled-release dosage forms comprise at least one controlled-release dosage form or at least one means for controllably releasing the tramadol from the first once daily controlled-release dosage form. The at least one means for controllably releasing the tramadol will desirably comprise at least one controlled-release matrix core, at least one insoluble matrix core, at least one swellable matrix core, at least one swellable and erodable matrix core, at least one hydrophobic matrix core, at least one hydrophilic matrix core, at least one erodable matrix core, at least one release-slowing coat, at least one delayed release coat, at least one release-slowing coat comprising at least one pH independent polymer, at least one release-slowing coat comprising at least one pH dependent polymer (such as for example enteric or reverse enteric types), at least one release-slowing coat comprising at least one soluble polymer, at least one release-slowing coat comprising at least one insoluble polymer (aqueous insoluble coat), at least one release-slowing coat comprising at least one swellable polymer, at least one release-slowing coat comprising at least one lipid or waxy material or any combination thereof. Those skilled in the pharmaceutical art and the design of medicaments are well aware of controlled-release dosage forms as well as the various means for controllably releasing an active drug conventionally used in oral pharmaceutical compositions adopted for controlled release and means for their preparation. Examples of controlled release matrices are described in U.S. Pat. Nos. 6,326,027; 6,340,475; 6,905,709; 6,645,527; 6,576,260; 6,326,027; 6,254,887; 6,306,438; 6,129,933; 6,088,855; 5,891,471; 5,849,240; 5,965,163; 6,162,467; 5,843,680; 5,567,439; 5,552,159; 5,510,114; 5,478,857; 5,476,528; 5,453,283; 5,451,424; 5,443,846; 5,403,593; 5,378,462; 5,350,584; 5,283,065; 5,273,758; 5,266,331; 5,202,128; 5,183,690; 5,178,868; 5,165,952; 5,126,145; 5,073,379; 5,023,089; 5,007,790; 4,970,075; 4,959,208; 4,959,208; 4,861,598; 4,844,909; 4,834,984; 4,828,836; 4,806,337; 4,801,460; 4,764,378; 4,421,736; 4,383,393; 4,344,431; 4,343,789; 4,346,709; 4,230,687; 4,132,753; 5,591,452; 5,965,161; 5,958,452; 6,254,887; 6,156,342; 5,395,626; 5,474,786; and 5,919,826.

Suitable excipient materials for use in such controlled-release dosage forms or in controlled-release dosage forms comprising at least one means for controllably releasing the tramadol include, by way of example, release-resistant or controlled release materials such as for example hydrophobic polymers; hydrophilic polymers; lipophilic or waxy materials; soluble polymers; swellable polymers; insoluble polymers; release-slowing coats; delayed-release coats or mixtures thereof.

In at least one embodiment of the invention, the first once daily controlled-release dosage form comprises a hydrophilic dosage form or a means for controllably releasing the tramadol from the first once daily controlled-release dosage form, wherein said means comprises a hydrophilic matrix core. In at least one embodiment of the invention, the hydrophilic dosage form or hydrophilic matrix core matrix core is comprised of a polymeric hydrophilic matrix core.

Hydrophilic polymers are polymers, which have an affinity to water. Hydrophilic polymers are water-soluble if their molecules contain sufficient hydrophilic groups. Accordingly, not all hydrophilic polymers are water-soluble. Hydrophilic polymers used to manufacture the hydrophilic matrix core will desirably therefore be chosen from water-soluble polymers, polymers which are insoluble or stable under acidic conditions exhibiting a pH of less than 5 and which are soluble or decomposed under conditions exhibiting a pH of 5 or more (enteric polymers), polymers which are insoluble or stable under weakly acidic or basic conditions exhibiting a pH of more than 6 and which are soluble or decomposed under conditions exhibiting a pH of 6 or less, polymers which are insoluble or stable under conditions exhibiting a pH of from more than 4.5 to less than 6 and which are soluble or decomposed under conditions exhibiting a pH of 4.5 or less or a pH of 6 or more, water-swellable polymers (i.e., polymers that will desirably form hydrogels, which will desirably erode) or any combination thereof.

In certain embodiments, the first once daily controlled-release dosage form comprises a hydrophilic matrix core, which hydrophilic matrix core comprises at least one water-soluble polymer. Controlled-release of an active drug in a hydrophilic matrix core comprising a water-soluble polymer relies on slow dissolution of the matrix. In embodiments of the invention where the hydrophilic matrix comprises at least one water-soluble polymer, the at least one water-soluble polymer will desirably be, for example, pullulan, dextrin, sodium and calcium polyacrylic acid, polyacrylic acid, polymethacrylic acid, polymethylvinylether co-maleic anhydride, polyvinylpyrrolidone, polyethylene oxide, polyethylene glycol, hydroxypropylcellulose, hydroxypropylmethylcellulose, hydroxyethylcellulose, hydroxymethyl methacrylate, sodium carboxymethylcellulose, calcium carboxymethylcellulose, methylcellulose, maltodextrin, xanthan gum, tragacanth gum, agar, gellan gum, kayara gum, alginic acids, pectins, pre-gelatinized starch, and polyvinyl alcohol, and blends of those polymers, such as for example, those which form association polymers in the low pH environment of the stomach, such as mixtures of polyacrylic acid and polyethylene oxide or mixtures of polyacrylic acid and polyvinylpyrrolidone and will desirably include such polymers of different viscosities. These polymers will desirably be used individually or in combination from about 5% to about 90%, such as for example, about 5, about 10, about 15, about 20, about 25, about 30, about 35, about 40, about 45, about 50, about 55, about 60, about 65, about 70, about 75, about 80, about 85, or about 90% of the matrix core dry weight.

In embodiments where the hydrophilic matrix core comprises at least one water-soluble polymer which is insoluble or stable under acidic conditions exhibiting a pH of less than 5 and which is soluble or decomposed under conditions exhibiting a pH of 5 or more, the polymer will desirably be an enteric polymer, such as for example, carboxymethylethylcellulose, cellulose acetate phthalate, cellulose acetate succinate, methylcellulose phthalate, hydroxymethylethylcellulose phthalate, hydroxypropylmethylcellulose phthalate, hydroxypropylmethylcellulose acetate succinate, polyvinyl alcohol phthalate, polyvinyl butylate phthalate, polyvinyl acetal phthalate, a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate and a copolymer of methacrylic acid/methyl methacrylate. These enteric polymers will desirably be used individually or in combination and will desirably be present from about 5% to about 90%, such as for example, about 5, about 10, about 15, about 20, about 25, about 30, about 35, about 40, about 45, about 50, about 55, about 60, about 65, about 70, about 75, about 80, about 85, or about 90% of the matrix core dry weight.

Polymers which are insoluble or stable under weakly acidic or basic conditions exhibiting a pH of more than 6 and which are soluble or decompose under conditions exhibiting a pH of 6 or less are normally soluble in the acidic environment of the stomach. In embodiments where the hydrophilic matrix core comprises such a polymer, the polymer will desirably be, for example, benzylaminomethylcellulose, diethylaminomethylcellulose, piperidylethylhydroxyethylcellulose, cellulose acetate dimethylaminoacetate, a copolymer of vinyl diethylamine/vinyl acetate, a copolymer of vinyl benzylamine/vinyl acetate, polyvinyl acetal diethylamino acetate, a copolymer of vinylpiperidyl acetoacetal/vinyl acetate, polydiethylaminomethylstyrene, a copolymer of methyl methacrylate/butyl methacrylate/dimethylaminoethyl methacrylate and polydimethylaminoethyl methacrylate. These soluble hydrophilic polymers will desirably be used individually or in combination and will desirably be present from about 5% to about 80%, such as for example, about 5, about 10, about 15, about 20, about 25, about 30, about 35, about 40, about 45, about 50, about 55, about 60, about 65, about 70, about 75, about 80% of the matrix core dry weight.

In embodiments where the hydrophilic matrix core comprises at least one hydrophilic soluble polymer which is insoluble or stable under conditions exhibiting a pH of from more than 4.5 to less than 6 and which are soluble or decomposed under conditions exhibiting a pH of 4.5 or less or a pH of 6 or more are normally soluble in the stomach and intestine. In embodiments where the hydrophilic matrix core comprises such a polymer, the polymer will desirably be, for example a copolymer of 2-methyl-5-vinylpyridine/methyl methacrylate/methacrylic acid, a copolymer of 2-methyl-5-vinylpyridine/methyl acrylate/methacrylic acid, a copolymer of 2-vinyl-5-ethylpyridine/methacrylic acid/styrene, a copolymer of 2-vinyl-5-ethylpyridine/methacrylic acid/methyl acrylate, a copolymer of 2-vinylpyridine/methacrylic acid/methyl acrylate, a copolymer of 2-vinylpyridine/methacrylic acid/acrylonitrile, carboxymethylpiperidyl starch, carboxymethylbenzylaminocellulose, poly(2-vinylphenylglycine) and a copolymer of N-vinylglycine/styrene. Such soluble hydrophilic polymers will desirably be used individually or in combination.

In certain embodiments of the invention, the first once daily controlled-release dosage form comprises at least one swellable dosage or swellable and erodable form or a means for controllably releasing the tramadol, wherein the means for controllably releasing the tramadol comprises a swellable or swellable and erodable matrix core. These controlled-release dosage forms comprise at least one hydrophilic polymer, which is swellable in an aqueous medium. Swellable or swellable and erodable dosage forms or controlled-release dosage forms comprising a swellable or swellable and erodable matrix core as the means for controllably releasing the tramadol are at times also referred to as “hydrophilic colloid matrix systems”, “hydrogels”, “polymeric matrices involving moving boundaries”, or “hydrocoifoid matrices”. These controlled-release dosage forms are commonly manufactured by the compression of hydrophilic microparticulate powders, however, the skilled person will appreciate that the hydrophilic swellable or swellable and erodable polymers will desirably also be granulated in an organic solvent, extruded and then spheronized to form microparticulates or will desirably be compressed into mini-tablets. The principles relating to the mechanism and factors controlling drug release from swellable or swellable and erodable dosage forms is well known in the art. Briefly, drug release from controlled-release dosage forms comprising swellable or swellable and erodable matrix cores is based on glassy-rubbery transition of polymer as a result of water penetration into the matrix core. It is known in the art that interactions between water, hydrophilic swellable polymer and drug are the primary factors for release control, various formulation variables, such as for example, polymer grade, active drug/hydrophilic swellable polymer ratio, solubility of the active drug, and particle size of the active drug and hydrophilic swellable polymer will desirably also influence drug release rate to a faster or lesser degree. However, the central element of the mechanism of drug release is the gel layer (rubbery layer), which is formed around the swellable matrix core. The gel layer is capable of preventing the matrix disintegration and further rapid water penetration. Water penetration, polymer swelling, drug dissolution, diffusion and matrix erosion are the phenomena determining gel layer thickness. Finally, drug release is controlled by drug diffusion through the gel layer and/or by erosion of the gel layer. It is well known that the gel layer is physically delimited by two sharp fronts that separate different matrix states, i.e., the boundaries separating swollen matrix from solvent and glassy from rubbery polymer. The possibility of a third front has, however, also been described, and has been termed “undissolved drug front” or “diffusion front” and turned out to be a function of drug solubility and loading. Its presence will desirably create conditions such that the release will be more controlled by drug dissolution than by polymer swelling. Thus, in controlled-release dosage forms comprising a swellable or swellable and erodable dosage form or a means for controllably releasing a drug wherein the means comprises a swellable or swellable and erodable matrix core, three fronts will desirably be expected: (i) the swelling front (i.e., the boundary between the still glassy polymer and its rubbery state), (ii) the diffusion front (i.e., the boundary in the gel layer between the solid as yet undissolved drug and the dissolved drug), and (iii) the erosion front (i.e., the boundary between the matrix and the aqueous medium. It thus stands to reason that attempts to control movement of the fronts will desirably influence drug release kinetics. For example, reduction of matrix swelling by coating the matrix core or dosage form with a semipermeable coat, release-slowing coat, delayed-release coat, or a slowly permeable polymer will desirably further modulate release of the drug from controlled-release dosage forms comprising a swellable or swellable and erodable dosage forms or from controlled-release dosage forms comprising at least one means for controllably releasing the drug, wherein the means comprises at least one swellable or swellable and erodable matrix core.

The water-swellable polymer forming the swellable dosage form, the swellable and erodable dosage from, the at least one means for controllably releasing the tramadol, wherein the means comprises at least one swellable matrix core or a swellable and erodable matrix core in accordance with certain embodiments of the invention will desirably be any hydrophilic polymer or combination of hydrophilic polymers, which when hydrated will desirably form a network of polymer fibrils that will desirably form a chemically (covalent) crosslinked, physically crosslinked (non-covalent) or combination of chemically or physically crosslinked polymeric gel structure for controlling the release of the tramadol. Such hydrophilic polymers are well known to one of ordinary skill in the art and include, for example, cellulose polymers and their derivatives (such as for example, hydroxyethylcellulose, hydroxypropylcellulose, carboxymethylcellulose, and microcrystalline cellulose), polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly (2-ethyl-2-oxazoline), poly(ethyleneimine), polyurethane hydrogels, and crosslinked polyacrylic acids and their derivatives, and mixtures thereof. Further examples include copolymers of the polymers listed in the preceding sentence, including block copolymers and grafted polymers. Specific examples of copolymers include PLURONIC® and TECTONIC®, which are polyethylene oxide-polypropylene oxide block copolymers.

The terms “cellulose” and “cellulosic”, as used within this section regarding the swellable matrix embodiments of the present invention, will desirably denote a linear polymer of anhydroglucose. Examples of cellulosic polymers include alkyl-substituted cellulosic polymers that ultimately dissolve in the gastrointestinal (GI) tract in a predictably delayed manner, such as for example, methylcellulose, hydroxymethylcellulose, hydroxyethylcellulose (NATRASOL® 250HX NF), hydroxypropylcellulose, hydroxypropylmethylcellulose, carboxymethylcellulose, and mixtures thereof. In terms of their viscosities, one class of alkyl-substituted celluloses includes those whose viscosity is within the range of about 100 to about 110,000 centipoise as a about 2% aqueous solution at about 20° C. Another class includes those whose viscosity is within the range of about 1,000 to about 4,000 centipoise as a about 1% aqueous solution at about 20° C.

Polyalkylene oxides that will desirably be used in certain embodiments of the swellable or swellable and erodable matrix cores, include those having the properties described above for alkyl-substituted cellulose polymers. In at least one embodiment the polyalkylene oxide is poly(ethylene oxide), which term is used herein to denote a linear polymer of unsubstituted ethylene oxide. In at least one embodiment the poly(ethylene oxide) polymers have molecular weights of about 4,000,000 and higher. For example, in certain embodiment the poly(ethylene oxide) polymers have molecular weights within the range of about 4,500,000 to about 10,000,000, and in other embodiments have molecular weights within the range of about 5,000,000 to about 8,000,000. In certain embodiments the poly(ethylene oxide)s are those with a weight-average molecular weight within the range of about 1×10⁵ to about 1×10⁷, and in other embodiments within the range of about 9×10⁵ to about 8×10⁶. Poly(ethylene oxide)s are often characterized by their viscosity in solution. For example, in certain embodiments the poly(ethylene oxide)s have a viscosity range of about 50 to about 2,000,000 centipoise for a about 2% aqueous solution at about 20° C. In at least one embodiment the poly(ethylene oxide) is one or more of POLYOX® NF, grade WSR Coagulant, molecular weight about 5 million, and grade WSR 303, molecular weight 7 million, and will desirably be used individually or in combination.

Polysaccharide gums, both natural and modified (semi-synthetic) will desirably be used in the swellable or swellable and erodable matrix embodiments of the present invention and include for example, dextran, xanthan gum, gellan gum, welan gum, rhamsan gum, and mixtures thereof.

Crosslinked polyacrylic acids that will desirably be used in the swellable matrices of the present invention include those whose properties are the same as those described above for alkyl-substituted cellulose and polyalkylene oxide polymers. In certain embodiments the crosslinked polyacrylic acids are those with a viscosity ranging from about 4,000 to about 40,000 centipoise for a about 1% aqueous solution at about 25° C. Examples of suitable crosslinked polyacrylic acids include CARBOPOL® NF grades 971P, 974P and 934P. Further examples of suitable crosslinked polyacrylic acids include polymers known as WATER LOCK®, which are starch/acrylates/acrylamide copolymers.

It is within the purview of the skilled artisan, without undue experimentation, to determine the amount of the hydrophilic swellable polymer relative to the tramadol and other pharmaceutically acceptable excipients to be used to manufacture the first once daily controlled-release dosage form, such that the desired in-vitro dissolution rate and the in-vivo pharmacokinetic parameters are met.

The hydrophilic water-swellable polymers will desirably be used individually or in combination. Certain combinations will often provide a more controlled release of the drug than their components when used individually. Examples include cellulose-based polymers combined with gums, such as hydroxyethylcellulose or hydroxypropyl cellulose combined with xanthan gum. Another example is poly(ethylene oxide) combined with xanthan gum.

The amount of hydrophilic swellable polymer will desirably range from about 5% to about 10%, such as for example, about 5, about 6, about 7, about 8, about 9 or about 10% of the matrix core dry weight, and be sufficient to retain about 78 to about 100% of tramadol within the first about 2 hours, about 70 to about 95% within the first about 4 hours, about 62 to about 85% within the first about 6 hours, and about 60% within the first about 8 hours when measured using a USP Type I, II or III apparatus (Rotating Basket Method) in 900 ml 0.1N HCl, water, 0.1N HCl+0.1% Cetrimide, USP Buffer pH 1.5, Acetate Buffer pH 4.5, Phosphate Buffer, pH 6.5 or Phosphate Buffer pH7.4 at 75 rpm at 37°±0.5° C. and exhibit a Cₘₐₓ of from about 75 to about 338 ng/ml of the tramadol and an AUC_{0-∞} of from about 2725 to about 7581 ng·hr/ml of tramadol under fed conditions or a Cₘₐₓ of from about 180 to about 333 ng/ml of tramadol and an AUC_{0-∞} of from about 3740 to about 7600 ng·hr/ml of tramadol under fasting conditions, and will desirably be bioequivalent according to FDA guidelines to a second orally administrable dosage form comprising tramadol also suitable for once daily administration in the fed or fasted state. The first once daily controlled-release dosage forms of the invention comprising a swellable or swellable and erodable dosage form or at least one means for controllably releasing the tramadol, wherein the means comprises at least one swellable or swellable and erodable matrix core has an amount of hydrophilic swellable polymer which will desirably also exhibit reduced potential for alcohol induced dose dumping.

In at least one embodiment of the invention, the first once daily controlled-release dosage form or the at least one means for controllably releasing the tramadol, wherein the at least one means comprises a hydrophilic matrix core, will desirably comprise at least one hydrophilic water-insoluble polymer. The release rate of the tramadol from a hydrophilic matrix core comprising at least one hydrophilic water-insoluble polymer depends on the active drug in aqueous solution diffusing through a network of passageways formed between compacted hydrophilic water-insoluble particles. The release rate of tramadol from the at least one water-insoluble polymer comprising hydrophilic matrix core will desirably be modified by changes in the porosity and tortuosity of the core. Processing parameters will desirably influence the release rate of the tramadol. For example, compaction forces will desirably control the porosity of the matrix core, which will desirably subsequently have an influence of the release rate of the tramadol. The addition of pharmaceutically acceptable excipients, such as for example, pore-forming hydrophilic materials, flux or channeling agents, or hydroattractants, will desirably also modulate rate of release of the tramadol. Water-soluble excipients, for example, would be expected to increase the porosity of the matrix core and water-insoluble excipients would be expected to decrease rate of release of tramadol by reducing penetration of the aqueous medium. Particle size of the at least one hydrophilic insoluble polymer or excipients will desirably also affect release rate of tramadol with larger particles resulting in an increase in release rate as matrices manufactured with larger particle size will desirably result in a matrix core with a more porous structure.

In embodiments of the invention where the first once daily controlled-release dosage form comprises a hydrophilic dosage form or the at least one means for controllably releasing the tramadol, wherein the at least one means comprises at least one hydrophilic matrix core, the hydrophilic dosage form or hydrophilic matrix core comprises at least one hydrophilic water-insoluble polymer, which will desirably be selected from, for example; vinyl-based polymers, such as for example, polyvinyl acetates, polyvinyl acetate dispersions; cellulosic polymers, such as for example, ethylcellulose, hydroxyethylcellulose, methylcellulose; polymethacrylates, such as for example, Eudragit RL, RS, NE30D (Rohm Pharma), Kollicoat EMM 30D (BASF), or combinations thereof. In certain embodiments, a flux enhancer or channeling agent will desirably be incorporated into the hydrophilic dosage form or hydrophilic matrix core comprising the at least one hydrophilic water-insoluble polymer. The amount of hydrophilic water-insoluble polymer will desirably range from about 5% to about 90%, such as for example about 5, about 10, about 15, about 20, about 25, about 30, about 35, about 40, about 45, about 50, about 55, about 60, about 65, about 70, about 75, about 80, about 85, or about 90% of the matrix core dry weight.

In at least one embodiment of the invention, the first once daily controlled-release dosage form comprising at least one controlled release matrix core comprises hydroxypropylmethylcellulose (HPMC). HPMC is an anhydroglucose in which some of the hydroxyl groups are substituted with methyl groups to form methyl ether moieties, and others are substituted with hydroxypropyl groups or with methoxypropyl groups to form hydroxypropyl ether or methoxypropyl ether moieties. Non-limiting examples of hydroxypropyl methylcelluloses that are commercially available include METHOCEL® E (USP type 2910), METHOCEL® F (USP type 2906), METHOCEL® J (USP type 1828), METHOCEL® K (USP type 2201), and METHOCEL® 310 Series, products of The Dow Chemical Company, Midland, Mich., USA. The average degree of methoxyl substitution in these products will desirably range from about 1.3 to about 1.9 (of the three positions on each unit of the cellulose polymer that are available for substitution) while the average degree of hydroxypropyl substitution per unit expressed in molar terms will desirably range from about 0.13 to about 0.82. The dosage form will desirably comprise the different HPMC grades having different viscosities. The size of a HPMC polymer is expressed not as molecular weight but instead in terms of its viscosity as a about 2% solution by weight in water. Different HPMC grades will desirably be combined to achieve the desired viscosity characteristics. For example, the at least one pharmaceutically acceptable polymer will desirably comprise two HPMC polymers such as for example Methocel® K3 LV (which has a viscosity of about 3 cps) and Methocel® K100M C (which has a viscosity of about 100,000 cps). In addition, the polymer will desirably comprise two hydroxypropylcellulose forms such as Klucel® LF and Klucel® EF. In addition, the at least one polymer will desirably comprise a mixture of a Klucel® and a Methocel®.

In at least one embodiment of the invention, the first once daily controlled-release dosage form comprising at least one controlled release matrix core comprises a polyethylene oxide (PEO). PEO is a linear polymer of unsubstituted ethylene oxide. In certain embodiments poly(ethylene oxide) polymers having viscosity-average molecular weights of about 100,000 daltons and higher are used. Non-limiting examples of poly(ethylene oxide)s that are commercially available include: POLYOX® NF, grade WSR Coagulant, molecular weight 5 million; POLYOX® grade WSR 301, molecular weight 4 million; POLYOX® grade WSR 303, molecular weight of about 7 million; POLYOX® grade WSR N-60K, molecular weight of about 2 million; and mixtures thereof. These particular polymers are products of Dow Chemical Company, Midland, Mich., USA. The desired molecular weight for the PEO will desirably be obtained by mixing PEO of differing molecular weights that are available commercially.

In at least one embodiment of the invention, the first once daily controlled-release dosage form comprising at least one controlled release matrix core comprises PEO and HPMC combined within the same controlled release matrix core. In certain embodiments, the poly(ethylene oxide)s have molecular weights ranging from about 2,000,000 to about 10,000,000 Da. For example, in at least one embodiment the polyethylene oxides have molecular weights ranging from about 4,000,000 to about 7,000,000 Da. In certain embodiments the HPMC polymers have a viscosity within the range of about 4,000 centipoise to about 200,000 centipoise. For example, in at least one embodiment the HPMC polymers have a viscosity of from about 50,000 centipoise to about 200,000 centipoise, and in other embodiments from about 80,000 centipoise to about 120,000 centipoise. The relative amounts of PEO and HPMC within the controlled release matrix core will desirably vary within the scope of the invention. In at least one embodiment the PEO:HPMC weight ratio is from about 1:3 to about 3:1. For example, in certain embodiments the PEO:HPMC weight ratio is from about 1:2 to about 2:1. As for the total amount of polymer relative to the entire controlled release matrix core, this will desirably vary as well and will desirably depend on the desired drug loading. In at least one embodiment the total amount of polymer in the controlled release matrix core will desirably constitute from about 15% to about 90% by weight of the matrix dosage form. For example, in certain embodiments the total amount of polymer in the controlled release matrix core is from about 20% to about 75%, in other embodiments from about 30% to about 60%, and in still other embodiments from about 10% to about 20% by weight of the first once daily controlled-release dosage form.

In at least one embodiment of the invention, the first once daily controlled-release dosage form comprises a hydrophobic dosage form or a means for controllably releasing the tramadol from the first once daily controlled-release dosage form, wherein said means comprises a hydrophobic matrix core.

In at least one embodiment of the invention, the hydrophobic dosage form comprises a lipid or wax dosage form. In at least one embodiment of the invention, the hydrophobic dosage form comprises at least one hydrophobic matrix core, which core is comprised of a lipid or wax material.

The first once daily controlled-release dosage form comprising a lipid or wax dosage form or at least one means for controllably releasing the tramadol, wherein the at least one means comprises a lipid or wax matrix core will desirably be prepared from blends of powdered components. The tramadol is contained in the lipid or wax dosage form or lipid or wax matrix core remains intact or will desirably erode during release of the tramadol. Release of the tramadol depends on an aqueous medium dissolving a channeling agent, which leaches out of the lipid or wax comprising dosage form or lipid or wax matrix core thereby forming a porous matrix. The tramadol dissolves in the aqueous medium and diffuses out of the matrix by way of the water-filled passageways.

Examples of lipids useful for the manufacture of the first once daily controlled-release dosage form comprising a lipid dosage form or at least one means for controllably releasing the tramadol, wherein the at least one means comprises a lipid matrix core will desirably include, for example, glyceryl monostearate, mixtures of glyceryl monostearate and glyceryl monopalmitate (Myvaplex, Eastman Fine Chemical Company), glycerylmonooleate, a mixture of mono, di and tri-glycerides (ATMUL 84S), glycerylmonolaurate, long chain carboxylic acids, long chain carboxylic acid esters, long chain carboxylic acid alcohols, and mixtures thereof. The long chain carboxylic acids will desirably contain from 6 to 30 carbon atoms; in certain embodiments at least 12 carbon atoms, and in other embodiments from 12 to 22 carbon atoms. In some embodiments this carbon chain is fully saturated and unbranched, while others comprise one or more double bonds. In at least one embodiment the long chain carboxylic acids contain 3-carbon rings or hydroxyl groups. Examples of saturated straight chain acids include n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. Also useful are unsaturated monoolefinic straight chain monocarboxylic acids. Non-limiting examples of these include oleic acid, gadoleic acid and erucic acid. Also useful are unsaturated (polyolefinic) straight chain monocaboxyic acids, examples of which include linoleic acid, linolenic acid, arachidonic acid and behenolic acid. Useful branched acids include, for example, diacetyl tartaric acid. Examples of long chain carboxylic acid esters include glyceryl monostearates; glyceryl monopalmitates; mixtures of glyceryl monostearate and glyceryl monopalmitate (Myvaplex 600, Eastman Fine Chemical Company); glyceryl monolinoleate; glyceryl monooleate; mixtures of glyceryl monopalmitate, glyceryl monostearate glyceryl monooleate and glyceryl monolinoleate (Myverol 18-92, Eastman Fine Chemical Company); glyceryl monogadoleate; mixtures of glyceryl monopalmitate, glyceryl monostearate, glyceryl monooleate, glyceryl monolinoleate, glyceryl monolinolenate and glyceryl monogadoleate (Myverol 18-99, Eastman Fine Chemical Company); acetylated glycerides such as distilled acetylated monoglycerides (Myvacet 5-07, 7-07 and 9-45, Eastman Fine Chemical Company); glyceryl behenate; mixtures of propylene glycol monoesters, distilled monoglycerides, sodium stearoyl lactylate and silicon dioxide (Myvatex TL, Eastman Fine Chemical Company); mixtures of propylene glycol monoesters, distilled monoglycerides, sodium stearoyl lactylate and silicon dioxide (Myvatex TL, Eastman Fine Chemical Company) d-alpha tocopherol polyethylene glycol 1000 succinate (Vitamin E TPGS, Eastman Chemical Company); mixtures of mono- and diglyceride esters such as Atmul (Humko Chemical Division of Witco Chemical); calcium stearoyl lactylate; ethoxylated mono- and di-glycerides; lactated mono- and di-glycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; cetearyl octanoate; C₁₀₋₃₀ cholesterol/lavosterol esters; sucrose long chain carboxylic acid esters; transesterified ethoxylated vegetable oil (Labrafil® M 2125 CS from Gattefossé); polyethoxylated unsaturated fatty acid triglycerides (Labrafil® M 1944 CS from Gattefossé); glyceryl palmitostearate (Precirol Ato 5® from Gattefossé); or mixtures thereof. The amount of the at least one lipid will desirably range from about 5% to about 80%, such as for example, about 5, about 10, about 15, about 20, about 25, about 30, about 35, about 40, about 45, about 50, about 55, about 60, about 65, about 70, about 75, about 80% of the matrix core dry weight.

Examples of waxes that will desirably used for the manufacture of the first once daily controlled-release dosage form comprising a wax dosage form or at least one means for controllably releasing the tramadol, wherein the at least one means comprises a wax matrix core will desirably include, for example, a natural or synthetic wax or oil, for example hydrogenated fats such as hydrogenated vegetable oil, hydrogenated castor oil, microcrystalline wax, normal waxes, insect and animal waxes, such as for example, chinese insect wax, beeswax, spermaceti, fats and wool wax; vegetable waxes, such as for example, bamboo leaf wax, candelilla wax, carnauba wax, Japan wax, ouricury wax, Jojoba wax, bayberry wax, Douglas-Fir wax, cotton wax, cranberry wax, cape berry wax, rice-bran wax, castor wax, Indian corn wax, hydrogenated vegetable oils (e.g., castor, palm, cottonseed, soybean), sorghum grain wax, Spanish moss wax, sugarcane wax, caranda wax, bleached wax, Esparto wax, flax wax, Madagascar wax, orange peel wax, shellac wax, sisal hemp wax and rice wax; mineral waxes, such as for example, Montan wax, peat waxes, petroleum wax, petroleum ceresin, ozokerite wax, microcrystalline wax and paraffins; synthetic waxes, such as for example, polyethylene wax, Fischer-Tropsch wax, chemically modified hydrocarbon waxes, cetyl esters wax, paraffin, or glyceryl monostearate, and suitably has a melting point of from 35 to 140° C., fatty alcohols, fatty acid esters, fatty acid glycerides (mono-, di-, and tri-glycerides), higher aliphatic (e.g., C₁₀₋₂₀) acids, alcohols, long chain fatty acids, and mixtures thereof. Useful water-insoluble wax-like substances may be those with a water-solubility that is lower than about 1:5,000 (w/w). The aliphatic alcohol will desirably be lauryl alcohol, myristyl alcohol or stearyl alcohol, cetyl alcohol, cetostearyl alcohol, or any combination thereof. The amount of the at least one wax will desirably range from about 5% to about 80%, such as for example, about 5, about 10, about 15, about 20, about 25, about 30, about 35, about 40, about 45, about 50, about 55, about 60, about 65, about 70, about 75, about 80% of the matrix core dry weight

In at least one embodiment of the invention, the hydrophobic dosage form or hydrophobic matrix core is comprised of at least one hydrophobic polymer, which will desirably range from about 5% to about 70%, such as for example, about 5, about 10, about 15, about 20, about 25, about 30, about 35, about 40, about 45, about 50, about 55, about 60, about 65, about 70% of the matrix core dry weight

Examples of hydrophobic polymers that will desirably used for the manufacture of the first once daily controlled-release dosage form comprising a hydrophobic dosage form or at least one means for controllably releasing the tramadol, wherein the at least one means comprises a hydrophobic polymer matrix core will desirably include, for example, one or more alkylcelluloses, such as for example, C₁₋₆ alkyl cellulose (e.g., ethylcellulose) and one or more C₁₂₋₃₆ aliphatic alcohols, a pharmaceutically acceptable acrylic polymer, such as for example, acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, poly(methyl methacrylate), poly(methyl methacrylate) copolymer, polyacrylamide, aminoalkyl methacrylate copolymer, poly(methacrylic acid anhydride), or glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups. Such an acrylic polymer is an acrylic resin lacquer commercially available from Rohm Pharma under the Tradename Eudragit®.

Other hydrophobic polymers, which will desirably be used in the manufacture of the hydrophobic dosage forms or the hydrophobic matrix cores of the present invention include cellulosic polymers, including other alkyl cellulosic polymers such as ethylcellulose.

The release of tramadol from the hydrophobic dosage forms or the at least one means for controllably releasing the tramadol, wherein the at least one means comprises at least one hydrophobic matrix core will desirably be adjusted to the desired rate, by the addition of one or more release-modifying agents into the hydrophobic dosage form or hydrophobic matrix core. The release-modifying agents may comprise one or more water-soluble hydrophilic polymers in order to modify the release characteristics of the hydrophobic dosage form or hydrophobic matrix core. Examples of suitable hydrophilic polymers include, for example, those described above. Semipermeable polymers may also be incorporated in the hydrophobic dosage form or hydrophobic matrix core to change the release characteristics of the tramadol. Such semipermeable polymers will desirably include, for example, cellulose acylates, acetates, and other semipermeable polymers such as those described in U.S. Pat. No. 4,285,987, as well as the selectively permeable polymers formed by the coprecipitation of a polycation and a polyanion as disclosed in U.S. Pat. Nos. 3,173,876; 3,276,586; 3,541,005; 3,541,006 and 3,546,142. At least one plasticizer will desirably also be incorporated into the hydrophobic dosage form or hydrophobic matrix core. Guidance for the use and manufacture of dosage forms comprising hydrophobic materials will desirably be found in U.S. Pat. No. 6,645,572 and in Obaidat & Obaidat, Controlled release of tramadol hydrochloride from matrices prepared using glyceryl behenate. Eur. J. Pharm Biopharm 52 (2001): 231-235.

In at least one embodiment of the invention, the first once daily controlled-release dosage form is comprised of a combination of a hydrophobic and hydrophilic dosage forms or a means for controllably releasing the tramadol, wherein the means comprises a combination of hydrophobic and hydrophilic matrix cores. The hydrophobic and hydrophilic dosage forms or hydrophobic and hydrophilic matrix cores will desirably comprise at least one hydrophobic and hydrophilic polymer and will desirably be chosen from the hydrophobic and hydrophilic polymers described above. For example, in one combination, the hydrophobic polymer will desirably be hydrogenated castor oil and ethylcellulose, while the hydrophilic polymer will desirably be hydroxypropylmethylcellulose. Guidance for the use and manufacture of dosage forms comprising such a hybrid system will desirably be found in Tiwari S. et al., Controlled Release Formulation of Tramadol Hydrochloride Using Hydrophilic and Hydrophobic Matrix System AAPS Pharm Sci Tech 4(3) (2003): 1-6.

The first once daily controlled-release dosage forms of the invention will desirably comprise a normal release matrix core designed such that the hydrophilic and or hydrophobic materials described above are in amounts or ratios which mimic immediate release of the tramadol comprising the normal release matrix core. The rate will desirably also be influenced by the inclusion of at least one pharmaceutically excipient, such as for example, channeling agents and gel modifiers as well as processing parameters, such as for example, compression forces and/or the particle size of the excipients and/or particle size of the tramadol. In order to obtain a controlled-release of the tramadol, comprising a normal-release matrix core, the core will desirably be coated with at least one release-slowing coat, at least one delayed-release coat, at least one semipermeable coat, at least one swellable coat, at least one erodable coat, at least one swellable and erodable coat, at least one hydrophobic coat, at least one hydrophilic coat, at least one coat comprising at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol or combinations thereof. The first once daily controlled-release dosage forms of the invention described above as well as the at least one means for controllably releasing the tramadol will desirably be further coated with at least one or more coats described above to control the release of tramadol such that the dosage form meets the dissolution rates and pharmacokinetic parameters described herein.

The semipermeable membrane comprises at least one pharmaceutically acceptable excipient, at least one polymer, wax, or combination thereof, although appropriately treated inorganic materials such as ceramics, metals or glasses will desirably be used. When the semipermeable membrane comprises at least one polymer, the molecular weight of the at least one polymer or combination of polymers should be such that the polymer or combination of polymers is solid at the temperature of use i.e., both in-vitro and in-vivo.

In certain embodiments of the invention, the at least one polymer comprising the semipermeable membrane will desirably be a cellulose ester, such as for example, cellulose acetate, cellulose acetate acetoacetate, cellulose acetate benzoate, cellulose acetate butylsulfonate, cellulose acetate butylate, cellulose acetate butylate sulfate, cellulose acetate butylate valerate, cellulose acetate caprate, cellulose acetate caproate, cellulose acetate caprylate, cellulose acetate carboxymethoxypropionate, cellulose acetate chloroacetate, cellulose acetate dimethaminoacetate, cellulose acetate dimethylaminoacetate, cellulose acetate dimethylsulfamate, cellulose acetate dipalmitate, cellulose acetate dipropylsulfamate, cellulose acetate ethoxyacetate, cellulose acetate ethyl carbamate, cellulose acetate ethyl carbonate, cellulose acetate ethyl oxalate, cellulose acetate furoate, cellulose acetate heptanoate, cellulose acetate heptylate, cellulose acetate isobutylate, cellulose acetate laurate, cellulose acetate methacrylate, cellulose acetate methoxyacetate, cellulose acetate methylcarbamate, cellulose acetate methylsulfonate, cellulose acetate myristate, cellulose acetate octanoate, cellulose acetate palmitate, cellulose acetate phthalate, cellulose acetate propionate, cellulose acetate propionate sulfate, cellulose acetate propionate valerate, cellulose acetate p-toluene sulfonate, cellulose acetate succinate, cellulose acetate sulfate, cellulose acetate trimellitate, cellulose acetate tripropionate, cellulose acetate valerate, cellulose benzoate, cellulose butylate napthylate, cellulose butylate, cellulose chlorobenzoate, cellulose cyanoacetates, cellulose dicaprylate, cellulose dioctanoate, cellulose dipentanate, cellulose dipentanlate, cellulose formate, cellulose methacrylates, cellulose methoxybenzoate, cellulose nitrate, cellulose nitrobenzoate, cellulose phosphate (sodium salt), cellulose phosphinates, cellulose phosphites, cellulose phosphonates, cellulose propionate, cellulose propionate crotonate, cellulose propionate isobutylate, cellulose propionate succinate, cellulose stearate, cellulose sulfate (sodium salt), cellulose triacetate, cellulose tricaprylate, cellulose triformate, cellulose triheptanoate, cellulose triheptylate, cellulose trilaurate, cellulose trimyristate, cellulose trinitrate, cellulose trioctanoate, cellulose tripalmitate, cellulose tripropionate, cellulose trisuccinate, cellulose trivalerate, cellulose valerate palmitate; a cellulose ether, such as for example, 2-cyanoethylcellulose, 2-hydroxybutyl methylcellulose, 2-hydroxyethylcellulose, 2-hydroxyethyl ethylcellulose, 2-hydroxyethyl methylcellulose, 2-hydroxypropyl cellulose, 2-hydroxypropyl methylcellulose, dimethoxyethylcellulose acetate, ethyl 2-hydroxylethylcellulose, ethylcellulose, ethylcellulose sulfate, ethylcellulose dimethylsulfamate, methylcellulose, methylcellulose acetate, methylcyanoethylcellulose, sodium carboxymethyl 2-hydroxyethylcellulose, sodium carboxymethylcellulose; a polysulfone, such as for example, polyethersulfones; a polycarbonate; a polyvinyl chloride; a polyurethane; a polyvinyl acetate; a polyvinyl acetate dispersion, such as for example Kollicoat® SR 30D; a polyvinyl alcohol; a polyester; a polyalkene such as polyethylene, ethylene vinyl alcohol copolymer, polypropylene, poly(1,2-dimethyl-1-butenylene), poly(1-bromo-1-butenylene), poly(1, butene), poly(1-chloro-1-butenylene), poly(1-decyl-1-butenylene), poly(1-hexane), poly(1-isopropyl-1-butenylene), poly(1-pentene), poly(3-vinylpyrene), poly(4-methoxyl 1-butenylene), poly(ethylene-co-methyl styrene), poly vinyl-chloride, poly(ethylene-co-tetrafluoroethylene), poly(ethylene-terephthalate), poly(dodecafluorobutoxylethylene), poly(hexafluoroprolylene), poly(hexyloxyethylene), poly(isobutene), poly(isobutene-co-isoprene), poly(isoprene), poly-butadiene, poly[(pentafluoroethyl)ethylene], poly[2-ethylhexyloxy)ethylene], poly(butylethylene), poly(tertbutylethylene), poly(cylclohexylethylene), poly[(cyclohexylmethyl)ethylene], poly(cyclopentylethylene), poly(decylethylene), poly(dodecylethylene), poly(neopentylethylene), poly(propylethylene); a polystyrene, such as for example, poly(2,4-dimethyl styrene), poly(3-methyl styrene), poly(4-methoxystyrene), poly(4-methoxystyrene-stat-styrene), poly(4-methyl styrene), poly(isopentyl styrene), poly(isopropyl styrene), polyvinyl esters or polyvinyl ethers, such as form example, poly(benzoylethylene), poly(butoxyethylene), poly(chloroprene), poly(cyclohexloxyethylene), poly(decyloxyethylene), poly(dichloroethylene), poly(difluoroethylene), poly(vinyl acetate), poly(vinyltrimethylstyrene); a polysiloxane, such as for example, poly(dimethylsiloxane); a polyacrylic acid derivative, such as for example, polyacrylates, polymethyl methacrylate, poly(acrylic acid) higher alkyl esters, poly(ethylmethacrylate), poly(hexadecyl methacrylate-co-methylmethacrylate), poly(methylacrylate-co-styrene), poly(n-butyl methacrylate), poly(n-butyl-acrylate), poly(cyclododecyl acrylate), poly(benzyl acrylate), poly(butylacrylate), poly(secbutylacrylate), poly(hexyl acrylate), poly(octyl acrylate), poly(decyl acrylate), poly(dodecyl acrylate), poly(2-methyl butyl acrylate), poly(adamantyl methacrylate), poly(benzyl methacrylate), poly(butyl methacrylate), poly(2-ethylhexyl methacrylate), poly(octyl methacrylate), acrylic resins; a polyamide, such as for example, poly(iminoadipoyliminododecamethylene), poly(iminoadipoyliminohexamethylene), polyethers, such as for example, poly(octyloxyethylene), poly(oxyphenylethylene), poly(oxypropylene), poly(pentyloxyethylene), poly(phenoxy styrene), poly(secbutroxylethylene), poly(tert-butoxyethylene); and combinations thereof will desirably range from about 2% to about 40%, such as for example, about 2, about 4, about 6, about 8, about 10, about 12, about 14, about 16, about 18, about 20, about 22, about 24, about 26, about 28, about 30, about 32, about 34, about 36, about 38, or about 40% of the semipermeable membrane.

In at least one embodiment of the invention, the at least one wax comprising the semipermeable membrane will desirably be, for example, insect and animal waxes, such as for example, chinese insect wax, beeswax, spermaceti, fats and wool wax; vegetable waxes, such as for example, bamboo leaf wax, candelilla wax, carnauba wax, Japan wax, ouricury wax, Jojoba wax, bayberry wax, Douglas-Fir wax, cotton wax, cranberry wax, cape berry wax, rice-bran wax, castor wax, Indian corn wax, hydrogenated vegetable oils (e.g., castor, palm, cottonseed, soybean), sorghum grain wax, Spanish moss wax, sugarcane wax, caranda wax, bleached wax, Esparto wax, flax wax, Madagascar wax, orange peel wax, shellac wax, sisal hemp wax and rice wax; mineral waxes, such as for example, Montan wax, peat waxes, petroleum wax, petroleum ceresin, ozokerite wax, microcrystalline wax and paraffins; synthetic waxes, such as for example, polyethylene wax, Fischer-Tropsch wax, chemically modified hydrocarbon waxes, cetyl esters wax; and combinations thereof and will desirably range from about 1% to about 40%, such as for example, about 1, about 2, about 4, about 6, about 8, about 10, about 12, about 14, about 16, about 18, about 20, about 22, about 24, about 26, about 28, about 30, about 32, about 34, about 36, about 38, or about 40% of the semipermeable membrane.

In at least one embodiment of the invention, the semipermeable membrane will desirably comprise a combination of at least one polymer, at least one lipid or wax, or combinations thereof and optionally at least one excipient. The total weight percent of all components comprising the semipermeable membrane is about 100%.

In embodiments of the invention where tramadol is released through the membrane or coat in a controlled manner by the combined mechanisms of diffusion and osmotic pumping, the membrane or coat will desirably comprise at least one of the above described polymers and/or waxes or a combination of polymers, such as for example, cellulose esters, copolymers of methacrylate salts and optionally a plasticizer.

Aside from the semipermeable membranes described above, asymmetric membranes will desirably also be used to surround the first once daily controlled-release dosage form or means for controllably releasing the tramadol and provide the in-vitro release rates described above and the therapeutically beneficial in-vivo pharmacokinetic parameters for the treatment or management of moderately to moderately severe pain. Such asymmetric membranes will desirably be permeable, semipermeable, perforated, or unperforated and will desirably deliver tramadol by osmotic pumping, diffusion or the combined mechanisms of diffusion and osmotic pumping. U.S. Pat. No. 5,612,059 provides guidance for the use and manufacture of asymmetric membranes for the controlled-release of an active through one or more asymmetric membranes by diffusion and/or osmotic pumping.

In certain embodiments of the invention, the semipermeable membrane comprises at least one flux enhancing, or channeling agent. The flux enhancing or channeling agent dissolves to form paths in the semipermeable membrane for the fluid to enter the first once daily controlled-release dosage form and the at least one means for controllably releasing the tramadol and dissolve tramadol in said dosage form and at least one means together with the osmagent, if one is present, but does not allow exist of tramadol. The flux-enhancing agent will desirably be any water-soluble material or an enteric material, which allows an increase in the volume of liquid, imbibed into said dosage form or at least one means but does not allow for the exit of tramadol. Such materials will desirably be, for example, sodium chloride, potassium chloride, sucrose, sorbitol, mannitol, polyethylene glycol, propylene glycol, hydroxypropyl cellulose, hydroxypropyl methylcellulose, hydroxypropyl methylcellulose phthalate, cellulose acetate phthalate, polyvinyl alcohols, methacrylic copolymers, and combinations thereof. Some plasticizers will desirably also function as flux enhancers by increasing the hydrophilicity of the semipermeable membrane and/or the first once daily controlled-release dosage form or the at least one means for controllably releasing the tramadol. The amount of the flux enhancer or channeling agent will desirably be from about 0% to about 20%, such as for example, about 0%, about 2%, about 4%, about 6%, about 8%, about 10%, about 12%, about 14%, about 16%, about 18%, or about 20% of the dosage form or from about 0% to about 40%, such as for example, about 0%, about 2%, about 4%, about 6%, about 8%, about 10%, about 12%, about 14%, about 16%, about 18%, about 20%, about 22%, about 24%, about 26%, about 28%, about 30%, about 32%, about 34%, about 36%, about 38% or about 40% of the semipermeable membrane. Flux enhancers or channeling agents will desirably also function as a means for the exit of tramadol from the first once daily controlled-release dosage form or the at least one means for controllably releasing the tramadol if the flux enhancing or channeling agent is used in a sufficient amount.

The expression “passageway” as used herein comprises means and methods suitable for the metered release of tramadol from the first once daily controlled-release dosage form or the at least one means for controllably releasing the tramadol. The means for the exit of tramadol comprises at least one passageway, including orifice, bore, aperture, pore, porous element, hollow fiber, capillary tube, porous overlay, porous network, that provides for the release of tramadol from the first once daily controlled-release dosage form or the at least one means for controllably releasing the tramadol. The means for the exit will desirably be linear or tortuous. The means for the exit includes a weakened area of the semipermeable membrane or a material that erodes or is leached from the membrane or coat in a fluid environment of use to produce at least one dimensioned passageway. The means for the exit of tramadol will desirably comprise at least one leachable material, which when leaches out of the semipermeable membrane forms a passageway suitable for the exit of tramadol from the first once daily controlled-release dosage form or the at least one means for controllably releasing the tramadol. Such leachable materials will desirably comprise, for example, a leachable poly(glycolic) acid or poly(lactic) acid polymer in the semipermeable membrane, a gelatinous filament, poly(vinyl alcohol), leachable polysaccharides, salts, oxides, sorbitol, or sucrose. The means for exit will desirably also comprise at least one flux enhancer or channeling agent if present in a sufficient amount. The means for the exit possesses controlled-release dimensions, such as round, triangular, square and elliptical, for the metered release of tramadol from the first once daily controlled-release dosage form or the at least one means for controllably releasing the tramadol. The dimensions of the means of the exit for tramadol is sized such so as to allow tramadol to pass through the means for the exit. The first once daily controlled-release dosage form or the at least one means for controllably releasing the tramadol will desirably be constructed with one or more means for the exit in spaced apart relationship on a single surface or on more than one surface of the membrane or coat. The expression “fluid environment” denotes an aqueous or biological fluid as in a human patient, including the gastrointestinal tract. The means for the exit will desirably be preformed e.g., by mechanical means after the semipermeable membrane is applied to the core of the osmotic dosage form, such as for example by mechanical perforation, laser perforation, or by using a properly sized projection on the interior of a tablet punch to form the means for the exit of tramadol, such as for example a cylindrical or frustoconical pin which is integral with the inside surface of the upper punch of a punch used to form the osmotic dosage form. Alternatively, incorporating a leachable material will desirably form the means for the exit of tramadol or pore-forming agent into the semipermeable composition before the semipermeable membrane is applied to the first once daily controlled-release dosage form or the at least one means for controllably releasing the tramadol. The means for the exit of tramadol will desirably comprise of a combination of the different exit means described above. The first once daily controlled-release dosage form or the at least one means for controllably releasing the tramadol will desirably comprise of more than one means for the exit of tramadol including two, three, four, five, six seven, eight, nine ten or more exit means and will desirably be formed in any place of the first once daily controlled-release dosage form or the at least one means for controllably releasing the tramadol. The various positions of the means for the exit are disclosed, for example, in U.S. Pat. No. 6,491,949. The type, number, and dimension(s) of the means for the exit of tramadol must be such that the first once daily controlled-release dosage form or the at least one means for controllably releasing the tramadol will desirably exhibit the desired in-vitro release rates and in-vivo pharmacokinetic parameters described herein and will desirably be determined by routine experimentation by those skilled in the pharmaceutical delivery arts. U.S. Pat. Nos. 3,845,770; 3,916,899; 4,034,758; 4,063,064; 4,077,407, 4,088,864; 4,200,098; 4,285,987; 4,783,337; 4,816,263; 5,071,607; provide guidance for forming the means for the exit and equipment for forming such means.

The first once daily controlled-release dosage form or the at least one means for controllably releasing the tramadol will desirably further comprise at least one release-slowing coat, delayed-release coat, or a coat comprising an aqueous dispersion of a neutral ester copolymer without any functional groups, a poly glycol having melting greater than 55° C., and one or more pharmaceutically acceptable excipients and cured at a temperature at least equal to or greater than the melting point of the poly glycol, surrounding the semipermeable membrane or coat. The delayed-release coat is soluble or erodable in intestinal juices, substantially pH neutral or basic fluids of fluids having a pH higher than gastric fluid, but for the most part insoluble in gastric juices or acidic fluids. The poly(methacrylate) copolymer salts used in the manufacturing of the membrane or coat will desirably be, for example, insoluble in water and in digestive fluids, but are permeable to different degrees. Non-limiting examples of such copolymers are carboxymethylethylcellulose, cellulose acetate phthalate, cellulose acetate succinate, methylcellulose phthalate, hydroxymethylethylcellulose phthalate, hydroxypropylmethylcellulose phthalate, hydroxypropylmethylcellulose acetate succinate, polyvinyl alcohol phthalate, polyvinyl butylate phthalate, polyvinyl acetal phthalate, a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate and a copolymer of methacrylic acid/methyl methacrylate, poly(ammonium methacrylate) copolymer RL (Eudragit™ RL), poly(ammonium methacrylate) copolymer (type A-USP/NF), poly(aminoalkyl methacrylate) copolymer RL-JSP I), and (ethyl acrylate)-(methyl methacrylate)-[(trimethylammonium)-ethylmethacrylate] (1:2:0.2) copolymer, MW 150,000. Other examples of such copolymers include those available from Rohm Pharma, Weiterstadt, such as for example, Eudragit™ RS 100: solid polymer, Eudragit™ RL 12.5:12.5% solution in solvent, Eudragit™ RL 30D: 30% aqueous dispersion, and other equivalent products. The following poly (ammonium methacrylate) copolymers will desirably also be used: ammonium methacrylate copolymer RS (Eudragit™ RS), poly(ammonium methacrylate) copolymer (type B-USP/NF), poly(aminoalkyl methacrylate) copolymer (RSL-JSP I), (ethyl acrylate)-(methyl methacrylate)-[(trimethylammonium)-ethyl methacrylate] (1:2:0.1) copolymer, PM 150,000. Specific polymers include (Rohm Pharma, Weiterstadt): Eudragit™ RS 100: solid polymer, Eudragit™ RS 12.5: 12.5% solution in solvent, Eudragit™ RS 30D: 30% aqueous dispersion and other equivalent products. RL is readily water permeable while Eudragit™ RS is hardly water permeable. These enteric polymers will desirably be used individually or in combination. For example, by employing mixtures of both Eudragit™ RL and Eudragit™ RS, membranes having the desired degree of permeability to achieve the in-vitro dissolution rates and in-vivo pharmacokinetic parameters will desirably be prepared. The amount of such copolymers will desirably be from about 0% to about 40%, such as for example, about 0%, about 2%, about 4%, about 6%, about 8%, 1 about 0%, about 12%, about 14%, about 16%, about 18%, about 20%, about 22%, about 24%, about 26%, about 28%, about 30%, about 32%, about 34%, about 36%, about 38%, or about 40% of the dosage form or from about 0% to about 90%, such as for example, about 0%, about 5%, about 10%, about 15%, about 20%, about 25%, about 30%, about 35%, about 40%, about 45%, about 50%, 55%, about 60%, about 65%, about 70%, about 75%, about 80%, about 85%, or about 90% of the delayed-release coat. The enteric coat will desirably also comprise dissolution aids, stability modifiers, and biowetting aids.

When the delayed-release coat is intended to be dissolved, eroded or become detached from the first once daily controlled-release dosage form or the at least one means for controllably releasing the tramadol, materials such as hydroxypropylcellulose, microcrystalline cellulose (MCC, Avicel.™. from FMC Corp.), poly (ethylene-vinyl acetate) (60:40) copolymer (EVAC from Aldrich Chemical Co.), 2-hydroxyethylmethacrylate (HEMA), MMA, terpolymers of HEMA: MMA:MA synthesized in the presence of N,N′-bis(methacryloyloxyethyloxycarbonylamino)-azobenzene, azopolymers, enteric coated timed release system (Time Clock® from Pharmaceutical Profiles, Ltd., UK) and calcium pectinate will desirably be used.

Polymers for use in the delayed-release coat will desirably be, for example, enteric materials that resist the action of gastric fluid avoiding permeation through the semipermeable membrane or coat while one or more of the materials in the first once daily controlled-release dosage form or the at least one means for controllably releasing the tramadol are solubilized in the intestinal tract thereby allowing delivery of tramadol to begin. A material that easily adapts to this kind of requirement will desirably be, for example, a poly(vinylpyrrolidone)-vinyl acetate copolymer, such as the material supplied by BASF under its Kollidon VA64 trademark, mixed with magnesium stearate and other similar excipients. The enteric coat will desirably also comprise povidone, which is supplied by BASF under its Kollidon K 30 trademark, and hydroxypropyl methylcellulose, which is supplied by Dow under its Methocel E-15 trademark. The materials will desirably be prepared in solutions of having different concentrations of polymer according to the desired solution viscosity. For example, a 10% P/V aqueous solution of Kollidon K 30 has a viscosity of about 5.5-8.5 cps at 20° C., and a 2% P/V aqueous solution of Methocel E-15 has a viscosity of about 13-18 cps at 20° C.

The release-slowing coat will desirably comprise one or more materials that do not dissolve, disintegrate, or change their structural integrity in the stomach and during the period of time that the tablet resides in the stomach. The one or more materials that do not dissolve, disintegrate, or change their structural integrity in the stomach and during the period of time that the tablet resides in the stomach will desirably be, for example, a member chosen from the group (a) keratin, keratin saridarac-tolu, salol (phenyl salicylate), salol beta-naphthylbenzoate and acetotannin, salol with balsam of Peru, salol with tolu, salol with gum mastic, salol and stearic acid, and salol and shellac; (b) a member chosen from the group of formalized protein, formalized gelatin, and formalized cross-linked gelatin and exchange resins; (c) a member chosen from the group of myristic acid-hydrogenated castor oil-cholesterol, stearic acid-mutton tallow, stearic acid-balsam of tolu, and stearic acid-castor oil; (d) a member chosen from the group of shellac, ammoniated shellac, ammoniated shellac-salol, shellac-wool fat, shellac-acetyl alcohol, shellac-stearic acid-balsam of tolu, and shellac n-butyl stearate; (e) a member chosen from the group of abietic acid, methyl abictate, benzoin, balsam of tolu, sandarac, mastic with tolu, and mastic with tolu, and mastic with acetyl alcohol; (f) acrylic resins represented by anionic polymers synthesized from methacrylate acid and methacrylic acid methyl ester, copolymeric acrylic resins of methacrylic and methacrylic acid and methacrylic acid alkyl esters, copolymers of alkacrylic acid and alkacrylic acid alkyl esters, acrylic resins such as dimethylaminoethylmethacrylate-butylmethacrylate-methylmethacrylate copolymer of about 150,000 molecular weight, methacrylic acid-methylmethacrylate 50:50 copolymer of about 135,000 molecular weight, methacrylic acid-methylmethacrylate-30:70-copolymer of about 135,000 mol. wt., methacrylic acid-dimethylaminoethyl-methacrylate-ethylacrylate of about 750,000 mol. wt., methacrylic acid-methylmethacrylate-ethylacrylate of about 1,000,000 mol. wt., and ethylacrylate-methylmethacrylate-ethylacrylate of about 550,000 mol. wt; and, (g) an enteric composition chosen from the group of cellulose acetyl phthalate, cellulose diacetyl phthalate, cellulose triacetyl phthalate, cellulose acetate phthalate, hydroxypropyl methylcellulose phthalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate diethyl phthalate, dibutyl phthalate, dialkyl phthalate wherein the alkyl comprises from 1 to 7 straight and branched alkyl groups, aryl phthalates, and other materials known to one or ordinary skill in the art.

In at least one embodiment of the invention, the release-slowing coat comprises a water-insoluble water-permeable film-forming polymer, water-soluble polymer, and optionally a plasticizer and/or a pore-forming agent. The water-insoluble, water-permeable film-forming polymers useful for the manufacture of the release-slowing coat will desirably be cellulose ethers, such as for example, ethylcelluloses chosen from the group of ethylcellulose grade PR100, ethylcellulose grade PR20 and any combination thereof; cellulose esters, and polyvinyl alcohol. The amount of the water-insoluble water-permeable film-forming polymer useful will desirably range form about 2% to about 40% such as for example, about 2%, about 4%, about 6%, about 8%, about 10%, about 12%, about 14%, about 16%, about 18%, about 20%, about 22%, about 24%, about 26%, about 28%, about 30%, about 32%, about 34%, about 36%, about 38%, or about 40% of the dosage form or from about 0% to about 85%, such as for example, about 0%, about 5%, about 10%, about 15%, about 20%, about 25%, about 30%, about 35%, about 40%, about 45%, about 50%, 55%, about 60%, about 65%, about 70%, about 75%, or about 80% of the release-slowing coat. The water-soluble polymers useful for the release-slowing coat will desirably be, for example, polyvinylpyrrolidone, hydroxypropyl methylcellulose and hydroxypropyl cellulose and will desirably be present in an amount that ranges from about 0% to about 20%, such as for example, about 0%, about 2%, about 4%, about 6%, about 8%, about 10%, about 12%, about 14%, about 16%, about 18%, or about 20% of the dosage form and from about 0% to about 85%, such as for example, about 0%, about 5%, about 10%, about 15%, about 20%, about 25%, about 30%, about 35%, about 40%, about 45%, about 50%, 55%, about 60%, about 65%, about 70%, about 75%, about 80% or about 85% of the release-slowing coat.

The skilled artisan will appreciate that that the desired in-vitro release rates and in-vivo pharmacokinetic profiles described herein for tramadol will desirably be achieved by controlling the permeability and/or the amount of coating applied to the first once daily controlled-release dosage form or the at least one means for controllably releasing the tramadol. The permeability of the control-releasing coat, will desirably be altered by varying the ratio of the water-insoluble, water-permeable film-forming polymer:water-soluble polymer:optionally the plasticizer and/or the quantity of coating applied to the core of the osmotic dosage form. A more extended release is generally obtained with a higher amount of water-insoluble, water-permeable film forming polymer. The addition of other excipients to the first once daily controlled-release dosage form or the at least one means for controllably releasing the tramadol may also alter the permeability of the release-slowing coat. For example, if the first once daily controlled-release dosage form or the at least one means for controllably releasing the tramadol comprises a swellable polymer, the amount of plasticizer in the release-slowing coat will desirably be increased to make the coat more pliable as the pressure exerted on a less pliable coat by the swellable polymer would rupture the coat. Further, the proportion of the water-insoluble water-permeable film forming polymer and water-soluble polymer may also have to be altered depending on whether a faster or slower in-vitro dissolution and/or pharmacokinetic profile is desired.

In at least one embodiment of the invention, the release-slowing coat comprises at least one aqueous dispersion of a neutral ester copolymer without any functional groups, a poly glycol having a melting point greater than 55° C., and one or more pharmaceutically acceptable excipients and cured at a temperature at least equal to or greater than the melting point of the poly glycol. The manufacture and use of such coating formulations are described in detail in US published patent application 20040037883A1, published on Feb. 26, 2004. In brief, examples of neutral ester copolymers without any functional groups comprising the coat will desirably be Eudragit® NE30D, Eudragit® NE40D (Röhm America LLC). This coat will desirably comprise hydrophilic agents to promote wetting of the coat when in contact with gastrointestinal fluids. Such hydrophilic agents include, for example, hydrophilic water-soluble polymers such as hydroxypropyl methylcellulose (HPMC), hydroxypropyl cellulose (HPC) and combinations thereof. The poly glycol will desirably be, for example, chosen from the group of polyethylene glycol 6000, polyethylene glycol 8000, polyethylene glycol 10000, polyethylene glycol 20000, Poloxamer 188, Poloxamer 338, Poloxamer 407, Polyethylene Oxides, Polyoxyethylene Alkyl Ethers, and Polyoxyethylene Stearates, and combinations thereof. This release-slowing coat comprises at least one pore-forming agent. The pore former or channeling agent, however, must be sufficiently insoluble in the aqueous dispersion, but must be sufficiently soluble in the environment of use.

In at least one embodiment of the invention, first once daily controlled-release dosage form or the at least one means for controllably releasing the tramadol comprises at least one water-soluble or rapidly dissolving coat between the semipermeable membrane and the release-slowing coat. The rapidly dissolving coat will desirably be soluble in the buccal cavity and/or upper GI tract, such as the stomach, duodenum, jejunum or upper small intestines. Materials suitable for the manufacture of the water-soluble coat are disclosed in U.S. Pat. Nos. 4,576,604 and 4,673,405, and the text Pharmaceutical Dosage Forms: Tablets Volume I, Second Edition. A. Lieberman. ed. 1989, Marcel Dekker, Inc. In certain embodiments, the rapidly dissolving coat will desirably be soluble in saliva, gastric juices, or acidic fluids.

Materials which are suitable for making the water-soluble coat or layer will desirably comprise, for example, water-soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as, for example, hydroxymethylcellulose, hydroxyethylcellulose, and hydroxypropylcellulose; synthetic water-soluble cellulose-based lamina formers such as, for example, methylcellulose and its hydroxyalkyl methylcellulose cellulose derivatives such as a member chosen from the group of hydroxyethyl methylcellulose, hydroxypropyl methylcellulose, and hydroxybutyl methylcellulose; croscarmellose sodium; other cellulose polymers such as sodium carboxymethylcellulose; and other materials known to those of ordinary skill in the art. Other lamina forming materials that will desirably be used for this purpose include, for example, poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer, or any combination thereof. The water-soluble coating will desirably comprise other pharmaceutical excipients, which do or do not alter the way in which the water-soluble coating behaves. The artisan of ordinary skill will recognize that the above-noted materials include film-forming polymers. The materials suitable for making the water-soluble coat or layer will desirably range from about 0% to about 40% such as for example about 0%, about 5%, about 10%, about 15%, about 20%, about 25%, about 30%, about 35%, or about 40% of the dosage form, or about 0% to about 85%, such as for example, about 0%, about 5%, about 10%, about 15%, about 20%, about 25%, about 30%, about 35%, about 40%, about 45%, about 50%, about 55%, about 60%, about 65%, about 70%, about 75%, or about 80% of the water-soluble coat.

In certain embodiments, a water-soluble coat covers a semipermeable membrane or coat and will desirably block the passageway or the at least one means for the exit of the tramadol. In such embodiments, the water-soluble coat is made of synthetic or natural material, which, through selective dissolution or erosion will desirably allow the passageway or at least one means for the exit of tramadol to be unblocked thus permitting exit of the tramadol. This water-soluble coat will desirably be impermeable to a first external fluid, while being soluble in a second external fluid. This property will desirably help to achieve a controlled and selective release of tramadol from the first once daily controlled-release dosage form or the at least one means for controllably releasing the tramadol so as to achieve the desired in-vitro release rates and in-vivo pharmacokinetic parameters.

In embodiments of the invention where the first once daily controlled-release dosage form or the at least one means for controllably releasing the tramadol does not comprise an osmagent and/or osmopolymer, the first once daily controlled-release dosage form or the at least one means for controllably releasing the tramadol will desirably comprise at least one osmotic subcoat, surrounding the first once daily controlled-release dosage form or the at least one means for controllably releasing the tramadol. The osmotic subcoat comprises at least one osmotic agent and at least one hydrophilic polymer. The osmotic subcoat of this embodiment provides for the substantial separation of tramadol from the osmotic agent into substantially separate compartments/layers. This separation will desirably increase the stability of tramadol by reducing possible unfavorable interactions between tramadol and the osmagent, and/or between tramadol and the components of the release-slowing coat. For example, the osmagent will desirably be hygroscopic in nature, and will desirably attract water that will desirably lead to the degradation of tramadol. Since the osmotic agent of these embodiments will desirably be substantially separated from tramadol, tramadol will desirably be less prone to degradation from the water drawn in by the osmagent. The release-slowing coat comprises a release-slowing material and optionally a plasticizer. The coated first once daily controlled-release dosage form or the at least one means for controllably releasing the tramadol will desirably be filled into capsules, or alternatively will desirably be compressed into tablets using suitable excipients. In these embodiments the multiparticulate first once daily controlled-release dosage form or the at least one means for controllably releasing the tramadol will desirably utilize both diffusion and osmosis to control drug release, and will desirably be incorporated into sustained release and/or delayed-release dosage forms. In addition, in certain embodiments the osmotic pressure gradient and rate of release of tramadol will desirably be controlled by varying the level of the osmotic agent and/or the level of the hydrophilic polymer in the osmotic subcoat, without the need for a seal coat around the osmotic subcoat.

The hydrophilic polymer used in an osmotic subcoat of certain embodiments of the present invention functions as a carrier for the osmotic agent. In certain embodiments of the invention the hydrophilic polymer in the osmotic subcoat does not substantially affect the drug release. In at least one embodiment of the invention, the hydrophilic polymer used in the osmotic subcoat does not act as a diffusion barrier to the release of tramadol. In at least one embodiment of the invention, the release profile of the osmotic agent is substantially the same as the release profile of tramadol. Such hydrophilic polymers useful in an osmotic subcoat of the present invention include by way of example, polyvinyl pyrrolidone, hydroxyethylcellulose, hydroxypropyl cellulose, low molecular weight hydroxypropyl methylcellulose (HPMC), polymethacrylate, ethylcellulose, and mixtures thereof. In at least one embodiment of the invention, the hydrophilic polymer of the osmotic subcoat is a low molecular weight and a low viscosity hydrophilic polymer. A wide variety of low molecular weight and low viscosity hydrophilic polymers will desirably be used in the osmotic subcoat. Examples of HPMC polymers that will desirably be used in the osmotic subcoat include Pharmacoat® 606, Pharmacoat® 606G, Pharmacoat® 603, Methocel® E3, Methocel® E5, Methocel® E6, and mixtures thereof. The hydrophilic polymer of the osmotic subcoat will desirably be present in an amount of from about 0% to about 95%, such as for example, about 0%, about 5%, about 10%, about 15%, about 20%, about 25%, about 30%, about 35%, about 40%, about 45%, about 50%, about 60%, about 65%, about 70%, about 75%, about 80%, about 85%, about 90%, or about 95% by weight of the osmotic subcoat composition. For example, in certain embodiments the hydrophilic polymer is present in an amount of from 1% to 20%, in other embodiments from 3% to 10%, and in still other embodiments 7% by weight of the osmotic subcoat composition.

In at least one embodiment of the invention, the osmotic subcoat comprises about 7% Pharmacoat® 606, about 1% sodium chloride, and about 92% water, by weight of the osmotic subcoat composition.

One method for producing the osmotic subcoat will desirably be as follows. The at least one osmotic agent, for example sodium chloride, is dissolved in water. The solution of osmotic agent and water is then heated to about 60° C. The hydrophilic polymer is then added gradually to the solution. A magnetic stirrer will desirably be used to aid in the mixing of the hydrophilic polymer to the solution of osmotic agent and water. The resultant osmotic subcoating solution will desirably then be used to coat the core of the osmotic dosage form in a fluidized bed granulator, such as a granulator manufactured by Glatt (Germany) or Aeromatic (Switzerland) to the desired weight gain.

An inlet temperature of from about 10° C. to about 70° C., such as for example, from about 30° C. to about 55° C. or from about 40° C. to about 45° C.; an outlet temperature of from about 10° C. to about 70° C., such as for example, from about 20° C. to about 45° C. or from about 30° C. to about 35° C.; a product temperature of from about 110° C. to about 70° C., such as for example, from about 20° C. to about 45° C. or from about 30° C. to about 35° C.; an air flow of from about 10 c.m/h to about 180 c.m/h; such as for example, from about 40 c.m/h to about 120 c.m/h or from about 60 c.m/h to about 80 c.m/h; an atomizing pressure of from about 0.5 bar to about 4.5 bar, such as for example, from about 1 bar to about 3 bar, or about 2 bar; a curing temperature of from about 10° C. to about 70° C., such as for example, from about 20° C. to about 50° C. or from about 30° C. to about 40° C.; and a curing time of from about 5 minutes to about 720 minutes, such as for example, from about 10 minutes to about 120 minutes, and more preferably 30 minutes. Any other technology resulting in the coating formulation of the osmotic subcoat consistent with the objects of the invention will desirably also be used.

In at least one embodiment of the invention, the first once daily controlled-release dosage form or the at least one means for controllably releasing the tramadol comprises at least one lipid or wax coat. Example of the lipids and waxes useful for the manufacture of the at least one lipid or wax coat will desirably be, for example, the lipids and waxes described above.

The amount of the at least one lipid will desirably range from about 1% to about 100% of the coat composition. In certain embodiments, the amount of lipid will desirably be about 1%, about 5%, about 10%, about 15%, about 20%, about 25%, about 30%, about 35%, about 40%, about 45%, about 50%, 55%, about 60%, about 65%, about 70%, about 75%, about 80%, about 85%, about 90%, about 95% or about 100% of the coat composition. The amount of the at least one wax will desirably range from about 1% to about 100% of the coat composition. In certain embodiments, the amount of wax will desirably be about 1%, about 5%, about 10%, about 15%, about 20%, about 25%, about 30%, about 35%, about 40%, about 45%, about 50%, 55%, about 60%, about 65%, about 70%, about 75%, about 80%, about 85%, about 90%, about 95% or about 100% of the coat composition.

In at least one embodiment of the invention, the first once daily controlled-release dosage form or the at least one means for controllably releasing the tramadol comprises at least one lipid and wax combination coat. The above described lipids and waxes will desirably be combined to manufacture such a hybrid coat.

In at least one embodiment of the invention, the first once daily controlled-release dosage form or the at least one means for controllably releasing the tramadol comprises at least one lipid and wax combination coat, which relates to aqueous dispersions of waxes, lipids or waxes and lipids for pharmaceutical coating. U.S. Pat. No. 5,023,108 provides guidance for the use and manufacture coatings comprising such lipid and/or wax dispersions.

In at least one embodiment of the invention, the first once daily controlled-release dosage form or the at least one means for controllably releasing the tramadol comprises at least one hydrophobic coat. Example of hydrophobic polymers, other than the lipids or waxes described above, useful for the manufacture of the at least one hydrophobic coat will desirably be, for example, the hydrophobic polymer described above. The amount of the at least one hydrophobic polymer will desirably range from about 30% to about 100% of the coat composition. In certain embodiments, the amount of hydrophobic polymer will desirably be about 30%, about 35%, about 40%, about 45%, about 50%, 55%, about 60%, about 65%, about 70%, about 75%, about 80%, about 90%, about 95%, or about 100% of the coat composition.

In at least one embodiment of the invention, the first once daily controlled-release dosage form or the at least one means for controllably releasing the tramadol comprises a dispersion of an anionic water insoluble hydrophilic polymer and a water insoluble hydrophobic film forming polymer, in a pharmaceutically acceptable organic solvent which prevents the release of the active ingredient in the gastric fluid thereby providing enteric protection to the first once daily controlled-release dosage form or the at least one means for controllably releasing the tramadol. Examples of the water insoluble anionic hydrophilic polymer of the coating composition include polyacrylic acids such as, for example, carboxyvinyl polymer, carbopol and polycarbofil; gums such as guar gum, xanthan gum, tragacanth gum, carragenan, locust bean gum; alginates; pectins and their metallic salts. Examples of the water insoluble hydrophobic film-forming polymers include cellulose ethers, shellac, zein, and waxes. The anionic hydrophilic polymer to hydrophobic film-forming polymer ratio will desirably range from about 1:9 to about 9:1. The coating composition will desirably optionally contain a plasticizer. For example, if the coating is to function as an enteric coating then plasticizer is not required. For targeting different parts of intestine different percentages of plasticizer may be added to attain the release at the desired site. Examples of plasticizers useful for use in such coating compositions is provide aove. Pigments, colorants, antifoam agents, antioxidants, waxes, monoglycerides, emulsifiers, surfactants and other additives may be added to the dispersion either to adjust its viscosity or to modify the resultant film properties. Any pharmaceutically acceptable organic solvent may be used for this coating composition and include those selected from the group consisting of isopropyl alcohol, ethanol, acetone, or mixtures thereof. Organic solvents may also be among themselves mixed with small amounts of water. The presence of the water insoluble hydrophobic film forming polymer along with an anionic water insoluble hydrophilic polymer creates an environment, which prevents the swelling of the anionic water insoluble hydrophilic polymer in the acidic pH of gastric media, as a result no water reaches the core. However, when the dosage form is placed in the intestinal media (alkaline pH), the anionic water insoluble hydrophilic polymer undergoes neutralization reaction with the basic moieties present in the media leading to the swelling. This swollen anionic hydrophilic polymer present at the surface causes the media to pass to anionic hydrophilic polymer present inside the coating, which continues till the media reaches the core. This results in the formation of the several pathways for the passage of the media from outside to inside of the system or device and passage of the drug from inside to the outside surrounding media. It will be apparent to the skilled artisan that the coating composition will desirably be modified by changing the ratio of the anionic water insoluble hydrophilic polymer to that of the water insoluble hydrophobic film forming polymer used; amount of the plasticizers; thickness of the coating. Guidance for the use and manufacture of such coatings is provided in U.S. Patent Application No. 20060073204 published Apr. 6, 2006.

In at least one embodiment of the invention, the first once daily controlled-release dosage form or the at least one means for controllably releasing the tramadol comprises at least one hydrophilic coat, which coat will desirably be comprises of a hydrophilic soluble polymer, a hydrophilic swellable or swellable and erodable polymer, or combination thereof. Examples of such polymers have been provided above. The amount of the at least one hydrophilic soluble polymer will desirably range from about 20% to about 100% of the coat composition. In certain embodiments, the amount of the hydrophilic soluble polymer will desirably be about 20%, about 25%, about 30%, about 35%, about 40%, about 45%, about 50%, 55%, about 60%, about 65%, about 70%, about 75%, about 80%, about 85%, about 90%, about 95%, or about 100% of the coat composition. The amount of the at least one hydrophilic swellable and swellable or erodable polymer will desirably range from about 20% to about 100% of the coat composition. In certain embodiments, the amount of the hydrophilic swellable or swellable and erodable polymer will desirably be about 20%, about 25%, about 30%, about 35%, about 40%, about 45%, about 50%, 55%, about 60%, about 65%, about 70%, about 75%, about 80%, about 85%, about 90%, about 95%, or about 100% of the coat composition.

In at least one embodiment, first once daily controlled-release dosage form or the at least one means for controllably releasing the tramadol comprises at least one taste-masking coating. In at least one embodiment the taste-masking coating formulations contain polymeric ingredients. It is contemplated that other excipients consistent with the objects of the present invention will desirably also be used in the taste-masking coating.

In at least one embodiment of the invention, the first once daily controlled-release dosage form or the at least one means for controllably releasing the tramadol comprises at least one tase-maskin coating, wherein said taste-masking coating comprises a polymer such as ethylcellulose, which will desirably be used as a dry polymer (such as Ethocel®, Dow Corning) solubilised in organic solvent prior to use, or as an aqueous dispersion. One commercially-available aqueous dispersion of ethylcellulose is Aquacoat® (FMC Corp., Philadelphia, Pa., U.S.A.). Aquacoat® will desirably be prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, the Aquacoat is intimately mixed with a suitable plasticizer prior to use. Another aqueous dispersion of ethylcellulose is commercially available as Surelease® (Colorcon, Inc., West Point, Pa., U.S.A.). This product will desirably be prepared by incorporating plasticizer into the dispersion during the manufacturing process. A hot melt of a polymer, plasticizer (e.g. dibutyl sebacate), and stabilizer (e.g. oleic acid) is prepared as a homogeneous mixture, which is then diluted with an alkaline solution to obtain an aqueous dispersion which will desirably be applied directly onto substrates.

In other embodiments of the invention, the first once daily controlled-release dosage form or the at least one means for controllably releasing the tramadol, polymethacrylate acrylic polymers will desirably be employed as taste masking polymers. In at least one embodiment, the taste masking coating is an acrylic resin lacquer used in the form of an aqueous dispersion, such as that which is commercially available from Rohm Pharma under the tradename Eudragit® or from BASF under the tradename Kollicoat®. In further preferred embodiments, the acrylic coating comprises a mixture of two acrylic resin lacquers commercially available from Rohm Pharma under the tradenames Eudragit® RL and Eudragit® RS, respectively. Eudragit® RL and Eudragit® RS are copolymers of acrylic and methacrylic esters with a low content of quaternary ammonium groups, the molar ratio of ammonium groups to the remaining neutral (meth)acrylic esters being 1:20 in Eudragit® RL and 1:40 in Eudragit® RS. The mean molecular weight is 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids. Eudragit® RL/RS dispersions or solutions of the present invention will desirably be mixed together in any desired ratio in order to ultimately obtain a taste masking coating having a desirable drug dissolution profile. Desirable controlled release formulations will desirably be obtained, for example, from a retardant coating derived from about 100% Eudragit® RL; about 50% Eudragit® RL with about 50% Eudragit® RS; and about 10% Eudragit® RL with about 90% Eudragit® RS.

In other embodiments of the matrix dosage form, the taste-masking polymer will desirably be an acrylic polymer, which is cationic in character based on dimethylaminoethyl methacrylate and neutral methacrylic acid esters (such as Eudragit® E, commercially available from Rohm Pharma). The hydrophobic acrylic polymer coatings of the present invention will desirably further include a neutral copolymer based on poly (meth)acrylates, such as Eudragit® NE (NE=neutral ester), commercially available from Rohm Pharma. Eudragit® NE 30D lacquer films are insoluble in water and digestive fluids, but permeable and swellable.

In other embodiments of the invention, the first once daily controlled-release dosage form or the at least one means for controllably releasing the tramadol of the matrix dosage form, the taste masking polymer is a dispersion of poly (ethylacrylate, methyl methacrylate) 2:1 (Kollicoat® EMM 30 D, BASF).

In other embodiments of the invention, the first once daily controlled-release dosage form or the at least one means for controllably releasing the tramadol the taste masking polymer will desirably be a polyvinyl acetate stabilized with polyvinylpyrrolidone and sodium lauryl sulfate such as Kollicoat® SR30D (BASF).

Other taste masking polymers used in the first once daily controlled-release dosage form or the at least one means for controllably releasing the tramadol include hydroxypropylcellulose (HPC); hydroxypropylmethylcellulose (HPMC); hydroxyethylcellulose; gelatin; gelatin/acacia; gelatin/acacia/vinvylmethylether maleic anhydride; gelatin/acacia/ethylenemaleic anhydride; carboxymethyl cellulose; polyvinvylalcohol; nitrocellulose; polyvinylalcohol-polyethylene glycol graft-copolymers; shellac; wax and mixtures thereof.

The taste-masking coatings will desirably be applied to the first once daily controlled-release dosage form or the at least one means for controllably releasing the tramadol from one or more organic or aqueous solvent solutions or suspensions. In at least one embodiment of the invention the first once daily controlled-release dosage form or the at least one means for controllably releasing the tramadol, the organic solvents that will desirably be used to apply the taste-masking coatings include one or more of acetone, lower alcohols such as ethanol, isopropanol and alcohol/water mixtures, chlorinated hydrocarbons, and the like. Devices used to coat the first once daily controlled-release dosage form or the at least one means for controllably releasing the tramadol of the invention with a taste-masking coating include those conventionally used in pharmaceutical processing, such as fluidized bed coating devices. The control-releasing coatings applied to the first once daily controlled-release dosage form or the at least one means for controllably releasing the tramadol will desirably contain ingredients other than the cellulosic polymers. One or more colorants, flavorants, sweeteners, will desirably also be used in the taste-masking coating.

In some embodiments of the invention, the incorporation of a pore former or channeling agent or channeling agent in the tase-masking coat is optional. The pore former or channeling agents will desirably be inorganic or organic, and may be particulate in nature and include materials that will desirably be dissolved, extracted or leached from the coating in the environment of use. Upon exposure to fluids in the environment of use, the pore former or channeling agents or channeling agents will desirably for example be dissolved, and channels and pores are formed that fill with the environmental fluid.

For example, the pore former or channeling agents of certain embodiments of the matrix dosage forms will desirably comprise one or more water-soluble hydrophilic polymers in order to modify the release characteristics of the formulation. Examples of suitable hydrophilic polymers used as pore former or channeling agents include hydroxypropylmetlhylcellulose, cellulose ethers and protein-derived materials of these polymers, the cellulose ethers, especially hydroxyalkylcelluloses and carboxyalkylcelluloses. Also, synthetic water-soluble polymers will desirably be used, examples of which include polyvinylpyrrolidone, cross-linked polyvinyl-pyrrolidone, polyethylene oxide, water-soluble polydextrose, saccharides and polysaccharides, such as pullulan, dextran, sucrose, glucose, fructose, mannitol, lactose, mannose, galactose, and sorbitol. In at least one embodiment, the hydrophilic polymer comprises hydroxypropyl-methylcellulose.

Other non-limiting examples of pore formers or channeling agents include alkali metal salts such as lithium carbonate, sodium chloride, sodium bromide, potassium chloride, potassium sulfate, potassium phosphate, sodium acetate, and sodium citrate. The pore-forming solids will desirably also be polymers, which are soluble in the environment of use, such as Carbowaxes, and Carbopol. In addition, the pore formers or channeling agents embrace diols, polyols, polyhydric alcohols, polyalkylene glycols, polyglycols, and poly(a-w)alkylenediols. Other pore formers or channeling agents which will desirably be useful in the formulations of the present invention include starch, modified starch, and starch derivatives, gums, including but not limited to xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, such as potassium polymethacrylate, carrageenan, kappa-carrageenan, lambda-carrageenan, gum karaya, biosynthetic gum, etc. Other pore formers or channeling agents include materials useful for making microporous lamina in the environment of use, such as polycarbonates comprised of linear polyesters of carbonic acid in which carbonate groups reoccur in the polymer chain, microporous materials such as bisphenol, a microporous poly(vinylchloride), micro-porous polyamides, microporous modacrylic copolymers, microporous styrene-acrylic and its copolymers, porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous hiomopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), and mixtures thereof.

In general, the amount of pore former or channeling agent included in the taste masking coatings of certain embodiments of the matrix dosage forms will desirably be from about 0.1% to about 80%, by weight, relative to the combined weight of polymer and pore former or channeling agent. The percentage of pore former or channeling agent as it relates to the dry weight of the taste-masking polymer will desirably have an influence on the drug release properties of the coated matrix. In at least one embodiment that uses water soluble pore former or channeling agents such as hydroxypropylmethylcellulose, a taste masking polymer: pore former or channeling agent dry weight ratio of between about 10:1 and about 1:1 will desirably be present. In certain embodiments the taste masking polymer: pore former or channeling agent dry weight ratio is from about 8:1 to about 1.5:1; and in other embodiments from about 6:1 to about 2:1. In at least one embodiment using Eudragit® NE30D as the taste masking polymer and a hydroxypropylmethylcellulose (approx 5 cps viscosity (in a 2% aqueous solution)) such as Methocel® E5, Pharmacoat 606G as the water soluble pore former or channeling agent, a taste masking polymer: pore former or channeling agent dry weight ratio of about 2:1 is present.

Colorants that will desirably be used in the taste-masking coating of certain embodiments of the matrix dosage forms include food, drug and cosmetic colors (FD&C), drug and cosmetic colors (D&C) or external drug and cosmetic colors (Ext. D&C). These colors are dyes, lakes, and certain natural and derived colorants. Useful lakes include dyes absorbed on aluminum hydroxide or other suitable carriers.

Flavorants that will desirably be used in the taste-masking coating of certain embodiments of the matrix dosage forms include natural and synthetic flavoring liquids. An illustrative list of such flavorants includes volatile oils, synthetic flavor oils, flavoring aromatics, oils, liquids, oleoresins and extracts derived from plants, leaves, flowers, fruits, stems and combinations thereof. A non-limiting representative list of these includes citric oils, such as lemon, orange, grape, lime and grapefruit, and fruit essences, including apple, pear, peach, grape, strawberry, raspberry, cherry, plum, pineapple, apricot, or other fruit flavors. Other useful flavorants include aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin); mixtures thereof and the like.

Sweeteners that will desirably be used in the taste-masking coating of certain embodiments of the matrix dosage forms include glucose (corn syrup), dextrose, invert sugar, fructose, and mixtures thereof (when not used as a carrier); saccharin and its various salts, such as sodium salt; dipeptide sweeteners such as aspartame; dihydrochalcone compounds, glycyrrhizin; Steva Rebaudiana (Stevioside); chloro derivatives or sucrose such as sucralose; and sugar alcohols such as sorbitol, mannitol, xylitol, and the like. Also contemplated are hydrogenated starch hydrolysates and the synthetic sweeteners such as 3,6-dihydro-6-methyl-1-1-1,2,3-oxathiazin-4-1-2,2-dioxide, particularly the potassium salt (acesulfame-K), and sodium and calcium salts thereof. The sweeteners will desirably be used alone or in any combination thereof.

Emulsifying agent(s), if used, will desirably be present in an amount of from about 0.01% to about 5% by weight of the taste masking polymer dry weight. For example, in certain embodiments the emulsifying agent is present in an amount of from about 0.05% to about 3%; in other embodiments from about 0.08% to about 1.5%, and in still other embodiments at about 0.1% by weight of the taste masking coat dry weight.

In at least one embodiment the anti-foaming agent to be used in the taste-masking coat is Simethicone C. The anti-foaming agent will desirably be present in an amount of from about 0.1% to about 10% of the matrix taste masking coat weight. For example, in certain embodiments the anti-foaming agent is present in an amount of from about 0.2% to about 5%; in other embodiments from about 0.3% to about 1%, and in still other embodiments at about 0.6% by weight of the matrix taste masking polymer dry weight.

The taste-masking coating will desirably be present in an amount of from about 1% to about 90% by weight of the first once daily controlled-release dosage form or the at least one means for controllably releasing the tramadol, depending upon the choice of polymer, the ratio of polymer:pore former or channeling agent, and the total surface area of the first once daily controlled-release dosage form or the at least one means for controllably releasing the tramadol formulation. Since a certain thickness of taste masking coating has to be achieved in order to achieve effective taste masking, the amount of taste masking polymer coating used during manufacture is related to the total surface area of the batch of uncoated first once daily controlled-release dosage form or the at least one means for controllably releasing the tramadol that requires a coating. For example, the taste masking polymer surface area coverage will desirably range from about 0.5 mg/cm² to about 20 mg/cm². For example, in certain embodiments the surface area coverage of the taste masking polymer is from about 0.6 mg/cm² to about 10 mg/cm², and in other embodiments is from about 1 mg/cm² to about 5 mg/cm². In at least one embodiment of the invention, Eudragit® E is employed as the taste masking polymer at a surface area coverage of about 4 mg/cm².

In the absence of an accurate determination of total surface area of a first once daily controlled-release dosage form or the at least one means for controllably releasing the tramadol, the amount of taste masking polymer to be applied will desirably be expressed as a percentage of the uncoated first once daily controlled-release dosage form or the at least one means for controllably releasing the tramadol. For example, in certain embodiments the taste-masking coating is present in an amount of from about 5% to about 60%, such as for example, about 5, about 10, about 15, about 20, about 25, about 30, about 35, about 40, about 45, about 50, about 55, or about 60% of the first once daily controlled-release dosage form or the at least one means for controllably releasing the tramadol.

The release-slowing coats, delayed-release coats, and other coats described herein will desirably be applied by methods known to the skill artisan, such as for example, two fluid atomized sparying or one fluid atomized spraying, dry coating, dry powder polymer coating, electrostatic deposition, coating by compression or melt coating or melt congealing.

The ratio of the components in the first once daily controlled-release dosage form or the at least one means for controllably releasing the tramadol and optionally, at least one semipermeable membrane, at least one release-slowing coat, at least one lipid or wax coat, at least one delayed-release coat, at least one swellable coat, at least one erodable coat, at least one swellable and erodable coat, at least one hydrophobic coat, at least one hydrophilic coat, at least one coat comprising at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol or combinations thereof, as well as the amount of the various membranes or coats applied will desirably be varied to control delivery of tramadol either predominantly by diffusion across the surface of the semipermeable membrane to predominantly by osmotic pumping through the at least one passageway in the semipermeable membrane, and combinations thereof such that the first once daily controlled-release dosage form or the at least one means for controllably releasing the tramadol will desirably exhibit a modified-release, controlled-release, sustained-release, extended-release, prolonged-release, bi-phasic release, delayed-release profile or a combination of release profiles wherein the in-vitro release rates of tramadol is such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released when measured using a USP Type I, II, or III apparatus (Rotating Basket Method) in 900 ml 0.1N HCl, water, 0.1N HCl+0.1% Cetrimide, USP Buffer pH 1.5, Acetate Buffer pH 4.5, Phosphate Buffer, pH 6.5 or Phosphate Buffer pH7.4 at 75 rpm at 37°±0.5° C., and when the osmotic dosage form is administered to a patient in need of such administration will desirably exhibit at steady state the following pharmacokinetic parameters in-vivo under fasting conditions: (i) an AUC₀₋₂₄ from about 2886 to about 10130 ng·h/ml, (ii) a Cₘₐₓ of about 171 to about 564 ng/ml, and will desirably be bioequivalent according to FDA guidelines to a second orally administrable dosage form comprising the same dose of tramadol also suitable for once daily administration in the fasted state.

In embodiments of the invention where the mode of exit of tramadol comprises a plurality of pores, the amount of pore forming agent employed to achieve the desired in-vitro dissolution rates and in-vivo pharmacokinetic parameters will desirably be readily determined by those skilled in the drug delivery art.

The at least one release-slowing or delayed-release coat to be applied will desirably surround the first once daily controlled-release dosage form or the at least one means for controllably releasing the at least one tramadol or will desirably be laminated onto the dosage form or means such that a portion of the dosage form or means for controllably releasing the tramadol is exposed to the aqueous dissolution or gastrointestinal medium. Guidance for the manufacture of such dosage forms is provided in U.S. Pat. No. 6,033,685.

In at least one embodiment of the invention, the first once daily controlled-release dosage form or means for controllably releasing the tramadol is in the form of a microparticulate. The microparticles will desirably be made from the tramadol, spheronization aids, and other excipient(s) coated with at least one release-slowing coat, delayed-release coat, or combinations thereof. The control-releasing coated microparticles will desirably then be combined with an excipient mass and/or other pharmaceutical excipients, and compressed into tablets. Conventional tablets will desirably be manufactured by compressing the coated microparticles with suitable excipients using known compression techniques. The dissolution profile of the control-releasing coated multiparticles is not substantially affected by the compression of the microparticles into a tablet. The resultant dosage forms enjoy the processing ease associated with the use of excipient masses and the release properties associated with control-releasing coated microparticles. Alternatively, the coated microparticles will desirably be filled into capsules. In embodiments of the invention where the first once daily controlled-release dosage form or the at least one means for controllably releasing the tramadol is in the form of a plurality of microparticles, the microparticles will desirably be packaged into either capsules or compressed into a tablet with at least one pharmaceutically excipient.

In certain embodiments of the invention, the at least one pharmaceutical excipient will desirably be a disintegrant. In embodiments of the invention comprising a tablet comprising a plurality of microparticles, the tablet comprises cushioning wax beads. Guidance for manufacture and use of cushioning wax beads is provided in U.S. Pat. No. 6,923,984.

The forms of administration according to the invention are suitable for oral administration. In certain embodiments the forms of administration are tablets and capsules. However, the composition of the invention will desirably also take the form of pellets, beads or microtablets, which will desirably then be packaged into capsules or compressed into a unitary solid dosage form. Other solid oral dosage forms as disclosed herein will desirably be prepared by the skilled artisan, despite the fact that such other solid oral dosage forms may be more difficult to commercially manufacture.

The present invention also contemplates combinations of differently coated microparticles into a dosage form to provide a variety of different release profiles. For example, in certain embodiments, microparticles with a delayed release profile will desirably be combined with other microparticles having a sustained release profile to provide a multiple component controlled release tramadol formulation. In addition, other embodiments will desirably include one or more further components of immediate release tramadol. The immediate release tramadol component will desirably take the form of uncoated tramadol microparticles or powders; tramadol microparticles coated with a soluble immediate release coating, such as an Opadry® type coating, as are known to those skilled in the art, or a combination of any of the foregoing. The multiple components will desirably then be blended together in the desired ratio and placed in a capsule, or formed into a tablet.

In certain embodiments of the present invention, a multiparticulate system is provided which contains multiple microparticles each containing an effective amount of tramadol and at least one pharmaceutically acceptable excipient. In at least one embodiment the tramadol comprises tramadol hydrochloride. The multiparticulates will desirably be contained within a capsule, or will desirably be compressed into a matrix or tablet, that upon ingestion dissolves into multiple units (e.g. pellets), wherein the sub-units or pellets possess the desired controlled release properties of the dosage form. The multiparticulates or the multiple unit dosage forms will desirably be surrounded by one or more coatings. Examples of such coatings are described above.

The tramadol in the microparticles will desirably be present in an effective amount of from about 70% to about 98% by weight of the microparticles. For example, in certain embodiments tramadol hydrochloride is present in the microparticles at about 70, about 72, about 74, about 76, about 78, about 80, about 82, about 84, about 86, about 88, about 90, about 92, about 94, about 96, or about 98% by weight of the microparticle.

In addition to the tramadol, the microparticles of the present invention also include at least one pharmaceutically acceptable excipient. Excipients will desirably be added to facilitate in the preparation, patient acceptability and functioning of the dosage form as a drug delivery system. Excipients include spheronization aids, solubility enhancers, disintegrating agents, diluents, lubricants, binders, fillers, glidants, suspending agents, emulsifying agents, anti-foaming agents, flavouring agents, colouring agents, chemical stabilizers, pH modifiers, etc. Depending on the intended main function, excipients to be used in formulating compositions are subcategorized into different groups. However, one excipient will desirably affect the properties of a composition in a series of ways, and many excipients used in compositions will desirably thus be described as being multifunctional.

The microparticles of the present invention will desirably be manufactured using standard techniques known to one of skill in the art. Useful microparticles include drug-layered microparticles and drug-containing microparticles.

Microparticles containing drug in the core will desirably be prepared by a number of different procedures. For example: In a spray drying process, an aqueous solution of core material and hot solution of polymer is atomized into hot air, the water then evaporates, and the dry solid is separated in the form of pellets, for example by air suspension. A spray-drying process will desirably produce hollow pellets when the liquid evaporates at a rate that is faster than the diffusion of the dissolved substances back into the droplet interior, or if due to capillary action the dissolved substance migrates out with the liquid to the droplet surface, leaving behind a void. Another example is a spray congealing process, where a slurry of drug material that is insoluble in a molten mass is spray congealed to obtain discrete particles of the insoluble materials coated with the congealed substance. A further example is a fluidized bed based granulation/pelletization process, where a dry drug is suspended in a stream of hot air to form a constantly agitated fluidized bed. An amount of binder or granulating liquid is then introduced in a finely dispersed form to cause pelletization.

The drug-containing microparticles of the present invention will desirably also be made by, for example, a spheronization process. One method of manufacturing the drug-containing microparticles is the applicant's proprietary CEFORM™ (Centrifugally Extruded & Formed Microspheres/Microparticles) technology, which is the simultaneous use of flash heat and centrifugal force, using proprietary designed equipment, to convert dry powder systems into microparticles of uniform size and shape. The production of microparticles containing an active drug using this CEFORM™ technology is described in U.S. Pat. No. 5,683,720. This patent deals with the use of LIQUIFLASH® processing to spheronize compositions containing one or more active drugs to form LIQUIFLASH® microparticles.

With the CEFORM™ technology, the processing of the drug-containing microparticles of the present invention is carried out in a continuous fashion, whereby a pre-blend of drug and excipients is fed into a spinning “microsphere head”, also termed as a “spheronizing head”. The microsphere head, which is a multi-aperture production unit, spins on its axis and is heated by electrical power. The drug and excipient(s) pre-blend is fed into the center of the head with an automated feeder. The material moves, via centrifugal force, to the outer rim where the heaters, located in the rim of the head, heat the material. Microparticles are formed when the molten material exits the head, which are then cooled by convection as they fall to the bottom of the Microparticle Chamber. The product is then collected and stored in suitable product containers. Careful selection of the types and levels of excipient(s) control microparticle properties such as sphericity, surface morphology, and dissolution rate. One advantage of such a process is that the microparticles are produced and collected from a dry feedstock without the use of any solvents.

There are at least two approaches that will desirably be used to produce drug-containing microparticles using the CEFORM process: (i) the encapsulation approach and (ii) the co-melt approach. In the encapsulation approach, the process is conducted below the melting point of the drug. Therefore, the excipients are designed to melt and entrain the drug particles on passing through the apertures to form microparticles. The resulting microparticles contain the drug, in its native state, essentially enveloped by or as an intimate matrix with the resolidified excipients. In the co-melt approach, the process is conducted above the melting point of the drug. In this case, the drug and the excipients melt or become fluid simultaneously upon exposure to the heat. The molten mixture exits the head and forms microparticles, which cool as they fall to the bottom of the collection bin where they are collected.

In at least one embodiment the microparticles are manufactured using the encapsulation approach. In the encapsulation approach the excipient(s) which are chosen have a lower melting point than the drug with which they will be combined. Therefore the spheronizing process will desirably be performed at lower temperatures, than the melting point of the drug. As a result, this will desirably reduce the risk of polymeric interconversion, which will desirably occur when using processing temperatures close to the melting point.

In a prophetic example of certain embodiments of the present invention, the manufacturing process for the microparticles will desirably hypothetically be as follows: Spheronization aid is screened through a 425 micron (μm) screen. In at least one embodiment, the spheronization aid is distilled glyceryl monostearate (i.e. DMG-03VF). 50% of the spheronization aid is added to a bowl in a high shear mixer. In at least one embodiment, the bowl is a 6 litre bowl and the high shear mixer is a Diosna P1-6 high speed mixer granulator. The tramadol is then added to the bowl of the mixer, and then the remainder of the spheronization aid is added. The material is then blended in the mixer for a time from about 1 minute to about 30 minutes; preferably from about 3 minutes to about 10 minutes; and more preferably about 6 minutes. The mixer motor speed is from about 50 rpm to about 2000 rpm; preferably from about 200 rpm to about 500 rpm; and more preferably about 300 rpm. The chopper motor speed is from about 50 rpm to about 2000 rpm; preferably from about 200 rpm to about 500 rpm; and more preferably about 400 rpm. The blended material is then spheronized in a CEFORM™ spheronizing head. The spheronizing head speed is from about 5 Hz to about 60 Hz; preferably from about 10 Hz to about 30 Hz; and more preferably about 15 Hz. In at least one embodiment the CEFORM™ spheronizing head is a 5 inch head. The spheronizing head temperature is maintained at a temperature from about 70° C. to about 130° C.; such as for example from about 90° C. to about 110° C. The microparticles obtained from the spinning process are then screened through a screen that is from 150 μm to 800 μm.

For microparticles manufactured using a spheronization process such as the CEFORM™ process, the microparticles include, in addition to the tramadol, at least one spheronization aid. Spheronization aids will desirably assist the drug-containing mix to form robust durable spherical particles. Some examples of materials useful as spheronization aids include, but are not limited to glyceryl monostearate, glyceryl behenate, glyceryl dibehenate, glyceryl palmitostearate, hydrogenated oils such as hydrogenated castor oil marketed under the name Cutina™ HR, fatty acid salts such as magnesium or calcium stearate, polyols such as mannitol, sorbitol, xylitol, stearic acid, palmitic acid, sodium lauryl sulfate, polyoxyethylene ethers, esterified polyoxyethylenes such as PEG-32 distearate, PEG-150 distearate, cetostearyl alcohol, waxes (e.g. carnauba wax, white wax, paraffin wax) and wax-like materials. Certain thermo-plastic or thermo-softening polymers will desirably also function as spheronization aids. Some non-limiting examples of such thermo-plastic or thermo-softening polymers include Povidone, cellulose ethers and polyvinylalcohols. Combinations of spheronization aids will desirably be used. In at least one embodiment, the spheronization aid is glyceryl monostearate (i.e. DMG-03VF). The spheronization aid will desirably be present in an amount of from about 0.1% to about 99% by weight of the microparticle. For example, in certain embodiments the spheronization aid is present in an amount of about 5% to about 90%; in other embodiments from about 10% to about 80%; in still other embodiments from about 20% to about 70%; and in even still other embodiments from about 30% to about 60% by weight of the microparticle.

In certain embodiments, each microparticle will desirably also include at least one solubility enhancer. Solubility enhancers will desirably be surfactants. Certain embodiments of the invention include a solubility enhancer that is a hydrophilic surfactant. Hydrophilic surfactants will desirably be used to provide any of several advantageous characteristics to the compositions, including: increased solubility of the tramadol in the microparticle; improved dissolution of the tramadol; improved solubilization of the tramadol upon dissolution; enhanced absorption and/or bioavailability of the tramadol. The hydrophilic surfactant will desirably be a single hydrophilic surfactant or a mixture of hydrophilic surfactants, and will desirably be ionic or non-ionic.

Likewise, various other embodiments of the invention include a lipophilic component, which will desirably be a lipophilic surfactant, including a mixture of lipophilic surfactants, a triglyceride, or a mixture thereof. The lipophilic surfactant will desirably provide any of the advantageous characteristics listed above for hydrophilic surfactants, as well as further enhancing the function of the surfactants. These various embodiments are described in more detail below.

As is well known in the art, the terms “hydrophilic” and “lipophilic” are relative terms. To function as a surfactant, a compound includes polar or charged hydrophilic moieties as well as non-polar hydrophobic (lipophilic) moieties; i.e., a surfactant compound is amphiphilic. An empirical parameter commonly used to characterize the relative hydrophilicity and lipophilicity of non-ionic amphiphilic compounds is the hydrophilic-lipophilic balance (the “HLB” value). Surfactants with lower HLB values are more lipophilic, and have greater solubility in oils, whereas surfactants with higher HLB values are more hydrophilic, and have greater solubility in aqueous solutions.

Using HLB values as a rough guide, hydrophilic surfactants will desirably generally be considered to be those compounds having an HLB value greater than about 10, as well as anionic, cationic, or zwitterionic compounds for which the HLB scale is not generally applicable. Similarly, lipophilic surfactants will desirably be compounds having an HLB value less than about 10.

It should be appreciated that the HLB value of a surfactant is merely a rough guide generally used to enable formulation of industrial, pharmaceutical and cosmetic emulsions. For many important surfactants, including several polyethoxylated surfactants, it has been reported that HLB values will desirably differ by as much as about 8 HLB units, depending upon the empirical method chosen to determine the HLB value (Schott, J. Pharm. Sciences, 79(1), 87-88 (1990)). Likewise, for certain polypropylene oxide containing block copolymers (poloxamers, available commercially as PLURONIC® surfactants, BASF Corp.), the HLB values may not accurately reflect the true physical chemical nature of the compounds. Finally, commercial surfactant products are generally not pure compounds, but are often complex mixtures of compounds, and the HLB value reported for a particular compound will desirably more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art will desirably readily identify surfactants having suitable hydrophilicity or lipophilicity for use in the present invention, as described herein.

Solubility enhancers will desirably be any surfactant suitable for use in pharmaceutical compositions. Suitable surfactants will desirably be anionic, cationic, zwitterionic or non-ionic. In addition, refined, distilled or fractionated surfactants, purified fractions thereof, or re-esterified fractions, are also within the scope of the invention.

Although polyethylene glycol (PEG) itself does not function as a surfactant, a variety of PEG-fatty acid esters have useful surfactant properties. Examples of polyethoxylated fatty acid monoester surfactants are shown in the following table:

PEG-Fatty Acid Monoester Surfactants

| Compound | Commercial Product (Supplier) | HLB |
|---|---|---|
| PEG 4-100 monolaurate | Crodet L series (Croda) | >9 |
| PEG 4-100 monooleate | Crodet O series (Croda) | >8 |
| PEG 4-100 monostearate | Crodet S series (Croda), Myrj Series (Atlas/ICI) | >6 |
| PEG 400 distearate | Cithrol 4DS series (Croda) | >10 |
| PEG 100, 200, 300 | Cithrol ML series (Croda) | >10 |
| monolaurate | | |
| PEG 100, 200, 300 | Cithrol MO series (Croda) | >10 |
| monooleate | | |
| PEG 400 dioleate | Cithrol 4DO series (Croda) | >10 |
| PEG 400-1000 | Cithrol MS series (Croda) | >10 |
| monostearate | | |
| PEG-1 stearate | Nikkol MYS-IEX (Nikko), Coster KI (Condea) | 2 |
| PEG-2 stearate | Nikkol MYS-2 (Nikko) | 4 |
| PEG-2 oleate | NikkoI MYO-2 (Nikko) | 4.5 |
| PEG-4 laurate | Mapeg ® 200 ML (PPG), | 9.3 |
| | Kessco ® PEG 200ML (Stepan), | |
| | LIPOPEG 2L (LIPO Chem.) | |
| PEG-4 oleate | Mapeg ® 200 MO (PPG), | 8.3 |
| | Kessco ® PEG200 MO (Stepan), | |
| PEG-4 stearate | Kessco ® PEG 200 MS (Stepan), | 6.5 |
| | Hodag 20 S (Calgene), Nikkol MYS-4 (Nikko) | |
| PEG-5 stearate | Nikkol TMGS-5 (Nikko) | 9.5 |
| PEG-5 oleate | Nikkol TMGO-5 (Nikko) | 9.5 |
| PEG-6 oleate | Algon OL 60 (Auschem SpA), | 8.5 |
| | Kessco ® PEG 300 MO (Stepan), | |
| | Nikkol MYO-6 (Nikko), | |
| | Emulgante A6 (Condea) | |
| PEG-7 oleate | Algon OL 70 (Auschem SpA) | 10.4 |
| PEG-6 laurate | Kessco ® PEG300 ML (Stepan) | 11.4 |
| PEG-7 laurate | Lauridac 7 (Condea) | 13 |
| PEG-6 stearate | Kessco ® PEG300 MS (Stepan) | 9.7 |
| PEG-8 laurate | Mapeg ® 400 ML (PPG), | 13 |
| | LIPOPEG 4DL(Lipo Chem.) | |
| PEG-8 oleate | Mapeg ® 400 MO (PPG), | 12 |
| | Emulgante A8 (Condea); | |
| | Kessco PEG 400 MO (Stepan) | |
| PEG-8 stearate | Mapeg ® 400 MS (PPG), Myrj 45 | 12 |
| PEG-9 oleate | Emulgante A9 (Condea) | >10 |
| PEG-9 stearate | Cremophor 59 (BASF) | >10 |
| PEG-10 laurate | Nikkol MYL-10 (Nikko), Lauridac 10 (Croda) | 13 |
| PEG-10 oleate | Nikkol MYO-10 (Nikko) | 11 |
| PEG-10 stearate | Nikkol MYS-10 (Nikko), Coster K100 (Condea) | 11 |
| PEG-12 laurate | Kessco ® PEG 600ML (Stepan) | 15 |
| PEG-12 oleate | Kessco ® PEG 600MO (Stepan) | 14 |
| PEG-12 ricinoleate | (CAS #9004-97-1) | >10 |
| PEG-12 stearate | Mapeg ® 600 MS (PPG), | 14 |
| | Kessco ® PEG 600MS (Stepan) | |
| PEG-15 stearate | Nikkol TMGS-15 (Nikko), Koster K15 (Condea) | 14 |
| PEG-15 oleate | Nikkol TMGO-15 (Nikko) | 15 |
| PEG-20 laurate | Kessco ® PEG 1000 ML (Stepan) | 17 |
| PEG-20 oleate | Kessco ® PEG 1000 MO (Stepan) | 15 |
| PEG-20 stearate | Mapeg ® 1000 MS (PPG), Kessco ® PEG 1000 MS | 16 |
| | (Stepan), Myrj 49 | |
| PEG-25 stearate | Nikkol MYS-25 (Nikko) | 15 |
| PEG-32 laurate | Kessco ® PEG 1540 ML (Stepan) | 16 |
| PEG-32 oleate | Kessco ® PEG 1540 MO (Stepan) | 17 |
| PEG-32 stearate | Kessco ® PEG 1540 MS (Stepan) | 17 |
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-40 stearate | Myrj 52, Emerest ® 2715 (Henkel), | >10 |
| | Nikkol MYS-40 (Nikko) | |
| PEG-45 stearate | Nikkol MYS-45 (Nikko) | 18 |
| PEG-50 stearate | Myrj 53 | >10 |
| PEG-55 stearate | Nikkol MYS-55 (Nikko) | 18 |
| PEG-100 oleate | Crodet 0-100 (Croda) | 18.8 |
| PEG-100 stearate | Myrj 59, Arlacel 165 (ICI) | 19 |
| PEG-200 oleate | Albunol 200 MO (Taiwan Surf.) | >10 |
| PEG-400 oleate | LACTOMUL (Henkel), Albunol 400 MO (Taiwan | >10 |
| | Surf.) | |
| PEG-600 oleate | Albunol 600 MO (Taiwan Surf) | >10 |

Polyethylene glycol (PEG) fatty acid diesters are also suitable for use as surfactants in the compositions of the present invention. Representative PEG-fatty acid diesters are shown in the following table:

PEG-Fatty Acid Diester Surfactants

| Compound | Commercial Product (Supplier) | HLB |
|---|---|---|
| PEG-4 dilaurate | Mapeg ® 200 DL (PPG), | 7 |
| | Kessco ® PEG 200 DL (Stepan), | 6 |
| | LIPOPEG 2-DL (Lipo Chem.) | |
| PEG-4 dioleate | Mapeg ® 200 DO (PPG), | 6 |
| PEG-4 distearate | Kessco ® 200 DS (Stepan) | 5 |
| PEG-6 dilaurate | Kessco ® PEG 300 DL (Stepan) | 9.8 |
| PEG-6 dioleate | Kessco ® PEG 300 DO (Stepan) | 7.2 |
| PEG-6 distearate | Kessco ® PEG 300 DS (Stepan) | 6.5 |
| PEG-8 dilaurate | Mapeg ® 400 DL (PPG), | 11 |
| | Kessco ® PEG 400 DL | |
| | (Stepan), LIPOPEG 4 DL (Lipo Chem.) | |
| PEG-8 dioleate | Mapeg ® 400 DO (PPG), | 8.8 |
| | Kessco ® PEG 400 DO | |
| | (Stepan), LIPOPEG 4 DO(Lipo Chem.) | |
| PEG-8 distearate | Mapeg ® 400 DS (PPG), CDS 400 (Nikkol) | 11 |
| PEG-10 dipalmitate | Polyaldo 2PKFG | >10 |
| PEG-12 dilaurate | Kessco ® PEG 600 DL (Stepan) | 11.7 |
| PEG-12 distearate | Kessco ® PEG 600 DS (Stepan) | 10.7 |
| PEG-12 dioleate | Mapeg ® 600 DO (PPG), | 10 |
| | Kessco ® 600 DO(Stepan) | |
| PEG-20 dilaurate | Kessco ® PEG 1000 DL (Stepan) | 15 |
| PEG-20 dioleate | Kessco ® PEG 1000 DO (Stepan) | 13 |
| PEG-20 distearate | Kessco ® PEG 1000 DS (Stepan) | 12 |
| PEG-32 dilaurate | Kessco ® PEG 1540 DL (Stepan) | 16 |
| PEG-32 dioleate | Kessco ® PEG 1540 DO (Stepan) | 15 |
| PEG-32 distearate | Kessco ® PEG 1540 DS (Stepan) | 15 |
| PEG-400 dioleate | Cithrol 4DO series (Croda) | >10 |
| PEG-400 distearate | Cithrol 4DS series (Croda) | >10 |

In general, mixtures of surfactants are also useful in the present invention, including mixtures of two or more commercial surfactant products. Several PEG-fatty acid esters are marketed commercially as mixtures or mono- and diesters. Representative surfactant mixtures are shown in the following table:

PEG-Fatty Acid Mono-and Diester Mixtures

| Compound | Commercial Product (Supplier) |
|---|---|
| PEG 4-150 mono, | Kessco ® PEG 200-6000 mono, dilaurate (Stepan) |
| dilaurate | |
| PEG 4-150 mono, | Kessco ® PEG 200-6000 mono, dioteate (Stepan) |
| dioleate | |
| PEG 4-150 mono, | Kessco ® 200-6000 mono, distearate (Stepan) |
| distearate | |

Suitable PEG glycerol fatty acid esters are shown in the following table:

PEG Glycerol Fatty Acid Esters

| Compound | Commercial Product (Supplier) | HLB |
|---|---|---|
| PEG-20 glyceryl laurate | Tagat ® L (Goldschmidt) | 16 |
| PEG-30 glyceryl laurate | Tagat ® L2 (Goldschmidt) | 16 |
| PEG-15 glyceryl laurate | Glycerox L series (Croda) | 15 |
| PEG-40 glyceryl laurate | Glycerox L series (Croda) | 15 |
| PEG-20 glyceryl stearate | Capmul ® EMG (ABITEC), | 13 |
| | Aldo ® MS-20 KFG (Lonza) | |
| PEG-20 glyceryl oleate | Tagat ® O (Goldschmidt) | >10 |
| PEG-30 glyceryl oleate | Tagat ® O2 (Goldschmidt) | >10 |

A large number of surfactants of different degrees of lipophilicity or hydrophilicity will desirably be prepared by reaction of alcohols or polyalcohols with a variety of natural and/or hydrogenated oils. In certain embodiments, the oils used are castor oil or hydrogenated castor oil or an edible vegetable oil such as corn oil, olive oil, peanut oil, palm kernel oil, apricot kernel oil, or almond oil. Examples of alcohols include glycerol, propylene glycol, ethylene glycol, polyethylene glycol, sorbitol, and pentaerythritol. Representative surfactants of this class suitable for use in the present invention are shown in the table below:

Transesterification Products of Oils and Alcohols

| Compound | Commercial Product (Supplier) | HLB |
|---|---|---|
| PEG-3 castor oil | Nikkol CO-3 (Nikko) | 3 |
| PEG-5, 9, and 16 castor | ACCONON CA series (ABITEC) | 6-7 |
| oil | | |
| PEG-20 castor oil | Emalex C-20 (Nihon Emulsion), Nikkol CO-20 TX | 11 |
| | (Nikko) | |
| PEG-23 castor oil | Emulgante EL23 | >10 |
| PEG-30 castor oil | Emalex C-30 (Nihon Emulsion), Alkamuls ® EL 620 | 11 |
| | (Rhone-Poulenc), Incrocas 30 (Croda) | |
| PEG-35 castor oil | Cremophor EL and EL-P (BASF), Emulphor EL, | |
| | Incrocas-35 (Croda), Emulgin RO 35 (Henkel) | |
| PEG-38 castor oil | Emulgante EL 65 (Condea) | |
| PEG-40 castor oil | Emalex C-40 (Nihon Emulsion), Alkamuls ® EL 719 | 13 |
| | (Rhone-Poulenc) | |
| PEG-50 castor oil | Emalex C-50 (Nihon Emulsion) | 14 |
| PEG-56 castor oil | Eumulgin ® PRT 56 (Pulcra SA) | >10 |
| PEG-60 castor oil | Nikkol CO-60TX (Nikko) | 14 |
| PEG-100 castor oil | Thornley | >10 |
| PEG-200 castor oil | Eumulgin ® PRT 200 (Pulcra SA) | >10 |
| PEG-5 hydrogenated | Nikkol HCO-5 (Nikko) | 6 |
| castor oil | | |
| PEG-7 hydrogenated | Simusol ® 989 (Seppic), Cremophor WO7 (BASF) | 6 |
| castor oil | | |
| PEG-10 hydrogenated | Nikkol HCO-10 (Nikko) | 6.5 |
| castor oil | | |
| PEG-20 hydrogenated | Nikkol HCO-20 (Nikko) | 11 |
| castor oil | | |
| PEG-25 hydrogenated | Simulsol ® 1292 (Seppic), Cerex ELS 250 (Auschem | 11 |
| castor oil | SpA) | |
| PEG-30 hydrogenated | Nikkol HCO-30 (Nikko) | 11 |
| castor oil | | |
| PEG-40 hydrogenated | Cremophor RH 40 (BASF), Croduret (Croda), | 13 |
| castor oil | Emulgin HRE 40 (Henkel) | |
| PEG-45 hydrogenated | Cerex ELS 450 (Auschem Spa) | 14 |
| castor oil | | |
| PEG-50 hydrogenated | Emalex HC-50 (Nihon Emulsion) | 14 |
| castor oil | | |
| PEG-60 hydrogenated | Nikkol HCO-60 (Nikko), Cremophor RH 60 (BASF) | 15 |
| castor oil | | |
| PEG-80 hydrogenated | Nikkol HCO-80 (Nikko) | 15 |
| castor oil | | |
| PEG-100 hydrogenated | Nikkol HCO-100 (Nikko) | 17 |
| castor oil | | |
| PEG-6 corn oil | Labrafil ® M 2125 CS (Gattefosse) | 4 |
| PEG-6 almond oil | Labrafil ® M 1966 CS (Gattefosse) | 4 |
| PEG-6 apricot kernel oil | Labrafil ® M 1944 CS (Gattefosse) | 4 |
| PEG-6 olive oil | Labrafil ® M 1980 CS (Gattefosse) | 4 |
| PEG-6 peanut oil | Labrafil ® M 1969 CS (Gattefosse) | 4 |
| PEG-6 hydrogenated | Labrafil ® M 2130 BS (Gattefosse) | 4 |
| palm kernel oil | | |
| PEG-6 palm kernel oil | Labrafil ® M 2130 CS (Gattefosse) | 4 |
| PEG-6 triolein | Labrafil ® M 2735 CS (Gattefosse) | 4 |
| PEG-8 corn oil | Labrafil ® WL 2609 BS (Gattefosse) | 6-7 |
| PEG-20 corn glycerides | Crovol M40 (Croda) | 10 |
| PEG-20 almond | Crovol A40 (Croda) | 10 |
| glycerides | | |
| PEG-25 trioleate | TAGAT ® TO (Goldschmidt) | 11 |
| PEG-40 palm kernel oil | Crovol PK-70 | >10 |
| PEG-60 corn glycerides | Crovol M70(Croda) | 15 |
| PEG-60 almond | Crovol A70 (Croda) | 15 |
| glycerides | | |
| PEG-4 caprylic/capric | Labrafac ® Hydro (Gattefosse), | 4-5 |
| triglyceride | | |
| PEG-8 caprylic/capric | Labrasol (Gattefosse), Labrafac CM 10 (Gattefosse) | >10 |
| glycerides | | |
| PEG-6 caprylic/capric | SOFTIGEN ® 767 (Huls), Glycerox 767 (Croda) | 19 |
| glycerides | | |
| Lauroyl macrogol-32 | GELUCIRE 44/14 (Gattefosse) | 14 |
| glyceride | | |
| Stearoyl macrogol | GELUCIRE 50/13 (Gattefosse) | 13 |
| glyceride | | |
| Mono, di, tri, tetra esters | SorbitoGlyceride (Gattefosse) | <10 |
| of vegetable oils and | | |
| sorbitol | | |
| Pentaerythrityl | Crodamol PTIS (Croda) | <10 |
| tetraisostearate | | |
| Pentaerythrityl distearate | Albunol DS (Taiwan Surf.) | <10 |
| Pentaerythrityl | Liponate PO-4 (Lipo Chem.) | <10 |
| tetraoleate | | |
| Pentaerythrityl | Liponate PS-4 (Lipo Chem.) | <10 |
| tetrastearate | | |
| Pentaerythrityl | Liponate PE-810 (Lipo Chem.), Crodamol PTC | <10 |
| tetracaprylate/ | (Croda) | |
| tetracaprate | | |
| Pentaerythrityl | Nikkol Pentarate 408 (Nikko) | |
| tetraoctanoate | | |

Polyglycerol esters of fatty acids are also suitable surfactants for the present invention. Examples of suitable polyglyceryl esters are shown in the following table:

Polyglycerized Fatty Acids

| Compound | Commercial Product (Supplier) | HLB |
|---|---|---|
| Polyglyceryl-2 stearate | Nikkol DGMS (Nikko) | 5-7 |
| Polyglyceryl-2 oleate | Nikkol DGMO (Nikko) | 5-7 |
| Polyglyceryl-2 | Nikkol DGMIS (Nikko) | 5-7 |
| isostearate | | |
| Polyglyceryl-3 oleate | Caprol ® 3G0 (ABITEC), | 6.5 |
| | Drewpol 3-1-O (Stepan) | |
| Polyglyceryl-4 oleate | Nikkol Tetraglyn 1-O (Nikko) | 5-7 |
| Polyglyceryl-4 stearate | Nikkol Tetraglyn 1-S (Nikko) | 5-6 |
| Polyglyceryl-6 oleate | Drewpol 6-1-O (Stepan), Nikkol | 9 |
| | Hexaglyn 1-O (Nikko) | |
| Polyglyceryl-10 laurate | Nikkol Decaglyn 1-L (Nikko) | 15 |
| Polyglyceryl-10 oleate | Nikkol Decaglyn 1-O (Nikko) | 14 |
| Polyglyceryl-10 stearate | Nikkol Decaglyn 1-S (Nikko) | 12 |
| Polyglyceryl-6 | Nikkol Hexaglyn PR-15 (Nikko) | |
| ricinoleate | | |
| Polyglyceryl-10 linoleate | Nikkol Decaglyn I-LN (Nikko) | 12 |
| Polyglyceryl-6 | Nikkol Hexaglyn S-O (Nikko) | <10 |
| pentaoleate | | |
| Polyglyceryl-3 dioleate | Cremophor G032 (BASF) | <10 |
| Polyglyceryl-3 distearate | Cremophor GS32 (BASF) | <10 |
| Polyglyceryl-4 | Nikkol Tetraglyn 5-O (Nikko) | <10 |
| pentaoleate | | |
| Polyglyceryl-6 dioleate | Caprol ® 6G20 (ABITEC); | 8.5 |
| | Hodag PGO-62 (Calgene), PLUROL | |
| | OLEIQUE CC 497 (Gattefosse) | |
| Polyglyceryl-2 dioleate | Nikkol DGDO (Nikko) | 7 |
| Polyglyceryl-10 trioleate | Nikkol Decaglyn 3-O (Nikko) | 7 |
| Polyglyceryl-10 | Nikkol Decaglyn 5-O (Nikko) | 3.5 |
| pentaoleate | | |
| Polyglyceryl-10 | Nikkol Decagtyn 7-O (Nikko) | 3 |
| septaoleate | | |
| Polyglyceryl-10 | Caprol ® 10G40 (ABITEC); | 6.2 |
| tetraoleate | Hodag PGO-62 (CALGENE), | |
| | Drewpol 10-4-O (Stepan) | |
| Polyglyceryl-10 | Nikkol Decaglyn 10-IS (Nikko) | <10 |
| decaisostearate | | |
| Polyglyceryl-10 | Drewpol 10-10-O (Stepan), | 3.5 |
| decaoleate | Caprol 10G10O (ABITEC), | |
| | Nikkol Decaglyn 10-O | |
| Polyglyceryl-10 mono, | Caprol ® PGE 860 (ABITEC) | 11 |
| dioleate | | |
| Polyglyceryl | Polymuls (Henkel) | 3-20 |
| polyricinoIeate | | |

Esters of propylene glycol and fatty acids are suitable surfactants for use in the present invention. Examples of surfactants of this class are given in the following table:

Propylene Glycol Fatty Acid Esters

| Compound | Commercial Product (Supplier) | HLB |
|---|---|---|
| Propylene glycol | Capryol 90 (Gattefosse), | <10 |
| monocaprylate | Nikkol Sefsol 218 (Nikko) | |
| Propylene glycol | Lauroglycol 90 (Gattefosse), | <10 |
| monolaurate | Lauroglycol FCC (Gattefosse) | |
| Propylene glycol oleate | Lutrol OP2000 (BASF) | <10 |
| Propylene glycol | Mirpyl | <10 |
| myristate | | |
| Propylene glycol | ADM PGME-03 (ADM), | 3-4 |
| monostearate | LIPO PGMS (Lipo Chem.), | |
| | Aldo ® PGHMS (Lonza) | |
| Propylene glycol | | <10 |
| hydroxy stearate | | |
| Propylene glycol | PROPYMULS (Henkel) | <10 |
| ricinoleate | | |
| Propylene glycol | | <10 |
| isostearate | | |
| Propylene glycol | Myverol P-06 (Eastman) | <10 |
| monooleate | | |
| Propylene glycol | Captex ® 200 (ABITEC), | >6 |
| dicaprylate/dicaprate | Miglyol ® 840 (Huls), | |
| Propylene glycol | Neobee ® M-20 (Stepan) | |
| dioctanoate | Captex ® 800 (ABITEC) | |
| Propylene glycol | LABRAFAC | >6 |
| caprylate/caprate | PG (Gattefosse) | |
| Propylene glycol | | >6 |
| dilaurate | | |
| Propylene glycol | Kessco ® PGDS (Stepan) | >6 |
| distearate | | |
| Propylene glycol | Nikkol Sefsol 228 (Nikko) | >6 |
| dicaprylate | | |
| Propylene glycol | Nikkol PDD (Nikko) | >6 |
| dicaprate | | |

In general, mixtures of surfactants are also suitable for use in the present invention. In particular, mixtures of propylene glycol fatty acid esters and glycerol fatty acid esters are suitable and are commercially available. Examples of these surfactants are shown in the table below:

Glycerol/Propylene Glycol Fatty Acid Esters

| Compound | Commercial Product (Supplier) | HLB |
|---|---|---|
| Oleic | ATMOS 300, ARLACEL 186 (ICI) | 3-4 |
| Stearic | ATMOS 150 | 3-4 |

Another class of surfactants is the class of mono- and diglycerides. These surfactants are generally lipophilic. Examples of these surfactants are given in the table below:

Mono- and Diglyceride Surfactants

| Compound | Commercial Product (Supplier) | HLB |
|---|---|---|
| Monopalmitolein | (Larodan) | <10 |
| (C16:1) | | |
| Monoelaidin | (Larodan) | <10 |
| (C18:1) | | |
| Monocaproin | (Larodan) | <10 |
| (C6) | | |
| Monocaprylin | (Larodan) | <10 |
| Monocaprin | (Larodan) | <10 |
| Monolaurin | (Larodan) | <10 |
| Glyceryl monomyristate | Nikkol MGM (Nikko) | 3-4 |
| (C14) | | |
| Glyceryl monooleate | PECEOL (Gattefosse), Hodag GMO-D, Nikkol MGO | 3-4 |
| (C18:1) | (Nikko) | |
| Glyceryl monooleate | RYLO series (Danisco), DIMODAN series (Danisco), | 3-4 |
| | EMULDAN (Danisco), ALDO ® MO FG (Lonza), | |
| | Kessco GMO (Stepan), MONOMULS ® series | |
| | (Henkel), TEGIN O, DREWMULSE GMO (Stepan), | |
| | Atlas G-695 (ICI), GMOrphic 80 (Eastman), ADM | |
| | DMG-40, 70, and 100 (ADM), Myverol (Eastman) | |
| Glycerol monooleate/ | OLICINE (Gattefosse) | 3-4 |
| linoleate | | |
| Glycerol monolinoleate | Maisine (Gattefosse), MYVEROL 18-92, Myverol 18- | 3-4 |
| | 06 (Eastman) | |
| Glyceryl ricinoleate | Softigen ® 701 (Huls), HODAG GMR-D (Calgene), | 6 |
| | ALDO ® MR (Lonza) | |
| Glyceryl monolaurate | ALDO ® MLD (Lonza), Hodag GML (Calgene) | 6.8 |
| Glycerol monopalmitate | Emalex GMS-P (Nihon) | 4 |
| Glycerol monostearate | Capmul ® GMS. (ABITEC), Myvaplex (Eastman), | 5-9 |
| | IMWITOR ® 191 (Huls), CUTINA GMS, Aldo ® MS | |
| | (Lonza), Nikkol MGS series (Nikko) | |
| Glyceryl mono-, dioleate | Capmul ® GMO-K (ABITEC) | <10 |
| Glyceryl palmitic/stearic | CUTINA MD-A, ESTAGEL-G18 | <10 |
| Glyceryl acetate | Lamegin ® EE (Grunau GmbH) | <10 |
| Glyceryl laurate | Inwitor ® 312 (Huls), Monomuls ® 90-45 (Grunau | 4 |
| | GmbH), Aldo ® MLD (Lonza) | |
| Glyceryl citrate/ | Imwitor ® 375 (Huls) | <10 |
| lactate/oleate/linoieate | | |
| Glyceryl caprylate | Imwitor ® 308 (Huls), Capmul ® MCMC8 (ABITEC) | 5-6 |
| Glyceryl | Capmul ® MCM (ABITEC) | 5-6 |
| caprylate/caprate | | |
| Caprylic acid mono, | Imwitor ® 988 (Huls) | 5-6 |
| diglycerides | | |
| Caprylic/capric | Imwitor ® 742 (Huls) | <10 |
| glycerides | | |
| Mono-and diacetylated | Myvacet ® 9-45, Myvacet ® 9-40, Myvacet ® 9-08 | 3.8-4 |
| monoglycerides | (Eastman), Lamegin ® (Grunau) | |
| Glyceryl monostearate | Aldo ® MS, Arlacel 129 (ICI), LIPO GMS (Lipo | 4.4 |
| | Chem.), Imwitor ® 191 (Huls), Myvaplex (Eastman) | |
| Lactic acid esters of | LAMEGIN GLP (Henkel) | <10 |
| mono, diglycerides | | |
| Dicaproin (C6) | (Larodan) | <10 |
| Dicaprin (C10) | (Larodan) | <10 |
| Dioctanoin (C8) | (Larodan) | <10 |
| Dimyristin (C14) | (Larodan) | <10 |
| Dipalmitin (C16) | (Larodan) | |
| Distearin | (Larodan) | <10 |
| Glyceryl dilaurate (C12) | Capmul ® GDL (ABITEC) | 3-4 |
| Glyceryl dioleate | Capmul ® GDO (ABITEC) | 3-4 |
| Glycerol esters of fatty | GELUCIRE 39/01 (Gattefosse), | 1 |
| acids | GELUCIRE 43/01 (Gattefosse) | 6 |
| | GELUCIRE 37/06 (Gattefosse) | |
| Dipalmitolein (C16:1) 1, | (Larodan) | <10 |
| 2 and 1,3-diolein | | |
| (C18:1) | | |
| Dielaidin (C18:1) | (Larodan) | <10 |
| Dilinolein (C18:2) | (Larodan) | <10 |

Sterols and derivatives of sterols are suitable surfactants for use in the present invention. These surfactants will desirably be hydrophilic or lipophilic. Examples of surfactants of this class are shown in the table below:

Sterol and Sterol Derivative Surfactants

| Compound | Commercial Product (Supplier) | HLB |
|---|---|---|
| Cholesterol, sitosterol, | | <10 |
| lanosterol | | |
| PEG-24 cholesterol ether | Solulan C-24 (Amerchol) | >10 |
| PEG-30 cholestanol | Nikkol DHC (Nikko) | >10 |
| Phytosterol | GENEROL series (Henkel) | <10 |
| PEG-25 phyto sterol | Nikkol BPSH-25 (Nikko) | >10 |
| PEG-5 soya sterol | Nikkol BPS-S (Nikko) | <10 |
| PEG-10 soya sterol | Nikkol BPS-10 (Nikko) | <10 |
| PEG-20 soya sterol | Nikkol BPS-20 (Nikko) | <10 |
| PEG-30 soya sterol | Nikkol BPS-30 (Nikko) | >10 |

A variety of PEG-sorbitan fatty acid esters are available and are suitable for use as surfactants in the present invention. In general, these surfactants are hydrophilic, although several lipophilic surfactants of this class will desirably be used. Examples of these surfactants are shown in the table below:

PEG-Sorbitan Fatty Acid Esters

| Compound | Commercial Product (Supplier) | HLB |
|---|---|---|
| PEG-10 sorbitan | Liposorb L-10 (Lipo Chem.) | >10 |
| laurate | | |
| PEG-20 sorbitan | Tween-20 (Atlas/ICI), Crillet 1 (Croda), | 17 |
| monolaurate | DACOL MLS 20 (Condea) | |
| PEG-4 sorbitan | Tween-21 (Atlas/ICI), Crillet 11 (Croda) | 13 |
| monolaurate | | |
| PEG-80 sorbitan | Hodag PSML-80 (Calgene); T-Maz 28 | >10 |
| monolaurate | | |
| PEG-6 sorbitan | Nikkol GL-1 (Nikko) | 16 |
| monolaurate | | |
| PEG-20 sorbitan | Tween-40 (Atlas/ICI), Crillet 2 (Croda) | 16 |
| monopalmitate | | |
| PEG-20 sorbitan | Tween-60 (Atlas/ICI), Crillet 3 (Croda) | 15 |
| monostearate | | |
| PEG-4 sorbitan | Tween-61 (Atlas/ICI), Crillet 31 (Croda) | 9.6 |
| monostearate | | |
| PEG-8 sorbitan | DACOL MSS (Condea) | >10 |
| monostearate | | |
| PBG-6 sorbitan | Nikkol TS106 (Nikko) | 11 |
| monostearate | | |
| PEG-20 sorbitan | Tween-65 (Atlas/ICI), Crillet 35 (Croda) | 11 |
| tristearate | | |
| PEG-6 sorbitan | Nikkol GS-6 (Nikko) | 3 |
| tetrastearate | | |
| PEG-60 sorbitan | Nikkol GS-460 (Nikko) | 13 |
| tetrastearate | | |
| PEG-5 sorbitan | Tween-81 (Atlas/ICI), Crillet 41 (Croda) | 10 |
| monooleate | | |
| PEG-6 sorbitan | Nikkol TO-106 (Nikko) | 10 |
| monooleate | | |
| PEG-20 sorbitan | Tween-80 (Atlas/ICI), Crillet 4 (Croda) | 15 |
| monooleate | | |
| PEG-40 sorbitan oleate | Emalex ET 8040, (Nihon Emulsion) | 18 |
| PEG-20 sorbitan | Tween-85 (Atlas/ICI), Crillet 45 (Croda) | 11 |
| trioleate | | |
| PEG-6 sorbitan | Nikkol GO-4 (Nikko) | 8.5 |
| tetraoleate | | |
| PEG-30 sorbitan | Nikkol GO-430 (Nikko) | 12 |
| tetraoleate | | |
| PEG-40 sorbitan | Nikkol GO-440 (Nikko) | 13 |
| tetraoleate | | |
| PEG-20 sorbitan | Tween-120 (Atlas/ICI), Crillet 6 (Croda) | >10 |
| monoisostearate | | |
| PEG sorbitol | Atlas G-1086 (ICI) | 10 |
| hexaoleate | | |
| PEG-6 sorbitol | Nikkol GS-6 (Nikko) | 3 |
| hexastearate | | |

Ethers of polyethylene glycol and alkyl alcohols are suitable surfactants for use in the present invention. Examples of these surfactants are shown in the table below:

Polyethylene Glycol Alkyl Ethers

| Compound | Commercial Product (Supplier) | HLB |
|---|---|---|
| PEG-2 oleyl ether, oleth-2 | Brij 92/93 (Atlas/ICI) | 4.9 |
| PEG-3 oleyl ether, oleth-3 | Volpo 3 (Croda) | <10 |
| PEG-5 oleyl ether, oleth-5 | Volpo 5 (Croda) | <10 |
| PEG-10 oleyl ether, | Volpo 10 (Croda), Brij 96/97 | 12 |
| oleth-10 | (Atlas/ICI) | |
| PEG-20 oleyl ether, | Volpo 20 (Croda), Brij 98/99 | 15 |
| oleth-20 | (Atlas/ICI) | |
| PEG-4 lauryl ether, | Brij 30 (Atlas/ICI) | 9.7 |
| laureth-4 | | |
| PEG-9 lauryl ether | | >10 |
| PEG-23 lauryl ether, | Brij 35 (Atlas/ICI) | 17 |
| laureth-23 | | |
| PEG-2 cetyl ether | Brij 52 (ICI) | 5.3 |
| PEG-10 cetyl ether | Brij 56 (ICI) | 13 |
| PEG-20 cetyl ether | Brij 58 (ICI) | 16 |
| PEG-2 stearyl ether | Brij 72 (ICI) | 4.9 |
| PEG-10 stearyl ether | Brij 76 (ICI) | 12 |
| PEG-20 stearyl ether | Brij 78 (ICI) | 15 |
| PEG-100 stearyl ether | Brij 700 (ICI) | >10 |

Esters of sugars are suitable surfactants for use in the present invention. Examples of such surfactants are shown in the table below:

Sugar Ester Surfactants

| Compound | Commercial Product (Supplier) | HLB |
|---|---|---|
| Sucrose distearate | SUCRO ESTER 7 (Gattefosse), | 3 |
| | Crodesta F-10 (Croda) | |
| Sucrose distearate/ | SUCRO ESTER 11 (Gattefosse), | 12 |
| monostearate | Crodesta F-110 (Croda) | |
| Sucrose dipalmitate | | 7.4 |
| Sucrose monostearate | Crodesta F-160 (Croda) | 15 |
| Sucrose monopalmitate | SUCRO ESTER 15 (Gattefosse) | >10 |
| Sucrose monolaurate | Saccharose monolaurate 1695 | 15 |
| | (Mitsubishi-Kasei) | |

Several hydrophilic PEG-alkyl phenol surfactants are available, and are suitable for use in the present invention. Examples of these surfactants are shown in the table below:

Polyethylene Glycol Alkyl Phenol Surfactants

| Compound | Commercial Product (Supplier) | HLB |
|---|---|---|
| PEG-10-100 | Triton X series (Rohm & Haas), Igepal CA series | >10 |
| nonyl phenol | (GAF, USA), Antarox CA series (GAF, UK) | |
| PEG-15-100 | Triton N-series (Rohm & Haas), Igepal CO series | >10 |
| octyl phenol | (GAF, USA), Antarox CO series (GAF, UK) | |
| ether | | |

The POE-POP block copolymers are a unique class of polymeric surfactants. The unique structure of the surfactants, with hydrophilic POE and lipophilic POP moieties in well-defined ratios and positions, provides a wide variety of surfactants suitable for use in the present invention. These surfactants are available under various trade names, including Synperonic™ PE series (ICI); Pluronic® series (BASF), Emkalyx™, Lutrol™ (BASF), Supronic™ Monolan™, Pluracare™, and Plurodac™. The generic term for these polymers is “poloxamer” (CAS 9003-11-6). These polymers have the formula:
HO(C₂H₄O)ₐ(C₃H₆O)_{b}(C₂H₄O)ₐH

where “a” and “b” denote the number of polyoxyethylene and polyoxypropylene units, respectively. Examples of suitable surfactants of this class are shown in the table below:

POE-POP Block Copolymers

| | | |
|---|---|---|
| | a, b values in | |
| Compound | HO(C₂H₄O)ₐ (C₃H₆O)_{b} (C₂H₄O)ₐH | HLB |

| | | | |
|---|---|---|---|
| Poloxamer 105 | a = 11 | b = 16 | 8 |
| Poloxamer 108 | a = 46 | b = 16 | >10 |
| Poloxamer 122 | a = 5 | b = 21 | 3 |
| Poloxamer 123 | a = 7 | b = 21 | 7 |
| Poloxamer 124 | a = 11 | b = 21 | >7 |
| Poloxamer 181 | a = 3 | b = 30 | |
| Poloxamer 182 | a = 8 | b = 30 | 2 |
| Poloxamer 183 | a = 10 | b = 30 | |
| Poloxamer 184 | a = 13 | b = 30 | |
| Poloxamer 185 | a = 19 | b = 30 | |
| Poloxamer 188 | a = 75 | b = 30 | 29 |
| Poloxamer 212 | a = 8 | b = 35 | |
| Poloxamer 215 | a = 24 | b = 35 | |
| Poloxamer 217 | a = 52 | b = 35 | |
| Poloxamer 231 | a = 16 | b = 39 | |
| Poloxamer 234 | a = 22 | b = 39 | |
| Poloxamer 235 | a = 27 | b = 39 | |
| Poloxamer 237 | a = 62 | b = 39 | 24 |
| Poloxamer 238 | a = 97 | b = 39 | |
| Poloxamer 282 | a = 10 | b = 47 | |
| Poloxamer 284 | a = 21 | b = 47 | |
| Poloxamer 288 | a = 122 | b = 47 | >10 |
| Poloxamer 331 | a = 7 | b = 54 | 0.5 |
| Poloxamer 333 | a = 20 | b = 54 | |
| Poloxamer 334 | a = 31 | b = 54 | |
| Poloxamer 335 | a = 38 | b = 54 | |
| Poloxamer 338 | a = 128 | b = 54 | |
| Poloxamer 401 | a = 6 | b = 67 | |
| Poloxamer 402 | a = 13 | b = 67 | |
| Poloxamer 403 | a = 21 | b = 67 | |
| Poloxamer 407 | a = 98 | b = 67 | |

Sorbitan esters of fatty acids are suitable surfactants for use in the present invention. Examples of these surfactants are shown in the table below:

Sorbitan Fatty Acid Ester Surfactants

| Compound | Commercial Product (Supplier) | HLB |
|---|---|---|
| Sorbitan monolaurate | Span-20 (Atlas/ICI), Crill 1 (Croda), | 8.6 |
| | Arlacel 20 (ICI) | |
| Sorbitan monopalmitate | Span-40 (Atlas/ICI), Crill 2 (Croda), | 6.7 |
| | Nikkol SP-10 (Nikko) | |
| Sorbitan monooleate | Span-80 (Atlas/ICI), Crill 4 (Croda), | 4.3 |
| | Crill 50 (Croda) | |
| Sorbitan monostearate | Span-60 (Atlas/ICI), Crill 3 (Croda), | 4.7 |
| | Nikkol SS-10 (Nikko) | |
| Sorbitan trioleate | Span-85 (Atlas/ICI), Crill 45 (Croda), | 4.3 |
| | Nikkol SO-30 (Nikko) | |
| Sorbitan sesquioleate | Arlacel-C (ICI), Crill 43 (Croda), | 3.7 |
| | Nikkol SO-15 (Nikko) | |
| Sorbitan tristearate | Span-65 (Atlas/ICI) Crill 35 (Croda), | 2.1 |
| | Nikkol SS-30 (Nikko) | |
| Sorbitan monoisostearate | Crill 6 (Croda), Nikkol SI-10 (Nikko) | 4.7 |
| Sorbitan sesquistearate | Nikkol SS-15 (Nikko) | 4.2 |

Esters of lower alcohols (C2 to C4) and fatty acids (C8 to C18) are suitable surfactants for use in the present invention. Examples of these surfactants are shown in the table below:

Lower Alcohol Fatty Acid Ester Surfactants

| Compound | Commercial Product (Supplier) | HLB |
|---|---|---|
| Ethyl oleate | Crodamol EO (Croda), Nikkol EOO (Nikko) | <10 |
| Isopropyl myristate | Crodamol IPM (Croda) | <10 |
| Isopropyl palmitate | Crodamol IPP (Croda) | <10 |
| Ethyl linoleate | Nikkol VF-E (Nikko) | <10 |
| Isopropyl linoleate | Nikkol VF-IP (Nikko) | <10 |

Ionic surfactants, including cationic, anionic and zwitterionic surfactants, are suitable hydrophilic surfactants for use in the present invention. In certain embodiments, the surfactant is an anionic surfactant such as a fatty acid salt, a bile salt, or a combination thereof. In other embodiments the surfactant is a cationic surfactant such as a carnitine. Examples of ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. Examples of such surfactants are shown in the table below:

Ionic Surfactants

| Compound | HLB |
|---|---|
| FATTY ACID SALTS | >10 |
| Sodium caproate | |
| Sodium caprylate | |
| Sodium caprate | |
| Sodium laurate | |
| Sodium myristate | |
| Sodium myristolate | |
| Sodium palmitate | |
| Sodium palmitoleate | |
| Sodium oleate | 18 |
| Sodium ricinoleate | |
| Sodium linoleate | |
| Sodium linolenate | |
| Sodium stearate | |
| Sodium lauryl sulfate (dodecyl) | 40 |
| Sodium tetradecyl sulfate | |
| Sodium lauryl sarcosinate | |
| Sodium dioctyl sulfosuccinate [sodium docusate (Cytec)] | |
| BILE SALTS | >10 |
| Sodium cholate | |
| Sodium taurocholate | |
| Sodium glycocholate | |
| Sodium deoxycholate | |
| Sodium taurodeoxycholate | |
| Sodium glycodeoxycholate | |
| Sodium ursodeoxycholate | |
| Sodium chenodeoxycholate | |
| Sodium taurochenodeoxycholate | |
| Sodium glycol cheno deoxycholate | |
| Sodium cholylsarcosinate | |
| Sodium N-methyl taurocholate | |
| Sodium lithocholate | |
| PHOSPHOLIPIDS | |
| Egg/Soy lecithin [Epikuron ™ (Lucas Meyer), | |
| Ovothin ™ (Lucas Meyer)] | |
| Lyso egg/soy lecithin | |
| Hydroxylated lecithin | |
| Lysophosphatidylcholine | |
| Cardiolipin | |
| Sphingomyelin | |
| Phosphatidylcholine | |
| Phosphatidyl ethanolamine | |
| Phosphatidic acid | |
| Phosphatidyl glycerol | |
| Phosphatidyl serine | |
| PHOSPHORIC ACID ESTERS | |
| Diethanolammonium polyoxyethylene-10 oleyl ether phosphate | |
| Esterification products of fatty alcohols or fatty alcohol | |
| ethoxylates with phosphoric acid or anhydride | |
| CARBOXYLATES | |
| Ether carboxylates (by oxidation of terminal OH group | |
| of fatty alcohol ethoxylates) | |
| Succinylated monoglycerides [LAMEGIN ZE (Henkel)] | |
| Sodium stearyl fumarate | |
| Stearoyl propylene glycol hydrogen succinate | |
| Mono/diacetylated tartaric acid esters of mono- and diglycerides | |
| Citric acid esters of mono-, diglycerides | |
| Glyceryl-lacto esters of fatty acids (CFR ref. 172.852) | |
| Acyl lactylates: | |
| lactylic esters of fatty acids calcium/sodium | |
| stearoyl-2-lactylate calcium/sodium | |
| stearoyl lactylate | |
| Alginate salts | |
| Propylene glycol alginate | |
| SULFATES AND SULFONATES | |
| Ethoxylated alkyl sulfates | |
| Alkyl benzene sulfones | |
| α-olefin sulfonates | |
| Acyl isethionates | |
| Acyl taurates | |
| Alkyl glyceryl ether sulfonates | |
| Octyl sulfosuccinate disodium | |
| Disodium undecylenamideo-MEA-sulfosuccinate | |
| CATIONIC Surfactants | >10 |
| Lauroyl carnitine | |
| Palmitoyl carnitine | |
| Myristoyl carnitine | |
| Hexadecyl triammonium bromide | |
| Decyl trimethyl ammonium bromide | |
| Cetyl trimethyl ammonium bromide | |
| Dodecyl ammonium chloride | |
| Alkyl benzyldimethylammonium salts | |
| Diisobutyl phenoxyethoxydimethyl benzylammonium salts | |
| Alkylpyridinium salts | |
| Betaines (trialkylglycine): | |
| Lauryl betaine (N-lauryl,N,N-dimethylglycine) | |
| Ethoxylated amines: | |
| Polyoxyethylene-15 coconut amine | |

For simplicity, typical counterions are shown in the entries in the table. It will be appreciated by one skilled in the art, however, that any bioacceptable counterion will desirably be used. For example, although the fatty acids are shown as sodium salts, other cation counterions will desirably also be used, such as alkali metal cations or ammonium. Unlike typical non-ionic surfactants, these ionic surfactants are generally available as pure compounds, rather than commercial (proprietary) mixtures. Because these compounds are readily available from a variety of commercial suppliers, such as Aldrich, Sigma, and the like, commercial sources are not generally listed in the table.

Derivatives of oil-soluble vitamins, such as vitamins A, D, E, K, etc., are also useful surfactants for the compositions of the present invention. An example of such a derivative is tocopheryl PEG-1000 succinate (TPGS, available from Eastman).

In certain embodiments, surfactants or mixtures of surfactants that solidify at ambient room temperature are used. In other embodiments, surfactants or mixtures of surfactants that solidify at ambient room temperature in combination with particular lipophilic components, such as triglycerides, or with addition of appropriate additives, such as viscosity modifiers, binders, thickeners, and the like, are used.

Examples of non-ionic hydrophilic surfactants include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated vegetable oils, and sterols; sugar esters, sugar ethers; sucroglycerides; polyethoxylated fat-soluble vitamins or derivatives; and mixtures thereof.

In certain embodiments, the non-ionic hydrophilic surfactant is selected from the group consisting of polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglyceryl fatty acid esters; polyoxyethylene glycerides; polyoxyethylene vegetable oils; and polyoxyethylene hydrogenated vegetable oils. The glyceride will desirably be a monoglyceride, diglyceride, triglyceride, or a mixture thereof.

In certain other embodiments, the surfactants used are non-ionic hydrophilic surfactants that are reaction mixtures of polyols and fatty acids, glycerides, vegetable oils, hydrogenated vegetable oils or sterols. These reaction mixtures are largely composed of the transesterification products of the reaction, along with often complex mixtures of other reaction products. The polyol will desirably be glycerol, ethylene glycol, polyethylene glycol, sorbitol, propylene glycol, pentaerythritol, a saccharide, or a mixture thereof.

The hydrophilic surfactant will desirably also be, or include as a component, an ionic surfactant. Examples of ionic surfactants include alkyl ammonium salts; bile acids and salts, analogues, and derivatives thereof; fusidic acid and derivatives thereof; fatty acid derivatives of amino acids, oligopeptides, and polypeptides; glyceride derivatives of amino acids, oligopeptides, and polypeptides; acyl lactylates; mono-diacetylated tartaric acid esters of mono-diglycerides; succinylated monoglycerides; citric acid esters of mono-diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof.

In certain embodiments the ionic surfactants include bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono-diacetylated tartaric acid esters of mono-diglycerides; succinylated monoglycerides; citric acid esters of mono-diglycerides; carnitines; and mixtures thereof.

Examples of ionic surfactants include lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

In certain embodiments, ionic surfactants used include lecithin, lysolecithin, phosphatidylcholine, phosphatidylethanolamine, phosphatidylglycerol, lysophosphatidylcholine, PEG-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, glycodeoxycholate, cholylsarcosine, caproate, caprylate, caprate, laurate, oleate, lauryl sulfate, docusate, and salts and mixtures thereof. In at least one embodiment, the ionic surfactant is selected from lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

Examples of lipophilic surfactants include alcohols; polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; and mixtures thereof.

As with the hydrophilic surfactants, lipophilic surfactants will desirably be reaction mixtures of polyols and fatty acids, glycerides, vegetable oils, hydrogenated vegetable oils, and sterols.

In certain embodiments, the lipophilic surfactants include one or more selected from the group consisting of fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid derivatives of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, sterols, and mixtures thereof.

In certain other embodiments, the lipophilic surfactants include one or more selected from the group consisting of lower alcohol fatty acids esters; polypropylene glycol fatty acid esters; propylene glycol fatty acid esters; glycerol fatty acid esters; acetylated glycerol fatty acid esters; lactic acid derivatives of mono/diglycerides; sorbitan fatty acid esters; polyoxyethylene vegetable oils; and mixtures thereof. Among the glycerol fatty acid esters, the esters will desirably be mono- or diglycerides, or mixtures of mono- and diglycerides, where the fatty acid moiety is a C6 to C22 fatty acid.

Other embodiments include lipophilic surfactants which are the reaction mixture of polyols and fatty acids, glycerides, vegetable oils, hydrogenated vegetable oils, and sterols. Examples of polyols are polyethylene glycol, sorbitol, propylene glycol, pentaerythritol, and mixtures thereof.

Combinations of solubility enhancers (i.e. surfactants) will desirably be used. Examples of macrogol fatty acid esters useful as solubility enhancers include Gelucire 50/13® and Gelucire 44/14®. In at least one embodiment the solubility enhancer is Gelucire 50/13®. The solubility enhancer will desirably be present in an amount of from 0.1% to 70% by weight of the microparticle. For example, in certain embodiments, the solubility enhancer is present in an amount of from 1% to 50%; in other embodiments from 10% to 30%; in still other embodiments from 15% to 25% by weight of the microparticle. In at least one embodiment the solubility enhancer is present in an amount of 20% by weight of the microparticle.

It is contemplated that in some embodiments, one or more other pharmaceutically acceptable excipients consistent with the objects of the present invention will desirably be used in the microparticles, such as a lubricant, a binder, a pH modifier, a filler and/or a glidant.

The process for manufacturing the drug-containing microparticles of the present invention by spheronization are not limited to the CEFORM™ technology, and any other technology resulting in the formation of the microparticles consistent with the objects of the present invention will desirably also be used. For example, microparticles of the invention will desirably also be manufactured by extrusion/spheronization, granulation or pelletization.

Extrusion/spheronization is a multi-step process used to make uniformly sized spherical particles. The technique offers the ability to incorporate high levels of active ingredients without producing excessively large particles. The main steps in the process are:
(i) Dry-mixing of ingredients to achieve a homogenous powder dispersion;
(ii) Wet massing using for example a high-shear wet granulator to form rod shaped particles of uniform diameter;
(iii) Extrusion to form rod-shaped particles of uniform diameter;
(iv) Spheronization to round off the rods into spherical particles;
(v) Screening to achieve the desired narrow particle size distribution.

The mixing vessel used for dry-mixing will desirably be of any size and shape compatible with the size of the formulation to be produced. For example, commercially available mixing devices such as planetary mixers, high shear mixers, or twin cone blenders will desirably be used. If relatively small quantities of formulation are to be prepared, a simple mortar and pestle will desirably be sufficient to mix the ingredients. The type of mixing vessel would be apparent to one skilled in the pharmaceutical art. The moistened mass formed by wet-massing in conventional granulation equipment is extruded through a perforated mesh in order to produce cylindrical filaments. The port of the meshes will desirably determine the diameter of the filaments. A port ranging from 0.2 mm to 3 mm will desirably be used in this process. In at least one embodiment utilizing this process, the port ranges from 0.4 mm to 2 mm. The extrusion will desirably be carried out using screw, double screw, “sieve and basket” kind, “roll extruder”, “ram extruder” extruders or any other pharmaceutically acceptable means to produce cylindrical filaments. In certain embodiments utilizing this extrusion/spheronization process, a double screw coaxial extruder is used. The spheronization device comprises a hollow cylinder with a horizontal rotating plate. The filaments are broken in short segments, which are transformed in spherical or quasi-spherical particles on the upper surface of the rotating plate at a velocity ranging from 200 rpm to 2,000 rpm. The particles will desirably be dried in any pharmaceutically acceptable way, such as for example by air drying in a static condition. The particles are used as they are or they are coated to obtain granules to use in tablets, capsules, packets or other pharmaceutical formulations.

A prophetic example of an extrusion/spheronization formulation comprising tramadol hydrochloride will desirably be as follows: In this example, the tramadol hydrochloride will desirably be present in an amount of from 1% to 80% w/w. In certain embodiments within this example, the tramadol hydrochloride is present in an amount of from 1% to 50% w/w; in other embodiments from 10% to 30%; and in still other embodiments 10% w/w. In this example, the filler will desirably be present in an amount of from 0% to 80% w/w. In certain embodiments of this example, the filler is present in an amount of from 10% to 60%; and in other embodiments at 40% w/w. In this example, the microcrystalline cellulose will desirably be present in an amount of from 10% to 90% w/w. In certain embodiments of this example, the microcrystalline cellulose is present in an amount of from 10% to 70%; and in other embodiments from 20% to 50% w/w. In this example, the binder will desirably be present in an amount of from 0% to 10% w/w. In certain embodiments of this example, the binder is present in an amount of from 1% to 8%; and in other embodiments from 2% to 4% w/w. In this example, water will desirably be present in an amount of from 10% to 80% w/w. In certain embodiments of this example, water is present in an amount of from 15% to 70%; and in other embodiments from 20% to 50% w/w. Suitable fillers in this example include but are not limited to calcium phosphate dibasic, tricalcium phosphate, calcium carbonate, starch (such as corn, maize, potato and rice starches), modified starches (such as carboxymethyl starch, etc.), microcrystalline cellulose, sucrose, dextrose, maltodextrins, lactose, and fructose. Suitable lubricants in this example include but are not limited to metal stearates (such as calcium, magnesium on zinc stearates), stearic acid, hydrogenated vegetable oils, talc, starch, light mineral oil, sodium benzoate, sodium chloride, sodium lauryl sulfate, magnesium lauryl sulfate, sodium stearyl fumarate, glyceryl behenate and polyethylene glycol (such as Carbowax™ 4000 and 6000). Suitable antiadherents in this example include but are not limited to colloidal silicon dioxide. Suitable binders in this example include but are not limited to ethyl cellulose, a polymethacrylate polymer, polyvinylalcohol, polyvinyl pyrrolidone, polyvinylpyrrolidone-vinylacetate copolymer (e.g. Kollidon VA64) hydroxyethylcellulose, low molecular weight hydroxypropylmethylcellulose (e.g. viscosity of 1-50 cps at 20o C; 2-12 cps at 20o C; or 4-6 cps at 20o C), hydroxypropylcellulose polymethacrylates, and mixtures thereof.

The drug-containing microparticles formed by extrusion/spheronization in this prophetic example will desirably be produced using cross-linked amphiphilic polymers by the following steps: (a) the mixing of one or more cross-linked amphiphilic polymers with tramadol and optionally other pharmaceutical excipients in order to obtain a uniform mixture in the form of dry powder to which a suitable amount of liquid is added to obtain a pasty consistency; (b) the extrusion of the mixture obtained from step (a) through a perforated mesh in order to obtain cylindrical filaments having desired length and diameter; (c) the spheronization of the filaments in order to obtain a product in the form of spherical multiparticulates; (d) the drying of the product; and (e) the optional depositing of a drug on the surface of the microparticles. “Cross-linked amphiphilic polymer” refers in this example to polymers showing characteristics of swellability in the whole pH range of aqueous solutions and also in solvents or solvent mixtures having different polarity characteristics. The polymers will desirably be cross-linked either physically through the interpenetration of the macromolecular meshes, or chemically, thus showing points of link among the macromolecular chains. Non-limiting examples of such polymers include cross-linked polyvinyl pyrrolidone, sodium carboxymethylcellulose, sodium glycolate starch and dextrans. Optional excipients include dispersing, emulsifying, wetting agents and colouring agents. The expression “uniform mixture” in this example means that the components of the mixture are uniformly dispersed in the formulation by a mixing process which assures the uniform distribution of each component. A reasonable mixing time will desirably range from 1 to 60 minutes using one of the mixing equipments conventionally used for the dry mixing of the powders (e.g. “V”, fixed body, rotating body, sigma mixers). The term “liquid” in this example means any liquid substance or mix (solution or emulsion) of liquids of normal pharmaceutical use able to moisten the powder mix, as for example water, aqueous solutions having different pH, organic solvents of normal pharmaceutical use (e.g. alcohols, chlorinated solvents), and oils. Among the oils and surfactants which will desirably be used in this example are: natural oils, either saturated or unsaturated (olive, peanut, soybean, corn, coconut, palm, sesame and similar oils); semisynthetic and synthetic mono-, di- and triglycerides containing saturated and/or unsaturated fatty acids and their polyhydroxyethylated derivatives (caprico-caprilic triglycerides [Mygliol™, Captex™, Labrafac™, Lipo], saturated or unsaturated polyhydroxylated triglycerides of various kind [Labrafil™, Labrafac™ Hydro, Gelucire™]); liquid waxes (isopropyl myristate, isopropyl-caprinate, -caprylate, -laurate, -palmitate, -stearate); fatty acids esters (ethyl oleate, oleyl oleate); silicone oils; polyethylene glycols (PEG 200, PEG 400, PEG 600, PEG 1000, and so on); polyglycolic glycerides (for example Labrasol™); polyglycols (propylene glycol, tetraglycol, and ethoxydiglycol (Transcutol™), sorbitan-esters of fatty acids (for example Span®, Arlacel®, Brij®), polyoxyethylenesorbitan esters of fatty acids (for example Tween®, Capmul®, Liposorb®), polypropylene oxide-polyethylene oxide (Poloxamer) copolymers, polyethylene glycol esters (PEG)-glycerol (Labrasol®, Labrafil®), PEG esters and long chain aliphatic acids or alcohols (for example Cremophor®), polyglycerid esters (Plurol®), saccharide and fatty acid esters (sucro-esters). Moreover, anionic surfactants (for example sodium lauryl sulfate, sodium stearate, sodium oleate) or cationic surfactants (for example tricetol), will desirably be used as well as lecithins, phospholipids and their semi-synthetic or synthetic derivatives. Also tramadol hydrochloride and/or excipients will desirably be dissolved, dispersed and/or emulsified in such liquids.

In a particular embodiment formed by an extrusion/spheronization process from the prophetic example described above, the moistening liquid comprises an oil/surfactant system wherein the tramadol hydrochloride optionally emulsified with an aqueous phase is dissolved or dispersed. The amount of liquid with respect to the solid used in the preparation of the mixture will desirably range from 1% to 80% by weight. As a prophetic example of this embodiment, a mixture of tramadol hydrochloride and Kollidon™ CL in a ratio equal to 1/3 by weight is co-milled obtaining the mixture in the form of powder having the 100% of granulometry lower than 50 microns. The mixture is moistened using a liquid demineralized water containing Kollidon™ 25 (polyvinyl pyrrolidone, BASF) in a solution 3% w/w. The extrusion is carried out forcing the moistened mass through a threader having diameter of the holes equal to 1 mm. The operative parameters in this prophetic example will desirably be as follows: powder flow rate: 4.5 kg/h; liquid flow rate: 4.1 kg/h; torsional stress: 27%; head temperature: 46° C.; and screw rotation velocity: 140 rpm. The extrusion filaments are then processed in a spheronizator adjusted at a velocity equal to 1,000 rpm for 2 minutes. The obtained microparticles are then dried in a fluid bed for 2 hours to a maximum temperature equal to 59° C. At the end of the drying the product is discharged and is mechanically screened separating the fraction ranging from 0.7 mm to 1.2 mm.

Another prophetic example of a drug-containing microparticle embodiment of the invention formed by an extrusion/spheronization process, uses a charged resin, the steps of which will desirably comprise: (a) adding the charged resin, tramadol hydrochloride and other excipients, to a mixing vessel; (b) mixing the ingredients to obtain a uniform mixture; (c) adding a granulating solution—a liquid capable of wetting the dry mixture. Liquids resulting in conversion of the dry powder mixture into a wet granulation that supports subsequent extrusion and spheronization (marumerization) are included. Typically, water or aqueous solutions are employed. Alcohols, typically ethanol or isopropanol, will desirably be included with the granulating water to enhance the workability of the granulation. In another embodiment of this invention, one or more of the components of the formulation is first dissolved in water and this solution is used to produce the wet granulation. An active ingredient or an excipient, which is present at very low concentration will desirably initially be dissolved or suspended in the granulating solvent to assure more uniform distribution throughout the formulation. (d) granulating the mixture until a uniform granulation results; (e) extruding the wet granulation through a screen to produce strands of granulation; (f) spheronizing the strands of granulation to produce spherical multiparticulates; and (g) collecting and drying the spherical multiparticulates. By “charged resin” is meant in this example to mean a polymer with ionizable functional groups that becomes useful in the embodiment of this invention. This broadly encompasses any polymer that upon ionization, is capable of producing cationic or anionic polymeric chains and which support spheronization. Typically from 10% to 70% by weight of the spherical multiparticulate is charged resin. Non limiting examples of these charged resins include sodium polystyrene sulfonate which is sold under the trade name AMBERLITE IP-69™ by Rohm and Haas, Co., Philadelphia, Pa.; the chloride salt of cholestyramine resin USP, sold as AMBERLITE IRP-276™ by Rohm and Haas, Co., Philadelphia, Pa.; the acid form of methacrylic acid-divinyl benzene, sold as AMBERLITE IRP-64™ by Rohm and Haas Co., Philadelphia, Pa.; carboxypolymethylenes sold under the trade names CARBOPOL™ 974P and CARBOPOL™ 934P by B. F. Goodrich, Inc., Brecksville, Ohio, and sodium polyacrylate, sold under the trade name AQUAKEEP™ J-550 by Seitetsu Kagaku, Japan. In order for the resin to maintain the desired degree of ionization, agents which produce an acidic or basic environment during granulation and spheronization will desirably be included within the formulation. Among the groups of compounds that will desirably exert this effect are acids, bases, and the salts of acids and bases such as adipic acid, citric acid, fumaric acid, tartaric acid, succinic acid, sodium carbonate, sodium bicarbonate, sodium citrate, sodium acetate, sodium phosphates, potassium phosphates, ammonium phosphate, magnesium oxide, magnesium hydroxide, sodium tartrate, and tromethamine. Certain compounds will desirably be added to the granulation to provide the proper degree of hydration of the charged resin, medicament and excipients. These hydrating agents include sugars such as lactose, sucrose, mannitol, sorbitol, pentaerythritol, glucose and dextrose. Polymers such as polyethylene glycol as well as surfactants and other organic and inorganic salts will desirably also be used to modulate polymer hydration.

Another embodiment of this invention involves the production of drug containing microparticles in the form of ‘pearls’. Pearls will desirably be manufactured by mixing tramadol hydrochloride with one or more pharmaceutical excipients in molten form; the melt is forced to pass through a nozzle which is subjected to a vibration; the pearls formed are allowed to fall in a tower countercurrentwise to a gas; and the solid pearls are collected in the bottom of the tower. In this example, the quantity of tramadol hydrochloride will desirably vary from 5% to 95% by weight; and in certain embodiments from 40% to 60% by weight. The additives which enable the crystallization of the supercooled product to be induced in this example will desirably be chosen from the following: fatty alcohols such as: cetyl alcohol, stearyl alcohol, fatty acids such as: stearic acid, palmitic acid, glycerol esters such as: glycerol palmitostearate, the glycerol stearate marketed under the mark Precirol™, the glycerol behenate marketed under the mark Compritol™, hydrogenated oils such as: hydrogenated castor oil marketed under the mark Cutina™ HR, fatty acid salts such as: magnesium or calcium stearate, polyols such as: mannitol, sorbitol, xylitol, waxes such as: white wax, carnauba wax, paraffin wax, polyoxyethylene glycols of high molecular weight, and esterified polyoxyethylenes such as: PEG-32 distearate, and PEG-150 distearate. To these crystallization additives it will desirably be desirable in this example to add polymers which are soluble or dispersible in the melt, and which provide a controlled and adjustable dissolution of the pearls when they are used, examples of which include: cellulose derivatives (hydroxypropyl cellulose, hydroxypropyl methyl cellulose, hydroxyethyl cellulose, ethyl cellulose, carboxymethyl cellulose), acrylic resins (marketed under the mark Eudragit®), polyvinyl acetates (marketed under the mark Rhodopas®), polyalkylene (ethylene propylene), polylactic, maleic anhydride and silicone resins. In addition, inorganic additives will desirably be added to accelerate the solidification of the active substances, examples of which include: silicas, inorganic oxides such as titanium or iron oxide, phosphates, carbonates, clays, and talc. In addition, a surface-active agent will desirably be added to improve the dispersion of the active substance in the crystallization additive, examples of which include: sorbitol esters, the polyoxyethylene polysorbates marketed under the mark Tween®, and glycols such as glycerine or propylene glycol. The process for the preparation of pearls comprise preparing a melt of the tramadol hydrochloride with one or more excipients. This melt will desirably be prepared by separately melting the various constituents and then mixing them or by melting the mixture of the constituents, possible insoluble compounds being added at the end of the melting so as to obtain a homogeneous mass. The nature of the constituents of the melt is chosen by the person skilled in the art, which is considered as a function of the compatibility of the constituents, the viscosity of the mixture of constituents, the nozzle diameter, the hydrophilicity of the active substance, the surface tension of the active substance, the particle size of the insoluble additives, the flow rate of the nozzle, the temperature of the tower, its height and, above all, the size of the desired pearls, the proportion of tramadol to be included therein and the desired release time of the active substance.

Alternative procedures other than extrusion or spheronization for manufacturing drug-containing microparticles will desirably include wet granulation, solvent granulation and melt granulation. All of these techniques involve the addition of an inactive binder to aggregate smaller particles into larger granules. For example, wet granulation and solvent granulation involve the addition of a liquid binder, which aggregates the active materials and excipients into granules. After granulation, the liquid will desirably be removed by a separate drying step. Melt granulation is similar to wet granulation, but uses a low melting point solid material as a binder. The solid binder in melt granulation is melted and acts as a liquid binder thereby aggregating the powdered active material and excipients into granules. The binder thereby, will desirably be incorporated into the granules when the granules cool.

Certain embodiments of the present invention include microparticles manufactured by a process for producing granules by rotomelt granulation that comprises mixing tramadol hydrochloride and a powdered excipient material that has a higher melting point than tramadol hydrochloride in a zone wherein both powdered materials are maintained in a fluidized state by a rising stream of gas in an apparatus having a rapidly rotating horizontal-disk located within a vertical vessel having a bottom surface; wherein said rapidly rotating disk is located on the bottom surface of the vertical vessel wherein said gas is at a temperature sufficient to cause the tramadol hydrochloride to at least partially melt thereby causing said powdered materials to aggregate and form granules. Other embodiments of the present invention include microparticles manufactured by a process for producing granules by rotomelt granulation comprising mixing powdered binder material and tramadol hydrochloride wherein the tramadol hydrochloride has a higher melting point than the powdered binder material in a zone wherein both powdered materials are maintained in a fluidized state by a rising stream of gas in an apparatus having a rapidly rotating horizontal-disk located within a vertical vessel having a bottom surface; and wherein said rapidly rotating disk is located on the bottom surface of the vertical vessel wherein said gas is at a temperature sufficient to cause the powdered binder material to at least partially melt thereby causing said powdered materials to aggregate and form granules.

In rotomelt granulation, one of the feed powders must have a lower melting point than the other powder in order to serve as a binder. The feed powders are introduced into a vertical vessel with rotatable horizontal-disk located in the bottom of the vessel. The powder is maintained in fluidized state by at least one stream of filtered air being circulated from the bottom of the vertical vessel through one or more inlets. The rotatable horizontal disk is then rotated while the air supplied to fluidize the powder is maintained at a temperature sufficient to soften or melt the lower melting point powder. The temperature to which the binder must be heated to soften will desirably be empirically determined by observing the formation of granules at various temperatures for various binders. It is presently believed that temperatures from 3° C. to 5° C. below the melting point or melting range provides sufficient softening to result in granule formation. The lower melting point powder then acts as a binding agent to promote the aggregation of powder particles into granules. Suitable powders for use in rotomelt granulation have a diameter size in the range of from 5 microns to 150 microns; and in certain embodiments have a diameter size in the range of 35 microns to 80 microns. The temperature, which the components will be exposed to depends on the binder employed to aggregate the powders. Generally, the melting point of the binder is above 30° C.; and in certain embodiments is below 100° C.

The powders used in these microparticles manufactured by rotomelt granulation will desirably be formed into granules by at least two alternative granulation mechanisms. The first mechanism for granule formation utilizes a larger particulate binder and a smaller particulate powder. The temperature during the rotomelt granulation is then elevated only to the point where the external surface of the binder particles become tacky. As the second powdered material of a smaller size is contacted with the tacky surface it forms a microlayer on the surface of the binder particle. This granulation mechanism results in granules which have size distribution similar to the original binder particles employed. Alternatively, the rotomelt granulation will desirably be conducted at a temperature at which the binder acts as a cement bridging the gaps between the unmelted particles (this is referred to as agglomeration). This mechanism results in the formation of granules where the components are intermingled. For each binder used the mechanism will desirably be controlled primarily by the temperature at which the rotomelt granulation is performed. Those skilled in the art will appreciate that the granules formed will desirably be observed by electron microscopy to determine the type of granulation process occurring. If one particular type of granule is desired, the process conditions or starting materials will desirably be varied to produce the desired granules.

In at least one embodiment of the present invention, tramadol hydrochloride is melted to act as a binding agent in the rotomelt granulation process. Examples of suitable excipients include those selected from the following: fillers, lubricants, glidants and antiadherents. Suitable fillers include but are not limited to calcium phosphate dibasic, tricalcium phosphate, calcium carbonate, starch (such as corn, maize, potato and rice starches), modified starches (such as carboxymethyl starch, etc.), microcrystalline cellulose, sucrose, dextrose, maltodextrins, lactose, and fructose. The amount of binder added to aggregate the particles into granules will desirably be in the range of from 10% w/w to 80% w/w; and in certain embodiments is in the range of from 30% w/w to 70% w/w of the powdered materials in the rotomelt granulation. The remaining weight percentage to provide a total of 100% w/w will desirably be one or more suitable powdered pharmaceutical actives. Optionally the rotomelt granulation will desirably also contain from 0% to 60% w/w of one or more powdered excipients wherein the total weight of all the powdered materials equals 100% w/w. The binder used in this embodiment of the invention will desirably be a pharmaceutically acceptable dry powder having a particle size in the range of from 5 μm to 150 μm; and in certain embodiments in the range of from 35 μm to 80 μm. Suitable binders for rotomelt granulation are low melting point powdered binders, examples of which include: polyethylene glycol 4000, polyethylene glycol 6000, stearic acid, and low melting point waxes. Suitable low melting point waxes include but are not limited to glyceryl monostearate, hydrogenated tallow, myristyl alcohol, myristic acid, stearyl alcohol, substituted monoglycerides, substituted diglycerides, substituted triglycerides, white beeswax, carnauba wax, castor wax, japan wax, acetylate monoglycerides and combinations thereof. The binders will desirably have a melting point of from 30° C. to 100° C.; and in certain embodiments from 40° C. to 85° C.

Other embodiments of the invention involve the formation of a microparticle that has a core, which includes tramadol hydrochloride and a compound which is sweet in taste and which has a negative heat of solution. Examples of compounds falling into this category include mannitol and sorbitol. Sugars or artificial sweeteners to which, for example, menthol has been added will desirably also work as well. A binder and/or other excipient will desirably also be disposed within the core. The amount of sweetening compound used will desirably depend on a number of factors including the size of the resulting microparticles, the size or volume of the resulting tablet, the sturdiness of the microparticle-coated microparticulant, the speed at which the tablet will disintegrate in the mouth, the degree of sweetness imparted by the particular sweetener used, either in the microparticle or in the tablet, or both, the amount of drug used, and the like. For example, particularly rugged microparticles will desirably be less likely to break during chewing and/or compression. Therefore, the amount of material provided to protect against the release of objectionably flavored material will desirably be lessened. In other cases a greater relative amount of sweetening compound will desirably be used. Generally, the amount of sweetening material used will range from greater than zero to 80% of the weight of the resulting microparticles. The sweetener and tramadol will desirably be combined in any number of known ways, such as for example by wet granulation, dry granulation, agglomeration, or spray coating. For example, the sweetener will desirably be used as an adsorbent for the active agent. Alternatively, particles of each will desirably also be simply mixed together. One or more binders, or other adjuvants will desirably also be used in the formulation of a tablet as well. Binders in these embodiments include, for example: starch (for example, in an amount of from 5% to 10% as an aqueous paste); pregelatinized starch (for example, in an amount of 5% to 10% added dry to powder); gelatin (for example, in an amount of from 2% to 10% as an aqueous solution, or 2% in starch paste); polyvinylpyrrolidone (for example, in an amount of from 2% to 20% in an aqueous or alcoholic solution); methylcellulose (for example, in an amount of from 2% to 10% as an aqueous solution); sodium carboxy methylcellulose (for example, in an amount of from 2% to 10% as an aqueous solution); ethylcellulose (for example, in an amount of from 5% to 10% as an alcohol or hydroalcoholic solution); polyacrylamides (Polymer JR) (for example, in an amount of from 2% to 8% as an aqueous solution); polyvinyloxoazolidone (Devlex) (for example, in an amount of from 5% to 10% as an aqueous or hydroalcoholic solution); and polyvinyl alcohols (for example, in an amount of from 5% to 20% in aqueous solutions). Other adjuvants will desirably also be used in forming the core of the microparticles of the present embodiments of the invention, non-limiting examples of which include: calcium sulfate NF, Dibasic Calcium phosphate NF, Tribasic calcium sulfate NF, starch, calcium carbonate, microcrystalline cellulose, modified starches, lactose, sucrose and the like, Sta-Rx™, Avicel™, Solka-Floc™ BW40, alginic acid, Explotab™, AUTOTAB™, guar gum, kaolin Vecgum™, and bentonite. These adjuvants will desirably be used in up to 20% w/w; and in certain embodiments are present in an amount of from 3% to 5% w/w.

Other embodiments of this invention involve the combined granulation and coating of tramadol into microparticles in which the drug is at least partly located within the microparticle core but capable of immediate release. To do this, the tramadol and a granular disintegrant are first dry-mixed; the powder obtained is then granulated, in the presence of a mixture of excipients comprising at least one binder capable of binding the particles together to give grains; the grains thus formed are then coated by spraying with a suspension comprising at least one coating agent and a membrane disintegrant; and then the coated granules obtained are dried. The distinction between the actual granulation and coating steps is relatively theoretical, insofar as, even though the primary function of the binder used in the granulation step is to bind together the particles, it nevertheless already partially coats the grains formed. Similarly, even though the primary function of the coating agent used in the actual coating step is to complete the final coating of each of the grains, it may, however, arbitrarily bind other coated grains by a mechanism of granular agglomeration. The binder and the coating agent are chosen from the group comprising cellulose polymers and acrylic polymers. However, even though the binder and the coating agent are chosen from the same group of compounds, they nevertheless differ from each other in their function as previously mentioned. Among the cellulose polymers that will desirably be advantageously chosen are ethylcellulose, hydroxypropylcellulose (HPC), carboxymethylcellulose (CMC) and hydroxypropylmethylcellulose (HPMC), or mixtures thereof. Among the acrylic polymers that will desirably be advantageously chosen are the ammonio-methacrylate copolymer (Eudragit® RL or RS), the polyacrylate (Eudragit® NE) and the methacrylic acid copolymer (Eudragit® L or S), Eudragit® being a registered trademark of Rohm. In at least one embodiment, the binder is of the same nature as the coating agent. To further accelerate the release of the tramadol hydrochloride, the coating suspension also comprises a permeabilizer which, on account of its intrinsic solubility properties, causes perforation of the membrane coating, thus allowing the tramadol hydrochloride to be released. Non-limiting examples of permeabilizers include povidone and its derivatives, polyethylene glycol, silica, polyols and low-viscosity cellulose polymers. Polymers of the type such as hypromellose, whose viscosity is equal to 6 centipoises, are used, for example, as low-viscosity cellulose polymer. In at least one embodiment, the dry-mixing of initial powder and the granulation, coating and drying steps are performed in a fluidized bed. In this case, the initial powder mixture is first fluidized before being granulated by spraying said powder with the excipient mixture comprising at least the binder, the grains obtained then being coated by spraying with the coating suspension, the coated granules formed finally being dried in the fluidized bed. In at least one embodiment, the mixture of excipients used during the granulation step and the coating suspension used during the coating step form a single mixture. In this case, the granulation step will desirably be distinguished from the spraying step by varying different parameters, such as the rate of spraying of the mixture and the atomization pressure of said mixture. Thus, only some of the mixture of excipients is used during the granulation step, while the other portion will desirably be used during the coating step. Thus, the rate of spraying of the coating suspension is higher during the granulation step than during the coating step, whereas the atomization pressure of the coating suspension is lower during the granulation step than during the coating step. In practice, at the laboratory scale in a fluidized-bed device, for example of the type such as Glatt GPCG1, during the granulation step, the rate of spraying of the coating suspension is between 10 grams/minute and 25 grams/minute, and the atomization pressure is between 1 bar and 1.8 bar. During the coating step, the rate of spraying of the coating suspension is between 5 grams/minute and 15 grams/minute, while the atomization pressure is between 1.5 bar and 2.5 bar. In at least one embodiment, between 10% and 20% of the mixture of excipients is sprayed during the granulation step, the remainder being sprayed during the coating step.

Other embodiments of the invention involve coating the tramadol, thereby forming a drug-containing microparticle. One such process for achieving this involves:
(i) Blending and fluidizing a powder mix of active principle and an adjuvant in order to obtain individual grains,
(ii) Separately liquefying under warm conditions a lipid matrix agent comprising either an ester of behenic acid and alcohol or an ester of palmitic/stearic acid and alcohol,
(iii) Coating the fluidized powder mix under warm conditions by spraying the lipid matrix agent over the individual grains,
(iv) Lowering the temperature of the combined product in order to allow the lipid matrix agent to solidify.

This process does not require an evaporation phase or a drying phase, since it does not require a wet-route or solvent-route granulation step, thus making it possible to be freed from any risk due to the presence of toxic residues in the final product. Furthermore, it is not necessary to carry out the quantitative determination of the traces of solvents, an analysis that will desirably be very expensive. According to the process of this embodiment of the invention, the spraying conditions and thus the coating characteristics will desirably be modified, in order to vary the release profile of tramadol, by varying several parameters, the adjustment characteristics of which remain simple. Thus, the spraying air pressure will desirably be increased in order to promote the formation of a homogeneous film of lipid matrix agent around the grains. Advantageously, the rate of spraying of the lipid matrix agent will desirably simultaneously be decreased. In this case, the tramadol release profile, that is to say a percentage of dissolution as a function of the time, is obtained which will desirably be low, corresponding to a slow release of the drug. Conversely, the spraying air pressure will desirably be decreased in order to promote the agglomeration of the grains with one another. Advantageously, the rate of spraying of the lipid matrix agent will desirably simultaneously be increased. In this case, a release profile of the grains obtained will desirably be obtained which is high, corresponding to a rapid release of tramadol. In practice and according to the mass of powder employed, the value of the rate of spraying of the lipid matrix agent will desirably be from two to four times higher when it is desired to promote the agglomeration of the grains with one another than when it is desired to promote the formation of a homogeneous film around the grains. On the other hand, the value of the spraying air pressure will desirably be from one to two times lower when it is desired to promote the agglomeration of the grains with one another than when it is desired to promote the formation of a homogeneous film around the grains. According to the process for manufacturing these embodiments, it is possible, after having determined a given drug release profile, to vary the values of spraying air pressure and of spraying rate throughout the coating stage, making it possible to promote the formation of a homogeneous film around the grains or to promote the agglomeration of the grains. Once the sequence of the duration of the spraying air pressure and of the spraying rate has been determined, the coating operation will desirably be carried out continuously and automatically. According to another characteristic of the process of manufacturing these embodiments, the temperature of the mixture of liquefied matrix agent and of spraying air is greater by 35° C. to 60° C. than the melting temperature of the lipid matrix agent. Likewise, the temperature of the fluidization air and that of the powder is approximately equal to the melting temperature of the lipid matrix agent, plus or minus 10° C. Furthermore, in order to obtain a mixture of individual grains, an air-operated fluidized bed device or a turbine device will desirably be used. Furthermore, the lipid matrix agent will desirably be sprayed by the air spray technique, that is to say liquid spraying under pressure in the presence of compressed air. According to at least one embodiment, use is made of a powder comprising the drug and the adjuvant. In other words, after mixing and fluidizing the combined constituents of the powder, the lipid matrix agent is sprayed over the individual grains obtained. In order to avoid adhesion of the coated grains obtained, whether in the case where all the grains are treated or whether in the case where only a portion of the grains is treated, a stage of lubrication of the grains is inserted between the coating stage and the stage of putting into a pharmaceutical form. Furthermore, in order to obtain greater stability of the pharmaceutical composition, that is to say in order to minimize modifications relating to the release of the tramadol over time, the granules or tablets obtained in certain embodiments of this example will desirably be subjected to a maturing stage in an oven, for at least 8 hours, at a temperature of between 45° C. and 60° C.; and in certain embodiments at 55° C.

As a prophetic example of these drug-containing microparticle embodiments that are formed by coating tramadol hydrochloride, the drug-containing microparticles will desirably be manufactured according to the following process: A mixture of powder is prepared comprising: tramadol hydrochloride; dicalcium phosphate dehydrate; and polyvinylpyrrolidone. Batches of granules are prepared by a process comprising the following stages: the mixture of powder obtained is sieved; the said powder is mixed, heating while by means of an air-operated fluidized bed, in order to obtain individual grains; the lipid matrix agent (glyceryl behenate, sold under the trade name Compritol® 880 ATO) is liquefied separately at 120° C.; the lipid matrix agent is sprayed over the heated powder mixture, and, finally, the temperature is lowered in order to allow the lipid matrix agent to solidify. These stages are carried out while varying various parameters, either in order to promote the formation of a homogeneous film around the grains or in order to promote the agglomeration of the grains, in accordance with the following table:

| Parameters | Batch 1 | Batch 2 | Batch 3 | Batch 4 |
|---|---|---|---|---|
| % by weight of lipid matrix | | | | |
| agent (Compritol ® 888 ATO) | 5 | 4 | 4 | 5 |
| Fluidization air flow rate (m³/h) | 80 | 110 | 80 | 80 |
| Agglomeration | | | | |
| Atomization air pressure (bar) | 2 | | 1.5 | 1.5 |
| Temperature of the powder bed | 70 | | 70 | 74 |
| (° C.) | | | | |
| Spraying rate for Compritol ® | 42 | | 40 | 40 |
| (g/min) | | | | |
| Coating | | | | |
| Atomization air pressure (bar) | 2.5 | 3.5 | 2 | 2 |
| Temperature of the powder bed | 70 | 66 | 71 | 70 |
| (° C.) | | | | |
| Spraying rate for Compritol ® | 41 | 20 | 40 | 40 |
| (g/min) | | | | |

Another embodiment of the invention for coating the tramadol hydrochloride material, thereby forming a drug-containing microparticle, involves the formation of coated microcrystals that will desirably subsequently be incorporated into a tablet. Through selection of the appropriate polymer the microcrystals will desirably possess diversified features such as gastroresistance and controlled release due to the fact that the said coated or non-coated microcrystals and microgranules preserve, after having been shaped in the form of a multiparticulate tablet, their initial properties amongst which are included masking of taste, gastroresistance and controlled release of the tramadol hydrochloride. In certain embodiments of this example, the following non-limiting list of polymers will desirably be selected for coating of the tramadol hydrochloride in conventional fluidized based coating equipment: ethylcellulose (EC); hydroxypropylcellulose (HPC); hydroxypropylmethylcellulose (HPMC); gelatin; gelatin/acacia; gelatin/acacia/vinvylmethylether maleic anhydride; gelatin/acacia/ethylenemaleic anhydride; carboxymethyl cellulose; polyvinvylalcohol; cellulose acetate phthalate; nitrocellulose; shellac; wax; polymethacrylate polymers such as Eudragit® RS; Eudragit® RL or combinations of both, Eudragit® E and Eudragit NE30D; Kollicoat™ SR30D; and mixtures thereof.

The present invention also contemplates an oral delivery system for delivering microparticles containing tramadol in admixture with a fluid. For example, an oral delivery system is provided which comprises a hollow drug formulation chamber. In at least one embodiment, the chamber has a first end and a second end and contains the first once daily controlled-release dosage form comprising tramadol in the form of microparticles. The system further comprises a fluid passing drug formulation retainer in the first end of the chamber. The retainer prevents release of the microparticles from the first end while permitting fluid entry into the chamber. In other embodiments, the microparticles contained within the chamber comprise tramadol and at least one other drug.

The present invention further provides a method for orally delivering microparticles containing the first once daily controlled-release dosage form comprising tramadol in admixture with a fluid. The method involves inserting the first once daily controlled-release dosage form comprising tramadol in the form of microparticles into a hollow drug delivery chamber of a drug delivery device. The chamber has a first end and a second end. The first end of the chamber has a fluid passing drug formulation retainer. The drug delivery device has a first and second end. The first end of the drug delivery device is inserted into a fluid and the second end is inserted into the mouth of a patient. The patient then applies suction to the second end of the device to cause delivery of the fluid and the first once daily controlled-release dosage form comprising tramadol into the patient's mouth.

The term “drug formulation retainer” as used herein, refers to a valve, plug or restriction, or the like that prevents passage of the drug formulation from the device. By “fluid passing drug formulation retainer” is intended a valve, plug or restriction or the like that allows for passage of fluids but does not allow for passage of other ingredients such as the first once daily controlled-release dosage form contained in the delivery device.

The dispensing device of this embodiment of the invention finds use where it is inconvenient or unsafe to use solid oral dosage forms such as capsules or tablets. The devices will desirably be particularly useful in geriatric or pediatric patient populations but they will desirably also be useful for those who have difficulty swallowing capsules or tablets. A single delivery device or several devices will desirably be administered to a patient during a therapeutic program.

Generally the device is in prepared form prior to placement in a fluid. In at least one embodiment the dispensing device comprises a hollow drug formulation chamber with a first end and a second end. Contained within the chamber are drug formulation and fluid passing drug formulation retainers. The fluid passing drug formulation retainer comprises a restriction and a one-way plug. The diameter of the opening is smaller than the plug. In at least one embodiment the restriction is made by crimping an end of the chamber. The second end of the chamber has a drug formulation retainer for preventing release of the plug. In at least one embodiment the retainer is prepared by crimping the end of the chamber. The first once daily controlled-release dosage form comprising tramadol in the form of microparticles are then placed in the chamber. An end-cap is placed over the second end of the chamber prior to use to prevent release of the drug formulation. In prepared form, the plug substantially seals the first end of the chamber, thereby preventing loss of the drug formulation from the first end.

The device will desirably be formed from any suitable material that is physically and/or chemically compatible with both the active drug and the liquid diluent to be mixed therein. In certain embodiments, representative materials for forming devices including the drug formulation chamber, the elongated tubular member, the end caps and tabs, include, without limitation, paper, plastic such as propylene/styrene copolymers, polyproylene, high density polyethylene, low density polyethylene and the like. The devices will desirably have an inner diameter of between 3 mm and 8 mm and a wall thickness of between 0.1 mm and 0.4 mm. The devices will desirably be between 10 cm and 30 cm in length.

The fluid passing drug formulation retainer permits the free flow of liquid medium but prohibits passage of the first once daily controlled-release dosage form comprising tramadol in the form of microparticles from the device prior to delivery. Where the retainer comprises a one-way plug or valve, the plug or valve will seal the straw at atmospheric pressure. When suction is applied, fluid will be drawn around the plug and into the drug formulation chamber. Further, the plug has a density of less than one so that it will ascend to the top as the drug formulation is delivered into the oral cavity. When suction is no longer applied, the plug will remain in the highest position it reached during sipping. The plug will desirably be prepared from closed cell polyethylene foam such as EthaFoam®. Other forms of one-way plugs will desirably be a balloon of elastomeric material, a one-way mechanical ball valve and the like.

Examples of fluid that will desirably be used for suspending the first once daily controlled-release dosage form comprising tramadol in the form of microparticles is any palatable liquid such as water, juice, milk, soda, coffee, tea etc.

In at least one embodiment, a dose sipping delivery device according to the present invention will desirably be prepared as follows. Jumbo size straws with an inside diameter of 0.21 inches and a length of 8 inches are heat sealed at one end. The seal is partially cut off so that the “one-way” plug cannot escape. The partially sealed end is enclosed by half of a size 1 hard gelatin capsule. The first once daily controlled-release dosage form comprising tramadol in the form of microparticles are then placed inside the open end of the straw. A “one-way” plug made of closed cell polyethylene foam, Microfoam® (DuPont) is trimmed to snugly fit inside the straw. The plug is then placed inside the straw, on top of the microparticles. During operation, the plug end of the straw is placed into a glass of water and the protective gelatin capsule on the top of the straw is removed. By slowly applying suction through the partially sealed end of the straw, the microparticles are sucked into the mouth and easily swallowed.

Prophetic examples of the first once daily controlled-release dosage forms or means for controllably releasing tramadol formulations are described below. It should be understood that these examples are intended to be exemplary and that the specific constituents, amounts thereof, and formulation methods may be varied therefrom by the skilled artisan based on his skill and knowledge in the art of drug delivery without undue experimentation in order to achieve the desired in-vitro dissolution and pharmacokinetic parameters described herein.

The above embodiments of the invention and variations thereof relating to the first once daily controlled-release dosage forms or first once daily controlled-release dosage forms comprising at least one means for controllably releasing the tramadol will be more apparent to those versed in the delivery arts from the following description, taken in conjunction with the accompanying claims.

### PROPHETIC EXAMPLES

### Example 1

### Unitary Osmotic System

| Tablet Core Ingredients | % of Tablet |
|---|---|
| Tramadol HCl | 74.0 |
| Colloidal Silicon Dioxide | 0.74 |
| Polyvinyl alcohol | 1.48 |
| D-Mannitol | 23.04 |
| Sodium Stearyl Fumarate | 0.74 |
| Semipermeable Membrane Ingredients | % of Coating |
| Cellulose Acetate | 84.50 |
| Hydroxypropyl Cellulose | 7.50 |
| Sodium Chloride | 8.00 |
| Organic Solvents (evaporated in process) | — |

Procedure
Granulate all tablet ingredients except D-mannitol and lubricant. Add D-mannitol and lubricant and compress using conventional means. Coat core with solution using vented pan coating process, to form a semipermeable membrane around core.

### Example 2

### Multiparticulate Osmotic System

| Microsphere Ingredients | % of Sphere |
|---|---|
| Tramadol HCl | 70 |
| Compritol ATO 888 | 12 |
| NaCl | 10 |
| Gelucire 50/13 | 8 |
| Sustained Release Coating Ingredients | % of Coating |
| Ethyl Cellulose | 60.9 |
| Hydroxypropyl cellulose | 26.1 |
| Talc-micronized | 13 |
| Isopropranol/Acetone (evaporated in process) | — |

Procedure
Blend microsphere ingredients and process using Ceform ™ technology. Place microspheres in Wurster based fluidized bed coater and apply sustained release coating.

### Example 3

### Hydrophobic Core Controlled Release System (Lipid)

| Tablet Core Ingredients | % of Tablet |
|---|---|
| Tramadol HCl | 60.0 |
| Hydrogenated Vegetable Oil (Lubritab) | 36.5 |
| Hydroxypropyl cellulose | 3.0 |
| Magnesium Stearate | 0.5 |
| Tablet Coating Ingredients | % of Coating |
| Opadry (Clear) 5% solution | 100 |
| Purified Water (evaporated in process) | — |

Procedure
Melt granulate the drug, Lubritab, and HPC above 80 degrees C. in jacketed high shear mixer. Congeal and screen/mill/size the granulate. Add lubricant and compress. Apply cosmetic coat to tablets using vented coating pan.

### Example 4

### Hydrophobic Core Controlled Release System (Wax)

| Tablet Core Ingredients | % of Tablet |
|---|---|
| Tramadol HCl | 59.35 |
| Carnauba Wax | 36.50 |
| Stearyl alcohol | 3.65 |
| Magnesium Stearate | 0.50 |
| Tablet Coating Ingredients | % of Coating |
| Opadry (Clear) 5% solution | 100 |
| Purified Water (evaporated) | — |

Procedure
Melt granulate the drug, carnauba wax, and stearyl alcohol at 95-100 degrees C. in jacketed high shear mixer. Congeal and screen/mill/size the granulate. Add lubricant and compress into tablets. Apply cosmetic coat to tablets using vented coating pan.

### Example 5

### Hydrophobic Core Controlled-Release System (Insoluble Polymer)

| Tablet Core Ingredients | % of Tablet |
|---|---|
| Tramadol HCl | 74.0 |
| Colloidal Silicon Dioxide | 0.74 |
| Polyvinyl alcohol | 1.48 |
| Ethyl Cellulose | 20.00 |
| Ludipress | 3.04 |
| Sodium Stearyl Fumarate | 0.74 |
| Tablet Coating Ingredients | % of Coating |
| Opadry (Clear) 5% solution | 100 |
| Purified Water (evaporated) | — |

Procedure
Granulate tramadol and silicon dioxide using PVA solution in fluid bed granulator using top-spray method. Compress granulate, ethyl cellulose, Ludipress, and lubricant into tablets using rotary compression. Coat with cosmetic coating using vented coating pan spray technology.

### Example 6

### Hydrophobic Coat (Lipid)

| Mini-Tablet Core Ingredients | % of Tablet |
|---|---|
| Tramadol HCl | 96.15 |
| Colloidal Silicon Dioxide | 0.96 |
| Polyvinyl alcohol | 1.92 |
| Sodium Stearyl Fumarate | 0.96 |
| Mini-Tablet Coating Ingredients | % of Coating |
| Glyceryl monostearate | 95.25 |
| Polyethylene Glycol 8000 | 4.75 |

Procedure
Granulate the tramadol with colloidal silicon dioxide using PVA solution, under top-spray fluid bed process. Add lubricant to granulate and compress using conventional rotary process. Coat mini-tablets with molten lipid-based coating in Wurster fluid-bed processor outfitted with hot melt coating apparatus.

### Example 7

### Hydrophobic Coat (Wax)

| Mini-Tablet Core Ingredients | % of Tablet |
|---|---|
| Tramadol HCl | 96.15 |
| Colloidal Silicon Dioxide | 0.96 |
| Polyvinyl alcohol | 1.92 |
| Sodium Stearyl Fumarate | 0.96 |
| Mini-Tablet Coating Ingredients | % of Coating |
| Hydrogenated Castor Oil (Castorwax) | 95.25 |
| Polyethylene Glycol 8000 | 4.75 |

Procedure
Granulate the tramadol with colloidal silicon dioxide using PVA solution, under top-spray fluid bed process. Add lubricant to granulate and compress using conventional rotary process. Coat mini-tablets with molten wax-based coating in Wurster fluid-bed processor outfitted with hot melt coating apparatus.

### Example 8

### Hydrophobic Coat (Insoluble Polymer)

| Tablet Core Ingredients | % of Tablet |
|---|---|
| Tramadol HCl | 96.15 |
| Colloidal Silicon Dioxide | 0.96 |
| Polyvinyl alcohol | 1.92 |
| Sodium Stearyl Fumarate | 0.96 |
| Tablet Coating Ingredients | % of Coating |
| Ethylcellulose | 84.09 |
| Hydroxypropyl Cellulose | 6.82 |
| Dibutyl Sebacate | 9.09 |
| Isopropanol/Acetone (evaporated) | — |

Procedure
Granulate the tramadol with colloidal silicon dioxide using PVA solution, under top-spray fluid bed process. Add lubricant to granulate and compress using conventional rotary process. Coat with solvent coating in conventional vented coating pan.

### Example 9

### Hydrophilic Core (Swellable)

| Tablet Core Ingredients | % of Tablet |
|---|---|
| Tramadol HCl | 63.12 |
| Colloidal Silicon Dioxide | 0.66 |
| Polyvinyl alcohol | 1.00 |
| Eudragit RL © powder | 34.26 |
| Sodium Stearyl Fumarate | 0.96 |
| Tablet Coating Ingredients | % of Coating |
| Opadry (Clear) 5% solution | 100 |
| Purified Water (evaporated in process) | — |

Procedure
Granulate all tablet ingredients except Eudragit RL © and lubricant in top spray fluid bed granulator. Add Eudragit RL © and lubricant and compress into tablet using conventional means. Apply cosmetic coat to tablets using vented coating pan.

### Example 10

### Hydrophilic Core (Soluble Polymer)

| Tablet Core Ingredients | % of Tablet |
|---|---|
| Tramadol HCl | 60.00 |
| Colloidal Silicon Dioxide | 0.66 |
| Polyvinyl alcohol | 1.00 |
| Hydroxypropyl Methylcellulose | 37.38 |
| Sodium Stearyl Fumarate | 0.96 |
| Tablet Coating Ingredients | % of Coating |
| Opadry (Clear) 5% solution | 100 |
| Purified Water (evaporated in process) | — |

Procedure
Granulate all tablet ingredients except HPMC and lubricant in top spray fluid bed granulator. Add HPMC and lubricant and compress using conventional means. Apply cosmetic coat to tablets using vented coating pan.

### Example 11

### Hydrophilic Coat (Swellable)

| Tablet Core Ingredients | % of Tablet |
|---|---|
| Tramadol HCl | 96.15 |
| Colloidal Silicon Dioxide | 0.96 |
| Polyvinyl alcohol | 1.92 |
| Sodium Stearyl Fumarate | 0.96 |
| Tablet Coating Ingredients | % of Coating |
| Eudragit RS © | 14.0 |
| Eudragit RL © | 56.0 |
| Acetyl Triethyl Citrate | 15.0 |
| Talc | 15.0 |
| Alcoholic/Acetone Solvents (evaporates) | — |

Procedure
Granulate the tramadol with colloidal silicon dioxide using PVA solution, under top-spray fluid bed process. Add lubricant to granulate and compress using conventional rotary process. Apply coating to tablets using vented coating pan..

### Example 12

### Hydrophilic Coat (Soluble Polymer)

| Tablet Core Ingredients | % of Tablet |
|---|---|
| Tramadol HCl | 96.15 |
| Colloidal Silicon Dioxide | 0.96 |
| Polyvinyl alcohol | 1.92 |
| Sodium Stearyl Fumarate | 0.96 |
| Tablet Coating Ingredients | % of Coating |
| Hydroxymethyl Cellulose | 62.0 |
| Hydroxyethyl Cellulose | 38.0 |
| Water (evaporated) | — |

Procedure
Granulate the tramadol with colloidal silicon dioxide using PVA solution, under top-spray fluid bed process. Add lubricant to granulate and compress using conventional rotary process. Coat with sufficient aqueous coating in conventional vented coating pan to sustain drug release.

### Example 13

### Tramadol AQ

| Tablet Core Ingredients | % of Tablet |
|---|---|
| Tramadol HCl | 93.15 |
| Colloidal Silicon Dioxide | 0.96 |
| Polyvinyl alcohol | 1.92 |
| Kollidon CL | 3.00 |
| Sodium Stearyl Fumarate | 0.96 |
| Tablet Coating Ingredients | % of Coating |
| Eudragit NE30D | 45.03 |
| | (as dry) |
| Hydroxypropyl Methylcellulose 6 cps | 18.01 |
| Polyethylene Glycol 8000 | 11.26 |
| Talc 400 | 20.26 |
| Titanium dioxide | 4.31 |
| Simethicone | 1.13 |

Procedure
Granulate the tramadol with colloidal silicon dioxide using PVA solution, under top-spray fluid bed process. Add lubricant to granulate and compress using conventional rotary process. Coat with aqueous-based coating dispersion/suspension in conventional vented coating pan.

### Example 14

### Delayed Release System (Enteric Coat, Hydrophobic Core)

| Tablet Core Ingredients | % of Tablet |
|---|---|
| Tramadol HCl | 64.0 |
| Colloidal Silicon Dioxide | 0.74 |
| Polyvinyl alcohol | 1.00 |
| Ethyl Cellulose 10 cps | 30.00 |
| Ludipress | 3.52 |
| Sodium Stearyl Fumarate | 0.74 |
| Tablet Coating Ingredients | % of Coating |
| Eudragit L100-55 | 66.9 |
| | (as dry) |
| Acetyl Triethyl Citrate | 10.0 |
| Talc 400 | 23.1 |

Procedure
Granulate the tramadol with colloidal silicon dioxide using PVA solution, under top-spray fluid bed process. Add ethyl cellulose, Ludipress, and lubricant to granulate and compress using conventional rotary process. Coat with aqueous-based enteric coating dispersion/suspension in conventional vented coating pan.

1. A first once daily controlled-release dosage form comprising tramadol comprising at least one means for controllably releasing the tramadol such that said first once daily controlled-release dosage form exhibits an in-vitro release rate such that after about 2 hours from about 0 to about 22% by weight of tramadol is released, after about 4 hours from about 5 to about 30% by weight of tramadol is released, after about 6 hours, from about 15 to about 38% by weight of tramadol is released, and after about 8 hours, more than about 40% by weight of tramadol is released, and when said first once daily controlled-release dosage form is administered to a patient in need of such administration under fed or fasted conditions and is bioequivalent according to FDA guidelines to a second orally administrable dosage form comprising the same dose of tramadol also suitable for once daily administration in the fed or fasted state.

2. The first once daily controlled-release dosage form of claim 1, wherein said first once daily controlled-release dosage form dosage form when administered to a patient in need of such administration exhibits following single-dose administration: (i) a Cₘₐₓ of from about 75 to about 338 ng/ml of the tramadol, (ii) an AUC_{0-•} of from about 2725 to about 7681 ng·hr/ml of tramadol under fed conditions, and is bioequivalent according to FDA guidelines to a second orally administrable dosage form comprising the same dose of tramadol also suitable for once daily administration in the fed state.

3. The first once daily controlled-release dosage form of claim 1, Wherein said first once daily controlled-release dosage form when administered once daily to a patient in need of such administration exhibits following single-dose administration: (i) a Cₘₐₓ of from about 180 to about 333 ng/ml of tramadol, (ii) an AUC_{0-•} of from about 3740 to about 7600 ng·hr/ml of tramadol under fasting conditions, and is bioequivalent according to FDA guidelines to a second orally administrable dosage form comprising the same dose of tramadol also suitable for once daily administration in the fasted state.

4. The first once daily controlled-release form of claim 1, wherein the in-vitro dissolution of the first once daily controlled-release dosage form comprising the at least one means for controllably releasing the tramadol is measured using a USP Type I, II, or III apparatus in dissolution medium chosen from 900 ml 0.1N HCl, water, 0.1N HCl+0.1% Cetrimide, USP Buffer pH 1.5, Acetate Buffer pH 4.5, Phosphate Buffer pH 6.5, or Phosphate Buffer pH7.4 at 75 rpm at 37°±0.5° C., and the tramadol released into the dissolution medium is assayed in a 10 ml UV cell at 271 nm.

5. The first once daily controlled-release form of claim 1, wherein the in-vitro dissolution of the first once daily controlled-release dosage form comprising the at least one means for controllably releasing the tramadol is measured using a USP Type I, II, or III apparatus in 900 ml 0.1N HCl 75 rpm at 37°±0.5° C., and the tramadol released into the dissolution medium is assayed in a 10 ml UV cell at 271 nm.

6. The first once daily controlled-release form of claim 1, wherein the in-vitro dissolution of the first once daily controlled-release dosage form comprising the at least one means for controllably releasing the tramadol is measured using a USP Type I, II, or III apparatus in water at 37°±0.5° C., and the tramadol released into the dissolution medium is assayed in a 10 ml UV cell at 271 nm.

7. The first once daily controlled-release form of claim 1, wherein the in-vitro dissolution of the first once daily controlled-release dosage form comprising the at least one means for controllably releasing the tramadol is measured using a USP Type I, II, or III apparatus in 0.1N HCl+0.1% Cetrimide at 37°±0.5° C., and the tramadol released into the dissolution medium is assayed in a 10 ml UV cell at 271 nm.

8. The first once daily controlled-release form of claim 1, wherein the in-vitro dissolution of the first once daily controlled-release dosage form comprising the at least one means for controllably releasing the tramadol is measured using a USP Type I, II or III apparatus in USP Buffer pH 1.5 at 37°±0.5° C., and the tramadol released into the dissolution medium is assayed in a 10 ml UV cell at 271 nm.

9. The first once daily controlled-release form of claim 1, wherein the in-vitro dissolution of the first once daily controlled-release dosage form comprising the at least one means for controllably releasing the tramadol is measured using a USP Type I, II, or III apparatus in Acetate buffer pH 4.5 at 37°±0.5° C., and the tramadol released into the dissolution medium is assayed in a 10 ml UV cell at 271 nm.

10. The first once daily controlled-release form of claim 1, wherein the in-vitro dissolution of the first once daily controlled-release dosage form comprising the at least one means for controllably releasing the tramadol is measured using a USP Type I, II, or III apparatus in Phosphate Buffer pH 6.5 at 37°±0.5° C. and the tramadol released into the dissolution medium is assayed in a 10 ml UV cell at 271 nm.

11. The first once daily controlled-release form of claim 1, wherein the in-vitro dissolution of the first once daily controlled-release dosage form comprising the at least one means for controllably releasing the tramadol is measured using a USP Type I, II, or III apparatus in Phosphate Buffer pH 7.4 at 37°±0.5° C., and the tramadol released into the dissolution medium is assayed in a 10 ml UV cell at 271 nm.

12. The first once daily controlled-release dosage form of claim 1, wherein said first once daily controlled-release dosage form comprising the at least one means for controllably releasing the tramadol has a reduced potential for alcohol induced dose dumping in the fed state.

13. The first once daily controlled-release dosage form of claim 1, wherein said first once daily controlled-release dosage form comprising the at least one means for controllably releasing the tramadol has a reduced potential for alcohol induced dose dumping in the fasted state.

14. The first once daily controlled-release dosage form of claim 1, wherein said first once daily controlled-release dosage form comprising the at least one means for controllably releasing the tramadol exhibits in the fed state a Tₘₐₓ of tramadol from about 4 to about 24 hr following single-dose administration and will desirably be bioequivalent according to FDA guidelines to the second orally administrable dosage form comprising the same dose of tramadol also suitable for once daily administration.

15. The first once daily controlled-release dosage form of claim 1, wherein said first once daily controlled-release dosage form comprising the at least one means for controllably releasing the tramadol exhibits at steady state the following pharmacokinetic parameters in-vivo under fasting conditions: (i) an AUC₀₋₂₄ from about 1635 to about 21000 ng·h/ml, and (ii) a Cₘₐₓ from about 117 to about 1230 ng/ml, and is bioequivalent according to FDA guidelines to the second orally administrable dosage form comprising tramadol also suitable for once daily administration in the fasted state.

16. The first once daily controlled-release dosage form of claim 1, wherein said first once daily controlled-release dosage form comprising the at least one means for controllably releasing the tramadol exhibits under fasting conditions a Tₘₐₓ of from about 9 to about 14 hours at steady state, and is bioequivalent according to FDA guidelines to the second orally administrable dosage form comprising the same dose of tramadol also suitable for once daily administration in the fasted state.

17. The first once daily controlled-release dosage form of claim 1, wherein said first once daily controlled-release dosage form comprising the at least one means for controllably releasing the tramadol exhibits under fasting conditions a degree of fluctuation of about 43 to about 141% at steady state and is bioequivalent according to FDA guidelines to the second orally administrable dosage form comprising the same dose of tramadol also suitable for once daily administration in the fasted state.

18. The first once daily controlled-release dosage form of claim 1, wherein said first once daily controlled-release dosage form comprising the at least one means for controllably releasing the tramadol exhibits a Cₘᵢₙ of from about 31 to about 652 ng/ml at steady state and is bioequivalent according to FDA guidelines to the second orally administrable dosage form comprising the same dose of tramadol also suitable for once daily administration in the fasted state.

19. The first once daily controlled-release dosage form of claim 1, wherein said first once daily controlled-release dosage form comprising the at least one means for controllably releasing the tramadol exhibits following single-dose administration under fasting conditions a Tₘₐₓ of tramadol of from about 10 to about 20 hr in the fasting state and is bioequivalent according to FDA guidelines to the second orally administrable dosage form comprising the same dose tramadol also suitable for once daily administration in the fasted state.

20. The first once daily controlled-release dosage form of claim 1, wherein the at least one means for controllably releasing tramadol is chosen from at least one controlled-release matrix core; at least one insoluble matrix core; at least one swellable matrix core; at least one swellable and erodable matrix core; at least one hydrophobic matrix core; at least one hydrophilic matrix core; at least one lipid matrix core; at least one wax matrix core; at least one erodable matrix core; at least one release-slowing coat; at least one delayed release coat; at least one release-slowing coat comprising at least one pH independent polymer; at least one release-slowing coat comprising at least one pH dependent polymer; at least one release-slowing coat comprising at least one soluble polymer, at least one release-slowing coat comprising at least one insoluble polymer; at least one release-slowing coat comprising at least one swellable polymer; at least one release-slowing coat comprising at least one hydrophobic polymer; at least one release-slowing coat comprising at least one hydrophilic polymer; at least one release-slowing coat comprising at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol; at least one release-slowing coat comprising at least one water-insoluble water-permeable film-forming polymer and at least one water-soluble-polymer; a release-slowing coat comprising at least one water-insoluble water-permeable film-forming polymer, at least one water-soluble polymer and at least one plasticizer; at least one means for the exit of tramadol; at least one means for increasing the hydrostatic pressure of the first once daily controlled-release dosage form comprising tramadol; at least one means for forcibly dispensing tramadol from the first once daily controlled-release dosage form; or any combination of thereof.

21. The first once daily controlled-release dosage form of claim 1, wherein the first once daily controlled-release dosage form comprises an effective amount of tramadol for the management of moderate to moderately severe pain.

22. The first once daily controlled-release dosage form of claim 1, wherein the first once daily controlled-release dosage form comprises from about 25 mg to about 800 mg of tramadol.

23. The first once daily controlled-release dosage form of claim 1, wherein first once daily controlled-release dosage form comprises about 150 mg of tramadol.

24. The first once daily controlled-release dosage form of claim 1, wherein the first once daily controlled-release dosage form comprises about 300 mg of tramadol.

25. The first once daily controlled-release dosage form of claim 1, wherein first once daily controlled-release dosage form comprises tramadol hydrochloride.

26. The first once daily controlled-release dosage form of claim 1, wherein the first once daily controlled-release dosage form comprises a mixture of an effective amount of at least two different tramadol salts, wherein one of the salts comprises tramadol hydrochloride.

27. The first once daily controlled-release dosage form of claim 1, wherein the first once daily controlled-release dosage form comprises 70 to 90% by weight of the core dry weight tramadol.

28. The first once daily controlled-release dosage form of claim 1, wherein the first once daily controlled-release dosage form comprises an immediate release coat comprising tramadol.

29. The first once daily controlled-release dosage form of claim 1, wherein the first once daily controlled-release dosage form comprises an immediate release coat comprising a salt of tramadol which is different from the salt of tramadol present in the core of the first once daily controlled-release dosage form.

30. The first once daily controlled-release dosage form of claim 1, wherein the at least one means for controllably releasing the tramadol comprises at least one controlled-release matrix core.

31. The first once daily controlled-release dosage form of claim 1, wherein the at least one means for controllably releasing the tramadol comprises at least one insoluble matrix core.

32. The first once daily controlled-release dosage form of claim 1, wherein the at least one means for controllably releasing the tramadol comprises at least one swellable matrix core.

33. The first once daily controlled-release dosage form of claim 1, wherein the at least one means for controllably releasing the tramadol comprises at least one swellable and erodable matrix core.

34. The first once daily controlled-release dosage form of claim 1, wherein the at least one means for controllably releasing the tramadol comprises at least one hydrophobic matrix core.

35. The first once daily controlled-release dosage form of claim 1, wherein the at least one means for controllably releasing the tramadol comprises at least one hydrophilic matrix core.

36. The first once daily controlled-release dosage form of claim 1, wherein the at least one means for controllably releasing the tramadol comprises a combination of a hydrophobic and hydrophilic matrix core.

37. The first once daily controlled-release dosage form of claim 1, wherein the at least one means for controllably releasing the tramadol comprises a lipid matrix core.

38. The first once daily controlled-release dosage form of claim 1, wherein the at least one means for controllably releasing the tramadol comprises a wax matrix core.

39. The first once daily controlled-release dosage form of claim 1, wherein the at least one means for controllably releasing the tramadol comprises at least one erodable matrix core.

40. The first once daily controlled-release dosage form of claim 1, wherein the at least one means for controllably releasing the tramadol comprises at least one release-slowing coat.

41. The first once daily controlled-release dosage form of claim 1, wherein the at least one means for controllably releasing the tramadol comprises at least one delayed-release coat.

42. The first once daily controlled-release dosage form of claim 1, wherein the at least one means for controllably releasing the tramadol comprises at least one release-slowing coat, which coat comprises at least one pH independent polymer.

43. The first once daily controlled-release dosage form of claim 1, wherein the at least one means for controllably releasing the tramadol comprises at least one release-slowing coat comprising at least one pH dependent polymer.

44. The first once daily controlled-release dosage form of claim 1, wherein the at least one means for controllably releasing the tramadol comprises at least one release-slowing coat, which coat comprises at least one soluble polymer.

45. The first once daily controlled-release dosage form of claim 1, wherein the at least one means for controllably releasing the tramadol comprises at least one release-slowing coat comprising at least one insoluble polymer.

46. The first once daily controlled-release dosage form of claim 1, wherein the at least one means for controllably releasing the tramadol comprises at least one release-slowing coat, which coat comprises at least one swellable polymer.

47. The first once daily controlled-release dosage form of claim 1, wherein the at least one means for controllably releasing the tramadol comprises at least one release-slowing coat, which coat comprises at least one hydrophobic material.

48. The first once daily controlled-release dosage form of claim 1, wherein the at least one means for controllably releasing the tramadol comprises at least one means for the exit of tramadol from the first once daily rate controlled-release dosage form, at least one means for increasing the hydrostatic pressure of the first once daily controlled-release dosage form, and at least one means for forcibly dispensing tramadol from the first once daily controlled-release dosage form.

49. The first once daily controlled-release dosage form of claim 1, wherein the at least one means for controllably releasing the tramadol comprises at least one aqueous dispersion of a neutral ester copolymer without any functional groups, a poly glycol having a melting point greater than 55° C., and one or more pharmaceutically acceptable excipients and is cured at a temperature at least equal to or greater than the melting point of the poly glycol.

50. The first once daily controlled-release dosage form of claim 1, wherein the at least one means for controllably releasing the tramadol comprises at least one release-slowing coat comprising a water-insoluble water-permeable film-forming polymer and at least one water-soluble polymer.

51. The first once daily controlled-release dosage form of claim 1, wherein the at least one means for controllably releasing the tramadol comprises at least one release-slowing coat comprising at least one water-insoluble water-permeable film-forming polymer, at least one water-soluble polymer and at least one plasticizer.

52. The first once daily controlled-release dosage form of claim 1, wherein said first once daily controlled-release dosage form is a tablet.
